(19) United States
(12) Patent Application Publication
(10) Pub. No.: US 2026/0035712 A1
(43) Pub. Date: Feb. 5, 2026

(54) PLANTS WITH REDUCED PLASTICITY

(71) Applicants: Donald Danforth Plant Science Center, St Louis, MO (US); The University Court of the University of Glasgow, Glasgow (GB)

(72) Inventors: Dmitri A. NUSINOW, St Louis, MO (US); Matthew A. Jones, Glasgow (GB)

(21) Appl. No.: 18/997,623

(22) PCT Filed: Jul. 24, 2023
(86) PCT No.: PCT/US2023/070851

§ 371 Date: Jan. 22, 2025

Related U.S. Application Data
(60) Provisional application No. 63/391,651, filed on Jul. 22, 2022.

(51) Int. Cl. C12N 15/82; C07K 14/415; C12N 9/22; C12N 15/11

(52) U.S. Cl. CPC C12N 15/8262; C07K 14/415; C12N 9/226; C12N 15/11; C12N 15/8213; C12N 2310/20

(57) ABSTRACT

Plants modified to exhibit reduced plasticity and related methods and compositions are disclosed. The plants have reduced developmental plasticity in response to changes in light and temperature conditions, and exhibit additional features such as earlier, more consistent and uniform flowering regardless of changes in day length and temperature, and reduced listing in the field.

### CROSS-REFERENCE TO RELATED APPLICATIONS

This application claims priority from Provisional Application No. 63/391,651, filed Jul. 22, 2022, the entire contents of which are hereby incorporated by reference.

### SEQUENCE LISTING

This application contains a Sequence Listing that has been submitted in .XML format via PatentCenter and is hereby incorporated by reference in its entirety. The .XMI is named 077875_767246_Sequence_Listing, and is 735 kilobytes in size.

### FIELD OF THE INVENTION

The present disclosure relates to modified plants, and methods and compositions for modifying plants, plant tissues, parts thereof, and seeds exhibiting reduced plasticity compared to wild type.

### BACKGROUND OF THE INVENTION

One goal of modern breeding programs is to increase the uniformity of crops so that harvesting time is more predictable and quality is consistent. This is true for intensive, precision outdoor farming and Total Controlled Environment Agriculture (TCEA, or vertical farming). However, plants are developmentally plastic and respond to changes in light and temperature to optimize their growth. Developmental plasticity is advantageous in natural conditions where resources and environmental stresses vary across seasons and location, but is disadvantageous in modern crop monoculture where fertilizers, pesticides, irrigation, etc., can be provided. Climate change offers additional challenges, with more varied weather and increasing temperatures disrupting plant development. Importantly, flowering in many crops is dependent on day length and highly influenced by ambient temperatures. This is disruptive to farmers who can only harvest at defined times of the year.

Accordingly, there is a need for plants with reduced developmental plasticity suitable for intensive, precision outdoor farming practices and TCEA or vertical farming.

### SUMMARY OF THE INVENTION

One aspect of the instant disclosure encompasses a modified plant or part thereof, plant cell, or seed exhibiting reduced developmental plasticity. The modified plant or part thereof, plant cell, or seed comprises (a) a modification of an endogenous polynucleotide sequence encoding a component of the circadian clock; and (b) an exogenous polynucleotide sequence encoding an environmental signal sensor. The modification of the endogenous polynucleotide sequence encoding the component of the circadian clock modifies expression of the component of the circadian clock; and the modification of the endogenous polynucleotide encoding the environmental signal sensor modifies expression of the environmental signal sensor. The modified expression of the component of the circadian clock and of the environmental signal sensor causes the plant to exhibit (i) reduced developmental plasticity in response to changes in environmental conditions during growth and (ii) modified circadian function.

The component of the circadian clock can be a component of the evening complex (EC). For instance, the component of the circadian clock is selected from EARLY FLOWERING 3 (ELF3), EARLY FLOWERING 4 (ELF4), LUX ARRHYTHMO (LUX), or any combination thereof. In some aspects, the component of the circadian clock is an ELF3 protein. In some aspects, the modification of a polynucleotide encoding the ELF3 protein is a loss-of-function mutation.

In some aspects, the plant or part thereof, plant cell, or seed is Arabidopsis thaliana (A. thaliana). When the plant is A. thaliana, the ELF3 protein can comprise an amino acid sequence comprising at least about 75% or more, at least about 85% or more, at least about 95% or more, or 100% sequence identity with an amino acid sequence of SEQ ID NO: 2. In some aspects, the polynucleotide encoding ELF3 comprises a nucleic acid sequence comprising at least about 75% or more, at least about 85% or more, at least about 95% or more, or 100% sequence identity with a nucleic acid sequence of SEQ ID NO: 1 and an ELF3 loss-of-function mutation comprises about 75% or more, at least about 85% or more, at least about 95% or more, or 100% sequence identity with a nucleic acid sequence of SEQ ID NO: 3 (elf3-1), SEQ ID NO: 4 (elf3-2), or a combination thereof.

In some aspects, the plant or part thereof, plant cell, or seed is Thlaspi arvense (T. arvense). When the plant is T. arvense, the ELF3 protein can comprise an amino acid sequence comprising at least about 75% or more, at least about 85% or more, at least about 95% or more, or 100% sequence identity with an amino acid sequence of SEQ ID NO: 21. In some aspects, the polynucleotide encoding the ELF3 protein comprises a nucleic acid sequence comprising at least about 75% or more, at least about 85% or more, at least about 95% or more, or 100% sequence identity with a nucleic acid sequence of SEQ ID NO: 20. The modification of the polynucleotide encoding the ELF3 protein can comprise a deletion of a nucleic acid segment in the polynucleotide encoding the ELF3 protein, wherein the nucleic acid segment can comprise about 75% or more, at least about 85% or more, at least about 95% or more, or 100% sequence identity with a nucleic acid sequence of SEQ ID NO: 22, a sequence comprising the AGCGCTA nucleic acid sequence, or a combination thereof.

In some aspects, the modification of the polynucleotide encoding the ELF3 protein comprises a modification introduced by a programmable nucleic acid modification system comprising a CRISPR/Cas nuclease system, wherein the CRISPR/Cas nuclease system comprises a CAS9 protein (dCAS9) and a guide RNA (gRNA). In some aspects, the Cas9 protein is encoded by a nucleic acid sequence comprising at least about 75% or more, at least about 85% or more, at least about 95% or more, or 100% sequence identity with a nucleic acid sequence of SEQ ID NO: 29 and the gRNA comprises a nucleic acid sequence of SEQ ID NO: 30, SEQ ID NO: 31, or a combination thereof. An expression construct expressing the Cas9 protein can comprise a nucleic acid sequence comprising at least about 75% or more, at least about 85% or more, at least about 95% or more, or 100% sequence identity with a nucleic acid sequence starting at base number 1552 to base number 7881 of SEQ ID NO: 32.

In some aspects, the plant or part thereof, plant cell, or seed is basil. When the plant is basil, the polynucleotide encoding ELF3 can comprise a nucleic acid sequence comprising at least about 75% or more, at least about 85% or more, at least about 95% or more, or 100% sequence identity with a nucleic acid sequence of SEQ ID NO: 13. In some aspects, the modification of the polynucleotide encoding the ELF3 protein comprises a modification introduced by a programmable nucleic acid modification system comprising a CRISPR/Cas nuclease system, wherein the CRISPR/Cas nuclease system comprises a CAS9 protein (dCAS9) and a guide RNA (gRNA). The Cas9 protein can be encoded by a nucleic acid sequence comprising at least about 75% or more, at least about 85% or more, at least about 95% or more, or 100% sequence identity with a nucleic acid sequence of SEQ ID NO: 29 and the gRNA comprises a nucleic acid sequence of SEQ ID NOs: 33-36, or any combination thereof. In some aspects, an expression construct expressing the Cas9 protein comprises a nucleic acid sequence comprising at least about 75% or more, at least about 85% or more, at least about 95% or more, or 100% sequence identity with a nucleic acid sequence starting at base number 1552 to base number 7881 of SEQ ID NO: 32.

The environmental signal can comprise photoperiod, light intensity and quality, temperature, chemicals, gravity, moisture, biotic or abiotic stress, oxygen and carbon dioxide concentrations, carbohydrate availability, or any combination thereof. For instance, the environmental signal sensor can be a photoreceptor, temperature sensor, CO₂ sensor, O₂ sensor, ethylene sensor, gravitropic sensor, or any combination thereof.

In some aspects, the environmental signal sensor is a photoreceptor. The photoreceptor can be a phytochrome; a cryptochrome, a phototropin, an F-box containing Flavin binding proteins; UVR8, or any combination thereof. In some aspects, the photoreceptor is a phytochrome photoreceptor. The polynucleotide encoding an environmental signal sensor can encode a phytochrome B photoreceptor. In some aspects, the phytochrome B photoreceptor comprises an amino acid sequence comprising at least about 75% or more, at least about 85% or more, at least about 95% or more, or 100% sequence identity with an amino acid sequence of SEQ ID NO: 6 or SEQ ID NO: 28. In other aspects, the polynucleotide encoding the phytochrome B photoreceptor comprises least about 75% or more, at least about 85% or more, at least about 95% or more, or 100% sequence identity with a nucleic acid sequence of SEQ ID NO: 5 or SEQ ID NO: 28.

The phytochrome B photoreceptor can be overexpressed, constitutively expressed, or constitutively active. In some aspects, the modification of a polynucleotide encoding a phyB photoreceptor encodes a constitutively active phyB photoreceptor. When the modification of the polynucleotide encoding a phyB photoreceptor encodes a constitutively active phyB photoreceptor, the phytochrome B photoreceptor comprises an amino acid sequence comprising at least about 75% or more, at least about 85% or more, at least about 95% or more, or 100% sequence identity with an amino acid sequence of SEQ ID NO: 8 SEQ ID NO: 12, SEQ ID NO: 26, or any combination thereof. In some aspects, the polynucleotide encoding the phytochrome B photoreceptor comprises least about 75% or more, at least about 85% or more, at least about 95% or more, or 100% sequence identity with a nucleic acid sequence of SEQ ID NO: 7, SEQ ID NO: 11, SEQ ID NO: 25, or any combination thereof.

In some aspects, the plant or part thereof, plant cell, or seed is Arabidopsis thaliana (A. thaliana). When the plant is A. thaliana, the modified A. thaliana plant can comprise a genetic modification of a polynucleotide encoding PhyB, wherein the polynucleotide comprising the genetic modification encodes a constitutively active PhyB photoreceptor. The constitutively active PhyB photoreceptor can comprise an amino acid sequence comprising at least about 75% or more, at least about 85% or more, at least about 95% or more, or 100% sequence identity with an amino acid sequence of SEQ ID NO: 8 SEQ ID NO: 12, SEQ ID NO: 26, or any combination thereof. In some aspects, the constitutively active PhyB photoreceptor is encoded by a nucleic acid sequence comprising at least about 75% or more, at least about 85% or more, at least about 95% or more, or 100% sequence identity with an amino acid sequence of SEQ ID NO: 7, SEQ ID NO: 11, SEQ ID NO: 25, or any combination thereof. In some aspects, the constitutively active PhyB photoreceptor comprises an amino acid sequence comprising at least about 75% or more, at least about 85% or more, at least about 95% or more, or 100% sequence identity with an amino acid sequence of SEQ ID NO: 8, SEQ ID NO: 12, or both. In other aspects, the constitutively active PhyB photoreceptor is encoded by a nucleic acid sequence comprising at least about 75% or more, at least about 85% or more, at least about 95% or more, or 100% sequence identity with an amino acid sequence of SEQ ID NO: 7, SEQ ID NO: 11, or both.

In some aspects, the plant or part thereof, plant cell, or seed is T. arvense. The modified T. arvensis plant can comprise a genetic modification of a polynucleotide encoding PhyB, wherein the polynucleotide comprising the genetic modification encodes a constitutively active PhyB photoreceptor. In some aspects, the constitutively active PhyB photoreceptor comprises an amino acid sequence comprising at least about 75% or more, at least about 85% or more, at least about 95% or more, or 100% sequence identity with an amino acid sequence of SEQ ID NO: 8 SEQ ID NO: 12, SEQ ID NO: 26, or any combination thereof. In some aspects, the constitutively active PhyB photoreceptor is encoded by a nucleic acid sequence comprising at least about 75% or more, at least about 85% or more, at least about 95% or more, or 100% sequence identity with an amino acid sequence of SEQ ID NO: 7, SEQ ID NO: 11, SEQ ID NO: 25, or SEQ ID NO: 27. In other aspects, the constitutively active PhyB photoreceptor comprises an amino acid sequence comprising at least about 75% or more, at least about 85% or more, at least about 95% or more, or 100% sequence identity with an amino acid sequence of SEQ ID NO: 26. The constitutively active PhyB photoreceptor can be encoded by a nucleic acid sequence comprising at least about 75% or more, at least about 85% or more, at least about 95% or more, or 100% sequence identity with an amino acid sequence of SEQ ID NO: 25.

In some aspects, the modified plant or part thereof, plant cell, or seed of can comprise (a) a modification of a polynucleotide encoding an ELF3 protein wherein the polynucleotide encoding the ELF3 protein comprises an ELF3 loss-of-function mutation; and (b) a modification of a polynucleotide encoding a phyB photoreceptor wherein the polynucleotide encodes a constitutively active PhyB photoreceptor. In some aspects, the modified plant comprises constitutively inactivated temperature input to the circadian clock and a constitutively activated light input to the circadian clock. In some aspects, the modified plant is not etiolated, flowers earlier and consistently regardless of changes in day length and has a reduced cellular elongation response to increasing temperature thereby reducing listing in the field, and any combination thereof.

In some aspects, the modified plant is plant is A. thaliana. The modified A. thaliana can comprise (a) a polynucleotide encoding an ELF3 protein wherein the polynucleotide encoding the ELF3 protein comprises an ELF3 loss-of-function mutation, wherein the polynucleotide comprises a nucleic acid sequence comprising at least about 75% or more, at least about 85% or more, at least about 95% or more, or 100% sequence identity with an amino acid sequence of SEQ ID NO: 3 (elf3-1), SEQ ID NO: 4 (elf3-2), or a combination thereof; and (b) a polynucleotide encoding a constitutively active PhyB photoreceptor, wherein the polynucleotide comprises a nucleic acid sequence comprising at least about 75% or more, at least about 85% or more, at least about 95% or more, or 100% sequence identity with an amino acid sequence of SEQ ID NO: 7, SEQ ID NO: 11, or a combination thereof.

In other aspects, the modified plant is plant is T. arvensis comprising (a) a polynucleotide encoding an ELF3 protein wherein the polynucleotide encoding the ELF3 protein comprises an ELF3 loss-of-function mutation, wherein the polynucleotide comprises a nucleic acid sequence comprising at least about 75% or more, at least about 85% or more, at least about 95% or more, or 100% sequence identity with an amino acid sequence of SEQ ID NO: 22, a sequence comprising the AGCGCTA nucleic acid sequence, or a combination thereof; and (b) a polynucleotide encoding a constitutively active PhyB photoreceptor, wherein the polynucleotide comprises a nucleic acid sequence comprising at least about 75% or more, at least about 85% or more, at least about 95% or more, or 100% sequence identity with an amino acid sequence of SEQ ID NO: 25.

Another aspect of the instant disclosure encompasses a system for modifying a component of the circadian clock, an environmental signal sensor, or both in a plant. The system can comprise a nucleic acid construct comprising at least one or both of: (a) a programmable nucleic acid modification system comprising a targeting nucleic acid sequence targeting a nucleic acid sequence in a polynucleotide sequence encoding a component of the circadian clock; and (b) an expression construct comprising a promoter operably linked to a polynucleotide encoding a polynucleotide encoding an environmental signal sensor; wherein (a) introduces a loss of function mutation into the polynucleotide sequence encoding the component of the circadian clock, and wherein (b) increases expression of the environmental signal sensor, thereby causing the plant to exhibit reduced developmental plasticity in response to changes in environmental conditions during growth and modified circadian function.

In some aspects, the programmable nucleic acid modification system introduces a loss of function mutation into a polynucleotide encoding an ELF3 protein. In some aspects, the programmable nucleic acid modification system comprises a CRISPR/Cas nuclease system comprising a CAS9 protein (dCAS9) and a guide RNA (gRNA).

The plant can be T. arvense. When the plant is T. arvense, the Cas9 protein can be encoded by a nucleic acid sequence comprising at least about 75% or more, at least about 85% or more, at least about 95% or more, or 100% sequence identity with a nucleic acid sequence of SEQ ID NO: 29 and the gRNA comprises a nucleic acid sequence of SEQ ID NO: 30, SEQ ID NO: 31, or both.

The plant can also be basil. When the plant is basil, the Cas9 protein can be encoded by a nucleic acid sequence comprising at least about 75% or more, at least about 85% or more, at least about 95% or more, or 100% sequence identity with a nucleic acid sequence of SEQ ID NO: 29 and the gRNA can comprise a nucleic acid sequence of SEQ ID NOs: 33-36, or any combination thereof.

In some aspects, the expression construct comprises a promoter operably linked to a polynucleotide encoding a polynucleotide encoding a PhyB photoreceptor, wherein the expression construct increases expression of PhyB. In some aspects, the expression construct comprising a promoter operably linked to a polynucleotide encoding an environmental signal sensor comprises a 35S promoter operably linked to a polynucleotide encoding the PhyB photoreceptor and the construct constitutively expresses the PhyB photoreceptor. In some aspects, the PhyB photoreceptor is constitutively active. When the PhyB photoreceptor is constitutively active, the expression construct can comprise a nucleic acid sequence comprising about 75% or more, at least about 85% or more, at least about 95% or more, or 100% sequence identity with SEQ ID NO: 24. In some aspects, the expression construct comprises a nucleic acid sequence comprising a nucleic acid sequence comprising about 75% or more, at least about 85% or more, at least about 95% or more, or 100% of a nucleic acid sequence starting at base 8148 to base 12020 of SEQ ID NO: 19.

Another aspect of the instant disclosure encompasses a modified plant or part thereof, plant cell, or seed comprising one or more systems for modifying a plant exhibiting reduced developmental plasticity. The systems can be as described herein above.

Yet another aspect of the instant disclosure encompasses one or more expression constructs for modifying a plant or part thereof, plant cell, or seed exhibiting reduced developmental plasticity, the one or more expression constructs comprising at least one or both of: (a) an expression construct for modifying the expression of a component of the circadian clock, and (b) an expression construct for modifying the expression of an environmental signal sensor.

The expression construct for modifying the expression of a component of the circadian clock can comprise a promoter operably linked to a polynucleotide encoding a programmable nucleic acid modification system targeted to a nucleic acid sequence in a polynucleotide encoding a component of the circadian clock. In some aspects, the programmable nucleic acid modification system comprises a CRISPR/Cas nuclease system and wherein the CRISPR/Cas nuclease system comprises a CAS9 protein (dCAS9) and a guide RNA (gRNA). In some aspects, an expression construct expressing the Cas9 protein comprises a nucleic acid sequence comprising at least about 75% or more, at least about 85% or more, at least about 95% or more, or 100% sequence identity with a nucleic acid sequence starting at base number 1552 to base number 7881 of SEQ ID NO: 32.

In some aspects, the plant is T. arvense. When the plant is T. arvense, the polynucleotide encoding a component of the circadian clock can encode ELF3, wherein the polynucleotide encoding ELF3 can comprise a nucleic acid sequence comprising at least about 75% or more, at least about 85% or more, at least about 95% or more, or 100% sequence identity with a nucleic acid sequence of SEQ ID NO: 20 and the gRNA can comprise a nucleic acid sequence of SEQ ID NO: 30, SEQ ID NO: 31, or both. In some aspects, the expression construct comprises a nucleic acid sequence comprising about 75% or more, at least about 85% or more, at least about 95% or more, or 100% sequence identity with a nucleic acid sequence starting at base 254 to base 1287 of SEQ ID NO: 32.

The plant can also be Basil. When the plant is basil, the polynucleotide encoding a component of the circadian clock can encode ELF3, the polynucleotide encoding ELF3 can comprise a nucleic acid sequence comprising at least about 75% or more, at least about 85% or more, at least about 95% or more, or 100% sequence identity with a nucleic acid sequence of SEQ ID NO: 13, and the gRNA can comprise a nucleic acid sequence of SEQ ID NOs: 33-36, or any combination thereof. In some aspects, an expression construct expressing the gRNA of SEQ ID NOs: 33-36 comprises a nucleic acid sequence comprising about 75% or more, at least about 85% or more, at least about 95% or more, or 100% sequence identity with the gRNA expression construct of SEQ ID NO: 37. In some aspects, an expression construct expressing the gRNA of SEQ ID NOs: 33-36 comprises about 75% or more, at least about 85% or more, at least about 95% or more, or 100% sequence identity with SEQ ID NO: 23. In some aspects, the expression construct expressing the gRNA of SEQ ID NOs: 33-36 comprises about 75% or more, at least about 85% or more, at least about 95% or more, or 100% of a nucleic acid sequence starting at base 695 to base 2799 of SEQ ID NO: 37.

In some aspects, the expression construct comprising a promoter operably linked to a polynucleotide encoding an environmental signal sensor comprises a constitutive expression promoter operably linked to a polynucleotide encoding a PhyB photoreceptor. In some aspects, the expression construct comprising a promoter operably linked to a polynucleotide encoding an environmental signal sensor comprises a 35S promoter operably linked to a polynucleotide encoding a constitutively active PhyB photoreceptor. The constitutively active PhyB photoreceptor can be encoded by a nucleic acid sequence comprising about 75% or more, at least about 85% or more, at least about 95% or more, or 100% sequence identity with a nucleic acid sequence of SEQ ID NO: 7, SEQ ID NO: 11, or both. In some aspects, the expression construct comprises a nucleic acid sequence comprising about 75% or more, at least about 85% or more, at least about 95% or more, or 100% sequence identity with SEQ ID NO: 24. In some aspects, the expression construct comprising a nucleic acid sequence comprising about 75% or more, at least about 85% or more, at least about 95% or more, or 100% of a nucleic acid sequence starting at base 8148 to base 12020 of SEQ ID NO: 19.

An additional aspect of the instant disclosure encompasses a modified plant or part thereof, plant cell, or seed comprising one or more expression constructs for modifying a plant exhibiting reduced developmental plasticity. The constructs can be as described herein above.

One aspect of the instant disclosure encompasses a method of improving performance of a plant or part thereof, plant cell, or seed grown under intensive, precision outdoor farming conditions, Total Controlled Environment Agriculture (TCEA), or vertical farming. The method comprises obtaining or having obtained a modified plant or part thereof, plant cell, or seed exhibiting reduced developmental plasticity; and cultivating the plant under intensive, precision outdoor farming conditions, TCEA, or vertical farming.

A yet additional aspect of the instant disclosure encompasses a method of reducing developmental plasticity of a plant or part thereof, plant cell, or seed. The method comprises generating a modified plant comprising one or more expression constructs for modifying a plant or part thereof, plant cell, or seed exhibiting reduced developmental plasticity. The plant or part thereof, plant cell, or seed can be as described herein above. The plant can be resistant to changes in growth conditions of the plant. In some aspects, the growth conditions comprise daylength and temperature.

One aspect of the instant disclosure encompasses a method of reducing a plastic response to competition among a co-cultivated group of plants or parts thereof, plant cells, or seeds. The method comprises, co-cultivating a group of modified plants or parts thereof, plant cells, or seeds exhibiting reduced developmental plasticity. The of plants or parts thereof, plant cells, or seeds can be as described herein above.

Another aspect of the instant disclosure encompasses a method of stabilizing production of a metabolite, nucleic acid, or protein in a plant or part thereof, plant cell, or seed. The method comprises obtaining or having obtained a modified plant or part thereof, plant cell, or seed exhibiting reduced developmental plasticity; cultivating the plant for a time sufficient to generate the metabolite, nucleic acid, or protein; and harvesting the metabolite, nucleic acid, or protein from the plant or part thereof, plant cell, or seed. In some aspects, the plant is grown under intensive, precision outdoor farming conditions, TCEA, or vertical farming. In some aspects, stabilizing production of a metabolite, nucleic acid, or protein can comprise equal production of the metabolite, nucleic acid, or protein irrespective of light period, temperature variation or both.

Another aspect of the instant disclosure encompasses a kit for modifying a plant or part thereof, plant cell, or seed exhibiting reduced developmental plasticity. The kit comprises: (a) one or more modified plant or part thereof, plant cell, or seed exhibiting reduced developmental plasticity; (b) one or more expression constructs for modifying a plant or part thereof, plant cell, or seed to exhibit reduced developmental plasticity; (c) one or more modified plants or parts thereof, plant cells, or seeds comprising one or more expression constructs for modifying a plant or part thereof, plant cell, or seed to exhibit reduced developmental plasticity; or any combination of (a) to (c). The plants or parts thereof, plant cells, or seeds and the constructs can be as described herein above.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a simplified representation of the plant circadian regulatory network. Similar genes operating at similar times during the day in a similar manner are grouped together in white circles. Black lines with blunt ends indicate genes function as repressors in the negative feedback loops. Gray lines and arrows indicate genes acting as activators in the regulatory network.

FIG. 2A is a schematic representation of how light signaling pathways integrate with the circadian clock regulatory network. The underlying clock network is the same as FIG. 1 with the light-signaling pathway linking to points in the circadian regulatory network. Red and blue pathways indicate how these different wavelengths of light are integrated into the clock at different points via independent pathways.

FIG. 2B is a schematic representation of how temperature signaling pathways integrate with the circadian clock regulatory network. The underlying clock network is the same as FIG. 1 with the temperature-signaling pathway linking to points in the circadian regulatory network. Different temperatures influence the same pathway (blue to orange shaded box), with cooler temperatures stabilizing complex formation and warmer temperatures releasing growth factors such as PIFs.

FIG. 3A is a plot showing the accumulation of PHYB and YHB transcript in wild-type, YHB, elf3-2, and PHYB::YHB(elf3) seedlings determined using qRT-PCR.

FIG. 3B is a photo or seedlings of WT, elf3-2, YHB, and YHB (elf3-2) E269.11 grown in constant darkness.

FIG. 3C is a plot showing hypocotyl elongation of seedlings grown under either constant darkness, short day (8:16) or long day (16:8) light:dark cycles for 6 days.

FIG. 3D is a plot showing hypocotyl elongation in response to temperature. Seedlings were grown in short day (8:16) light:dark cycles at the indicated temperature for 6 days.

FIG. 3E are photographs of plants showing the rosette phenotype of adult PHYB::YHB seedlings following growth under short day conditions.

FIG. 3F is a plot showing flowering phenotype of PHYB::YHB seedlings grown under short- or long-day conditions.

FIG. 4A is a plot showing the hypocotyl elongation response of wild type, elf3-1, phyb-9, elf3-1 phyB-9, and elf3-1 phyb-9 YHB plants when combined in response to daylength. Seedlings were grown under either short day (8 light:16 dark), day neutral (12 light:12 dark) or long day (16 light:8 dark) cycles for 6 days at 22° C. at ˜80 μmol m-2 s-1.

FIG. 4B is a plot showing the hypocotyl elongation response of wild type, elf3-1, phyb-9, elf3-1 phyB-9, and elf3-1 phyb-9 YHB plants when combined in response to temperature. Seedlings were grown under day neutral (12 light:12 dark) cycles at ˜80 μmol m-2 s-1 at either 16° C., 22° C., or 28° C. for 6 days before measurement.

FIG. 5A is a plot showing the circadian rhythms of wild type, elf3-2, PHYB::YHB and PHYB::YHB elf3-2 plants in constant darkness, measured by luciferase bioluminescence driven by the circadian CCA1 promoter. White boxes indicate periods of light irradiation in light:dark cycles prior to transfer to constant darkness. Dark grey segments indicate periods of darkness; light grey segments highlight periods of subjective daytime when held in constant darkness. Zeitgeber time is hours since last dawn.

FIG. 5B is a plot showing the circadian rhythms of wild type, elf3-1, elf3-1 phyb-9, and elf3-1 phyb-9 35S::YHB plants in constant darkness, measured by luciferase bioluminescence driven by the circadian CCA1 promoter. White boxes indicate periods of light irradiation in light:dark cycles prior to transfer to constant darkness. Dark grey segments indicate periods of darkness; light grey segments highlight periods of subjective daytime when held in constant darkness. Zeitgeber time is hours since last dawn.

FIG. 6A. The combination of YHB and elf3 alleles alters gene expression additively compared to either allele alone. Heatmap showing fold change of transcript abundance (log 2FC) for each core circadian clock gene (list of circadian genes taken from Hsu and Harmer, 2014) in dark treated elf3-2, YHB, and YHB(elf3-2) seedlings respective to the expression in wild type (Col-0).

FIG. 6B. The combination of YHB and elf3 alleles alters gene expression additively compared to either allele alone. Venn diagram of highly differentially expressed (log 2FC>1.0, p<0.05) circadian genes in one or more mutant genotypes with respect to wild type.

FIG. 6C. The combination of YHB and elf3 alleles alters gene expression additively compared to either allele alone. Heatmap showing highly differentially expressed (log 2FC>1.0, p<0.05) genes associated with photomorphogenesis (list of photomorphogenesis genes taken from TAIR keywords in either of dark treated YHB, YHB(elf3-2) or elf3-2 seedlings respective to the expression in wild type (Col-0).

FIG. 6D. The combination of YHB and elf3 alleles alters gene expression additively compared to either allele alone. Venn diagram of highly differentially expressed (log 2FC>1.0, p<0.05) genes associated with photomorphogenesis in one or more mutant genotypes with respect to wild type. Normalized transcript abundance in wild type samples was used to calculate fold change difference of transcript abundance. Data shows the average of the three independent biological replicates.

FIG. 6E. ELF3 is required to enable YHB-driven circadian rhythms of GIGANTEA (GI) in constant darkness. Real-time reverse transcription polymerase chain reaction showing steady-state accumulation of GI transcripts in wild type, YHB, YHB(elf3-2) and elf3-2 seedlings. Seedlings were grown for 12 days under 12 hrs:12 hrs light:dark cycles on 0.5MS plates before transfer to constant darkness at ZT12. RNA was extracted from pools of ca. 20 seedlings for each genotype and used to synthesise cDNA. qRT-PCR data was normalised to the expression of three internal control genes (APA1, APX3 and IPP2), and shows the average expression from three independent experiments.

FIG. 6F. ELF3 is required to enable YHB-driven circadian rhythms of PRR9 in constant darkness. Real-time reverse transcription polymerase chain reaction showing steady-state accumulation of PRR9 transcripts in wild type, YHB, YHB(elf3-2) and elf3-2 seedlings. Seedlings were grown for 12 days under 12 hrs:12 hrs light:dark cycles on 0.5MS plates before transfer to constant darkness at ZT12. RNA was extracted from pools of ca. 20 seedlings for each genotype and used to synthesise cDNA. qRT-PCR data was normalised to the expression of three internal control genes (APA1, APX3 and IPP2), and shows the average expression from three independent experiments.

FIG. 6G. ELF3 is required to enable YHB-driven circadian rhythms of CCA1 in constant darkness. Real-time reverse transcription polymerase chain reaction showing steady-state accumulation of CCA1 transcripts in wild type, YHB, YHB(elf3-2) and elf3-2 seedlings. Seedlings were grown for 12 days under 12 hrs:12 hrs light:dark cycles on 0.5MS plates before transfer to constant darkness at ZT12. RNA was extracted from pools of ca. 20 seedlings for each genotype and used to synthesise cDNA. qRT-PCR data was normalised to the expression of three internal control genes (APA1, APX3 and IPP2), and shows the average expression from three independent experiments.

FIG. 6H. ELF3 is required to enable YHB-driven circadian rhythms of LHY in constant darkness. Real-time reverse transcription polymerase chain reaction showing steady-state accumulation of LHY transcripts in wild type, YHB, YHB(elf3-2) and elf3-2 seedlings. Seedlings were grown for 12 days under 12 hrs:12 hrs light:dark cycles on 0.5MS plates before transfer to constant darkness at ZT12. RNA was extracted from pools of ca. 20 seedlings for each genotype and used to synthesise cDNA. qRT-PCR data was normalised to the expression of three internal control genes (APA1, APX3 and IPP2), and shows the average expression from three independent experiments.

FIG. 7A. Characterisation of additional YHB elf3 alleles. Relative gene expression of wild type PHYB and YHB transcript in 6 day old Col-0 (Wild type), PHYB::YHB, elf3-2, and four independently-transformed PHYB::YHB(elf3-2) lines.

FIG. 7B. Characterisation of additional YHB elf3 alleles. Relative gene expression of wild type PHYB and YHB transcript in 6 day old Col-0 (Wild type), phyb-9, elf3-1, and a 35S::YHB(phyb-9 elf3-1) line.

FIG. 7C. Characterisation of additional YHB elf3 alleles. Assessment of circadian rhythms following transfer to constant darkness in additional PHYB::YHB (elf3-2) transformed lines. White boxes indicate periods of light irradiation in light:dark cycles prior to transfer to constant darkness. Dark grey segments indicate periods of darkness; light grey segments highlight periods of subjective daytime when held in constant darkness. Zeitgeber time is hours since last dawn.

FIG. 7D. Characterisation of additional YHB elf3 alleles. Assessment of circadian rhythms following transfer to constant darkness in 35S::YHB (phyb-9 elf3-1) seedlings. White boxes indicate periods of light irradiation in light:dark cycles prior to transfer to constant darkness. Dark grey segments indicate periods of darkness; light grey segments highlight periods of subjective daytime when held in constant darkness. Zeitgeber time is hours since last dawn.

FIG. 8A. Venn diagram highlighting circadian-regulated promoters bound by phyB or ELF3. Promoters bound by either phyB (red) or ELF3 (blue) are highlighted. ‘Core’ circadian gene list taken from Hsu and Harmer 2014.

FIG. 8B. Patterns of gene expression correlate with reported binding patterns for phyB and ELF3. Binding patterns of phyB (red) and ELF3 (blue) at CCA1, LHY, PRR9 and GI loci at different temperatures (phyB data) or throughout the day (ELF3 data).

FIG. 9A. YHB elf3 plants retain daily variation of gene expression in light:dark cycles. Luciferase bioluminescence reporting activity of the circadian-regulated CCA1 promoter in transgenic Arabidopsis (CCA1::LUC2). Rhythms are maintained in all genotypes in driven light:dark cycles (indicated by black bars), but are lost in constant light (subjective night indicated by grey bars).

FIG. 9B. YHB elf3 plants retain daily variation of gene expression in light:dark cycles. Daily patterns of gene expression (represented by CCA1-driven bioluminescence) are maintained in square-wave light conditions. White box indicates relative light intensity (right hand axis).

FIG. 9C. YHB elf3 plants retain daily variation of gene expression in light:dark cycles. Daily patterns of gene expression (represented by CCA1-driven bioluminescence) are maintained in fluctuating light conditions. White box indicates relative light intensity (right hand axis).

FIG. 10A. YHB elf3 plants demonstrate less developmental plasticity in response to varying environmental cues. Quantification of hypocotyl length of plants grown in constant darkness, short days, long days, or varied light cycles. Asterisks indicate a significantly different hypocotyl length than plants of the same genotype grown in constant darkness (p<0.05). Short days describes daily cycles of 8 hrs:16 hrs light:dark (square-form), long days describes daily cycles of 16 hrs:8 hrs light:dark (square-form), varied light describes daily cycles of 1 hrs 10 μmol m-2 s-1, 8 hrs 40 μmol m-2 s-1, 5 hrs 30 μmol m-2 s-1, 4 hrs 10 μmol m-2 s-1 white light followed by 6 hrs of darkness.

FIG. 10B. YHB elf3 plants demonstrate less developmental plasticity in response to varying environmental cues. Representative images of wild type (Col-0), YHB, YHB(elf3-2) and elf3-2 seedlings grown for 21 days under long days. Short days describes daily cycles of 8 hrs:16 hrs light:dark (square-form), long days describes daily cycles of 16 hrs:8 hrs light:dark (square-form), varied light describes daily cycles of 1 hrs 10 μmol m-2 s-1, 8 hrs 40 μmol m-2 s-1, 5 hrs 30 μmol m-2 s-1, 4 hrs 10 μmol m-2 s-1 white light followed by 6 hrs of darkness.

FIG. 10C. YHB elf3 plants demonstrate less developmental plasticity in response to varying environmental cues. Rosette diameter at flowering of Arabidopsis plants grown under either long days or short days light. Short days describes daily cycles of 8 hrs:16 hrs light:dark (square-form), long days describes daily cycles of 16 hrs:8 hrs light:dark (square-form).

FIG. 10D. YHB elf3 plants demonstrate less developmental plasticity in response to varying environmental cues. Flowering time of wild type, YHB, YHB(elf3-2) and elf3-2 plants grown under long (16 hr:8 hr light:dark) or short (8 hr:16 hr light:dark) days. Asterisks indicate a significant difference in flowering time between plants of the given genotype grown in long and short days (p<0.0001). Short days describes daily cycles of 8 hrs:16 hrs light:dark (square-form), long days describes daily cycles of 16 hrs:8 hrs light:dark (square-form).

FIG. 10E. YHB elf3 plants demonstrate less developmental plasticity in response to varying environmental cues. Flowering time of plants grown under 12:12 light dark cycles with 160 μmol m-2 s-1 white light. After germination and establishment for 7 days at 22° C., plants were transferred to a constant temperature of either 22° C. or 27° C. as indicated.

FIG. 10F. YHB elf3 plants demonstrate less developmental plasticity in response to varying environmental cues. Average rosette area over time of Arabidopsis plants grown under short days. Short days describes daily cycles of 8 hrs:16 hrs light:dark (square-form),

FIG. 10G. YHB elf3 plants demonstrate less developmental plasticity in response to varying environmental cues. Average rosette area over time of plants grown under long days. Long days describes daily cycles of 16 hrs:8 hrs light:dark (square-form).

FIG. 10H. YHB elf3 plants demonstrate less developmental plasticity in response to varying environmental cues. Expanded graph of FIG. 10F shows reduced growth in YHB elf3 during the first 35 days of growth in short day conditions. Short days describes daily cycles of 8 hrs:16 hrs light:dark (square-form).

FIG. 11A. YHB and YHB (elf3) seedlings retain responses to light and temperature. Phase response curves of Col-0, YHB, YHB(elf3-2) and elf3-2 seedlings. Seedlings were held in constant light for the indicated time (y axis) prior to release into constant darkness for imaging. Phase is reported relative to CTO (dawn for entrained plants) and was defined as the time until maximal CCA1-driven bioluminescence.

FIG. 11B. YHB and YHB (elf3) seedlings retain responses to light and temperature. Circadian free running period estimates of plants transferred to varied intensities of white light at different constant temperatures. Wild type, YHB, YHB(elf3-2) and elf3-2 seedlings expressing a CCA1::LUC2 reporter, entrained for 7 days under 12 hr:12 hr light:dark cycles at 22° C. before transfer to constant light at a temperature of 12° C. Plants were imaged every 2 hours after transfer to constant light at the intensity indicated on the x-axis of each plot. Data shows the average free running periods from two independent experiments.

FIG. 11C. YHB and YHB (elf3) seedlings retain responses to light and temperature. Circadian free running period estimates of plants transferred to varied intensities of white light at different constant temperatures. Wild type, YHB, YHB(elf3-2) and elf3-2 seedlings expressing a CCA1::LUC2 reporter, entrained for 7 days under 12 hr:12 hr light:dark cycles at 22° C. before transfer to constant light at a temperature of 17° C. Plants were imaged every 2 hours after transfer to constant light at the intensity indicated on the x-axis of each plot. Data shows the average free running periods from two independent experiments.

FIG. 11D. YHB and YHB (elf3) seedlings retain responses to light and temperature. Circadian free running period estimates of plants transferred to varied intensities of white light at different constant temperatures. Wild type, YHB, YHB(elf3-2) and elf3-2 seedlings expressing a CCA1::LUC2 reporter, entrained for 7 days under 12 hr:12 hr light:dark cycles at 22° C. before transfer to constant light at a temperature of 22° C. Plants were imaged every 2 hours after transfer to constant light at the intensity indicated on the x-axis of each plot. Data shows the average free running periods from two independent experiments.

FIG. 11E. YHB and YHB (elf3) seedlings retain responses to light and temperature. Circadian free running period estimates of plants transferred to varied intensities of white light at different constant temperatures. Wild type, YHB, YHB(elf3-2) and elf3-2 seedlings expressing a CCA1::LUC2 reporter, entrained for 7 days under 12 hr:12 hr light:dark cycles at 22° C. before transfer to constant light at a temperature of 27° C. Plants were imaged every 2 hours after transfer to constant light at the intensity indicated on the x-axis of each plot. Data shows the average free running periods from two independent experiments.

FIGS. 12A-12D. Additional description of data presented in FIGS. 11B-11E. Data from FIGS. 11B-11E is replotted as temperature response curves.

FIG. 13. Diagrammatic representation of a nucleic acid modification in a polynucleotide encoding an elf3 protein in pennycress generated using CRISPR editing. Two independent editing events resulting in either a 7 bp or 1683 bp deletion are shown as a comparison of the sequencing chromatogram (below) to the genomic sequence (above) starting from the start of translation in exon 1. The gene structure of pennycress ELF3 is represented by exons as thick black rectangles and introns as thin black lines.

### DETAILED DESCRIPTION

The present disclosure is based in part on the surprising demonstration of reduced developmental plasticity obtained when a combination of two or more genetic modifications in the environmental sensing and circadian clock mechanisms of the plant provides advantages over each genetic modification individually. More specifically, the inventors surprisingly discovered that ability of the plant to sense and respond to environmental cues, when combined with a modification of a circadian function of the plant, can reduce the plasticity of the plant in response to changes in environmental conditions during growth all while exhibiting improved agronomic traits regardless of changing environmental conditions. For instance, plants comprising both genetic modifications can flower consistently and uniformly regardless of differences in day length, at varied latitudes, in the face of climate change, increasing temperatures and disrupting weather patterns, or any combination thereof. The genetic modifications increase the uniformity of crops so that harvesting time is more predictable and quality is consistent in intensive, precision outdoor farming practices and Total Controlled Environment Agriculture (TCEA, or vertical farming).

### I. Genetically Modified Plants

One aspect of the present disclosure encompasses a genetically modified plant, plant tissues, part thereof, plant cell, or seed (hereinafter referred to as “genetically modified plants” or “modified plants”) having reduced developmental plasticity and improved agronomic traits. The modified plants comprise at least two genetic modifications that modify the expression of two or more components of the circadian system. The main components of a circadian system are the central oscillator (the circadian clock) that controls circadian functions by maintaining a roughly 24-h rhythm even in the absence of input signals, the input signals from the environment that reset the clock, and the output signals that generate daily rhythms in pathways associated with development, physiology, and metabolism. As used herein, the term “circadian function” is defined as the effect on plant growth and development resulting from the circadian system in response to sensed environmental factors. Non-limiting examples of circadian functions include transitions from shoot elongation, regulation of root gravitropism, altered flowering time, growth cessation of leaves, and timing of germination, the synthesis of chlorophyll, de-etiolation (when a seedling emerges into the light and starts its photo-autotrophic life style), stomata development, transition to flowering, senescence, shade avoidance, elongation of seedlings, size, shape, number, and movement of leaves, and the timing of flowering in adult plants

In some aspects, the modified plants comprise a nucleic acid modification in a polynucleotide encoding a component of the circadian clock and a nucleic acid modification of a polynucleotide encoding an environmental signal sensor. The nucleic acid modifications modify the expression of the component of the circadian clock and the environmental signal sensor. As used herein, “expression” includes but is not limited to one or more of the following: transcription of a gene into precursor mRNA; splicing and other processing of the precursor mRNA to produce mature mRNA; mRNA stability; translation of the mature mRNA into protein (including codon usage and tRNA availability); production of a mutant protein comprising a mutation that modifies the activity of the protein; and glycosylation and/or other modifications of the translation product, if required for proper expression and function.

Components of the circadian clock and genetic modifications in components of the circadian clock are described in Section I(a). Environmental signal sensors and genetic modifications that modify expression of environmental signal sensors are described in Section I(b).

### (a) Circadian Clock

A modified plant of the instant disclosure comprises a genetic modification of a polynucleotide encoding a component of the circadian clock. The genetic modification modifies a function of the circadian system. In some aspects, the modification that modifies a function of the circadian system comprises a modification of an endogenous polynucleotide sequence encoding a component of the circadian clock.

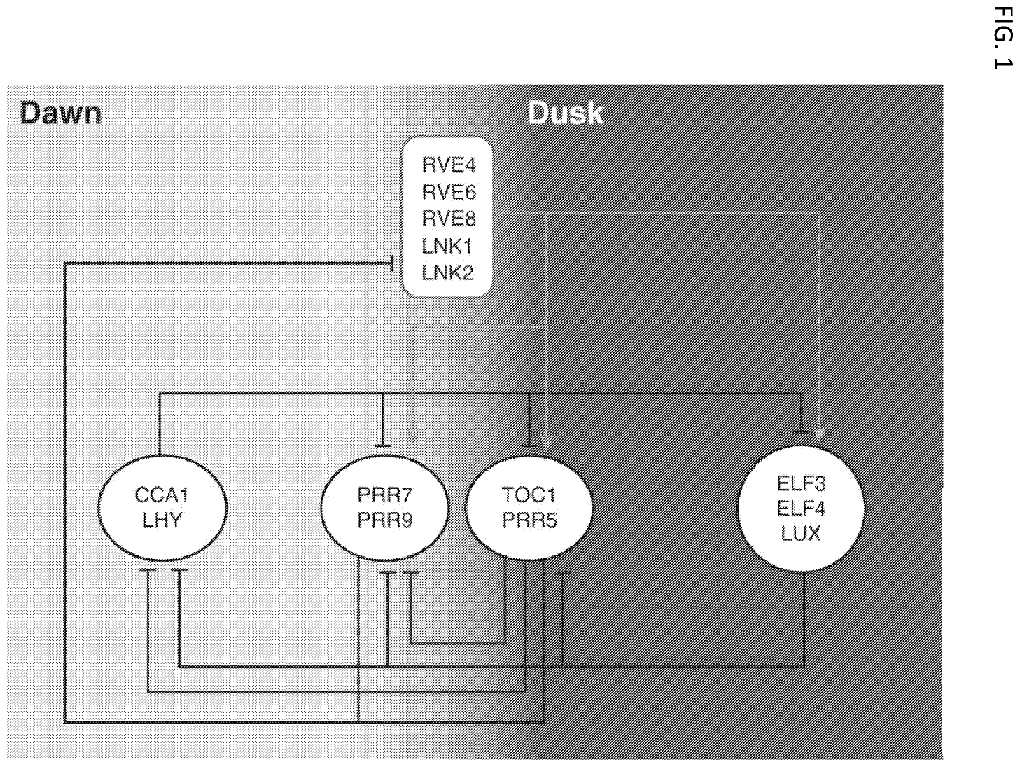

In plants, the circadian clock is a complex gene regulatory network of repressors and activators that form multiple interlocking feedback loops (FIG. 1). These clock genes are expressed at specific times of the day, regulate each other's expression, and influence multiple pathways and mechanisms of development, physiology, and metabolism. The circadian clock ensures that a plant is most responsive to light during daylight hours, to growth hormones during the night, and to environmental stresses at times when adverse conditions are most likely. To appropriately regulate plant responses to external factors, the clock is directly linked with the light and temperature-signaling pathways, which also ensures synchronicity between the external and internal rhythms (See Section I(b) herein below). The cross talk between these regulatory pathways also provides seasonal information to the plant, allowing for example the determination of day length for the appropriate control of the transition to flowering.

A genetic modification of the instant disclosure that modifies a function of the circadian clock can modify any component of the circadian clock. The network of intertwined feedback loops of the plant circadian clock comprises repressor and activator transcription factors among other factors. Levels of these proteins are in constant flux, each peaking at a specific time of day and feeding back to regulate each other's expression. In Arabidopsis, the morning-expressed MYB-like transcription factors CCA1 (CIRCADIAN CLOCK ASSOCIATED1) and LHY (LATE ELOGATED HYPOCOTYL) repress the afternoon expressed PSEUDO-RESPONSE REGULATOR (PRR) genes, including PRR1/TOC1 (TIMING OF CAB EXPRESSION 1), PRR5, PRR7, and PRR9. TOC1, along with the other PRR proteins, in turn, repress the expression of CCA1 and LHY, closing this feedback loop. CCA1/LHY are themselves primarily repressors of transcription and bind to a cis-motif termed the evening element (EE) found in the regulatory regions of many clock genes, including the PRRs. Other direct targets of CCA1/LHY activity include genes that encode members of the transcriptional regulatory evening complex, ELF3 (EARLY FLOWERING3), ELF4, and LUX (LUX ARRHYTHMO). These three genes are expressed at night, at which time the evening complex feeds back to repress multiple morning- and afternoon-expressed genes to complete another feedback loop in the network (FIG. 1).

The circadian regulatory network in plants also comprises a second set of midday-expressed MYB-like transcription factors, REVEILLE4 (RVE4), RVE6, and RVE8, have been shown to activate expression of several clock genes including TOC1, the PRRs and the evening complex genes. To activate gene expression, RVE8 forms a complex with LNK1 (NIGHT LIGHT-INDUCIBLE AND CLOCK-REGULATED1) and LNK2 and associates with the promoters of TOC1 and PRR5. The RVE activator proteins are not simply a second layer of regulation on top of the core circadian clock but are connected and embedded into the clock regulatory network (FIG. 1). RVE8 expression is repressed by TOC1 and the PRRs, forming yet another negative feedback loop in this network.

Land plants contain an additional family of LOV UV-A/blue light photoreceptors/clock proteins comprising ZEITLUPE (ztl), FLAVIN-BINDING, KELCH REPEAT, F-BOX (fkf1) and LOV KELCH PROTEIN 2 (Ikp2) proteins (collectively referred to as Zeitlupes). In contrast to the phototropins, Zeitlupes contain a single FMN-binding LOV domain followed by an F-box and six Kelch repeats. As suggested by their domain organization, Zeitlupes form SCF E3 ubiquitin ligase complexes directly controlling light-mediated protein degradation. LOV domains are also found associated with other output domains including DNA binding b-ZIP in aureochromes from photosynthetic stramenopile algae. Therefore, LOV domains are versatile light absorption modules mediating different biochemical outputs.

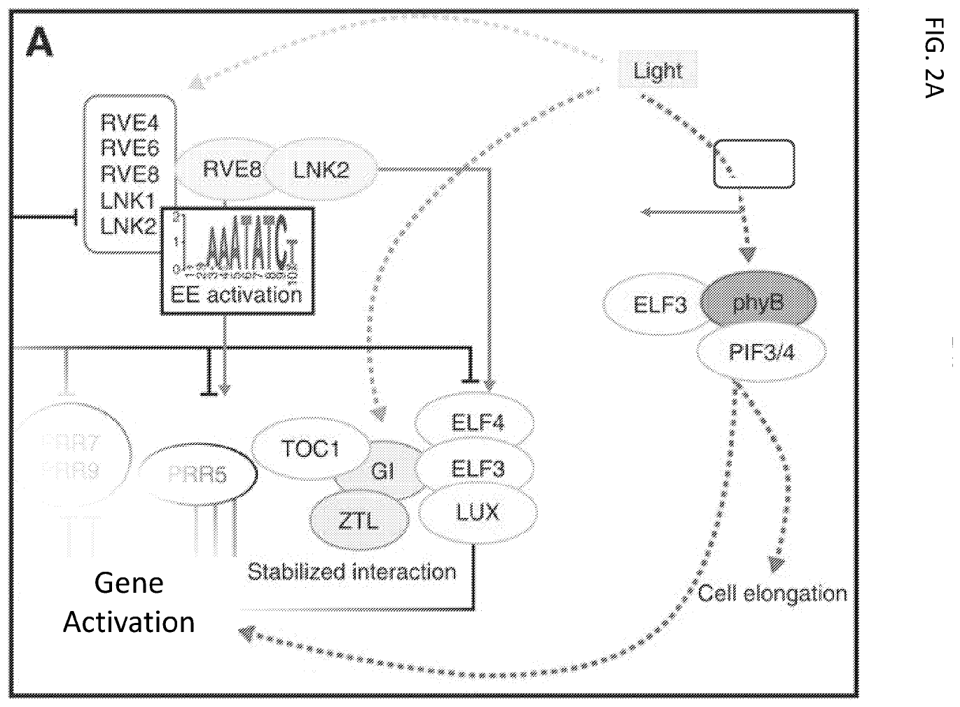

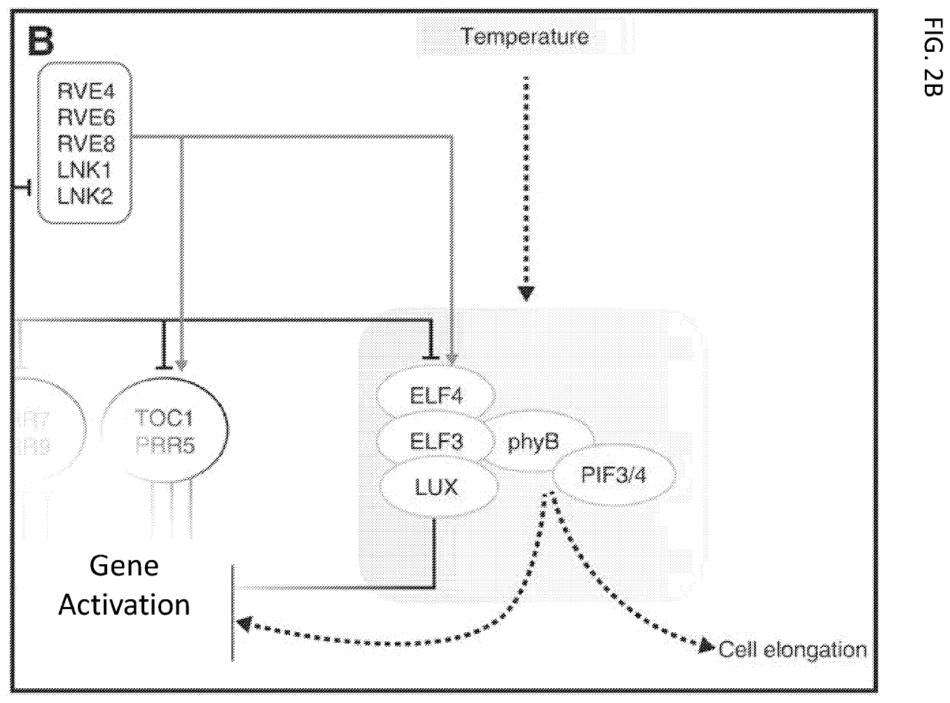

ZEITLUPE (ZTL) is unique in being both a component of the plant clock and a blue-light photoreceptor. ZTL interacts directly with GIGANTEA (GI), another clock component, and this interaction is stabilized by blue light via the photosensory LOV domain of ZTL. This ZTL-GI complex can maintain circadian rhythms by influencing the stability of both TOC1 and GI proteins. GI stability is also affected by a second protein complex that includes ELF3 and COP1 (CONSTITUTIVE PHOTOMORPHOGENIC1) that acts downstream from the blue light photoreceptor CRY2. The ELF3-COP1 complex targets GI for degradation and represents yet another point at which light signals are integrated into the circadian clock network. The LNK2 and RVE8 complex also appears to play a role in the integration of the clock and light-signaling pathways (FIG. 2A). It is possible that clock entrainment relies on the induction of LNK expression by phytochromes in conjunction with the early morning expression of CCA1 and LHY.

A modified plant of the instant disclosure can comprise nucleic acid modifications that modify expression of any protein or factor that functions in the circadian clock of a plant. Accordingly, a modified plant of the instant disclosure can comprise a genetic modification of a polynucleotide encoding an MYB-like transcription factor, a PSEUDO-RESPONSE REGULATOR (PRR) protein, a protein of the evening complex (EC), a zeitlupe protein, or any combination thereof. In some aspects, a modified plant of the instant disclosure comprises a genetic modification of a polynucleotide encoding an MYB-like transcription factor that modifies the expression of the MYB-like transcription factor. In other aspects, a modified plant of the instant disclosure comprises a genetic modification of a polynucleotide encoding a PRR protein that modifies the expression of the MYB-like transcription factor PRR protein. In yet other aspects, a modified plant of the instant disclosure comprises a genetic modification of a polynucleotide encoding a protein of the evening complex that modifies the expression of the protein of the evening complex. In some aspects, a modified plant of the instant disclosure comprises a genetic modification of a zeitlupe protein that modifies the expression of the zeitlupe protein.

In some aspects, a modified plant of the instant disclosure comprises a genetic modification of a polynucleotide encoding CCA1, LHY, PRR1/TOC1, PRR5, PRR7, PRR9, ELF3, ELF4, LUX, ZTL, FKF1, LKP2, GI, COP1, LNK2, REV4, REV6, RVE8, LNK1, LNK2, TOC1, or any combination thereof. In other aspects, a modified plant of the instant disclosure comprises a genetic modification of a polynucleotide encoding ELF3, ELF4, LUX, or any combination thereof. In some aspects, the modified plant comprises a genetic modification of a polynucleotide encoding ELF4. In some aspects, the modified plant comprises a genetic modification of a polynucleotide encoding LUX.

In some aspects, the modified plant comprises a genetic modification of a polynucleotide encoding ELF3. In some aspects, a genetic modification of a polynucleotide encoding ELF3 modifies expression of ELF3. In some aspects, a genetic modification of a polynucleotide encoding ELF3 reduces expression of ELF3. Methods of reducing expression of a protein or RNA in a plant are known in the art and include, without limitation, introducing a genetic modification in an endogenous nucleic acid sequence that regulates expression of the protein, introducing a programmable transcription regulation system, or modifying the protein to generate a defective protein. In some aspects, expression of the ELF3 protein is reduced. In some aspects, expression of the ELF3 protein is reduced by introducing into the plant a loss of function mutation in an endogenous polynucleotide expressing ELF3.

In some aspects, the modified plant is a modified Arabidopsis thaliana (A. thaliana). When the plant is A. thaliana, the polynucleotide encoding ELF3 can comprise a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 1. In some aspects, the polynucleotide encoding ELF3 comprises a nucleic acid sequence comprising at least about 75% or more, at least about 85% or more, at least about 95% or more, or 100% sequence identity with a nucleic acid sequence of SEQ ID NO: 1. In some aspects, the polynucleotide encoding ELF3 comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 2. In some aspects, the polynucleotide encoding ELF3 comprises an amino acid sequence comprising at least about 75% or more, at least about 85% or more, at least about 95% or more, or 100% sequence identity with an amino acid sequence of SEQ ID NO: 2.

In some aspects, a modified plant of the instant disclosure comprises a genetic modification of a polynucleotide comprising a loss-of-function mutation in a nucleic acid sequence encoding ELF3. In some aspects, a nucleic acid sequence encoding ELF3 and comprising a loss-of-function mutation comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 3, SEQ ID NO: 4, or a combination thereof. In some aspects, a nucleic acid sequence encoding ELF3 and comprising a loss-of-function mutation comprises about 75% or more, at least about 85% or more, at least about 95% or more, or 100% sequence identity with a nucleic acid sequence of SEQ ID NO: 3, SEQ ID NO: 4, or a combination thereof.

In some aspects, the modified plant is a modified Thlaspi arvense (T. arvense; pennycress). In some aspects, the polynucleotide encoding ELF3 comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 20. In some aspects, the polynucleotide encoding ELF3 comprises a nucleic acid sequence comprising at least about 75% or more, at least about 85% or more, at least about 95% or more, or 100% sequence identity with a nucleic acid sequence of SEQ ID NO: 20.

In some aspects, the polynucleotide encoding ELF3 comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 21. In some aspects, the polynucleotide encoding ELF3 comprises an amino acid sequence comprising at least about 75% or more, at least about 85% or more, at least about 95% or more, or 100% sequence identity with an amino acid sequence of SEQ ID NO: 21.

In some aspects, a modified plant of the instant disclosure comprises a genetic modification of a polynucleotide comprising a loss-of-function mutation in a nucleic acid sequence encoding ELF3. In some aspects, the genetic modification of the polynucleotide encoding the ELF3 protein comprises a deletion of a nucleic acid segment in the polynucleotide encoding the ELF3 protein, wherein the nucleic acid segment comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 22, a sequence comprising the AGCGCTA nucleic acid sequence, or a combination thereof. In some aspects, the genetic modification of the polynucleotide encoding the ELF3 protein comprises a deletion of a nucleic acid segment in the polynucleotide encoding the ELF3 protein, wherein the nucleic acid segment comprises about 75% or more, at least about 85% or more, at least about 95% or more, or 100% sequence identity with a nucleic acid sequence of SEQ ID NO: 22, a sequence comprising the AGCGCTA nucleic acid sequence, or a combination thereof.

In some aspects, the genetic modification of the polynucleotide encoding the ELF3 protein comprises a genetic modification introduced by a programmable nucleic acid modification system comprising a CRISPR/Cas nuclease system, wherein the CRISPR/Cas nuclease system comprises a CAS9 protein (dCAS9) and a guide RNA (gRNA). In some aspects, the Cas9 protein is encoded by a a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 29 and the gRNA comprises a nucleic acid sequence of SEQ ID NO: 30, SEQ ID NO: 31, or a combination thereof. In some aspects, the Cas9 protein is encoded by a a nucleic acid sequence comprising at least about 75% or more, at least about 85% or more, at least about 95% or more, or 100% sequence identity with a nucleic acid sequence of SEQ ID NO: 29 and the gRNA comprises a nucleic acid sequence of SEQ ID NO: 30, SEQ ID NO: 31, or a combination thereof.

In some aspects, the modified plant is a modified basil plant. In some aspects, the polynucleotide encoding ELF3 comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 13. In some aspects, the polynucleotide encoding ELF3 comprises a nucleic acid sequence comprising at least about 75% or more, at least about 85% or more, at least about 95% or more, or 100% sequence identity with a nucleic acid sequence of SEQ ID NO: 13.

In some aspects, the genetic modification of the polynucleotide encoding the ELF3 protein comprises a genetic modification introduced by a programmable nucleic acid modification system comprising a CRISPR/Cas nuclease system, wherein the CRISPR/Cas nuclease system comprises a CAS9 protein (dCAS9) and a guide RNA (gRNA). In some aspects, the Cas9 protein is encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 29 and the gRNA comprises a nucleic acid sequence of SEQ ID NOs: 33-36, or any combination thereof. In some aspects, the Cas9 protein is encoded by a nucleic acid sequence comprising at least about 75% or more, at least about 85% or more, at least about 95% or more, or 100% sequence identity with a nucleic acid sequence of SEQ ID NO: 29 and the gRNA comprises a nucleic acid sequence of SEQ ID NOs: 33-36, or any combination thereof.

### (b) Environmental Signal

A modified plant of the instant disclosure comprises a genetic modification of a polynucleotide encoding a component of the circadian clock. The genetic modification of the polynucleotide encoding a component of the circadian clock modifies the ability of a plant to receive environmental signal, respond to a received environmental signal, or both. In some aspects, a genetic modification of the polynucleotide encoding a component of the circadian clock modifies expression of the environmental signal sensor. In some aspects, the modification of a polynucleotide encoding a component of the circadian clock comprises a modification of an endogenous polynucleotide sequence encoding an environmental signal sensor. In some aspects, modification of the endogenous polynucleotide encoding the environmental signal sensor modifies expression of the environmental signal sensor. In some aspects, the modification of a polynucleotide encoding a component of the circadian clock comprises an introduced exogenous polynucleotide sequence encoding an environmental signal sensor. In some aspects, the introduced exogenous polynucleotide sequence encoding an environmental signal sensor modifies expression of the environmental signal sensor.

Circadian clocks must be continually adjusted by environmental cues so that the processes they control are appropriately timed even as temperature, day-length, and other environmental cues change throughout the life of the plant. Non-limiting examples of environmental signals that can influence the circadian clock include photoperiod, light intensity and quality, temperature, chemicals, gravity, moisture, biotic or abiotic stress, oxygen and carbon dioxide concentrations, carbohydrate availability, or any combination thereof.

The plant clock uses multiple mechanisms to sense and integrate external environmental signals into the feedback loops described above (FIG. 2A-B). Non-limiting examples of environmental signal sensors through which plants sense and communicate the environmental signal to the circadian clock include photoreceptors, temperature sensors, CO₂ sensors, O₂ sensors, ethylene sensors, gravitropic sensors, or any combination thereof. Accordingly, a modified plant of the instant disclosure can comprise a genetic modification of a polynucleotide encoding one or more of a photoreceptor, a temperature sensor, or any combination thereof.

### A. Photoreceptors

In some aspects, a modified plant of the instant disclosure comprises a genetic modification of a polynucleotide encoding a photoreceptor, wherein the nucleic acid modification modifies the expression of the photoreceptor and the response of the plant to changes in photoperiod, intensity, and quality, or any combination thereof. Photoreceptors can sense environmental cues, such as irradiance, day-night transition, photoperiods, and light quality. Non-limiting examples of photoreceptors include phytochromes, phototropins, cryptochromes, F-box containing Flavin binding proteins (e.g., ZEITLUPE, FKF1/LKP2), UV-B resistance 8 (UVR8), or any combination thereof.

In some aspects, a modified plant of the instant disclosure comprises a genetic modification of a polynucleotide encoding a phototropin. Phototropins are photoreceptor proteins (more specifically, flavoproteins) that mediate phototropism responses in higher plants. Phototropins can be important for the opening of stomata and the movement of chloroplasts. These blue light receptors are seen across the entire green plant lineage. Phototropins are part of the phototropic sensory system in plants that causes various environmental responses in plants. Phototropins specifically will cause stems to bend towards light and stomata to open. Phototropins have been shown to impact the movement of chloroplast inside the cell. In addition, phototropins mediate the first changes in stem elongation in blue light prior to cryptochrome activation. Phototropins are also required for blue light mediated transcript destabilization of specific mRNAs in the cell. Non-limiting examples of phototropins include PHOT1 and PHOT2, or any combination thereof. In some aspects, a modified plant of the instant disclosure comprises a genetic modification of a polynucleotide encoding PHOT1 that modifies the expression of PHOT1. In some aspects, a modified plant of the instant disclosure comprises a genetic modification of a polynucleotide encoding PHOT2 that modifies the expression of PHOT2.

In some aspects, a modified plant of the instant disclosure comprises a genetic modification of a polynucleotide encoding a cryptochrome, wherein the nucleic acid modification modifies the expression of the cryptochrome. Cryptochromes (CRY) are UV-A/blue photoreceptors ubiquitously found in bacteria, fungi, animals, and plants sharing a common evolutionary ancestor with DNA photolyases. Cryptochromes regulate growth and development in plants and the circadian clock in plants and animals. The Arabidopsis genome encodes three cryptochrome genes, CRY1, CRY2, and CRY3. CRY1 and CRY2 act primarily in the nucleus, whereas CRY3 likely functions in chloroplasts and mitochondria. Arabidopsis CRY1 and CRY2 mediate primarily blue light regulation of de-etiolation and photoperiodic control of flowering, respectively. In addition, these two photoreceptors regulate other aspects of plant growth and development, including entrainment of the circadian clock, guard cell development and stomatal opening, root growth, plant height, fruit and ovule size, tropic growth, apical dominance, apical meristem activity, programmed cell death, the high-light stress response, osmotic stress response, shade avoidance, and responses to bacterial and viral pathogens. Arabidopsis CRY3 belongs to the CRY-DASH clade of the photolyase/cryptochrome superfamily, and it is known to act as a single-stranded DNA repairing enzyme. However, CRY-DASH of some organisms have been reported to possess both DNA-repairing enzyme activity and photosensory activity. Arabidopsis CRY3 can also act as a dual function photoreceptor in mitochondria and chloroplasts. In some aspects, a modified plant of the instant disclosure comprises a genetic modification of a polynucleotide encoding CRY1 that modifies the expression of CRY1. In some aspects, a modified plant of the instant disclosure comprises a genetic modification of a polynucleotide encoding CRY2 that modifies the expression of CRY2. In some aspects, a modified plant of the instant disclosure comprises a genetic modification of a polynucleotide encoding CRY3 that modifies the expression of CRY3.

In some aspects, a modified plant of the instant disclosure comprises a genetic modification of a polynucleotide encoding a ZEITLUPE (ZTL) protein that modifies the expression of zeitlupe protein. Zeitlupes can be as described in Section 1(a) herein above.

In some aspects, a modified plant of the instant disclosure comprises a genetic modification of a polynucleotide encoding a UVR8 protein that modifies the expression of the phytochrome UVR8 protein. UVR8 is a UV-B—sensing protein found in plants. It is responsible for sensing ultraviolet light in the range 280-315 nm and initiating the plant stress response. UVR8 has also been shown to be functionally linked with the circadian clock in plants.

In some aspects, a modified plant of the instant disclosure comprises a genetic modification of a polynucleotide encoding a phytochrome that modifies the expression of the phytochrome. The phytochrome-signaling pathway is one of the main mechanisms through which plants sense and respond to changes in red light availability and is directly linked to the clock regulatory network. Phytochromes (phys) are the primary red and far-red light photoreceptors in plants and consist of a five-member protein family in Arabidopsis; PhyA, PhyB, PhyC, PhyD, and PhyE. Phys reversibly switch between Pr and Ptr forms upon absorption of red or far-red light, respectively, and the ratio of these forms within the cell controls the shade avoidance response and contributes to light perception. PhyA is the most divergent phy, with a specialized role as a far-red light sensor, whereas phyB is the predominant red light sensor in Arabidopsis. Although oscillation of circadian transcripts is dampened under constant far-red light, phyA retains photoregulatory control of these and other genes under these conditions. By contrast, phyA, phyB, phyC, and phyD each appear to contribute to maintenance of circadian rhythms under constant red light. PhyD single mutants have a wild-type circadian phenotype but have an additive effect when introgressed into a phyB background.

Of the phytochrome receptors, phytochrome B (phyB) is the main red-light receptor and its effects on plant growth and development have been extensively studied. PhyB photoconverts from an inactive form (Pr) to an active form (Pfr) on absorption of red light. Pfr interacts with the PHYTOCHROME-INTERACTING FACTORS (PIFs) and targets these transcription factors for degradation during the day to limit cell elongation to the nighttime hours. PIF proteins have been established as transcriptional regulators of morning expressed LHY and CCA1, directly linking the light and clock regulatory networks. Recently, PIFs have also been shown to mediate metabolic signals to the circadian oscillator. Another link between the clock- and light-signaling pathways occurs via interactions between phyB and the evening complex protein ELF3. ELF3 also binds to PIF4 independently of the other evening complex components to repress PIF4 function, thus regulating a light-signaling component controlling hypocotyl elongation. Similarly, TOC1 and other PRR proteins have been shown to bind directly to PIF3 and PIF4 and inhibit their ability to activate transcription. Thus, the association of PRR factors with PIFs on the G-box elements of target promoters serves to limit PIF transactivation function to the predawn hours. The PRRs and the evening complex have also been shown to regulate transcription of PIF genes. Thus, both ELF3 and the PRR proteins limit the function and expression of the important growth regulatory PIF factors and provide further links between clock and light regulation of growth (FIG. 2A).

Accordingly, a modified plant of the instant disclosure can comprise a genetic modification of a polynucleotide encoding one or more of phyA, phyB, phyC, phyD, phyE, a PIF protein, or any combination thereof. In some aspects, a modified plant of the instant disclosure comprises a genetic modification of a polynucleotide encoding phyA. In some aspects, a modified plant of the instant disclosure comprises a genetic modification of a polynucleotide encoding phyC. In some aspects, a modified plant of the instant disclosure comprises a genetic modification of a polynucleotide encoding phyD. In some aspects, a modified plant of the instant disclosure comprises a genetic modification of a polynucleotide encoding phyE.

In some aspects, a modified plant of the instant disclosure comprises a genetic modification of a polynucleotide encoding PhyB. In some aspects, the genetic modification of the polynucleotide encoding the PhyB photoreceptor modifies expression of the PhyB photoreceptor. In some aspects, the polynucleotide encoding PhyB comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 5 or SEQ ID NO: 27. In some aspects, the polynucleotide encoding PhyB comprises a nucleic acid sequence comprising at least about 75% or more, at least about 85% or more, at least about 95% or more, or 100% sequence identity with a nucleic acid sequence of SEQ ID NO: 5 or SEQ ID NO: 27. In some aspects, the polynucleotide encoding PhyB comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 6 or SEQ ID NO: 28. In some aspects, the polynucleotide encoding PhyB comprises an amino acid sequence comprising at least about 75% or more, at least about 85% or more, at least about 95% or more, or 100% sequence identity with a nucleic acid sequence of SEQ ID NO: 6 or SEQ ID NO: 28.

In some aspects, a genetic modification of a polynucleotide encoding PhyB increases expression of PhyB. For instance, a genetic modification can increase expression of PhyB by, without limitation, overexpressing PhyB, constitutively expressing PhyB, expressing a constitutively active PhyB photoreceptor, or any combination thereof. In some aspects, a genetic modification can increase expression of PhyB by overexpressing PhyB. Methods of overexpressing a protein or RNA in a plant are known in the art and include, without limitation, introducing an expression construct expressing the protein to thereby generate a modified plant comprising multiple copies of the polypeptide encoding the protein, introducing an expression construct expressing the protein under the control of a constitutive promoter, introducing a genetic modification in an endogenous nucleic acid sequence that regulates expression of the protein, introducing a programmable transcription regulation system, or modifying the protein to generate a constitutively active protein. In some aspects, the phytochrome B photoreceptor is overexpressed, constitutively expressed, or constitutively active, or any combination thereof. In some aspects, the phytochrome B receptor is overexpressed, constitutively expressed, and constitutively active.

In some aspects, a genetic modification of a polynucleotide encoding a phyB photoreceptor encodes a constitutively active phyB photoreceptor. In some aspects, a genetic modification of a polynucleotide encoding a phyB photoreceptor encodes a constitutively expressed and constitutively active phyB photoreceptor.

The constitutively active phyB photoreceptor can be encoded by a polynucleotide comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 7, SEQ ID NO: 11, SEQ ID NO: 25, or any combination thereof. In some aspects, the constitutively active phyB photoreceptor is encoded by a polynucleotide comprising at least about 75% or more, at least about 85% or more, at least about 95% or more, or 100% sequence identity with a nucleic acid sequence of SEQ ID NO: 7, SEQ ID NO: 11, SEQ ID NO: 25, or any combination thereof. The constitutively active phyB photoreceptor can comprise an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 8 SEQ ID NO: 12, SEQ ID NO: 26, or any combination thereof. In some aspects, the constitutively active phyB photoreceptor comprises an amino acid sequence comprising at least about 75% or more, at least about 85% or more, at least about 95% or more, or 100% sequence identity with an amino acid sequence of SEQ ID NO: 8 SEQ ID NO: 12, SEQ ID NO: 26, or any combination thereof.

In some aspects, the modified plant is a modified A. thaliana. When the plant is A. thaliana, the plant can comprise a genetic modification of a polynucleotide encoding a phyB photoreceptor comprising a modified activity. In some aspects, a modified plant of the instant disclosure comprises a genetic modification of a polynucleotide encoding a constitutively active phyB photoreceptor. In some aspects, the constitutively active phyB photoreceptor is YHB and is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 7, SEQ ID NO: 11, SEQ ID NO: 25, or any combination thereof. In some aspects, the constitutively active phyB photoreceptor is YHB and is encoded by a nucleic acid sequence comprising about 75% or more, at least about 85% or more, at least about 95% or more, or 100% sequence identity with a nucleic acid sequence of SEQ ID NO: 7, SEQ ID NO: 11, SEQ ID NO: 25, SEQ ID NO: 27, or any combination thereof. In some aspects, the amino acid sequence of the constitutively active YHB comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 8 SEQ ID NO: 12, SEQ ID NO: 26, or any combination thereof. In some aspects, the amino acid sequence of YHB comprises about 75% or more, at least about 85% or more, at least about 95% or more, or 100% sequence identity with an amino acid sequence of SEQ ID NO: 8 SEQ ID NO: 12, SEQ ID NO: 26, or any combination thereof.

In some aspects, a modified plant of the instant disclosure comprises a genetic modification of a polynucleotide encoding a phyB photoreceptor comprising a modified activity. In some aspects, a modified plant of the instant disclosure comprises a genetic modification of a polynucleotide encoding a constitutively active phyB photoreceptor. In some aspects, the constitutively active phyB photoreceptor is YHB and is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 7, SEQ ID NO: 11, or a combination thereof. In some aspects, the constitutively active phyB photoreceptor is YHB and is encoded by a nucleic acid sequence comprising about 75% or more, at least about 85% or more, at least about 95% or more, or 100% sequence identity with a nucleic acid sequence of SEQ ID NO: 7, SEQ ID NO: 11, or a combination thereof. In some aspects, the amino acid sequence of the constitutively active YHB comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 8 SEQ ID NO: 12, or a combination thereof. In some aspects, the amino acid sequence of YHB comprises about 75% or more, at least about 85% or more, at least about 95% or more, or 100% sequence identity with an amino acid sequence of SEQ ID NO: 8 SEQ ID NO: 12, or a combination thereof.

In some aspects, the modified plant is a modified T. arvense. In some aspects, the modified plant is Thlaspi arvensis (T. arvensis). In some aspects, the modified T. arvensis plant comprises a genetic modification of a polynucleotide encoding PhyB, wherein the polynucleotide comprising the genetic modification encodes a constitutively active PhyB photoreceptor.

In some aspects, the constitutively active PhyB photoreceptor comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 8 SEQ ID NO: 12, SEQ ID NO: 26, or any combination thereof. In some aspects, the constitutively active PhyB photoreceptor comprises an amino acid sequence comprising at least about 75% or more, at least about 85% or more, at least about 95% or more, or 100% sequence identity with an amino acid sequence of SEQ ID NO: 8 SEQ ID NO: 12, SEQ ID NO: 26, or any combination thereof.

In some aspects, the constitutively active PhyB photoreceptor is encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 7, SEQ ID NO: 11, SEQ ID NO: 25, or any combination thereof. In some aspects, the constitutively active PhyB photoreceptor is encoded by a nucleic acid sequence comprising at least about 75% or more, at least about 85% or more, at least about 95% or more, or 100% sequence identity with an amino acid sequence of SEQ ID NO: 7, SEQ ID NO: 11, SEQ ID NO: 25, or any combination thereof.

In some aspects, the constitutively active PhyB photoreceptor comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 26. In some aspects, the constitutively active PhyB photoreceptor comprises an amino acid sequence comprising at least about 75% or more, at least about 85% or more, at least about 95% or more, or 100% sequence identity with an amino acid sequence of SEQ ID NO: 26.

In some aspects, the constitutively active PhyB photoreceptor is encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 25. In some aspects, the constitutively active PhyB photoreceptor is encoded by a nucleic acid sequence comprising at least about 75% or more, at least about 85% or more, at least about 95% or more, or 100% sequence identity with an amino acid sequence of SEQ ID NO: 25.

### B. Temperature Sensors

Temperature is another environmental signal that is integrated into the clock network (FIG. 2B). Accordingly, a modified plant of the instant disclosure can comprise a modified response to temperature changes.

A temperature sensor can be a photoreceptor as plant photoreceptors can also function as temperature receptors. One such receptor is phyB, with the rate of reversion from the active Pfr form to the inactive Pr form increasing at higher temperatures.

In some aspects, a modified plant of the instant disclosure comprising a modified response to temperature changes comprises a genetic modification in a polynucleotide encoding a phyB protein that modifies the activity of the phyB protein, thereby modifying the response of the modified plant to temperature changes. In some aspects, a modified plant of the instant disclosure comprises a genetic modification of a polynucleotide encoding a phyB photoreceptor comprising a modified activity. In some aspects, a modified plant of the instant disclosure comprises a genetic modification of a polynucleotide encoding a constitutively active phyB photoreceptor. PhyB photoreceptors, genetic modifications of polynucleotides encoding PhyB photoreceptors, and modified plants comprising the polynucleotides can be as described in Section I(b)(A) herein above.

PIF4 has also been shown to respond to changes in temperature to alter plant development and morphology. Because degradation of PIF4 is promoted by Pfr, the increased rate of Pfr to Pr reversion at higher temperatures may account for the warm temperature-induced posttranscriptional accumulation of PIF4 protein. Accordingly, a modified plant of the instant disclosure comprising a modified response to temperature changes can comprise a genetic modification in a polynucleotide encoding a PIF4 protein that modifies the expression of the PIF4 protein thereby modifying the response of the modified plant to temperature changes.

Temperature has also been shown to regulate the activity of the evening complex. At higher temperatures, association of ELF3 with target promoters is reduced. Thus, in warm conditions, evening-complex mediated repression of targets such as the clock genes PRR7, PRR9, GI, LUX, and the growth regulating PIF4 is relieved, leading to elevated levels of these transcripts during warm nights. Integration of cold temperature cues into the clock network can occur via CBF1/DREB1a (COLD-INDUCIBLE C-REPEAT/DROUGHT-RESPONSIVE ELEMENT-BINDING FACTOR). CBF1 expression is highly induced by cold, and this factor has been shown to bind directly to the promoter of the evening complex component LUX and promote its high-amplitude rhythmic expression at cold temperatures. Intriguingly, phyB and PIF proteins have been reported to repress CBF1 expression, suggesting links between distinct temperature-sensing pathways. Finally, expression of CBF1 is regulated by a number of clock components including the PRRs, the evening complex, and CCA1/LHY, providing yet another example of the ubiquitous feedback loops found in the circadian system. Accordingly, a modified plant of the instant disclosure comprising a modified response to temperature changes can comprise a genetic modification in a polynucleotide encoding any of the above cited proteins thereby modifying the response of the modified plant to temperature changes.

### (c) Genetically Modified Plants

As sessile organisms, plants are unable to seek out environmental conditions optimal for their growth and development. Instead, plants rely on their remarkable developmental plasticity to adapt to their environment throughout the life of the plant. By integrating environmental information into the regulation of growth and developmental processes, plant can modulate growth and development according to the environment in which the plant is growing. In this way, the final morphology of the plant depends on the environment, defining it as plastic. Plant development starts with a seedling lacking most species-specific characteristics of the adult as a result of plant embryogenesis. Spreading developmental decisions throughout the life cycle provides the opportunity to integrate environmental information into the regulation of these growth and developmental processes, to thereby tune body form and function to the changing environment. For instance, when faced with a spell of unusually elevated temperatures before or during flower initiation and/or development, a plant may respond by reducing the number of flowers or seeds in anticipation of further scarcity of resources.

While this is advantageous in natural conditions where resources and environmental stresses vary across seasons, location, and in response to unusual weather patterns, plasticity can be disadvantageous under cultivated conditions, resulting in art least reduced yields. In modern crop monoculture, plants are grown under intensive agricultural practices, a plant may not need to adjust in anticipation of harsh conditions as the plant is provided with sufficient resources. In at least that sense, plants with reduced plasticity such as plants of the instant disclosure are well suited for intensive, precision outdoor farming, TCEA, and/or vertical farming). For instance, in one aspect, the modified plants of the instant disclosure flower consistently and uniformly regardless of differences in day length, at varied latitudes, in the face of climate change, increasing temperatures and disrupting weather patterns, or any combination thereof. Importantly, genetically modified plants of the instant disclosure comprising the combination of modifications can provide beneficial agronomic traits without any of the deleterious effects that can be associated with each genetic modification individually. For instance, plants comprising a constitutively active phyB photoreceptor when combined with a loss-of-function mutation in an ELF3 gene is not etiolated (a phenotype of a loss-of-function mutation in an ELF3 gene without a constitutively active phyB) and can flower earlier than plants comprising either mutation alone.

Plants of the instant disclosure comprise genetic modifications that modify expression of a component of the circadian clock and expression of an environmental signal sensing function. A genetic modification that modifies expression of a component of the circadian clock can results in a modification of a circadian function. A circadian function can be any developmental, physiological, and/or metabolic function that could be affected by the circadian clock. Non-limiting examples of circadian functions include transitions from shoot elongation, regulation of root gravitropism, altered flowering time, growth cessation of leaves, and timing of germination, the synthesis of chlorophyll, de-etiolation (when a seedling emerges into the light and starts its photo-autotrophic life style), stomata development, transition to flowering, senescence, shade avoidance, elongation of seedlings, size, shape, number, and movement of leaves, and the timing of flowering in adult plants.

Further the resulting plants exhibit agronomic traits beneficial for crop production using modern agricultural practices. Non-limiting examples of beneficial or desirable agronomic traits include accelerated or delayed flowering, consistent flowering, seed quality, seed protein content, seed protein composition, seed oil content, seed oil composition, yield, seed set, response to photoperiod, abiotic stress tolerance, biotic stress tolerance, flowering time and maturity, regulation of circadian clock light response-related flowering, high latitude adaptation, earlier flowering, reduced cellular elongation response to increasing temperature, a uniform time of flowering, consistent secondary metabolite production or any combination thereof. The genetic modifications can stabilize the production of valuable metabolites, compounds, and proteins by reducing fluctuations in metabolism and protein synthesis in plants that produce such molecules for chemistry and pharmaceutical uses. The genetic modifications can reduce temperature-induced architectural changes in plant height, petiole, internode, and organ elongation, allowing plants to maintain their ideotype under temperature changes. The genetic modifications can reduce responsiveness to reduced light quality and shade conditions, preventing unnecessary elongation and competition with neighboring plants.

In some aspects, modified plants of the instant disclosure comprise a genetic modification of a polynucleotide encoding a component of the circadian clock that modifies expression of the component of the circadian clock; and a genetic modification of a polynucleotide encoding an environmental signal sensor that modifies expression of the environmental signal sensor. The modified expression of the component of the circadian clock and the modified expression of the environmental signal sensor causes the plant to have reduced developmental plasticity in response to changes in environmental conditions during growth and modified circadian function. In some aspects, the component of the circadian clock comprising modified expression is an ELF3 protein and the environmental signal sensor is a PhyB photoreceptor. In some aspects, when the component of the circadian clock comprising modified expression is an ELF3 protein and the environmental signal sensor is a PhyB photoreceptor, the modified plant of the instant disclosure comprises a polynucleotide encoding an ELF3 protein wherein the polynucleotide encoding the ELF3 protein comprises an ELF3 loss-of-function mutation and a polynucleotide encoding a constitutively active PhyB photoreceptor. ELF3 proteins, PhyB photoreceptors, polynucleotides encoding the ELF3 proteins and PhyB photoreceptors, genetic modifications of polynucleotides encoding the ELF3 proteins and PhyB photoreceptors, and modified plants comprising the modified polynucleotides can be as described in Sections I(a) and I(b) herein above.

In some aspects, a modified plant of the instant disclosure is A. thaliana. When the modified plant is A. thaliana, the plant can comprise a polynucleotide comprising an ELF3 loss-of-function mutation, wherein the polynucleotide comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 3 (elf3-1), SEQ ID NO: 4 (elf3-2), or a combination thereof. In some aspects, the polynucleotide comprises a nucleic acid sequence comprising at least about 75% or more, at least about 85% or more, at least about 95% or more, or 100% sequence identity with an amino acid sequence of SEQ ID NO: 3 (elf3-1), SEQ ID NO: 4 (elf3-2), or a combination thereof. Further, when the modified plant is A. thaliana, the plant can comprise a polynucleotide encoding a constitutively active PhyB photoreceptor, wherein the polynucleotide comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence SEQ ID NO: 7, SEQ ID NO: 11, or a combination thereof. In some aspects, the polynucleotide comprises a nucleic acid sequence comprising at least about 75% or more, at least about 85% or more, at least about 95% or more, or 100% sequence identity with an amino acid sequence of SEQ ID NO: 7, SEQ ID NO: 11, or a combination thereof. Additionally, when the modified plant is A. thaliana, the plant can comprise a polynucleotide encoding a constitutively active PhyB photoreceptor, wherein the polynucleotide comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence SEQ ID NO: 7. In some aspects, the polynucleotide comprises a nucleic acid sequence comprising at least about 75% or more, at least about 85% or more, at least about 95% or more, or 100% sequence identity with an amino acid sequence of SEQ ID NO: 7.

In some aspects, a modified plant of the instant disclosure is T. arvensis. When the modified plant is T. arvensis, the plant can comprise a polynucleotide comprising an ELF3 loss-of-function mutation, wherein the polynucleotide comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 22, a sequence comprising the AGCGCTA nucleic acid sequence, or a combination thereof. In some aspects, the polynucleotide comprises a nucleic acid sequence comprising at least about 75% or more, at least about 85% or more, at least about 95% or more, or 100% sequence identity with an amino acid sequence of SEQ ID NO: 22, a sequence comprising the AGCGCTA nucleic acid sequence, or a combination thereof. Further, when the modified plant is T. arvensis, the plant can comprise a polynucleotide encoding a constitutively active PhyB photoreceptor, wherein the polynucleotide comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence SEQ ID NO: 25, SEQ ID NO: 27, or a combination thereof. In some aspects, the polynucleotide comprises a nucleic acid sequence comprising at least about 75% or more, at least about 85% or more, at least about 95% or more, or 100% sequence identity with an amino acid sequence of SEQ ID NO: 25, SEQ ID NO: 27, or a combination thereof.

### II. Engineered Nucleic Acid Modification System

One aspect of the present disclosure encompasses a system for modifying an environmental signal sensor in a plant. Non-limiting examples of suitable systems for modifying an environmental signal sensor in a plant include programmable nucleic acid modification systems, an expression constructs comprising a promoter operably linked to a polynucleotide encoding a polypeptide or polynucleotide, or any combination thereof. In some aspects, the system comprises a nucleic acid construct comprising at least one or both of a programmable nucleic acid modification system comprising a targeting nucleic acid sequence targeting a nucleic acid sequence in a polynucleotide sequence encoding a component of the circadian clock; and an expression construct comprising a promoter operably linked to a polynucleotide encoding a polynucleotide encoding an environmental signal sensor. The programmable nucleic acid modification system introduces a loss of function mutation into the polynucleotide sequence encoding the component of the circadian clock, and the expression construct comprising a promoter operably linked to a polynucleotide encoding a polynucleotide encoding an environmental signal sensor increases expression of the environmental signal sensor, thereby causing the plant to exhibit reduced developmental plasticity in response to changes in environmental conditions during growth, and modified circadian function.

In some aspects, the nucleic acid modification system is an expression construct comprising a promoter operably linked to a polynucleotide encoding a component of the circadian clock, an expression construct comprising a promoter operably linked to a polynucleotide encoding an environmental signal sensor, or a combination thereof. In some aspects, the nucleic acid modification system is an expression construct comprising a promoter operably linked to a polynucleotide encoding an environmental signal sensor. Environmental signal sensors, polynucleotides encoding the environmental signal sensors, genetic modifications of polynucleotides encoding the environmental signal sensors, and modified plants comprising the modified polynucleotides can be as described in Section I(b) herein above. Expression constructs can be as described in Section III herein below.

In other aspects, the nucleic acid modification system is a programmable nucleic acid modification system targeted to a nucleic acid sequence in a polynucleotide encoding a component of the circadian clock, encoding an environmental signal sensor, or a combination thereof. In some aspects, the expression construct comprising a promoter operably linked to a polynucleotide encoding a polynucleotide encoding an environmental signal sensor increases expression of the environmental signal sensor. Programmable nucleic acid modification systems are described in Section II(a) herein below.

### (a) Programmable Nucleic Acid Modification Systems

As used herein, a “programmable nucleic acid modification system” is a system capable of targeting a nucleic acid sequence in a polynucleotide and modifying the nucleic acid sequence of the polynucleotide or modifying the expression of the polynucleotide. Accordingly, a programmable nucleic acid modification system can be used to alter a polynucleotide or to alter a protein encoded by the nucleic acid sequence. A programmable nucleic acid modification system can also be used to modify the expression of a nucleic acid sequence encoded by the polynucleotide or modify the expression of a polypeptide encoded by the polynucleotide. In the context of a genetic modification of polynucleotides encoding components of the circadian clock, and environmental signal sensors, the programmable nucleic acid modification system specifically targets a nucleic acid sequence in a polynucleotide encoding the components of the circadian clock, environmental signal sensors, or any combination thereof. The programmable nucleic acid modification system can comprise an interfering nucleic acid molecule or a nucleic acid editing system.

### A. RNAi

In some aspects, the programmable expression modification system comprises an interfering nucleic acid (RNAi) molecule having a nucleotide sequence complementary to a target sequence within a gene encoding the polypeptide or polynucleotide used to inhibit expression of the polypeptide or polynucleotide. RNAi molecules generally act by forming a heteroduplex with a target RNA molecule, which is selectively degraded or “knocked down,” hence inactivating the target RNA. Under some conditions, an interfering RNA molecule can also inactivate a target transcript by repressing transcript translation and/or inhibiting transcription. An interfering RNA is more generally said to be “targeted against” a biologically relevant target, such as a protein, when it is targeted against the nucleic acid encoding the target. For example, an interfering RNA molecule has a nucleotide (nt) sequence which is complementary to an endogenous mRNA of a target gene sequence. Thus, given a target gene sequence, an interfering RNA molecule can be prepared which has a nucleotide sequence at least a portion of which is complementary to a target gene sequence. When introduced into cells, the interfering RNA binds to the target mRNA, thereby functionally inactivating the target mRNA and/or leading to degradation of the target mRNA.

Interfering RNA molecules include, inter alia, small interfering RNA (siRNA), microRNA (miRNA), piwi-interacting RNA (piRNA), long non-coding RNAs (long ncRNAs or lncRNAs), and small hairpin RNAs (shRNA). lncRNAs are widely expressed and have key roles in gene regulation. Depending on their localization and their specific interactions with DNA, RNA and proteins, lncRNAs can modulate chromatin function, regulate the assembly and function of membraneless nuclear bodies, alter the stability and translation of cytoplasmic mRNAs, and interfere with signaling pathways. Piwi-interacting RNA (piRNA) is the largest class of small non-coding RNA molecules expressed in animal cells. piRNAs regulate gene expression through interactions with piwi-subfamily Argonaute proteins. SiRNA are double-stranded RNA molecules, preferably about 19-25 nucleotides in length. When transfected into cells, siRNA inhibit the target mRNA transiently until they are also degraded within the cell. MiRNA and siRNA are biochemically and functionally indistinguishable. Both are about the same in nucleotide length with 5′-phosphate and 3′-hydroxyl ends, and assemble into an RNA-induced silencing complex (RISC) to silence specific gene expression. siRNA and miRNA are distinguished based on origin. siRNA is obtained from long double-stranded RNA (dsRNA), while miRNA is derived from the double-stranded region of a 60-70 nt RNA hairpin precursor. Small hairpin RNAs (shRNA) are sequences of RNA, typically about 50-80 base pairs, or about 50, 55, 60, 65, 70, 75, or about 80 base pairs in length, that include a region of internal hybridization forming a stem loop structure consisting of a base-pair region of about 19-29 base pairs of double-strand RNA (the stem) bridged by a region of single-strand RNA (the loop) and a short 3′ overhang. shRNA molecules are processed within the cell to form siRNA which in turn knock down target gene expression. shRNA can be incorporated into plasmid vectors and integrated into genomic DNA for longer-term or stable expression, and thus longer knockdown of the target mRNA.

Interfering nucleic acid molecules can contain RNA bases, non-RNA bases, or a mixture of RNA bases and non-RNA bases. For example, interfering nucleic acid molecules provided herein can be primarily composed of RNA bases but also contain DNA bases or non-naturally occurring nucleotides. The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone.

### B. Programmable Nucleic Acid Editing Systems

In some aspects, the programmable nucleic acid modification system is a programmable nucleic acid editing system. Editing systems generally comprise a programmable nucleic acid binding domain and a nuclease domain to generate a programmable nucleic acid nuclease system or a transcription factor domain to create a programmable transcription regulation system. A programmable nucleic acid binding domain of an editing system is capable of targeting a nucleic acid sequence in a polynucleotide, a nuclease is capable of facilitating modification of the nucleic acid sequence of the polynucleotide, and a transcription factor is capable of regulating transcription and thus expression of the polynucleotide.

Programmable nucleic acid binding domains rely for specificity on the delivery of exogenous protein(s), and/or a guide RNA (gRNA), deadRNA (dRNA), or single guide RNA (sgRNA) having a sequence which binds specifically to a gene sequence of interest. Non-limiting examples of programmable nucleic acid binding domains that can be used in a programmable nucleic acid editing system include, without limit, a programmable nucleic acid domain of RNA-guided clustered regularly interspersed short palindromic repeats (CRISPR)/CRISPR-associated (Cas) (CRISPR/Cas) nuclease systems, a CRISPR/Cpf1 nuclease system, a zinc finger nuclease (ZFN), a transcription activator-like effector (TALE), a meganuclease, and a ribozyme. Other suitable programmable nucleic acid binding domains will be recognized by individuals skilled in the art.

When the programmable nucleic acid modification system comprises more than one component, such as a binding protein, a nuclease, a transcription factor or a guide nucleic acid, the multi-component modification system can be modular, in that the different components can optionally be distributed among two or more nucleic acid constructs as described herein. The system components can be delivered by a plasmid or viral vector or as a synthetic oligonucleotide. More detailed descriptions of non-limiting examples of programmable nucleic acid editing systems can be as described further below. All the systems described herein below can be modified to comprise a transcription factor, a nuclease, or both.

i. CRISPR Nuclease Systems.

The programmable targeting nuclease can be an RNA-guided CRISPR endonuclease system. The CRISPR system comprises a guide RNA or sgRNA to a target sequence at which a protein of the system introduces a double-stranded break in a target nucleic acid sequence, and a CRISPR-associated endonuclease. The gRNA is a short synthetic RNA comprising a sequence necessary for endonuclease binding, and a preselected ˜20 nucleotide spacer sequence targeting the sequence of interest in a genomic target. Non-limiting examples of endonucleases include Cas1, Cas1 B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof.

The CRISPR nuclease system can be derived from any type of CRISPR system, including a type I (i.e., IA, IB, IC, ID, IE, or IF), type II (i.e., IIA, IIB, or IIC), type III (i.e., IIIA or IIIB), or type V CRISPR system. The CRISPR/Cas system can be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR systems include CRISPR/Cas systems, CRISPR/Cpf systems, CRISPR/Cmr systems, CRISPR/Csa systems, CRISPR/Csb systems, CRISPR/Csc systems, CRISPR/Cse systems, CRISPR/Csf systems, CRISPR/Csm systems, CRISPR/Csn systems, CRISPR/Csx systems, CRISPR/Csy systems, CRISPR/Csz systems, and derivatives or variants thereof. Preferably, the CRISPR system can be a type II Cas9 protein, a type V Cpf1 protein, or a derivative thereof. In some aspects, the CRISPRICas nuclease is Streptococcus pyogenes Cas9 (SpCas9), Streptococcus thermophilus Cas9 (StCas9), Campylobacter jejuni Cas9 (CjCas9), Francisella novicida Cas9 (FnCas9), or Francisella novicida Cpf1 (FnCpf1).

In general, a protein of the CRISPR system comprises an RNA recognition and/or RNA binding domain, which interacts with the guide RNA. A protein of the CRISPR system also comprises at least one nuclease domain having endonuclease activity. For example, a Cas9 protein can comprise a RuvC-like nuclease domain and an HNH-like nuclease domain, and a Cpf1 protein can comprise a RuvC-like domain. A protein of the CRISPR system can also comprise DNA binding domains, helicase domains, RNase domains, protein-protein interaction domains, dimerization domains, as well as other domains.

A protein of the CRISPR system can be associated with guide RNAs (gRNA). The guide RNA can be a single guide RNA (i.e., sgRNA), or can comprise two RNA molecules (i.e., crRNA and tracrRNA). The guide RNA interacts with a protein of the CRISPR system to guide it to a target site in the DNA. The target site has no sequence limitation except that the sequence is bordered by a protospacer adjacent motif (PAM). For example, PAM sequences for Cas9 include 3′-NGG, 3′-NGGNG, 3-NNAGAAW, and 3′-ACAY, and PAM sequences for Cpf1 include 5′-TTN (wherein N is defined as any nucleotide, W is defined as either A or T, and Y is defined as either C or T). Each gRNA comprises a sequence that is complementary to the target sequence (e.g., a Cas9 gRNA can comprise GN17-20GG). The gRNA can also comprise a scaffold sequence that forms a stem loop structure and a single-stranded region. The scaffold region can be the same in every gRNA. In some aspects, the gRNA can be a single molecule (i.e., sgRNA). In other aspects, the gRNA can be two separate molecules. Those skilled in the art are familiar with gRNA design and construction, e.g., gRNA design tools are available on the internet or from commercial sources.

A CRISPR system can comprise one or more nucleic acid binding domains associated with one or more, or two or more selected guide RNAs used to direct the CRISPR system to one or more, or two or more selected target nucleic acid loci. For instance, a nucleic acid binding domain can be associated with one or more, or two or more selected guide RNAs, each selected guide RNA, when complexed with a nucleic acid binding domain, causing the CRISPR system to localize to the target of the guide RNA.

The programmable targeting nuclease can also be a CRISPR nickase system. CRISPR nickase systems are similar to the CRISPR nuclease systems described above except that a CRISPR nuclease of the system is modified to cleave only one strand of a double-stranded nucleic acid sequence. Thus, a CRISPR nickase, in combination with a guide RNA of the system, can create a single-stranded break or nick in the target nucleic acid sequence. Alternatively, a CRISPR nickase in combination with a pair of offset gRNAs can create a double-stranded break in the nucleic acid sequence.

A CRISPR nuclease of the system can be converted to a nickase by one or more mutations and/or deletions. For example, a Cas9 nickase can comprise one or more mutations in one of the nuclease domains, wherein the one or more mutations can be D10A, E762A, and/or D986A in the RuvC-like domain, or the one or more mutations can be H840A (or H839A), N854A and/or N863A in the HNH-like domain.

A nuclease of a CRISPR nuclease system can be inactivated to obtain a programmable targeting protein that can be associated with a transcription factor capable of regulating expression of a polynucleotide. For instance, a CRISPR/Cas system can comprise a nuclease-deficient CAS9 protein (dCAS9) fused to a transcriptional activator and a guide RNA (gRNA).

ii. ssDNA-Guided Argonaute Systems.

Alternatively, the programmable targeting nuclease can comprise a single-stranded DNA-guided Argonaute (Ago) protein. Ago proteins are ubiquitously expressed and bind to siRNAs or miRNAs to guide post-transcriptional gene silencing either by destabilization of the mRNA or by translational repression.

Some prokaryotic Agos use single-stranded guide DNAs and create double-stranded breaks in nucleic acid sequences. The ssDNA-guided Ago endonuclease can be associated with a single-stranded guide DNA.

The Ago programmable protein can be derived from Alistipes sp., Aquifex sp., Archaeoglobus sp., Bacteroides sp., Bradyrhizobium sp., Burkholderia sp., Cellvibrio sp., Chlorobium sp., Geobacter sp., Mariprofundus sp., Natronobacterium sp., Parabacteriodes sp., Parvularcula sp., Planctomyces sp., Pseudomonas sp., Pyrococcus sp., Thermus sp., or Xanthomonas sp. For instance, the Ago protein can be Natronobacterium gregoryi Ago (NgAgo). Alternatively, the Ago protein can be Thermus thermophilus Ago (TtAgo). The Ago protein can also be Pyrococcus furiosus (PfAgo).

The single-stranded guide DNA (gDNA) of an ssDNA-guided Argonaute system is complementary to the target site in the nucleic acid sequence. The target site has no sequence limitations and does not require a PAM. The gDNA generally ranges in length from about 15-30 nucleotides. The gDNA can comprise a 5′ phosphate group. Those skilled in the art are familiar with ssDNA oligonucleotide design and construction.

iii. Zinc Finger Programmable Targeting Proteins.

A ZFN programmable nucleic acid binding protein comprises a DNA-binding zinc finger region that can comprise from about two to seven zinc fingers, for example, about four to six zinc fingers, wherein each zinc finger binds three nucleotides. The zinc finger region can be engineered to recognize and bind to any DNA sequence. Zinc finger design tools or algorithms are available on the internet or from commercial sources. The zinc fingers can be linked together using suitable linker sequences. ZFN programmable nucleic acid binding proteins can be linked to a nuclease or a transcription factor to generate a programmable nucleic acid modification system comprising targeting a specific nucleic acid sequence.

iv. Transcription Activator-Like Effector Nuclease Systems.

The programmable targeting protein can also be a transcription activator-like effector (TALE) or the like. TALENs comprise a DNA-binding domain composed of highly conserved repeats derived from transcription activator-like effectors (TALEs) that are linked to a nuclease domain. TALEs are proteins secreted by plant pathogen Xanthomonas to alter transcription of genes in host plant cells. TALE repeat arrays can be engineered via modular protein design to target any DNA sequence of interest. Other transcription activator-like effector nuclease systems can comprise, but are not limited to, the repetitive sequence, transcription activator like effector (RipTAL) system from the bacterial plant pathogenic Ralstonia solanacearum species complex (Rssc). The nuclease domain of TALEs can be any nuclease domain as described above in Section IV(a)(i).

v. Meganucleases or Rare-Cutting Endonuclease Systems.

The programmable targeting nuclease can also be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. Non-limiting examples of meganucleases that can be suitable for the instant disclosure include I-SceI, I-CreI, I-DmoI, or variants and combinations thereof. A meganuclease can be targeted to a specific nucleic acid sequence by modifying its recognition sequence using techniques well known to those skilled in the art.

The programmable targeting nuclease can be a rare-cutting endonuclease or derivative thereof. Rare-cutting endonucleases are site-specific endonucleases whose recognition sequence occurs rarely in a genome, such as only once in a genome. The rare-cutting endonuclease can recognize a 7-nucleotide sequence, an 8-nucleotide sequence, or longer recognition sequence. Non-limiting examples of rare-cutting endonucleases include NotI, AscI, PacI, AsiSI, SbfI, and FseI.

vi. Endonuclease Domains

Non-limiting examples of endonucleases include Cas1, Cas1 B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof.

vii. Transcription Factor Domains

Non-limiting examples of suitable transcription transcriptional activation domains include, without limit, herpes simplex virus VP16 domain, VP64 (which is a tetrameric derivative of VP16), VP160 (i.e., 10×VP16), VP128, p65 activation domain from NFKB, p53 activation domains 1 and 2, heat-shock factor 1 (HSF1) activation domain, MyoD1 activation domain, GCN4 peptide, 10×GCN4, viral R transactivator (Rta), VPR (a fusion of VP64-p65-Rta), p53 activation domains 1 and 2, CREB (cAMP response element binding protein) activation domains, E2A activation domains, activation domains from human heat-shock factor 1 (HSF1), NFAT (nuclear factor of activated T-cells) activation domains, a histone acetyltransferase, activation domains from the Arabidopsis thaliana MYB46, HAM1, HAM2, MYB112, WRKY11, ERF6, or a combination thereof. Engineered transcription activation systems may comprise one transcription activation domain, two transcription activation domains, three transcription activation domains, or more than three transcription activation domains.

Non-limiting examples of transcription repressor domains include Kruppel-associated Box (KRAB), EAR-repression domain (SRDX), and BRD.

viii. Optional Additional Domains.

The programmable targeting nuclease can further comprise at least one nuclear localization signal (NLS), at least one cell-penetrating domain, at least one reporter domain, and/or at least one linker.

In general, an NLS comprises a stretch of basic amino acids. Nuclear localization signals are known in the art (see, e.g., Lange et al., J. Biol. Chem., 2007, 282:5101-5105). The NLS can be located at the N-terminus, the C-terminal, or in an internal location of the fusion protein.

A cell-penetrating domain can be a cell-penetrating peptide sequence derived from the HIV-1 TAT protein. The cell-penetrating domain can be located at the N-terminus, the C-terminal, or in an internal location of the fusion protein.

A programmable targeting nuclease can further comprise at least one linker. For example, the programmable targeting nuclease, the nuclease domain of the targeting nuclease, and other optional domains can be linked via one or more linkers. The linker can be flexible (e.g., comprising small, non-polar (e.g., Gly) or polar (e.g., Ser, Thr) amino acids). Examples of suitable linkers are well known in the art, and programs to design linkers are readily available (Crasto et al., Protein Eng., 2000, 13(5):3096-312). In alternate aspects, the programmable targeting nuclease, the cell cycle regulated protein, and other optional domains can be linked directly.

A programmable targeting nuclease can further comprise an organelle localization or targeting signal that directs a molecule to a specific organelle. A signal can be a polynucleotide or polypeptide signal, or can be an organic or inorganic compound sufficient to direct an attached molecule to a desired organelle. Organelle localization signals can be as described in U.S. Patent Publication No. 20070196334, the disclosure of which is incorporated herein in its entirety.

### (b) Aspects of Engineered Nucleic Acid Modification Systems

In some aspects, the engineered nucleic acid modification system is a programmable nucleic acid editing system. Programmable nucleic acid editing systems can be as described in Section II(a) herein above. In some aspects, the programmable nucleic acid modification system comprises a CRISPR/Cas nuclease system comprising a CAS9 protein (dCAS9) and a guide RNA (gRNA). In some aspects, an expression construct expressing the Cas9 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence starting at base number 1552 to base number 7881 of SEQ ID NO: 32. In some aspects, an expression construct expressing the Cas9 protein comprises a nucleic acid sequence comprising at least about 75% or more, at least about 85% or more, at least about 95% or more, or 100% sequence identity with a nucleic acid sequence starting at base number 1552 to base number 7881 of SEQ ID NO: 32.

In some aspects, the programmable nucleic acid editing system is targeted to a nucleic acid sequence in a polynucleotide encoding a component of the circadian clock. In some aspects, the programmable nucleic acid modification system introduces a loss of function mutation into a polynucleotide encoding a component of the circadian clock. In some aspects, the component of the circadian clock is an ELF3 protein and the programmable nucleic acid editing system is targeted to a nucleic acid sequence in a polynucleotide encoding the ELF3 protein. In some aspects, the programmable nucleic acid editing system introduces a loss of function mutation into a polynucleotide encoding an ELF3 protein. In some aspects, the programmable nucleic acid modification system is a CRISPR/Cas system comprising a Cas9 nuclease and a guide RNA (gRNA) comprising a sequence complementary to a target sequence within a nucleic acid sequence in a polynucleotide encoding an ELF3 protein.

In some aspects, the modified plant is T. arvense. When the modified plant is T. arvense, the programmable nucleic acid modification system can comprise a Cas9 protein encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 29 and one or more gRNAs comprising a nucleic acid sequence of SEQ ID NO: 30, SEQ ID NO: 31, or a combination thereof. In some aspects, when the modified plant is T. arvense, the programmable nucleic acid modification system comprises a Cas9 protein encoded by a nucleic acid sequence comprising at least about 75% or more, at least about 85% or more, at least about 95% or more, or 100% sequence identity with a nucleic acid sequence of SEQ ID NO: 29 and one or more gRNAs comprising a nucleic acid sequence of SEQ ID NO: 30, SEQ ID NO: 31, or a combination thereof.

In some aspects, the modified plant is basil. When the modified plant is basil, the programmable nucleic acid modification system can comprise a Cas9 protein encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 29 and one or more gRNAs comprising a nucleic acid sequence of SEQ ID NOs: 33-36, or any combination thereof. In some aspects, when the modified plant is basil, the programmable nucleic acid modification system comprises a Cas9 protein encoded by a nucleic acid sequence comprising at least about 75% or more, at least about 85% or more, at least about 95% or more, or 100% sequence identity with a nucleic acid sequence of SEQ ID NO: 29 and one or more gRNAs comprising a nucleic acid sequence of SEQ ID NOs: 33-36, or any combination thereof.

### III. Nucleic Acid Constructs

Another aspect of the present disclosure encompasses one or more nucleic acid constructs for genetically modifying a plant. In some aspects, the one or more constructs can modify a component of the circadian clock, modify an environmental signal sensor, or both in a plant.

Any of the multi-component systems described herein are to be considered modular, in that the different components can optionally be distributed among two or more nucleic acid constructs as described herein. The nucleic acid constructs can be DNA or RNA, linear or circular, single-stranded or double-stranded, or any combination thereof. The nucleic acid constructs can be codon-optimized for efficient translation into protein, and possibly for transcription into an RNA donor polynucleotide transcript in the cell of interest. Codon optimization programs are available as freeware or from commercial sources.

The nucleic acid constructs can be used to express one or more components of the system for later introduction into a cell to be modified. Alternatively, the nucleic acid constructs can be introduced into the cell to be modified for expression of the components of the system in the cell. In some aspects, the nucleic acid constructs transiently express the various components of the system. Transiently expressing the system in a plant overcomes the cumbersome regulatory hurdles required for traditionally modified crops. In some aspects, the engineered nucleic acid modification system is expressed in male reproductive tissues, modifies expression of various factors described herein above in male reproductive tissues, or both.

Expression constructs generally comprise DNA coding sequences operably linked to at least one promoter control sequence for expression in a cell of interest. Promoter control sequences can control expression of a system for modifying a component of the circadian clock, a system for modifying an environmental signal sensor, or both in in bacterial (e.g., E. coli) cells or eukaryotic (e.g., yeast, insect, mammalian, or plant) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters.

Suitable eukaryotic constitutive promoter control sequences include, but are not limited to, pU10, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, NphsI promoter, OG-2 promoter, SP-B promoter, SYN1 promoter, and WASP promoter.

Promoters can also be plant-specific promoters, or promoters that can be used in plants. A wide variety of plant promoters are known to those of ordinary skill in the art, as are other regulatory elements that can be used alone or in combination with promoters.

Promoters can be divided into two types, namely, constitutive promoters and non-constitutive promoters. Constitutive promoters are classified as providing for a range of constitutive expression. Thus, some are weak constitutive promoters, and others are strong constitutive promoters. Non-constitutive promoters include tissue-preferred promoters, tissue-specific promoters, cell-type specific promoters, and inducible promoters. Suitable plant-specific constitutive promoter control sequences include, but are not limited to, a CaMV35S promoter, CaMV 19S, GOS2, Arabidopsis At6669 promoter, Rice cyclophilin, Maize H3 histone, Synthetic Super MAS, an opine promoter, a plant ubiquitin (Ubi) promoter, an actin 1 (Act-1) promoter, pEMU, Cestrum yellow leaf curling virus promoter (CYMLV promoter), and an alcohol dehydrogenase 1 (Adh-1) promoter. Other constitutive promoters include those in U.S. Pat. Nos. 5,659,026; 5,608,149; 5,608,144; 5,604,121; 5,569,597; 5,466,785; 5,399,680; 5,268,463; and 5,608,142.

Regulated plant promoters respond to various forms of environmental stresses, or other stimuli, including, for example, mechanical shock, heat, cold, flooding, drought, salt, anoxia, pathogens such as bacteria, fungi, and viruses, and nutritional deprivation, including deprivation during times of flowering and/or fruiting, and other forms of plant stress. For example, the promoter can be a promoter which is induced by one or more, but not limited to one of the following: abiotic stresses such as wounding, cold, desiccation, ultraviolet-B, heat shock or other heat stress, drought stress or water stress. The promoter can further be one induced by biotic stresses including pathogen stress, such as stress induced by a virus or fungi, stresses induced as part of the plant defense pathway or by other environmental signals, such as light, carbon dioxide, hormones or other signaling molecules such as auxin, hydrogen peroxide and salicylic acid, sugars and gibberellin or abscisic acid and ethylene. Suitable regulated plant promoter control sequences include, but are not limited to, salt-inducible promoters such as RD29A; drought-inducible promoters such as maize rab17 gene promoter, maize rab28 gene promoter, and maize Ivr2 gene promoter; heat-inducible promoters such as heat tomato hsp80-promoter from tomato.

Tissue-specific promoters can include, but are not limited to, fiber-specific, green tissue-specific, root-specific, stem-specific, flower-specific, callus-specific, pollen-specific, egg-specific, promoters specific to male or female reproductive tissues, and seed coat-specific. Suitable tissue-specific plant promoter control sequences include, but are not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993], seed-preferred promoters [e.g., from seed-specific genes (Simon et al., Plant Mol. Biol. 5. 191, 1985; Scofield et al., J. Biol. Chem. 262: 12202, 1987; Baszczynski et al., Plant Mol. Biol. 14: 633, 1990), Brazil Nut albumin (Pearson et al., Plant Mol. Biol. 18: 235-245, 1992), legumin (Ellis et al., Plant Mol. Biol. 10: 203-214, 1988), Glutelin (rice) (Takaiwa et al., Mol. Gen. Genet. 208: 15-22, 1986; Takaiwa et al., FEBS Letts. 221: 43-47, 1987), Zein (Matzke et al., Plant Mol Biol, 143: 323-32, 1990), napA (Stalberg et al., Planta 199: 515-519, 1996), Wheat SPA (Albanietal, Plant Cell, 9: 171-184, 1997), sunflower oleosin (Cummins et al., Plant Mol. Biol. 19: 873-876, 1992)], endosperm specific promoters [e.g., wheat LMW and HMW, glutenin-1 (Mol Gen Genet 216:81-90, 1989; NAR 17:461-2), wheat a, b, and g gliadins (EMBO3:1409-15, 1984), Barley Itrl promoter, barley B1, C, D hordein (Theor Appl Gen 98:1253-62, 1999; Plant J 4:343-55, 1993; Mol Gen Genet 250:750-60, 1996), Barley DOF (Mena et al., The Plant Journal, 116(1): 53-62, 1998), Biz2 (EP99106056.7), Synthetic promoter (Vicente-Carbajosa et al., Plant J. 13: 629-640, 1998), rice prolamin NRP33, rice-globulin Glb-1 (Wu et al., Plant Cell Physiology 39(8) 885-889, 1998), rice alpha-globulin REB/OHP-1 (Nakase et al., Plant Mol. Biol. 33: 513-S22, 1997), rice ADP-glucose PP (Trans Res 6:157-68, 1997), maize ESR gene family (Plant J 12:235-46, 1997), sorgum gamma-kafirin (PMB 32:1029-35, 1996)], embryo-specific promoters [e.g., rice OSH1 (Sato et al., Proc. Natl. Acad. Sci. USA, 93: 8117-8122), KNOX (Postma-Haarsma et al., Plant Mol. Biol. 39:257-71, 1999), rice oleosin (Wu et al., J. Biochem., 123:386, 1998)], and flower-specific promoters [e.g., AtPRP4, chalene synthase (chsA) (Van der Meer et al., Plant Mol. Biol. 15, 95-109, 1990), LAT52 (Twell et al., Mol. Gen Genet. 217:240-245; 1989), apetala-3].

Any of the promoter sequences can be wild type or can be modified for more efficient or efficacious expression. The DNA coding sequence also can be linked to a polyadenylation signal (e.g., SV40 polyA signal, bovine growth hormone (BGH) polyA signal, etc.) and/or at least one transcriptional termination sequence. In some situations, the complex or fusion protein can be purified from the bacterial or eukaryotic cells.

Nucleic acids encoding one or more components of an engineered DNA methylation system and/or transcription activation system can be present in a construct. Suitable constructs include plasmid constructs, viral constructs, and self-replicating RNA (Yoshioka et al., Cell Stem Cell, 2013, 13:246-254). For instance, the nucleic acid encoding one or more components of an engineered DNA methylation system and/or transcription activation system can be present in a plasmid construct.

Non-limiting examples of suitable plasmid constructs include pUC, pBR322, pET, pBluescript, and variants thereof. Alternatively, the nucleic acid encoding one or more components of an engineered DNA methylation system and/or transcription activation system can be part of a viral vector (e.g., lentiviral vectors, adeno-associated viral vectors, adenoviral vectors, and so forth).

The plasmid or viral vector can comprise additional expression control sequences (e.g., enhancer sequences, Kozak sequences, polyadenylation sequences, transcriptional termination sequences, etc.), selectable reporter sequences (e.g., antibiotic resistance genes), origins of replication, T-DNA border sequences, and the like. The plasmid or viral vector can further comprise RNA processing elements such as glycine tRNAs, or Csy4 recognition sites. Such RNA processing elements can, for instance, intersperse polynucleotide sequences encoding multiple gRNAs under the control of a single promoter to produce the multiple gRNAs from a transcript encoding the multiple gRNAs. When a cys4 recognition cite is used, a vector can further comprise sequences for expression of Csy4 RNAse to process the gRNA transcript. Additional information about vectors and use thereof can be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001.

The plasmid or viral vector can also comprise a transit peptide for targeting of a protein product, particularly to a chloroplast, leucoplast or other plastid organelle or vacuole or an extracellular location. For descriptions of the use of chloroplast transit peptides, see U.S. Pat. Nos. 5,188,642 and 5,728,925, herein incorporated by reference in their entirety. Many chloroplast-localized proteins are expressed from nuclear genes as precursors and are targeted to the chloroplast by a chloroplast transit peptide (CTP). Examples of other such isolated chloroplast proteins include, but are not limited to those associated with the small subunit (SSU) of ribulose-1,5-bisphosphate carboxylase, ferredoxin, ferredoxin oxidoreductase, the light-harvesting complex protein I and protein II, thioredoxin F, enolpyruvyl shikimate phosphate synthase (EPSPS) and transit peptides described in U.S. Pat. No. 7,193,133, herein incorporated by reference. It has been demonstrated in vivo and in vitro that non-chloroplast proteins can be targeted to the chloroplast by use of protein fusions with a heterologous CTP and that the CTP is sufficient to target a protein to the chloroplast. Incorporation of a suitable chloroplast transit peptide, such as, the Arabidopsis thaliana EPSPS CTP (CTP2, Klee et al., Mol. Gen. Genet. 210:437-442), and the Petunia hybrida EPSPS CTP (CTP4, della-Cioppa et al., Proc. Natl. Acad. Sci. USA 83:6873-6877) has been show to target heterologous EPSPS protein sequences to chloroplasts in transgenic plants. The production of glyphosate tolerant plants by expression of a fusion protein comprising an amino-terminal CTP with a glyphosate resistant EPSPS enzyme is well known by those skilled in the art, (U.S. Pat. Nos. 5,627,061, 5,633,435, 5,312,910, EP 0218571, EP 189707, EP 508909, and EP 924299).

In some aspects, the one or more constructs comprise one or more expression constructs encoding a system for modifying a component of the circadian clock, a system for modifying an environmental signal sensor, or both in a plant. The constructs can comprise a construct comprising an expression construct for modifying the expression of a component of the circadian clock; a construct comprising an expression construct for modifying the expression of an environmental signal sensor; or a construct comprising an expression construct for modifying the expression of a component of the circadian clock and an expression construct for modifying the expression of an environmental signal sensor.

In some aspects, an expression construct comprises a promoter operably linked to a polynucleotide encoding a component of the circadian clock, an environmental signal sensor, or both. In some aspects, the expression construct comprises a promoter operably linked to a polynucleotide encoding an environmental signal sensor. In some aspects, the environmental sensor is phyB. In one aspect, the phyB photoreceptor is a constitutively expressed phytochrome B photoreceptor. In some aspects, the expression construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 24 or a nucleic acid sequence comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of a nucleic acid sequence starting at base 8148 to base 12020 of SEQ ID NO: 19. In some aspects, the expression construct comprises a nucleic acid sequence comprising about 75% or more, at least about 85% or more, at least about 95% or more, or 100% sequence identity with SEQ ID NO: 24 or a nucleic acid sequence comprising a nucleic acid sequence comprising about 75% or more, at least about 85% or more, at least about 95% or more, or 100% of a nucleic acid sequence starting at base 8148 to base 12020 of SEQ ID NO: 19.

In other aspects, an expression construct comprises a promoter operably linked to a nucleic acid sequence encoding a programmable nucleic acid modification system targeted to a nucleic acid sequence in a polynucleotide encoding a component of the circadian clock, an environmental signal sensor, or both. As used herein, a “programmable nucleic acid modification system” is a system capable of targeting and modifying the nucleic acid or modifying the expression or stability of a nucleic acid to alter a polynucleotide sequence or a protein or the expression of a polynucleotide sequence or protein encoded by the nucleic acid. Programmable nucleic acid modification systems can be as described in Section II herein above.

In some aspects, the programmable nucleic acid modification system is CRISPR/Cas system comprising a Cas9 nuclease and a guide RNA (gRNA) comprising a sequence complementary to a target sequence within the polynucleotide encoding a component of the circadian clock; a gRNA comprising a sequence complementary to a target sequence within the polynucleotide encoding a component of the circadian clock; or a gRNA comprising a sequence complementary to a target sequence within the polynucleotide encoding a component of the circadian clock and a gRNA comprising a sequence complementary to a target sequence within the polynucleotide encoding a component of the circadian clock. In some aspects, the Cas9 nuclease comprises a Cas9 nuclease comprising a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 29. In some aspects, the Cas9 nuclease comprises a Cas9 nuclease comprising a nucleic acid sequence comprising at least about 75% or more, at least about 85% or more, at least about 95% or more, or 100% sequence identity with a nucleic acid sequence of SEQ ID NO: 29.

In some aspects, the plant is Thlaspi arvense. When the plant is Thlaspi arvense, the programmable nucleic acid modification system can be a CRISPR/Cas system comprising a Cas9 nuclease and a gRNA comprising a sequence complementary to a target sequence within a polynucleotide encoding ELF3, and the polynucleotide encoding ELF3 comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 20. In some aspects, the polynucleotide encoding ELF3 comprises a nucleic acid sequence comprising at least about 75% or more, at least about 85% or more, at least about 95% or more, or 100% sequence identity with a nucleic acid sequence of SEQ ID NO: 20. In some aspects, the gRNA comprises a nucleic acid sequence of SEQ ID NO: 30 (gRNA1), SEQ ID NO: 31 (gRNA2), or a combination thereof.

When the plant is Thlaspi arvense, the programmable nucleic acid modification system can be a CRISPR/Cas system comprising a Cas9 nuclease and a gRNA comprising a sequence complementary to a target sequence within a polynucleotide encoding ELF3, and an expression construct encoding SEQ ID NO: 30 (gRNA1) and SEQ ID NO: 31 (gRNA2) comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence starting at base 254 to base 1287 of SEQ ID NO: 32. In some aspects, the expression construct encoding SEQ ID NO: 30 (gRNA1) and SEQ ID NO: 31 (gRNA2) comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence starting at base 254 to base 1287 of SEQ ID NO: 32.

In some aspects, the polynucleotide encoding ELF3 comprises a nucleic acid sequence comprising at least about 75% or more, at least about 85% or more, at least about 95% or more, or 100% sequence identity with a nucleic acid sequence of SEQ ID NO: 20. In some aspects, the gRNA comprises a nucleic acid sequence of SEQ ID NO: 30 (gRNA1), SEQ ID NO: 31 (gRNA2), or a combination thereof.

In some aspects, the plant is Basil. When the plant is Basil, the programmable nucleic acid modification system can be a CRISPR/Cas system comprising a Cas9 nuclease and a gRNA comprising a sequence complementary to a target sequence within a polynucleotide encoding ELF3, and the polynucleotide encoding ELF3 comprises a nucleic acid sequence comprising at least about 75% or more, at least about 85% or more, at least about 95% or more, or 100% sequence identity with a nucleic acid sequence of SEQ ID NO: SEQ ID NO: 13. In some aspects, the gRNA comprises a nucleic acid sequence of SEQ ID NO: 33 to SEQ ID NO: 36, or a combination thereof.

In some aspects, an expression construct expressing the gRNAs of SEQ ID NOs: 33-36 comprises about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 23 or a nucleic acid sequence comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of a nucleic acid sequence starting at base 695 to base 2799 of SEQ ID NO: 37. In some aspects, an expression construct expressing the gRNAs of SEQ ID NOs: 33-36 comprises about 75% or more, at least about 85% or more, at least about 95% or more, or 100% sequence identity with SEQ ID NO: 23 or a nucleic acid sequence comprising a nucleic acid sequence comprising about 75% or more, at least about 85% or more, at least about 95% or more, or 100% of a nucleic acid sequence starting at base 695 to base 2799 of SEQ ID NO: 37.

### IV. Methods

A further aspect of the present disclosure encompasses a method of improving performance of a plant grown under intensive, precision outdoor farming conditions and Total Controlled Environment Agriculture (TCEA), or vertical farming. The method comprises obtaining or having obtained a modified (modified plant) plant having reduced developmental plasticity; and cultivating the plant under intensive, precision outdoor farming conditions, TCEA, or vertical farming. Modified plants can be as described in Section I herein above.

As explained above, the inventors surprisingly discovered that ability of the plant to sense and respond to environmental cues, when combined with a modification of a circadian function of the plant, can reduce the plasticity of the plant in response to changes in environmental conditions during growth all while exhibiting improved agronomic traits regardless of changing environmental conditions. As the pathways and components of pathways for sensing and responding to environmental cues, and circadian functions can be conserved in plants, any plant can be modified using methods of the instant disclosure to generate comprising a modified ability to sense and respond to environmental cues, a modification of a circadian function of the plant. In some aspects, plants of the instant disclosure are A. thaliana. In some aspects, plants of the instant disclosure are T. arvense. In some aspects, plants of the instant disclosure are basil.

In some aspects, methods of the instant disclosure comprise introducing one or more expression constructs for modifying a plant exhibiting reduced developmental plasticity. The one or more expression constructs can comprise at least one or both of an expression construct for expressing a programmable nucleic acid modification system comprising a targeting nucleic acid sequence targeting a nucleic acid sequence in a polynucleotide sequence encoding a component of the circadian clock; and an expression construct for expressing an environmental signal sensor. The plant or plant cell is then grown under conditions whereby the one or more nucleic acid expression constructs express at least one or both of the programmable nucleic acid modification system; and the environmental signal sensor. In some aspects, the method can further comprise introducing the expression constructs. Nucleic acid constructs can be as described in Section III. Programmable nucleic acid modification systems can be as described in Section II herein above.

The expression constructs can comprise an exogenous nucleic acid molecule such as a chimeric nucleic acid of the disclosure. The term “exogenous” as used herein refers to a nucleic acid molecule originating from outside the plant cell. An exogenous nucleic acid molecule can be, for example, the coding sequence of a sterility factor or a fertility restorer. An exogenous nucleic acid molecule can have a naturally occurring or non-naturally occurring nucleotide sequence and can be a heterologous nucleic acid molecule derived from a different organism or a different plant species than the plant cell into which the nucleic acid molecule is introduced or can be a nucleic acid molecule derived from the same plant species as the plant cell into which it is introduced. The exogenous nucleic acid can or cannot be integrated in the plant cell's genome. When said exogenous nucleic acid/gene is not integrated, transient expression of the nucleic acid/gene occurs in the plant cell.

The one or more nucleic acid constructs can be introduced into the cell by a variety of means. Suitable delivery means include microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, viral vectors, magnetofection, lipofection, impalefection, optical transfection, Agrobacterium tumefaciens mediated foreign gene transformation, proprietary agent-enhanced uptake of nucleic acids, and delivery via liposomes, immunoliposomes, virosomes, or artificial virions. The choice of means of introducing the system into a cell can and will vary depending on the cell, or the system or nucleic acid nucleic acid constructs encoding the system, among other variables.

The method can further comprise culturing a cell under conditions suitable for expressing the components of the systems and nucleic acid constructs of the instant disclosure. Methods of culturing cells are known in the art. In some aspects, the cell is from an animal, fungi, oomycete or prokaryote. In some aspects, the cell is a plant cell, plant, or plant part. When the cell is in tissue ex vivo, or in vivo within a plant or within a plant part, the plant part and/or plant can also be maintained under appropriate conditions for insertion of the donor polynucleotide. In general, the plant, plant part, or plant cell is maintained under conditions appropriate for cell growth and/or maintenance. Those of skill in the art appreciate that methods for culturing plant cells are known in the art and can and will vary depending on the cell type. Routine optimization can be used, in all cases, to determine the best techniques for a particular cell type. See for example, in Santiago et al. (2008) PNAS 105:5809-5814; Moehle et al. (2007) PNAS 104:3055-3060; Urnov et al. (2005) Nature 435:646-651; Lombardo et al. (2007) Nat. Biotechnology 25:1298-1306; and Taylor et al. (2012) Tropical Plant Biology 5:127-139.

Another aspect of the instant disclosure encompasses a method of reducing developmental plasticity of a plant. The method comprises generating a modified plant having reduced developmental plasticity. In some aspects, the plant is resistant to changes in growth conditions, which comprises daylength and temperature. Modified plants can be as described in Section I herein above. In some aspects, the plant is resistant to changes in growth conditions of the plant. In other aspects, the growth conditions comprise daylength and temperature.

Yet another aspect of the instant disclosure encompasses a method of reducing a plastic response to competition among a co-cultivated group of plants. The method comprising, co-cultivating a group of plants having reduced developmental plasticity. Modified plants can be as described in Section I herein above.

A yet additional aspect of the instant disclosure encompasses a method of stabilizing production of a metabolite, nucleic acid, or protein in a plant. The method comprising, generating or having generated a plant having reduced developmental plasticity; cultivating the plant for a time sufficient to generate the metabolite, nucleic acid, or protein; and harvesting the metabolite, nucleic acid, or protein from the plant. In some aspects, the plant is grown under intensive, precision outdoor farming conditions, TCEA, or vertical farming.

### V. Kits

A further aspect of the present disclosure provides kits for generating a modified, plant tissue, part thereof, plant cell, or seed having reduced developmental plasticity and improved agronomic traits. The kit can comprise one or more constructs for genetically modifying a plant to generate having reduced developmental plasticity and improved agronomic traits. The one or more constructs can modify a component of the circadian clock, modify an environmental signal sensor, or both in a plant. The kit can further comprise one or more modified plants, plant cells, or parts thereof comprising the one or more constructs can modify a component of the circadian clock, modify an environmental signal sensor, or both in a plant. The modified plants can be as described in Section I and the constructs can be as described in Sections III.

The kits can further comprise transfection reagents, cell growth media, selection media, in vitro transcription reagents, nucleic acid purification reagents, protein purification reagents, buffers, and the like. The kits provided herein generally include instructions for carrying out the methods detailed below. Instructions included in the kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “entrain” as used in the context of a circadian clock refers to the coupling of one rhythm to the same period as another cycle, for example, the resetting of a circadian rhythm from its free-running period to exactly the 24-h period of the day/night cycle.

A “genetically modified” or “modified” plant refers to a plant in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

As used herein, the term “gene” refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, the term “engineered” when applied to a targeting protein refers to targeting proteins modified to specifically recognize and bind to a nucleic acid sequence at or near a target nucleic acid locus.

The term “nucleic acid modification” refers to processes by which a specific nucleic acid sequence in a polynucleotide is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

As used herein, “protein expression” includes but is not limited to one or more of the following: transcription of a gene into precursor mRNA; splicing and other processing of the precursor mRNA to produce mature mRNA; mRNA stability; translation of the mature mRNA into protein (including codon usage and tRNA availability); production of a mutant protein comprising a mutation that modifies the activity of the protein; and glycosylation and/or other modifications of the translation product, if required for proper expression and function.

The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

The term “allele” as used herein refers to one of two or more different nucleotide sequences that occur at a specific locus.

The terms “phenotype”, or “phenotypic trait” or “trait” refer to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait”. In other cases, a phenotype is the result of several genes.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple led, or, more generally, the term genotype can be used to refer to an individual's genetic make-up for all the genes in its genome.

The term “heterozygous” means a genetic condition wherein different alleles reside at corresponding loci on homologous chromosomes.

The term “homozygous” means a genetic condition wherein identical alleles reside at corresponding loci on homologous chromosomes.

The term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines.

“Hybridization” or “nucleic acid hybridization” refers to the pairing of complementary RNA and DNA strands as well as the pairing of complementary DNA single strands.

The term “hybridize” means the formation of base pairs between complementary regions of nucleic acid strands.

The term “inbred” means a line that has been bred for genetic homogeneity.

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

As various changes could be made in the above-described cells and methods without departing from the scope of the invention, it is intended that all matter contained in the above description and in the examples given below, shall be interpreted as illustrative and not in a limiting sense.

SEQUENCES

| SEQ | | | |
|---|---|---|---|
| ID | | | |
| NO. | Sequence | | Description |
| 1 | 1 | atgaagagag ggaaagatga ggagaagata ttggaaccta tgtttcctcg gcttcatgtg | Wild type A. |
| | 61 | aatgatgcag ataaaggagg gcctagagct cctcctagaa acaagatggc tctttatgag | thaliana ELF3 |
| | 121 | cagcttagta ttccttctca gaggtttggt gatcatggaa cgatgaattc tcgtagtaac | cDNA sequence |
| | 181 | aacacaagca ctttggttca tcctggacca tctagtcagc cttgtggtgt ggaaagaaac | |
| | 241 | ttatctgtcc agcatcttga ttcttcagcc gcaaaccaag caactgagaa gtttgtctcc | |
| | 301 | caaatgtcct tcatggaaaa tgtgagatct tcggcacagc atgatcagag gaaaatggtg | |
| | 361 | agagaggaag aagattttgc agttccagta tatattaact caagaagatc tcagtctcat | |
| | 421 | ggcagaacca agagtggtat tgagaaggaa aaacacaccc caatggtggc acctagctct | |
| | 481 | catcactcca ttcgatttca agaagtgaat cagacaggct caaagcaaaa cgtatgtttg | |
| | 541 | gctacttgtt caaaacctga agttagggat caggtcaagg cgaatgcaag gtcaggtggc | |
| | 601 | tttgtaatct ctttagatgt atcagtcaca gaggagattg atctcgaaaa atcagcatca | |
| | 661 | agtcatgata gagtaaatga ttataatgct tccttgagac aagagtctag aaatcggtta | |
| | 721 | taccgagatg gtggcaaaac tcgtctgaag gacactgata atggagctga atctcacttg | |
| | 781 | gcaacggaaa atcattcaca agagggtcat ggcagtcctg aagacattga taatgatcgt | |
| | 841 | gaatacagca aaagcagagc atgcgcctct ctgcagcaga taaatgaaga ggcaagtgat | |
| | 901 | gacgtttctg atgattcgat ggtggattct atatccagca tagatgtctc tcccgatgat | |
| | 961 | gttgtgggta tattaggtca aaaacgtttc tggagagcaa ggaaagccat tgccaatcaa | |
| | 1021 | caaagagtat ttgctgttca actatttgag ttgcacagac tgattaaggt tcaaaaactt | |
| | 1081 | attgctgcat caccggatct cttgctcgat gagatcagtt ttcttggaaa agtttctgct | |
| | 1141 | aaaagctatc cagtgaagaa gctccttcca tcagaatttc tggtaaagcc tcctctacca | |
| | 1201 | catgttgtcg tcaaacaaag gggtgactcg gagaagactg accaacataa aatggaaagc | |
| | 1261 | tcagctgaga acgtagttgg gaggttgtca aatcaaggtc atcatcaaca atccaactac | |
| | 1321 | atgccttttg caaacaaccc accggcttca ccggctccaa atggatattg ctttcctcct | |
| | 1381 | cagcctcctc cttcaggaaa tcatcagcaa tggttgatcc ctgtaatgtc tccctcggaa | |
| | 1441 | ggactgatat acaagcctca cccaggtatg gcacacacgg ggcattatgg aggatattat | |
| | 1501 | ggtcattata tgcctacacc aatggtaatg cctcaatatc accccggcat gggattccca | |
| | 1561 | cctcctggta atggctactt ccctccatat ggaatgatgc ccaccataat gaacccatat | |
| | 1621 | tgttcaagcc aacaacaaca acaacaacaa cccaatgagc aaatgaacca gtttggacat | |
| | 1681 | cctggaaatc ttcagaacac ccaacaacaa caacagagat ctgataatga acctgctcca | |
| | 1741 | cagcaacagc aacagccaac aaagtcttat ccgcgagcaa gaaagagcag gcaagggagc | |
| | 1801 | acaggaagca gtccaagtgg gccacaggga atctctggta gcaagtcctt tcggccattc | |
| | 1861 | gcagccgttg atgaggacag caacatcaac aatgcacctg agcaaacgat gacaacaacc | |
| | 1921 | acaacgacga caagaacaac tgttactcag acaacaagag atgggggagg agtgacgaga | |
| | 1981 | gtgataaagg tggtacctca caacgcaaag ctcgcgagtg agaatgctgc cagaattttc | |
| | 2041 | cagtcaatac aagaagaacg taaacgctat gactcctcta agccttaa | |
| 2 | MKRGKDEEKILEPMFPRLHVNDADKGGPRAPPRNKMALYEQLSIPSQRFGDHGTMNSRSNNTSTLVHPGPS | | Wild type A. |
| | SQPCGVERNLSVQHLDSSAANQATEKFVSQMSFMENVRSSAQHDQRKMVREEEDFAVPVYINSRRSQSHGR | | thaliana ELF3 |
| | TKSGIEKEKHTPMVAPSSHHSIRFQEVNQTGSKQNVCLATCSKPEVRDQVKANARSGGFVISLDVSVTEEI | | protein |
| | DLEKSASSHDRVNDYNASLRQESRNRLYRDGGKTRLKDTDNGAESHLATENHSQEGHGSPEDIDNDREYSK | | |
| | SRACASLQQINEEASDDVSDDSMVDSISSIDVSPDDVVGILGQKRFWRARKAIANQQRVFAVQLFELHRLI | | |
| | KVQKLIAASPDLLLDEISFLGKVSAKSYPVKKLLPSEFLVKPPLPHVVVKQRGDSEKTDQHKMESSAENVV | | |
| | GRLSNQGHHQQSNYMPFANNPPASPAPNGYCFPPQPPPSGNHQQWLIPVMSPSEGLIYKPHPGMAHTGHYG | | |
| | GYYGHYMPTPMVMPQYHPGMGFPPPGNGYFPPYGMMPTIMNPYCSSQQQQQQQPNEQMNQFGHPGNLQNTQ | | |
| | QQQQRSDNEPAPQQQQQPTKSYPRARKSRQGSTGSSPSGPQGISGSKSFRPFAAVDEDSNINNAPEQTMTT | | |
| | TTTTTRTTVTQTTRDGGGVTRVIKVVPHNAKLASENAARIFQSIQEERKRYDSSKP- | | |
| 3 | 1 | atgaagagag ggaaagatga ggagaagata ttggaaccta tgtttcctcg gcttcatgtg | Elf3-1 mutant |
| | 61 | aatgatgcag ataaaggagg gcctagagct cctcctagaa acaagatggc tctttatgag | allele of A. |
| | 121 | cagcttagta ttccttctca gaggtttggt gatcatggaa cgatgaattc tcgtagtaac | thaliana |
| | 181 | aacacaagca ctttggttca tcctggacca tctagtcagc cttgtggtgt ggaaagaaac | (converts |
| | 241 | ttatctgtcc agcatcttga ttcttcagcc gcaaaccaag caactgagaa gtttgtctcc | nucleotide |
| | 301 | caaatgtcct tcatggaaaa tgtgagatct tcggcacagc atgatcagag gaaaatggtg | 1309C to T, |
| | 361 | agagaggaag aagattttgc agttccagta tatattaact caagaagatc tcagtctcat | converts Q 437 |
| | 421 | ggcagaacca agagtggtat tgagaaggaa aaacacaccc caatggtggc acctagctct | to stop) |
| | 481 | catcactcca ttcgatttca agaagtgaat cagacaggct caaagcaaaa cgtatgtttg | |
| | 541 | gctacttgtt caaaacctga agttagggat caggtcaagg cgaatgcaag gtcaggtggc | |
| | 601 | tttgtaatct ctttagatgt atcagtcaca gaggagattg atctcgaaaa atcagcatca | |
| | 661 | agtcatgata gagtaaatga ttataatgct tccttgagac aagagtctag aaatcggtta | |
| | 721 | taccgagatg gtggcaaaac tcgtctgaag gacactgata atggagctga atctcacttg | |
| | 781 | gcaacggaaa atcattcaca agagggtcat ggcagtcctg aagacattga taatgatcgt | |
| | 841 | gaatacagca aaagcagagc atgcgcctct ctgcagcaga taaatgaaga ggcaagtgat | |
| | 901 | gacgtttctg atgattcgat ggtggattct atatccagca tagatgtctc tcccgatgat | |
| | 961 | gttgtgggta tattaggtca aaaacgtttc tggagagcaa ggaaagccat tgccaatcaa | |
| | 1021 | caaagagtat ttgctgttca actatttgag ttgcacagac tgattaaggt tcaaaaactt | |
| | 1081 | attgctgcat caccggatct cttgctcgat gagatcagtt ttcttggaaa agtttctgct | |
| | 1141 | aaaagctatc cagtgaagaa gctccttcca tcagaatttc tggtaaagcc tcctctacca | |
| | 1201 | catgttgtcg tcaaacaaag gggtgactcg gagaagactg accaacataa aatggaaagc | |
| | 1261 | tcagctgaga acgtagttgg gaggttgtca aatcaaggtc atcatcaata atccaactac | |
| | 1321 | atgccttttg caaacaaccc accggcttca ccggctccaa atggatattg ctttcctcct | |
| | 1381 | cagcctcctc cttcaggaaa tcatcagcaa tggttgatcc ctgtaatgtc tccctcggaa | |
| | 1441 | ggactgatat acaagcctca cccaggtatg gcacacacgg ggcattatgg aggatattat | |
| | 1501 | ggtcattata tgcctacacc aatggtaatg cctcaatatc accccggcat gggattccca | |
| | 1561 | cctcctggta atggctactt ccctccatat ggaatgatgc ccaccataat gaacccatat | |
| | 1621 | tgttcaagcc aacaacaaca acaacaacaa cccaatgagc aaatgaacca gtttggacat | |
| | 1681 | cctggaaatc ttcagaacac ccaacaacaa caacagagat ctgataatga acctgctcca | |
| | 1741 | cagcaacagc aacagccaac aaagtcttat ccgcgagcaa gaaagagcag gcaagggagc | |
| | 1801 | acaggaagca gtccaagtgg gccacaggga atctctggta gcaagtcctt tcggccattc | |
| | 1861 | gcagccgttg atgaggacag caacatcaac aatgcacctg agcaaacgat gacaacaacc | |
| | 1921 | acaacgacga caagaacaac tgttactcag acaacaagag atgggggagg agtgacgaga | |
| | 1981 | gtgataaagg tggtacctca caacgcaaag ctcgcgagtg agaatgctgc cagaattttc | |
| | 2041 | cagtcaatac aagaagaacg taaacgctat gactcctcta agccttaa | |
| 4 | 1 | atgaagagag ggaaagatga ggagaagata ttggaaccta tgtttcctcg gcttcatgtg | ELF3-2 mutant |
| | 61 | aatgatgcag ataaaggagg gcctagagct cctcctagaa acaagatggc tctttatgag | sequence of A. |
| | 121 | cagcttagta ttccttctca gaggtttggt gatcatggaa cgatgaattc tcgtagtaac | thaliana |
| | 181 | aacacaagca ctttggttca tcctggacca tctagtcagc cttgtggtgt ggaaagaaac | (nucleotide 101 |
| | 241 | ttatctgtcc agcatcttga ttcttcagcc gcaaaccaag caactgagaa gtttgtctcc | 7 T to G |
| | 301 | caaatgtcct tcatggaaaa tgtgagatct tcggcacagc atgatcagag gaaaatggtg | converting N339 |
| | 361 | agagaggaag aagattttgc agttccagta tatattaact caagaagatc tcagtctcat | to K, |
| | 421 | ggcagaacca agagtggtat tgagaaggaa aaacacaccc caatggtggc acctagctct | and Nucleotide |
| | 481 | catcactcca ttcgatttca agaagtgaat cagacaggct caaagcaaaa cgtatgtttg | 1018 C to T, |
| | 541 | gctacttgtt caaaacctga agttagggat caggtcaagg cgaatgcaag gtcaggtggc | converts Q340 |
| | 601 | tttgtaatct ctttagatgt atcagtcaca gaggagattg atctcgaaaa atcagcatca | to stop, |
| | 661 | agtcatgata gagtaaatga ttataatgct tccttgagac aagagtctag aaatcggtta | followed by |
| | 721 | taccgagatg gtggcaaaac tcgtctgaag gacactgata atggagctga atctcacttg | insertion of |
| | 781 | gcaacggaaa atcattcaca agagggtcat ggcagtcctg aagacattga taatgatcgt | unknown length) |
| | 841 | gaatacagca aaagcagagc atgcgcctct ctgcagcaga taaatgaaga ggcaagtgat | |
| | 901 | gacgtttctg atgattcgat ggtggattct atatccagca tagatgtctc tcccgatgat | |
| | 961 | gttgtgggta tattaggtca aaaacgtttc tggagagcaa ggaaagccat tgccaagtaa | |
| | 1021 | gttcactaga aatttacagt ttggttattt attctccgct ctttctattt atctccttct | |
| | 1081 | ttgataccaa cattttttgc ttgaaagaag ttaatattta agcattgttc cgtagtctta | |
| | 1141 | ctgaagcttt ttcctctgtt gttttttgct attttcattg aggactgtgg tagggcatat | |
| | 1201 | ttcactatca ccaaatttca aatttctaga acactctcct tcatattttt ttttcatgat | |
| | 1261 | taatgctgca attgattgct gatatacata tatgactatt tgtttcagct cgagaagaaa | |
| | 1321 | caaatactca tggaaacata tgagaaggtt tctgaatgac tttaccttaa tcagtctggt | |
| | 1381 | gcaactcaaa tagttgaaca gcaaaatact ctttggtgac tggtaatgga gaaacagagt | |
| | 1441 | c | |
| 5 | 1 | atggtttccg gagtcggggg tagtggcggt ggccgtggcg gtggccgtgg cggagaagaa | A. thaliana |
| | 61 | gaaccgtcgt caagtcacac tcctaataac cgaagaggag gagaacaagc tcaatcgtcg | PhyB wild type |
| | 121 | ggaacgaaat ctctcagacc aagaagcaac actgaatcaa tgagcaaagc aattcaacag | |
| | 181 | tacaccgtcg acgcaagact ccacgccgtt ttcgaacaat ccggcgaatc agggaaatca | |
| | 241 | ttcgactact cacaatcact caaaacgacg acgtacggtt cctctgtacc tgagcaacag | |
| | 301 | atcacagctt atctctctcg aatccagcga ggtggttaca ttcagccttt cggatgtatg | |
| | 361 | atcgccgtcg atgaatccag tttccggatc atcggttaca gtgaaaacgc cagagaaatg | |
| | 421 | ttagggatta tgcctcaatc tgttcctact cttgagaaac ctgagattct agctatggga | |
| | 481 | actgatgtga gatctttgtt cacttcttcg agctcgattc tactcgagcg tgctttcgtt | |
| | 541 | gctcgagaga ttaccttgtt aaatccggtt tggatccatt ccaagaatac tggtaaaccg | |
| | 601 | ttttacgcca ttcttcatag gattgatgtt ggtgttgtta ttgatttaga gccagctaga | |
| | 661 | actgaagatc ctgcgctttc tattgctggt gctgttcaat cgcagaaact cgcggttcgt | |
| | 721 | gcgatttctc agttacaggc tcttcctggt ggagatatta agcttttgtg tgacactgtc | |
| | 781 | gtggaaagtg tgagggactt gactggttat gatcgtgtta tggtttataa gtttcatgaa | |
| | 841 | gatgagcatg gagaagttgt agctgagagt aaacgagatg atttagagcc ttatattgga | |
| | 901 | ctgcattatc ctgctactga tattcctcaa gcgtcaaggt tcttgtttaa gcagaaccgt | |
| | 961 | gtccgaatga tagtagattg caatgccaca cctgttcttg tggtccagga cgataggcta | |
| | 1021 | actcagtcta tgtgcttggt tggttctact cttagggctc ctcatggttg tcactctcag | |
| | 1081 | tatatggcta acatgggatc tattgcgtct ttagcaatgg cggttataat caatggaaat | |
| | 1141 | gaagatgatg ggagcaatgt agctagtgga agaagctcga tgaggctttg gggtttggtt | |
| | 1201 | gtttgccatc acacttcttc tcgctgcata ccgtttccgc taaggtatgc ttgtgagttt | |
| | 1261 | ttgatgcagg ctttcggttt acagttaaac atggaattgc agttagcttt gcaaatgtca | |
| | 1321 | gagaaacgcg ttttgagaac gcagacactg ttatgtgata tgcttctgcg tgactcgcct | |
| | 1381 | gctggaattg ttacacagag tcccagtatc atggacttag tgaaatgtga cggtgcagca | |
| | 1441 | tttctttacc acgggaagta ttacccgttg ggtgttgctc ctagtgaagt tcagataaaa | |
| | 1501 | gatgttgtgg agtggttgct tgcgaatcat gcggattcaa ccggattaag cactgatagt | |
| | 1561 | ttaggcgatg cggggtatcc cggtgcagct gcgttagggg atgctgtgtg cggtatggca | |
| | 1621 | gttgcatata tcacaaaaag agactttctt ttttggtttc gatctcacac tgcgaaagaa | |
| | 1681 | atcaaatggg gaggcgctaa gcatcatccg gaggataaag atgatgggca acgaatgcat | |
| | 1741 | cctcgttcgt cctttcaggc ttttcttgaa gttgttaaga gccggagtca gccatgggaa | |
| | 1801 | actgcggaaa tggatgcgat tcactcgctc cagcttattc tgagagactc ttttaaagaa | |
| | 1861 | tctgaggcgg ctatgaactc taaagttgtg gatggtgtgg ttcagccatg tagggatatg | |
| | 1921 | gcgggggaac aggggattga tgagttaggt gcagttgcaa gagagatggt taggctcatt | |
| | 1981 | gagactgcaa ctgttcctat attcgctgtg gatgccggag gctgcatcaa tggatggaac | |
| | 2041 | gctaagattg cagagttgac aggtctctca gttgaagaag ctatggggaa gtctctggtt | |
| | 2101 | tctgatttaa tatacaaaga gaatgaagca actgtcaata agcttctttc tcgtgctttg | |
| | 2161 | agaggggacg aggaaaagaa tgtggaggtt aagctgaaaa ctttcagccc cgaactacaa | |
| | 2221 | gggaaagcag tttttgtggt tgtgaatgct tgttccagca aggactactt gaacaacatt | |
| | 2281 | gtcggcgttt gttttgttgg acaagacgtt actagtcaga aaatcgtaat ggataagttc | |
| | 2341 | atcaacatac aaggagatta caaggctatt gtacatagcc caaaccctct aatcccgcca | |
| | 2401 | atttttgctg ctgacgagaa cacgtgctgc ctggaatgga acatggcgat ggaaaagctt | |
| | 2461 | acgggttggt ctcgcagtga agtgattggg aaaatgattg tcggggaagt gtttgggagc | |
| | 2521 | tgttgcatgc taaagggtcc tgatgcttta accaagttca tgattgtatt gcataatgcg | |
| | 2581 | attggtggcc aagatacgga taagttccct ttcccattct ttgaccgcaa tgggaagttt | |
| | 2641 | gttcaggctc tattgactgc aaacaagcgg gttagcctcg agggaaaggt tattggggct | |
| | 2701 | ttctgtttct tgcaaatccc gagccctgag ctgcagcaag ctttagcagt ccaacggagg | |
| | 2761 | caggacacag agtgtttcac gaaggcaaaa gagttggctt atatttgtca ggtgataaag | |
| | 2821 | aatcctttga gcggtatgcg tttcgcaaac tcattgttgg aggccacaga cttgaacgag | |
| | 2881 | gaccagaagc agttacttga aacaagtgtt tcttgcgaga aacagatctc aaggatcgtc | |
| | 2941 | ggggacatgg atcttgaaag cattgaagac ggttcatttg tgctaaagag ggaagagttt | |
| | 3001 | ttccttggaa gtgtcataaa cgcgattgta agtcaagcga tgttcttatt aagggacaga | |
| | 3061 | ggtcttcagc tgatccgtga cattcccgaa gagatcaaat caatagaggt ttttggagac | |
| | 3121 | cagataagga ttcaacagct cctggctgag tttctgctga gtataatccg gtatgcacca | |
| | 3181 | tctcaagagt gggtggagat ccatttaagc caactttcaa agcaaatggc tgatggattc | |
| | 3241 | gccgccatcc gcacagaatt cagaatggcg tgtccaggtg aaggtctgcc tccagagcta | |
| | 3301 | gtccgagaca tgttccatag cagcaggtgg acaagccctg aaggtttagg tctaagcgta | |
| | 3361 | tgtcgaaaga ttttaaagct aatgaacggt gaggttcaat acatccgaga atcagaacgg | |
| | 3421 | tcctatttcc tcatcattct ggaactccct gtacctcgaa agcgaccatt gtcaactgct | |
| | 3481 | agtggaagtg gtgacatgat gctgatgatg ccatattag | |
| 6 | MVSGVGGSGGGRGGGRGGEEEPSSSHTPNNRRGGEQAQSSGTKSLRPRSNTESMSKAIQQYTVDARLHAVE | | A. thaliana |
| | EQSGESGKSFDYSQSLKTTTYGSSVPEQQITAYLSRIQRGGYIQPFGCMIAVDESSFRIIGYSENAREMLG | | PhyB wild typc |
| | IMPQSVPTLEKPEILAMGTDVRSLFTSSSSILLERAFVAREITLLNPVWIHSKNTGKPFYAILHRIDVGVV | | prctcin |
| | IDLEPARTEDPALSIAGAVQSQKLAVRAISQLQALPGGDIKLLCDTVVESVRDLTGYDRVMVYKFHEDEHG | | |
| | EVVAESKRDDLEPYIGLHYPATDIPQASRFLFKONRVRMIVDCNATPVLVVODDRLTQSMCLVGSTLRAPH | | |
| | GCHSQYMANMGSIASLAMAVIINGNEDDGSNVASGRSSMRLWGLVVCHHTSSRCIPFPLRYACEFLMQAFG | | |
| | LOLNMELQLALQMSEKRVLRTQTLLCDMLLRDSPAGIVTQSPSIMDLVKCDGAAFLYHGKYYPLGVAPSEV | | |
| | QIKDVVEWLLANHADSTGLSTDSLGDAGYPGAAALGDAVCGMAVAYITKRDFLFWFRSHTAKEIKWGGAKH | | |
| | HPEDKDDGORMHPRSSFQAFLEVVKSRSQPWETAEMDAIHSLOLILRDSFKESEAAMNSKVVDGVVQPCRD | | |
| | MAGEQGIDELGAVAREMVRLIETATVPIFAVDAGGCINGWNAKIAELTGLSVEEAMGKSLVSDLIYKENEA | | |
| | TVNKLLSRALRGDEEKNVEVKLKTFSPELQGKAVFVVVNACSSKDYLNNIVGVCFVGQDVTSQKIVMDKFI | | |
| | NIQGDYKAIVHSPNPLIPPIFAADENTCCLEWNMAMEKLTGWSRSEVIGKMIVGEVFGSCCMLKGPDALTK | | |
| | EMIVLHNAIGGQDTDKFPFPFFDRNGKFVQALLTANKRVSLEGKVIGAFCFLQIPSPELQQALAVORRODT | | |
| | ECFTKAKELAYICQVIKNPLSGMRFANSLLEATDLNEDQKOLLETSVSCEKQISRIVGDMDLESIEDGSFV | | |
| | LKREEFFLGSVINAIVSQAMFLLRDRGLOLIRDIPEEIKSIEVFGDQIRIQQLLAEFLLSIIRYAPSQEWV | | |
| | EIHLSQLSKQMADGFAAIRTEFRMACPGEGLPPELVRDMFHSSRWTSPEGLGLSVCRKILKLMNGEVQYIR | | |
| | ESERSYFLIILELPVPRKRPLSTASGSGDMMLMMPY- | | |
| 7 | 1 | atggtttccg gagtcggggg tagtggcggt ggccgtggcg gtggccgtgg cggagaagaa | YHB allele of |
| | 61 | gaaccgtcgt caagtcacac tcctaataac cgaagaggag gagaacaagc tcaatcgtcg | A. thaliana |
| | 121 | ggaacgaaat ctctcagacc aagaagcaac actgaatcaa tgagcaaagc aattcaacag | phyB. Comprises |
| | 181 | tacaccgtcg acgcaagact ccacgccgtt ttcgaacaat ccggcgaatc agggaaatca | a Y276H |
| | 241 | ttcgactact cacaatcact caaaacgacg acgtacggtt cctctgtacc tgagcaacag | mutation. |
| | 301 | atcacagctt atctctctcg aatccagcga ggtggttaca ttcagccttt cggatgtatg | Constitutively |
| | 361 | atcgccgtcg atgaatccag tttccggatc atcggttaca gtgaaaacgc cagagaaatg | active |
| | 421 | ttagggatta tgcctcaatc tgttcctact cttgagaaac ctgagattct agctatggga | |
| | 481 | actgatgtga gatctttgtt cacttcttcg agctcgattc tactcgagcg tgctttcgtt | |
| | 541 | gctcgagaga ttaccttgtt aaatccggtt tggatccatt ccaagaatac tggtaaaccg | |
| | 601 | ttttacgcca ttcttcatag gattgatgtt ggtgttgtta ttgatttaga gccagctaga | |
| | 661 | actgaagatc ctgcgctttc tattgctggt gctgttcaat cgcagaaact cgcggttcgt | |
| | 721 | gcgatttctc agttacaggc tcttcctggt ggagatatta agcttttgtg tgacactgtc | |
| | 781 | gtggaaagtg tgagggactt gactggttat gatcgtgtta tggttcataa gtttcatgaa | |
| | 841 | gatgagcatg gagaagttgt agctgagagt aaacgagatg atttagagcc ttatattgga | |
| | 901 | ctgcattatc ctgctactga tattcctcaa gcgtcaaggt tcttgtttaa gcagaaccgt | |
| | 961 | gtccgaatga tagtagattg caatgccaca cctgttcttg tggtccagga cgataggcta | |
| | 1021 | actcagtcta tgtgcttggt tcgttctact cttagggctc ctcatggttg tractctcag | |
| | 1081 | tatatggcta acatgggatc tattgcgtct ttagcaatgg cggttataat caatggaaat | |
| | 1141 | gaagatgatg ggagcaatgt agctagtgga agaagctcga tgaggctttg gggtttggtt | |
| | 1201 | gtttgccatc acacttcttc tcgctgcata ccgtttccgc taaggtatgc ttgtgagttt | |
| | 1261 | ttgatgcagg ctttcggttt acagttaaac atggaattgc agttagcttt gcaaatgtca | |
| | 1321 | gagaaacgcg ttttgagaac gcagacactg ttatgtgata tgcttctgcg tgactcgcct | |
| | 1381 | gctggaattg ttacacagag tcccagtatc atggacttag tgaaatgtga cggtgcagca | |
| | 1441 | tttctttacc acgggaagta ttacccgttg ggtgttgctc ctagtgaagt tcagataaaa | |
| | 1501 | gatgttgtgg agtggttgct tgcgaatcat gcggattcaa ccggattaag cactgatagt | |
| | 1561 | ttaggcgatg cggggtatcc cggtgcagct gcgttagggg atgctgtgtg cggtatggca | |
| | 1621 | gttgcatata tcacaaaaag agactttctt ttttggtttc gatctcacac tgcgaaagaa | |
| | 1681 | atcaaatggg gaggcgctaa gcatcatccg gaggataaag atgatgggca acgaatgcat | |
| | 1741 | cctcgttcgt cctttcaggc ttttcttgaa gttgttaaga gccggagtca gccatgggaa | |
| | 1801 | actgcggaaa tggatgcgat tcactcgctc cagcttattc tgagagactc ttttaaagaa | |
| | 1861 | tctgaggcgg ctatgaactc taaagttgtg gatggtgtgg ttcagccatg tagggatatg | |
| | 1921 | gcgggggaac aggggattga tgagttaggt gcagttgcaa gagagatggt taggctcatt | |
| | 1981 | gagactgcaa ctgttcctat attcgctgtg gatgccggag gctgcatcaa tggatggaac | |
| | 2041 | gctaagattg cagagttgac aggtctctca gttgaagaag ctatggggaa gtctctggtt | |
| | 2101 | tctgatttaa tatacaaaga gaatgaagca actgtcaata agcttctttc tcgtgctttg | |
| | 2161 | agaggggacg aggaaaagaa tgtggaggtt aagctgaaaa ctttcagccc cgaactacaa | |
| | 2221 | gggaaagcag tttttgtggt tgtgaatgct tgttccagca aggactactt gaacaacatt | |
| | 2281 | gtcggcgttt gttttgttgg acaagacgtt actagtcaga aaatcgtaat ggataagttc | |
| | 2341 | atcaacatac aaggagatta caaggctatt gtacatagcc caaaccctct aatcccgcca | |
| | 2401 | atttttgctg ctgacgagaa cacgtgctgc ctggaatgga acatggcgat ggaaaagctt | |
| | 2461 | acgggttggt ctcgcagtga agtgattggg aaaatgattg tcggggaagt gtttgggagc | |
| | 2521 | tgttgcatgc taaagggtcc tgatgcttta accaagttca tgattgtatt gcataatgcg | |
| | 2581 | attggtggcc aagatacgga taagttccct ttcccattct ttgaccgcaa tgggaagttt | |
| | 2641 | gttcaggctc tattgactgc aaacaagcgg gttagcctcg agggaaaggt tattggggct | |
| | 2701 | ttctgtttct tgcaaatccc gagccctgag ctgcagcaag ctttagcagt ccaacggagg | |
| | 2761 | caggacacag agtgtttcac gaaggcaaaa gagttggctt atatttgtca ggtgataaag | |
| | 2821 | aatcctttga gcggtatgcg tttcgcaaac tcattgttgg aggccacaga cttgaacgag | |
| | 2881 | gaccagaagc agttacttga aacaagtgtt tcttgcgaga aacagatctc aaggatcgtc | |
| | 2941 | ggggacatgg atcttgaaag cattgaagac ggttcatttg tgctaaagag ggaagagttt | |
| | 3001 | ttccttggaa gtgtcataaa cgcgattgta agtcaagcga tgttcttatt aagggacaga | |
| | 3061 | ggtcttcagc tgatccgtga cattcccgaa gagatcaaat caatagaggt ttttggagac | |
| | 3121 | cagataagga ttcaacagct cctggctgag tttctgctga gtataatccg gtatgcacca | |
| | 3181 | tctcaagagt gggtggagat ccatttaagc caactttcaa agcaaatggc tgatggattc | |
| | 3241 | gccgccatcc gcacagaatt cagaatggcg tgtccaggtg aaggtctgcc tccagagcta | |
| | 3301 | gtccgagaca tgttccatag cagcaggtgg acaagccctg aaggtttagg tctaagcgta | |
| | 3361 | tgtcgaaaga ttttaaagct aatgaacggt gaggttcaat acatccgaga atcagaacgg | |
| | 3421 | tcctatttcc tcatcattct ggaactccct gtacctcgaa agcgaccatt gtcaactgct | |
| | 3481 | agtggaagtg gtgacatgat gctgatgatg ccatattag | |
| 8 | MVSGVGGSGGGRGGGRGGEEEPSSSHTPNNRRGGEQAQSSGTKSLRPRSNTESMSKAIQQYTVDARLHAVE | | A. thaliana |
| | EQSGESGKSFDYSQSLKTTTYGSSVPEQQITAYLSRIQRGGYIQPFGCMIAVDESSFRIIGYSENAREMLG | | PHY-B protein |
| | IMPQSVPTLEKPEILAMGTDVRSLFTSSSSILLERAFVAREITLLNPVWIHSKNTGKPFYAILHRIDVGVV | | encoddd by YHB |
| | IDLEPARTEDPALSIAGAVQSQKLAVRAISQLQALPGGDIKLLCDTVVESVRDLTGYDRVMVHKFHEDEHG | | allele of phyB |
| | EVVAESKRDDLEPYIGLHYPATDIPOASRFLFKONRVRMIVDCNATPVLVVQDDRLTQSMCLVGSTLRAPH | | |
| | GCHSQYMANMGSIASLAMAVIINGNEDDGSNVASGRSSMRLWGLVVCHHTSSRCIPFPLRYACEFLMQAFG | | |
| | LOLNMELQLALQMSEKRVLRTOTLLCDMLLRDSPAGIVTQSPSIMDLVKCDGAAFLYHGKYYPLGVAPSEV | | |
| | QIKDVVEWLLANHADSTGLSTDSLGDAGYPGAAALGDAVCGMAVAYITKRDFLFWFRSHTAKEIKWGGAKH | | |
| | HPEDKDDGQRMHPRSSFQAFLEVVKSRSQPWETAEMDAIHSLQLILRDSFKESEAAMNSKVVDGVVQPCRD | | |
| | MAGEQGIDELGAVAREMVRLIETATVPIFAVDAGGCINGWNAKIAELTGLSVEEAMGKSLVSDLIYKENEA | | |
| | TVNKLLSRALRGDEEKNVEVKLKTFSPELQGKAVFVVVNACSSKDYLNNIVGVCFVGQDVTSQKIVMDKFI | | |
| | NIQGDYKAIVHSPNPLIPPIFAADENTCCLEWNMAMEKLTGWSRSEVIGKMIVGEVFGSCCMLKGPDALTK | | |
| | FMIVLHNAIGGQDTDKFPFPFFDRNGKFVQALLTANKRVSLEGKVIGAFCFLQIPSPELQQALAVORRODT | | |
| | ECFTKAKELAYICQVIKNPLSGMRFANSLLEATDLNEDQKOLLETSVSCEKQISRIVGDMDLESIEDGSFV | | |
| | LKREEFFLGSVINAIVSQAMFLLRDRGLQLIRDIPEEIKSIEVFGDQIRIQQLLAEFLLSIIRYAPSQEWV | | |
| | EIHLSQLSKOMADGFAAIRTEFRMACPGEGLPPELVRDMFHSSRWTSPEGLGLSVCRKILKLMNGEVQYIR | | |
| | ESERSYFLIILELPVPRKRPLSTASGSGDMMLMMPY- | | |
| 9 | 1 | atggtttccg gagtcggggg tagtggcggt ggccgtggcg gtggccgtgg cggagaagaa | A. t. loss of |
| | 61 | gaaccgtcgt caagtcacac tcctaataac cgaagaggag gagaacaagc tcaatcgtcg | function phyB 9 |
| | 121 | ggaacgaaat ctctcagacc aagaagcaac actgaatcaa tgagcaaagc aattcaacag | mutant cDNA |
| | 181 | tacaccgtcg acgcaagact ccacgccgtt ttcgaacaat ccggcgaatc agggaaatca | sequence has a |
| | 241 | ttcgactact cacaatcact caaaacgacg acgtacggtt cctctgtacc tgagcaacag | G to A mutation |
| | 301 | atcacagctt atctctctcg aatccagcga ggtggttaca ttcagccttt cggatgtatg | at nucleotide |
| | 361 | atcgccgtcg atgaatccag tttccggatc atcggttata gtgaaaacgc cagagaaatg | 3489 that |
| | 421 | ttagggatta tgcctcaatc tgttcctact cttgagaaac ctgagattct agctatggga | changes a |
| | 481 | actgatgtga gatctttgtt cacttcttcg agctcgattc tactcgagcg tgctttcgtt | tryptophan |
| | 541 | gctcgagaga ttaccttgtt aaatccggtt tggatccatt ccaagaatac tggtaaaccg | codon to a stop |
| | 601 | ttttacgcca ttcttcatag gattgatgtt ggtgttgtta ttgatttaga gccagctaga | codon at amino |
| | 661 | actgaagatc ctgcgctttc tattgctggt gctgttcaat cgcagaaact cgcggttcgt | acid position |
| | 721 | gcgatttctc agttacaggc tcttcctggt ggagatatta agcttttgtg tgacactgtc | 398 |
| | 781 | gtggaaagtg tgagggactt gactggttat gatcgtgtta tggtttataa gtttcatgaa | |
| | 841 | gatgagcatg gagaagttgt agctgagagt aaacgagatg atttagagcc ttatattgga | |
| | 901 | ctgcattatc ctgctactga tattcctcaa gcgtcaaggt tcttgtttaa gcagaaccgt | |
| | 961 | gtccgaatga tagtagattg caatgccaca cctgttcttg tggtccagga cgataggcta | |
| | 1021 | actcagtcta tgtgcttggt tggttctact cttagggctc ctcatggttg tcactctcag | |
| | 1081 | tatatggcta acatgggatc tattgcgtct ttagcaatgg cggttataat caatggaaat | |
| | 1141 | gaagatgatg ggagcaatgt agctagtgga agaagctcga tgaggctttg aggtttggtt | |
| | 1201 | gtttgccatc acacttcttc tcgctgcata ccgtttccgc taaggtatgc ttgtgagttt | |
| | 1261 | ttgatgcagg ctttcggttt acagttaaac atggaattgc agttagcttt gcaaatgtca | |
| | 1321 | gagaaacgcg ttttgagaac gcagacactg ttatgtgata tgcttctgcg tgactcgcct | |
| | 1381 | gctggaattg ttacacagag tcccagtatc atggacttag tgaaatgtga cggtgcagca | |
| | 1441 | tttctttacc acgggaagta ttacccgttg ggtgttgctc ctagtgaagt tcagataaaa | |
| | 1501 | gatgttgtgg agtggttgct tgcgaatcat gcggattcaa ccggattaag cactgatagt | |
| | 1561 | ttaggcgatg cggggtatcc cggtgcagct gcgttagggg atgctgtgtg cggtatggca | |
| | 1621 | gttgcatata tcacaaaaag agactttctt ttttggtttc gatctcacac tgcgaaagaa | |
| | 1681 | atcaaatggg gaggcgctaa gcatcatccg gaggataaag atgatgggca acgaatgcat | |
| | 1741 | cctcgttcgt cctttcaggc ttttcttgaa gttgttaaga gccggagtca gccatgggaa | |
| | 1801 | actgcggaaa tggatgcgat tcactcgctc cagcttattc tgagagactc ttttaaagaa | |
| | 1861 | tctgaggcgg ctatgaactc taaagttgtg gatggtgtgg ttcagccatg tagggatatg | |
| | 1921 | gcgggggaac aggggattga tgagttaggt gcagttgcaa gagagatggt taggctcatt | |
| | 1981 | gagactgcaa ctgttcctat attcgctgtg gatgccggag gctgcatcaa tggatggaac | |
| | 2041 | gctaagattg cagagttgac aggtctctca gttgaagaag ctatggggaa gtctctggtt | |
| | 2101 | tctgatttaa tatacaaaga gaatgaagca actgtcaata agcttctttc tcgtgctttg | |
| | 2161 | agaggggacg aggaaaagaa tgtggaggtt aagctgaaaa ctttcagccc cgaactacaa | |
| | 2221 | gggaaagcag tttttgtggt tgtgaatgct tgttccagca aggactactt gaacaacatt | |
| | 2281 | gtcggcgttt gttttgttgg acaagacgtt actagtcaga aaatcgtaat ggataagttc | |
| | 2341 | atcaacatac aaggagatta caaggctatt gtacatagcc caaaccctct aatcccgcca | |
| | 2401 | atttttgctg ctgacgagaa cacgtgctgc ctggaatgga acatggcgat ggaaaagctt | |
| | 2461 | acgggttggt ctcgcagtga agtgattggg aaaatgattg tcggggaagt gtttgggagc | |
| | 2521 | tgttgcatgc taaagggtcc tgatgcttta accaagttca tgattgtatt gcataatgcg | |
| | 2581 | attggtggcc aagatacgga taagttccct ttcccattct ttgaccgcaa tgggaagttt | |
| | 2641 | gttcaggctc tattgactgc aaacaagcgg gttagcctcg agggaaaggt tattggggct | |
| | 2701 | ttctgtttct tgcaaatccc gagccctgag ctgcagcaag ctttagcagt ccaacggagg | |
| | 2761 | caggacacag agtgtttcac gaaggcaaaa gagttggctt atatttgtca ggtgataaag | |
| | 2821 | aatcctttga gcggtatgcg tttcgcaaac tcattgttgg aggccacaga cttgaacgag | |
| | 2881 | gaccagaagc agttacttga aacaagtgtt tcttgcgaga aacagatctc aaggatcgtc | |
| | 2941 | ggggacatgg atcttgaaag cattgaagac ggttcatttg tgctaaagag ggaagagttt | |
| | 3001 | ttccttggaa gtgtcataaa cgcgattgta agtcaagcga tgttcttatt aagggacaga | |
| | 3061 | ggtcttcagc tgatccgtga cattcccgaa gagatcaaat caatagaggt ttttggagac | |
| | 3121 | cagataagga ttcaacagct cctggctgag tttctgctga gtataatccg gtatgcacca | |
| | 3181 | tctcaagagt gggtggagat ccatttaagc caactttcaa agcaaatggc tgatggattc | |
| | 3241 | gccgccatcc gcacagaatt cagaatggcg tgtccaggtg aaggtctgcc tccagagcta | |
| | 3301 | gtccgagaca tgttccatag cagcaggtgg acaagccctg aaggtttagg tctaagcgta | |
| | 3361 | tgtcgaaaga ttttaaagct aatgaacggt gaggttcaat acatccgaga atcagaacgg | |
| | 3421 | tcctatttcc tcatcattct ggaactccct gtacctcgaa agcgaccatt gtcaactgct | |
| | 3481 | agtggaagtg gtgacatgat gctgatgatg ccatattag | |
| 10 | MVSGVGGSGGGRGGGRGGEEEPSSSHTPNNRRGGEQAQSSGTKSLRPRSNTESMSKAIQQYTVDARLHAVE | | A.t. phyB |
| | EQSGESGKSFDYSQSLKTTTYGSSVPEQQITAYLSRIQRGGYIQPFGCMIAVDESSFRIIGYSENAREMLG | | protein |
| | IMPQSVPTLEKPEILAMGTDVRSLFTSSSSILLERAFVAREITLLNPVWIHSKNTGKPFYAILHRIDVGVV | | fragment |
| | IDLEPARTEDPALSIAGAVQSQKLAVRAISQLQALPGGDIKLLCDTVVESVRDLTGYDRVMVYKFHEDEHG | | encoded by the |
| | EVVAESKRDDLEPYIGLHYPATDIPQASRFLFKQNRVRMIVDCNATPVLVVQDDRLTQSMCLVGSTLRAPH | | phyB-9 loss-of- |
| | GCHSQYMANMGSIASLAMAVIINGNEDDGSNVASGRSSMRL- | | function allele |
| | | | of phyB (SEQ ID |
| | | | NO: 9). |
| 11 | 1 | ATGGTTTCCG GAGTCGGGGG TAGTGGCGGT GGCCGTGGCG GTGGCCGTGG CGGAGAAGAA | A. thaliana |
| | 61 | GAACCGTCGT CAAGTCACAC TCCTAATAAC CGAAGAGGAG GAGAACAAGC TCAATCGTCG | phyB Y303V cDNA |
| | 121 | GGAACGAAAT CTCTCAGACC AAGAAGCAAC ACTGAATCAA TGAGCAAAGC AATTCAACAG | (a different |
| | 181 | TACACCGTCG ACGCAAGACT CCACGCCGTT TTCGAACAAT CCGGCGAATC AGGGAAATCA | version of YHB) |
| | 241 | TTCGACTACT CACAATCACT CAAAACGACG ACGTACGGTT CCTCTGTACC TGAGCAACAG | |
| | 301 | ATCACAGCTT ATCTCTCTCG AATCCAGCGA GGTGGTTACA TTCAGCCTTT CGGATGTATG | |
| | 361 | ATCGCCGTCG ATGAATCCAG TTTCCGGATC ATCGGTTACA GTGAAAACGC CAGAGAAATG | |
| | 421 | TTAGGGATTA TGCCTCAATC TGTTCCTACT CTTGAGAAAC CTGAGATTCT AGCTATGGGA | |
| | 481 | ACTGATGTGA GATCTTTGTT CACTTCTTCG AGCTCGATTC TACTCGAGCG TGCTTTCGTT | |
| | 541 | GCTCGAGAGA TTACCTTGTT AAATCCGGTT TGGATCCATT CCAAGAATAC TGGTAAACCG | |
| | 601 | TTTTACGCCA TTCTTCATAG GATTGATGTT GGTGTTGTTA TTGATTTAGA GCCAGCTAGA | |
| | 661 | ACTGAAGATC CTGCGCTTTC TATTGCTGGT GCTGTTCAAT CGCAGAAACT CGCGGTTCGT | |
| | 721 | GCGATTTCTC AGTTACAGGC TCTTCCTGGT GGAGATATTA AGCTTTTGTG TGACACTGTC | |
| | 781 | GTGGAAAGTG TGAGGGACTT GACTGGTTAT GATCGTGTTA TGGTTTATAA GTTTCATGAA | |
| | 841 | GATGAGCATG GAGAAGTIGT AGCTGAGAGT AAACGAGATG ATTTAGAGCC TTATATTGGA | |
| | 901 | CTGCATGTTC CTGCTACTGA TATTCCTCAA GCGTCAAGGT TCTTGTTTAA GCAGAACCGT | |
| | 961 | GTCCGAATGA TAGTAGATTG CAATGCCACA CCTGTTCTTG TGGTCCAGGA CGATAGGCTA | |
| | 1021 | ACTCAGTCTA TGTGCTTGGT TGGTTCTACT CTTAGGGCTC CTCATGGTTG TCACTCTCAG | |
| | 1081 | TATATGGCTA ACATGGGATC TATTGCGTCT TTAGCAATGG CGGTTATAAT CAATGGAAAT | |
| | 1141 | GAAGATGATG GGAGCAATGT AGCTAGTGGA AGAAGCTCGA TGAGGCTTTG GGGTTTGGTT | |
| | 1201 | GTTTGCCATC ACACTTCTTC TCGCTGCATA CCGTTTCCGC TAAGGTATGC TTGTGAGTTT | |
| | 1261 | TTGATGCAGG CTTTCGGTTT ACAGTTAAAC ATGGAATTGC AGTTAGCTTT GCAAATGTCA | |
| | 1321 | GAGAAACGCG TTTTGAGAAC GCAGACACTG TTATGTGATA TGCTTCTGCG TGACTCGCCT | |
| | 1381 | GCTGGAATTG TTACACAGAG TCCCAGTATC ATGGACTTAG TGAAATGTGA CGGTGCAGCA | |
| | 1441 | TTTCTTTACC ACGGGAAGTA TTACCCGTTG GGTGTTGCTC CTAGTGAAGT TCAGATAAAA | |
| | 1501 | GATGTTGTGG AGTGGTTGCT TGCGAATCAT GCGGATTCAA CCGGATTAAG CACTGATAGT | |
| | 1561 | TTAGGCGATG CGGGGTATCC CGGTGCAGCT GCGTTAGGGG ATGCTGTGTG CGGTATGGCA | |
| | 1621 | GTTGCATATA TCACAAAAAG AGACTTTCTT TTTTGGTTTC GATCTCACAC TGCGAAAGAA | |
| | 1681 | ATCAAATGGG GAGGCGCTAA GCATCATCCG GAGGATAAAG ATGATGGGCA ACGAATGCAT | |
| | 1741 | CCTCGTTCGT CCTTTCAGGC TTTTCTTGAA GTTGTTAAGA GCCGGAGTCA GCCATGGGAA | |
| | 1801 | ACTGCGGAAA TGGATGCGAT TCACTCGCTC CAGCTTATTC TGAGAGACTC TTTTAAAGAA | |
| | 1861 | TCTGAGGCGG CTATGAACTC TAAAGTTGTG GATGGTGTGG TTCAGCCATG TAGGGATATG | |
| | 1921 | GCGGGGGAAC AGGGGATTGA TGAGTTAGGT GCAGTTGCAA GAGAGATGGT TAGGCTCATT | |
| | 1981 | GAGACTGCAA CTGTTCCTAT ATTCGCTGTG GATGCCGGAG GCTGCATCAA TGGATGGAAC | |
| | 2041 | GCTAAGATTG CAGAGTTGAC AGGTCTCTCA GTTGAAGAAG CTATGGGGAA GTCTCTGGTT | |
| | 2101 | TCTGATTTAA TATACAAAGA GAATGAAGCA ACTGTCAATA AGCTTCTTTC TCGTGCTTTG | |
| | 2161 | AGAGGGGACG AGGAAAAGAA TGTGGAGGTT AAGCTGAAAA CTTTCAGCCC CGAACTACAA | |
| | 2221 | GGGAAAGCAG TTTTTGTGGT TGTGAATGCT TGTTCCAGCA AGGACTACTT GAACAACATT | |
| | 2281 | GTCGGCGTTT GTTTTGTTGG ACAAGACGTT ACTAGTCAGA AAATCGTAAT GGATAAGTTC | |
| | 2341 | ATCAACATAC AAGGAGATTA CAAGGCTATT GTACATAGCC CAAACCCTCT AATCCCGCCA | |
| | 2401 | ATTTTTGCTG CTGACGAGAA CACGTGCTGC CTGGAATGGA ACATGGCGAT GGAAAAGCTT | |
| | 2461 | ACGGGTTGGT CTCGCAGTGA AGTGATTGGG AAAATGATTG TCGGGGAAGT GTTTGGGAGC | |
| | 2521 | TGTTGCATGC TAAAGGGTCC TGATGCTTTA ACCAAGTTCA TGATTGTATT GCATAATGCG | |
| | 2581 | ATTGGTGGCC AAGATACGGA TAAGTTCCCT TTCCCATTCT TTGACCGCAA TGGGAAGTTT | |
| | 2641 | GTTCAGGCTC TATTGACTGC AAACAAGCGG GTTAGCCTCG AGGGAAAGGT TATTGGGGCT | |
| | 2701 | TTCTGTTTCT TGCAAATCCC GAGCCCTGAG CTGCAGCAAG CTTTAGCAGT CCAACGGAGG | |
| | 2761 | CAGGACACAG AGTGTTTCAC GAAGGCAAAA GAGTTGGCTT ATATTTGTCA GGTGATAAAG | |
| | 2821 | AATCCTTTGA GCGGTATGCG TTTCGCAAAC TCATTGTTGG AGGCCACAGA CTTGAACGAG | |
| | 2881 | GACCAGAAGC AGTTACTTGA AACAAGTGTT TCTTGCGAGA AACAGATCTC AAGGATCGTC | |
| | 2941 | GGGGACATGG ATCTTGAAAG CATTGAAGAC GGTTCATTTG TGCTAAAGAG GGAAGAGTTT | |
| | 3001 | TTCCTTGGAA GTGTCATAAA CGCGATTGTA AGTCAAGCGA TGTTCTTATT AAGGGACAGA | |
| | 3061 | GGTCTTCAGC TGATCCGTGA CATTCCCGAA GAGATCAAAT CAATAGAGGT TTTTGGAGAC | |
| | 3121 | CAGATAAGGA TTCAACAGCT CCTGGCTGAG TTTCTGCTGA GTATAATCCG GTATGCACCA | |
| | 3181 | TCTCAAGAGT GGGTGGAGAT CCATTTAAGC CAACTTTCAA AGCAAATGGC TGATGGATTC | |
| | 3241 | GCCGCCATCC GCACAGAATT CAGAATGGCG TGTCCAGGTG AAGGTCTGCC TCCAGAGCTA | |
| | 3301 | GTCCGAGACA TGTTCCATAG CAGCAGGTGG ACAAGCCCTG AAGGTTTAGG TCTAAGCGTA | |
| | 3361 | TGTCGAAAGA TTTTAAAGCT AATGAACGGT GAGGTTCAAT ACATCCGAGA ATCAGAACGG | |
| | 3421 | TCCTATTTCC TCATCATTCT GGAACTCCCT GTACCTCGAA AGCGACCATT GTCAACTGCT | |
| | 3481 | AGTGGAAGTG GTGACATGAT GCTGATGATG CCATATTAG | |
| 12 | MVSGVGGSGGGRGGGRGGEEEPSSSHTPNNRRGGEQAQSSGTKSLRPRSNTESMSKAIQQYTVDARLHAVE | | Another A.t. |
| | EQSGESGKSFDYSQSLKTTTYGSSVPEQQITAYLSRIQRGGYIQPFGCMIAVDESSFRIIGYSENAREMLG | | YHB allele |
| | IMPQSVPTLEKPEILAMGTDVRSLFTSSSSILLERAFVAREITLLNPVWIHSKNTGKPFYAILHRIDVGVV | | (phyB Y303V) |
| | IDLEPARTEDPALSIAGAVQSQKLAVRAISQLQALPGGDIKLLCDTVVESVRDLTGYDRVMVYKFHEDEHG | | protein |
| | EVVAESKRDDLEPYIGLHVPATDIPOASRFLFKONRVRMIVDCNATPVLVVODDRLTOSMCLVGSTLRAPH | | (encoded by SEQ |
| | GCHSQYMANMGSIASLAMAVIINGNEDDGSNVASGRSSMRLWGLVVCHHTSSRCIPFPLRYACEFLMQAFG | | ID NO: 11) |
| | LOLNMELQLALQMSEKRVLRTQTLLCDMLLRDSPAGIVTQSPSIMDLVKCDGAAFLYHGKYYPLGVAPSEV | | |
| | QIKDVVEWLLANHADSTGLSTDSLGDAGYPGAAALGDAVCGMAVAYITKRDFLFWFRSHTAKEIKWGGAKH | | |
| | HPEDKDDGQRMHPRSSFQAFLEVVKSRSQPWETAEMDAIHSLQLILRDSFKESEAAMNSKVVDGVVQPCRD | | |
| | MAGEQGIDELGAVAREMVRLIETATVPIFAVDAGGCINGWNAKIAELTGLSVEEAMGKSLVSDLIYKENEA | | |
| | TVNKLLSRALRGDEEKNVEVKLKTFSPELQGKAVFVVVNACSSKDYLNNIVGVCFVGQDVTSQKIVMDKFI | | |
| | NIQGDYKAIVHSPNPLIPPIFAADENTCCLEWNMAMEKLTGWSRSEVIGKMIVGEVFGSCCMLKGPDALTK | | |
| | FMIVLHNAIGGODTDKFPFPFFDRNGKFVQALLTANKRVSLEGKVIGAFCFLQIPSPELQQALAVORRODT | | |
| | ECFTKAKELAYICQVIKNPLSGMRFANSLLEATDLNEDQKQLLETSVSCEKQISRIVGDMDLESIEDGSFV | | |
| | LKREEFFLGSVINAIVSQAMFLLRDRGLQLIRDIPEEIKSIEVFGDQIRIQQLLAEFLLSIIRYAPSQEWV | | |
| | EIHLSQLSKQMADGFAAIRTEFRMACPGEGLPPELVRDMFHSSRWTSPEGLGLSVCRKILKLMNGEVQYIR | | |
| | ESERSYFLIILELPVPRKRPLSTASGSGDMMLMMPY- | | |
| 13 | ATGAAGAGGGGAAAAGATGAGGGAAAGATTATGGGGCCTATGTTTCCGAGGCTCCATGTGAATGATACCGA | | A basil elf3 |
| | GAAAGGGGGGCCAAGAGCCCCTCCTAGAAACAAGATGGCTCTTTATGAGCAGCTCAGCATTCCTTCACAGA | | isoform |
| | GACTCAGTAATTCTGTTAGACCCCCGAATTCGGAGCACGCTTCGGATCAGGGGGCTGCAAACGAAAGGGGT | | |
| | GCGTTTTTCTCGTATCAGCTGCCTAACAGACATAAGTCAGATAAGATAAGCAGCCAATATTCCGATTTCAG | | |
| | CGCTCCATTCACGCCACTTGGCCGGAGAAAGAAATCCGATGAAGAGGAATTCAGAGTTCCCGTTTTCCACC | | |
| | ATTCCATCCAAAATCAAGAATGCGGTGATTATGTTAAGAAGGATAACCCCGTGCATGAAAAAGTCTCCGCT | | |
| | TCCAACCCCTCTAGTCTCAATCGTCCGTCGAAATTCCATAAATTCAAAAGCCAGACAGACAAGAATTCAAA | | |
| | AGAGTTCCCCTCTGATCGAACAAAAGTTGTGTCAAATTCATCCATCATCGAGAAGCAAATTGATTCAGCTT | | |
| | TTTGTCGTGAGCCAACAGATTGTCAAGCTAGTAGTATTGGCAGATTACAGACCCGCGATATGAGTGAGCCA | | |
| | GAGCTACGTGCTGATTCTGAGCATGATGAAATTAGGCGTGCTAACTCTGTGGTAATGAAGAATTTTCCAAC | | |
| | CTTAGATGAAAGAAATTCTAAAGAATTGGGAAATGATGAAGGATCTCAGAAAAGATTGGTACCAATGGGAG | | |
| | ACCTTGAGAGGGCTGACAGTGTGTCTGAGATTTCGGTTGTCGAGTCCATTGCTGGGCTCGATATGACACCC | | |
| | GACGACGTGGTGGGAATAATTGGTCAGAAACATTTTTGGAAGGCCAGAAAGAATATTCTTAATCAACAAAG | | |
| | AGTTTTTGCTGTTCAAGTATTCGAGTTGCATCGATTGGTTAAGGTCCAGAGATTGATCGCTGCATTGCCAC | | |
| | AGTTCTTGCTCGACAACACGGCCTTCGTCGACAAACCAAACGCTGCGCCCGGACCAAAAGTCCCATCGGTT | | |
| | AGTCCCCTCAAAGCCGCGAATCCCGAAATTCCAAAGCGAAAAGGCGAACTCCAGAAGCCGAGCCATAACAA | | |
| | GGAAAGCTCCGCCGAAAACACTGCCGAGAAACCATCGTTCTCGTCCATTCAAAACGGCGAGCTCCCGTCTG | | |
| | GCTACGGGCCGTTGACAGGCAACTCTCTGGGCCCTCCCGTTTCGAGCAATCCAAATACCGGGCCCGGGCCG | | |
| | TGGTGCTTCAACCAACCTCAAGGGCACCAATGGTTGATTCCTGTGATGACACCTTCAGAAGGACTAGTTTA | | |
| | CAAGCCATATCCCGGGCCGGGCTTCGTGGGCCCAGCCTGCGGCGGCTGTGGCCCACCCGCGCCAATTCCAG | | |
| | CGTATGGAATCCCGGCCCCACATCCTCATTATCAGCTCCCATCGTTCCCGCACGGCTACTTCCCGACGTAC | | |
| | GGCATGCCGATGATGAACACGGCCGCAGTGTCGGGCTCGTCCGTCGAGCAGACGAACCCTCAACCTGTTCC | | |
| | TAGCCAAGCTTCGGGAGGCGGAACCGGTGCCAGCTTTCAACATCATCAGAACTTGCCTTCGACACCAAGCC | | |
| | GCAAGAATCGAGCAGATCCTGACAGGACGAATGCGTCGAAGAACTACGAACTTGTTGAGACTAGCAGAGGA | | |
| | AGTAGCCCCGTTGAAAGATTGGTCGATAATGGAGGAGGAAGCGATGACGGGGAAAGAGGGAATATGCTCTC | | |
| | ACTTTTCCCAATGGCCCCATCTTCCGATGAGCCTGGTTCGAGCCCAGCCTGTACGAAGCCCGATCGGCCGA | | |
| | CTCAAGTGGTCAGGGTTGTGCCTCATAATGCAAGATCTGCCACTGAATCTGCTGCAAGGATTTTTAGATTT | | |
| | ATACAAGAAGGAAGAAAACAGTATGATTCTGTGTGA | | |
| 14 | ATGAAGAGAGGGAGAGATGAGGGGAAGATTATGGGGCCAATGTTTCCAAGGCTACATGTGAATGACACTGA | | |
| | GAAAGGAGGGCCAAGAGCCCCTCCCAGAAACAAAATGGCTCTGTATGAGCAGCTCACCATCCCTTCCCACA | | |
| | GACTCACCGATTCCGCCCAGGGTGTTTCAACTGATACTGGTGCAATTTTCCCATACAAGCCGTCTAACAGA | | |
| | CATCAGTCCGAGAATCTATGCGGTCAATATTCCGATTTCAGTATACCGTCGACGCCAGTCAGCTGGAGAAA | | |
| | GAAATCGGACGAAGAGGATTTCAGAGTTCCCATTTTCAACGATTCTGTCCGAAGTCAAGAACGTAGAAAAT | | |
| | GTGGTAATGACCCGGTGCAGGAAAAGGTCAATCACCCGTCGAAATTCCATAAATTCAAAAGGACAGGCATG | | |
| | CTTGAAAGTAACATGAGGCAAGAAGGCAAAAGCCAGACAGAAAAGAATTCACAAGCGCTTGCATTCGATCG | | |
| | AGCAAAAGCTTTATCAAACTCATCTAGCATCGAGAAGCAGAATGGTTCACCTATATTTCGTGAACGGAGAG | | |
| | ATTGTCCTGCTAAAAGTGTCGACAGATTACAGACCCGCGATGCCATTGAGCCGAAGCTCCGTCCTGATTCA | | |
| | GAGCGTGCTGGAAACGGGTGCACTACCTCGCTGTTGGAGAAGGCTTCGGCAACCTTAGATGAAAGAAACTC | | |
| | TTCTGCTCCATTCAGAGATCAGAGAAATTCTTGTGATTTGGGAAACAATCGCAAAAGGTCAGTGGCGGTGA | | |
| | CAAATCTTGATATGGCTGACAGTGTGTCGGAGATCTCGCTTGTGGAGTCCATCGCCGGACCGGATATGACG | | |
| | CCTGATGACGTGGTGGGAATAATCGGTCCGAAACATTTTTGGAAAGCCAGAAAAGCCATTCTCAACCAACA | | |
| | AAGAGTTTTCGCGATCCAAGTATTCGAGTTGCATCGACTGATAAAGGTCCAGAGATCGATCGCCGAATCAC | | |
| | CCCAGATGTTGGCCGACAACTCGGTTTTCGTCGACAAACCAGACTTGGCTAGTCCCGTCAAACCTATTCCC | | |
| | AATGCTTCCAAGCGTACGGCCAGCCCCATCAAAGCATTTCCCGCCGTTTCCAAGAAAAAACCCGAACCCAA | | |
| | AAAGGCGAGTCAGAACAAGGAAAGCCCCGTCGAAAAGGCCTCTTTTTCGACGGGCCATGGCGGGCCGTGGT | | |
| | GCTTCAACCAGCCTCAAGGCCACCAATGGCTGATTCCTGTGATGACTCCTTCGGAGGGACTCGTGTATAAG | | |
| | CCATATCCCGGGCCGGGCTTCGTTGGCCCGAATCCGACAATGGGCAGTTTCTTGCCTCCTCCGGGATATGG | | |
| | AATCCCACCACACTATCAGGGCTGCTACTTTTCGCCGTATGCAATGTCGGCGGCTGCAGTGTCGGGCTCGT | | |
| | CTGTCGAGCAGACGAACCCTCCCCAATCAAGCAGAGCAAGTAGCCCTGTTGAGAGATTGGTGGATAGTGGA | | |
| | GGAAGCAATGATATGGAAAGAGGGAATGTGCTGCCACTTTTCCCCGTGGCGCCCGACTCAAGCCCGGCCCG | | |
| | GTCCAAGCCCGAGTCCGAGCCCGATCGCCCTGCTCGGGTGATAAGAGTTGTGCCTCATAATGCAACTTCAG | | |
| | CAACTGAATCTGCTGCAAGAATTTTCAGGTCTATTCAAGAGGAAAGAAGACGGTATGATTCTGTGTAA | | |
| 15 | ATGAAGAGAGGGAGAGATGAGGGGAAGATTATGGGGCCAATGTTTCCAAGGCTACATGTGAATGACACTGA | | |
| | GAAAGGAGGGCCAAGAGCCCCTCCCAGAAACAAAATGGCTCTGTATGAGCAGCTCACCATCCCTTCCCACA | | |
| | GACTCACCGATTCCGCCCAGACATCAGTCCGAGAATCTATGCGGTCAATATTCCGATTTCAGTATACCGTC | | |
| | GACGCCAGTCAGCTGGAGAAAGAAATCGGACGAAGAGGATTTCAGAGTTCCCATTTTCAACGATTCTGTCC | | |
| | GAAGTCAAGAACGTAG | | |
| 16 | ATGAAGAGAAATAATGAAGGGGAGAAGATAATGGGGGCCATGTTTCCTAGGCTTCATGTGAACGACACCGA | | |
| | AAAGGGGGGGCCACGAGCACCCCCGAGAAACAAGATGGCTTTATACGAGCAGCTTAGCATACCTTCACACA | | |
| | GACTGAATCATGCTGTGATGCCCCGAAACCCGACTAACAACGCCACTAGCACGGCCCCTTCATCGAGAATG | | |
| | GGCGGTGGAAATCATCGGGGTTCGTTCAGTGCTTGTGTACGGCGTCGTGCATATCCATCTCAGGATCAATA | | |
| | TACCCAATATTCAGAATCGAGCACTTCACCACCACCACAAGTAGAGCCAAGAAAGAAAATCTACGACAATG | | |
| | ACAATAGGGTTCCTATTTTCATCCAGCCCATACCTCAAGAATTTGATAAATACAGCAGTGGCTTTCTCTCC | | |
| | AATCCTCCATATTTGGACCCTCCATTGAAATTTCATCAGACCAATGAAATGAGCACGCTTGAAGTTAGTAT | | |
| | TGGAGATGAGGGGCAAATCAAGAAGAGAGAAAATTCAAATGCAGTGAGCTCGGAGAAGGATGATACTGCCT | | |
| | CTGAAACTTCGATTTTGGATACGGAATCAGCCCTCGAAATAACGCCCGATGATGTTGTGGGTGCAATGGGG | | |
| | CAAGAACATTTTTGGAAAGCCAGAAGAGCAATCATGAATCAGCAAAGAGTGTTTGCAGTTCAAGTATTCGA | | |
| | GTTGCATAGATTGATAAAGGTCCAGAGATCGATCGCTGCATCGCCACATCTTTTGCACGACGAAGCTGCTT | | |
| | ATCTCGACGACCCCACAAAACCATTACCGGCAAAGAAACGGCCGCTCGATTGCACTGATAATGCGGTCTTG | | |
| | AACGCTGAGAACCCGAGACACAAGAAGGAGCGGTCCGCCAAAAACACGGTCGAGAAAGAGAAAACGCCTCT | | |
| | TTCTTCAGCTCGTCCGGCTTCGACCGTCGTGAGCACTGATTGGAGCAACGCTCGAACATCATATTTCAATG | | |
| | ATCCCCTGCCACACGGACACCAATGGTTGATTCCTATAATGTCTCCTTCAGAAGGATTAGTATACAAGCCG | | |
| | TGTTCGGGCCCCGGATTTCTCGGCCCGATAGGGTTGAATCCGACAACGATCGGAAACTTCGTAGCTCCCGT | | |
| | ATACGGCCTCCCGCCACCGTCGCATCCGTTCCCACCTTTTTACGGGCCTCATTACAGCATGCCGGTTATGA | | |
| | GCTCTGCAGCCTTCTCGGCCCGTGAATCCACGTTGGATCGGCCTGCCTTGAAAGCCGGACCGTGCCAACGT | | |
| | GCCACGGAGGAGATCAAAAGTGGCCATAGTAGGGTTAGCAACGGCATTCTTCAACGTTTCCCGATATCGTC | | |
| | TCCGGCCACCACCGATGCCTCGGAATCGAGCTGTCGCGTTCGTGCGGCGGGGCCCGGCCCGGCCCGAGGGT | | |
| | GAT | | |
| 17 | ATGAAGAGAGGGAATGATGGGGAGAAGATAATGGGCCCTATGTTCCCTAGGCTTCATGTAAATGATGCTGA | | |
| | TAAGGGCGGGCCCAGAGCTCCTCCAAGGAATAAGATGGCCTTGTATGAGCAGCTCAGCATTCCTTCTCAGA | | |
| | GATTCAGTCATGCCGTGCCGCCCCATAATAGGAATAATAACATGGCCACTTCAGTGCCCAAGTCGAGCCTG | | |
| | AGCGGTGGGAACGATAGGGGCGCGTTCAGTGCTCGGCAGGTGCATCGAGCATATCCATATGAGATTGAGAA | | |
| | GCAATACAGGGACTGCTCGGAATTGAGTGCTCCGCTGGCACAAGTAAAGCAAAGAAAGAAAATCGACGAAG | | |
| | ATGACTATGCAGTTCCTGTTTTCATCCAGCCTAAACCTTCAGAATTCGAAAACTACAGCAAGGAAAAACTC | | |
| | TCACCCTCCAATCCTCCTTATTTGCACCCTTCGTTGAAATCTCAGCTCTCCAACGAAATAGACACGCTCGA | | |
| | AATTAGTATCGGACGAGAAGGGGGAAGCAAGAAGAGAGAAAATTCAGACGAGTACGTGACTGGGAGTAAAG | | |
| | AAACTGTTTCCTCAATTAGTCATGAATTTGGAGCAAATCTCGTCAACAGCGTCGACATATTAAGGAATGAT | | |
| | GACATGGCTGCGGAAAAAAGAAATCCATCTAATTTAGGGGAAGTTCTACAATCGGAGGATCAGAATACAGT | | |
| | CCATGATCTTAGTTATGAGACGGGGTCTGATGAAGATGGGTCTTTGCAAGAAGGGAAATTGGAAAGGCGAG | | |
| | ATAGTGCCTCGGAAACATCCGTTTTGGATACGGAATCTGCCATGGAAATAACGCCCGACGACGTTGTGGGT | | |
| | ACGATAGGCCAGAAGCATTTTTGGAAAGCCAGAAGAGCTATCATGAAAATATTAATTCCTCATAGTGAGGA | | |
| | AATTTATTTCTTCAGCCACCTTTGAT | | |
| 18 | ATGAAGAGAGGGAATGATGGGGAGAAGATAATGGGCCCTATGTTCCCTAGGCTTCATGTAAATGATGCTGA | | |
| | TAAGGGCGGGCCCAGAGCTCCTCCAAGGAATAAGATGGCCTTGTATGAGCAGCTCAGCATTCCTTCTCAGA | | |
| | GATTCAGTCATGCCGTGCCGCCCCATAATAGGAATAATAACATGGCCACTTCAGTGCCCAAGTCGAGCCTG | | |
| | AGCGGTGGGAACGATAGGGGCGCGTTCAGTGCTCGGCAGGTGCATCGAGCATATCCATATGAGATTGAGAA | | |
| | GCAATACAGGGACTGCTCGGAATTGAGTGCTCCGCTGGCACAAGTAAAGCAAAGAAAGAAAATCGACGAAG | | |
| | ATGACTATGCAGTTCCTGTTTTCATCCAGCCTAAACCTTCAGAATTCGAAAACTACAGCAAGGAAAAACTC | | |
| | TCACCCTCCAATCCTCCTTATTTGCACCCTTCGTTGAAATCTCAGCTCTCCAACGAAATAGACACGCTCGA | | |
| | AATTAGTATCGGACGAGAAGGGGGAAGCAAGAAGAGAGAAAATTCAGACGAGTACGTGACTGGGAGTAAAG | | |
| | AAACTGTTTCCTCAATTAGTCATGAATTTGGAGCAAATCTCGTCAACAGCGTCGACATATTAAGGAATGAT | | |
| | GACATGGCTGCGGAAAAAAGAAATCCATCTAATTTAGGGGAAGTTCTACAATCGGAGGATCAGAATACAGT | | |
| | CCATGATCTTAGTTATGAGACGGGGTCTGATGAAGATGGGTCTTTGCAAGAAGGGAAATTGGAAAGGCGAG | | |
| | ATAGTGCCTCGGAAACATCCGTTTTGGATACGGAATCTGCCATGGAAATAACGCCCGACGACGTTGTGGGT | | |
| | ACGATAGGCCAGAAGCATTTTTGGAAAGCCAGAAGAGCTATCATGAAGTAA | | |
| 19 | 1 | ggccctctag aggatccccg ggtaccctcg aattatcata catgagaatt aagggagtca | pK7-35S::YHB- |
| | 61 | cgttatgacc cccgccgatg acgcgggaca agccgtttta cgtttggaac tgacagaacc | mCer-SHH. |
| | 121 | gcaacgttga aggagccact cagccgcggg tttctggagt ttaatgagct aagcacatac | Plasmid |
| | 181 | gtcagaaacc attattgcgc gttcaaaagt cgcctaaggt cactatcagc tagcaaatat | comprising |
| | 241 | ttcttgtcaa aaatgctcca ctgacgttcc ataaattccc ctcggtatcc aattagagtc | expression |
| | 301 | tcatattcac tctcaactcg atcgaggcat gattgaacaa gatggattgc acgcaggttc | construct for |
| | 361 | tccggccgct tgggtggaga ggctattcgg ctatgactgg gcacaacaga caatcggctg | A.t. YHB which |
| | 421 | ctctgatgcc gccgtgttcc ggctgtcagc gcaggggcgc ccggttcttt ttgtcaagac | has a sequence |
| | 481 | cgacctgtcc ggtgccctga atgaactcca agacgaggca gcgcggctat cgtggctggc | of SEQ ID NO: |
| | 541 | cacgacgggc gttccttgcg cagctgtgct cgacgttgtc actgaagcgg gaagggactg | 24 |
| | 601 | gctgctattg ggcgaagtgc cggggcagga tctcctgtca tctcaccttg ctcctgccga | |
| | 661 | gaaagtatcc atcatggctg atgcaatgcg gcggctgcat acgcttgatc cggctacctg | |
| | 721 | cccattcgac caccaagcga aacatcgcat cgagcgagca cgtactcgga tggaagccgg | |
| | 781 | tcttgtcgat caggatgatc tggacgaaga gcatcagggg ctcgcgccag ccgaactgtt | |
| | 841 | cgccaggctc aaggcgcgga tgcccgacgg cgaggatctc gtcgtgaccc acggcgatgc | |
| | 901 | ctgcttgccg aatatcatgg tggaaaatgg ccgcttttct ggattcatcg actgtggccg | |
| | 961 | gctgggtgtg gcggaccgct atcaggacat agcgttggct acccgtgata ttgctgaaga | |
| | 1021 | gcttggcggc gaatgggctg accgcttcct cgtgctttac ggtatcgccg ctcccgattc | |
| | 1081 | gcagcgcatc gccttctatc gccttcttga cgagttcttc tgagcgggac tctggggttc | |
| | 1141 | ggactctagc tagagtcaag cagatcgttc aaacatttgg caataaagtt tcttaagatt | |
| | 1201 | gaatcctgtt gccggtcttg cgatgattat catataattt ctgttgaatt acgttaagca | |
| | 1261 | tgtaataatt aacatgtaat gcatgacgtt atttatgaga tgggttttta tgattagagt | |
| | 1321 | cccgcaatta tacatttaat acgcgataga aaacaaaata tagcgcgcaa actaggataa | |
| | 1381 | attatcgcgc gcggtgtcat ctatgttact agatcgaccg gcatgcaagc tgataattca | |
| | 1441 | attcggcgtt aattcagtac attaaaaacg tccgcaatgt gttattaagt tgtctaagcg | |
| | 1501 | tcaatttgtt tacaccacaa tatatcctgc caccagccag ccaacagctc cccgaccggc | |
| | 1561 | agctcggcac aaaatcacca ctcgatacag gcagcccatc agtccgggac ggcgtcagcg | |
| | 1621 | ggagagccgt tgtaaggcgg cagactttgc tcatgttacc gatgctattc ggaagaacgg | |
| | 1681 | caactaagct gccgggtttg aaacacggat gatctcgcgg agggtagcat gttgattgta | |
| | 1741 | acgatgacag agcgttgctg cctgtgatca attcgggcac gaacccagtg gacataagcc | |
| | 1801 | tcgttcggtt cgtaagctgt aatgcaagta gcgtaactgc cgtcacgcaa ctggtccaga | |
| | 1861 | accttgaccg aacgcagcgg tggtaacggc gcagtggcgg ttttcatggc ttcttgttat | |
| | 1921 | gacatgtttt tttggggtac agtctatgcc tcgggcatcc aagcagcaag cgcgttacgc | |
| | 1981 | cgtgggtcga tgtttgatgt tatggagcag caacgatgtt acgcagcagg gcagtcgccc | |
| | 2041 | taaaacaaag ttaaacatca tgggggaagc ggtgatcgcc gaagtatcga ctcaactatc | |
| | 2101 | agaggtagtt ggcgtcatcg agcgccatct cgaaccgacg ttgctggccg tacatttgta | |
| | 2161 | cggctccgca gtggatggcg gcctgaagcc acacagtgat attgatttgc tggttacggt | |
| | 2221 | gaccgtaagg cttgatgaaa caacgcggcg agctttgatc aacgaccttt tggaaacttc | |
| | 2281 | ggcttcccct ggagagagcg agattctccg cgctgtagaa gtcaccattg ttgtgcacga | |
| | 2341 | cgacatcatt ccgtggcgtt atccagctaa gcgcgaactg caatttggag aatggcagcg | |
| | 2401 | caatgacatt cttgcaggta tcttcgagcc agccacgatc gacattgatc tggctatctt | |
| | 2461 | gctgacaaaa gcaagagaac atagcgttgc cttggtaggt ccagcggcgg aggaactctt | |
| | 2521 | tgatccggtt cctgaacagg atctatttga ggcgctaaat gaaaccttaa cgctatggaa | |
| | 2581 | ctcgccgccc gactgggctg gcgatgagcg aaatgtagtg cttacgttgt cccgcatttg | |
| | 2641 | gtacagcgca gtaaccggca aaatcgcgcc gaaggatgtc gctgccgact gggcaatgga | |
| | 2701 | gcgcctgccg gcccagtatc agcccgtcat acttgaagct agacaggctt atcttggaca | |
| | 2761 | agaagaagat cgcttggcct cgcgcgcaga tcagttggaa gaatttgtcc actacgtgaa | |
| | 2821 | aggcgagatc accaaggtag tcggcaaata atgtctagct agaaattcgt tcaagccgac | |
| | 2881 | gccgcttcgc cggcgttaac tcaagcgatt agatgcacta agcacataat tgctcacagc | |
| | 2941 | caaactatca ggtcaagtct gcttttatta tttttaagcg tgcataataa gccctacaca | |
| | 3001 | aattgggaga tatatcatgc atgaccaaaa tcccttaacg tgagttttcg ttccactgag | |
| | 3061 | cgtcagaccc cgtagaaaag atcaaaggat cttcttgaga tccttttttt ctgcgcgtaa | |
| | 3121 | tctgctgctt gcaaacaaaa aaaccaccgc taccagcggt ggtttgtttg ccggatcaag | |
| | 3181 | agctaccaac tctttttccg aaggtaactg gcttcagcag agcgcagata ccaaatactg | |
| | 3241 | tccttctagt gtagccgtag ttaggccacc acttcaagaa ctctgtagca ccgcctacat | |
| | 3301 | acctcgctct gctaatcctg ttaccagtgg ctgctgccag tggcgataag tcgtgtctta | |
| | 3361 | ccgggttgga ctcaagacga tagttaccgg ataaggcgca gcggtcgggc tgaacggggg | |
| | 3421 | gttcgtgcac acagcccagc ttggagcgaa cgacctacac cgaactgaga tacctacagc | |
| | 3481 | gtgagctatg agaaagcgcc acgcttcccg aagggagaaa ggcggacagg tatccggtaa | |
| | 3541 | gcggcagggt cggaacagga gagcgcacga gggagcttcc agggggaaac gcctggtatc | |
| | 3601 | tttatagtcc tgtcgggttt cgccacctct gacttgagcg tcgatttttg tgatgctcgt | |
| | 3661 | caggggggcg gagcctatgg aaaaacgcca gcaacgcggc ctttttacgg ttcctggcct | |
| | 3721 | tttgctggcc ttttgctcac atgttctttc ctgcgttatc ccctgattct gtggataacc | |
| | 3781 | gtattaccgc ctttgagtga gctgataccg ctcgccgcag ccgaacgacc gagcgcagcg | |
| | 3841 | agtcagtgag cgaggaagcg gaagagcgcc tgatgcggta ttttctcctt acgcatctgt | |
| | 3901 | gcggtatttc acaccgcata tggtgcactc tcagtacaat ctgctctgat gccgcatagt | |
| | 3961 | taagccagta tacactccgc tatcgctacg tgactgggtc atggctgcgc cccgacaccc | |
| | 4021 | gccaacaccc gctgacgcgc cctgacgggc ttgtctgctc ccggcatccg cttacagaca | |
| | 4081 | agctgtgacc gtctccggga gctgcatgtg tcagaggttt tcaccgtcat caccgaaacg | |
| | 4141 | cgcgaggcag ggtgccttga tgtgggcgcc ggcggtcgag tggcgacggc gcggcttgtc | |
| | 4201 | cgcgccctgg tagattgcct ggccgtaggc cagccatttt tgagcggcca gcggccgcga | |
| | 4261 | taggccgacg cgaagcggcg gggcgtaggg agcgcagcga ccgaagggta ggcgcttttt | |
| | 4321 | gcagctcttc ggctgtgcgc tggccagaca gttatgcaca ggccaggcgg gttttaagag | |
| | 4381 | ttttaataag ttttaaagag ttttaggcgg aaaaatcgcc ttttttctct tttatatcag | |
| | 4441 | tcacttacat gtgtgaccgg ttcccaatgt acggctttgg gttcccaatg tacgggttcc | |
| | 4501 | ggttcccaat gtacggcttt gggttcccaa tgtacgtgct atccacagga aagagacctt | |
| | 4561 | ttcgaccttt ttcccctgct agggcaattt gccctagcat ctgctccgta cattaggaac | |
| | 4621 | cggcggatgc ttcgccctcg atcaggttgc ggtagcgcat gactaggatc gggccagcct | |
| | 4681 | gccccgcctc ctccttcada tcgtactccg gcaggtcatt tgacccgatc agcttgcgca | |
| | 4741 | cggtgaaaca gaacttcttg aactctccgg cgctgccact gcgttcgtag atcgtcttga | |
| | 4801 | acaaccatct ggcttctgcc ttgcctgcgg cgcggcgtgc caggcggtag agaaaacggc | |
| | 4861 | cgatgccggg atcgatcaaa aagtaatcgg ggtgaaccgt cagcacgtcc gggttcttgc | |
| | 4921 | cttctgtgat ctcgcggtac atccaatcag ctagctcgat ctcgatgtac tccggccgcc | |
| | 4981 | cggtttcgct ctttacgatc ttgtagcggc taatcaaggc ttcaccctcg gataccgtca | |
| | 5041 | ccaggcggcc gttcttggcc ttcttcgtac gctgcatggc aacgtgcgtg gtgtttaacc | |
| | 5101 | gaatgcaggt ttctaccagg tcgtctttct gctttccgcc atcggctcgc cggcagaact | |
| | 5161 | tgagtacgtc cgcaacgtgt ggacggaaca cgcggccggg cttgtctccc ttcccttccc | |
| | 5221 | ggtatcggtt catggattcg gttagatggg aaaccgccat cagtaccagg tcgtaatccc | |
| | 5281 | acacactggc catgccggcc ggccctgcgg aaacctctac gtgcccgtct ggaagctcgt | |
| | 5341 | agcggatcac ctcgccagct cgtcggtcac gcttcgacag acggaaaacg gccacgtcca | |
| | 5401 | tgatgctgcg actatcgcgg gtgcccacgt catagagcat cggaacgaaa aaatctggtt | |
| | 5461 | gctcgtcgcc cttgggcggc ttcctaatcg acggcgcacc ggctgccggc ggttgccggg | |
| | 5521 | attctttgcg gattcgatca gcggccgctt gccacgattc accggggcgt gcttctgcct | |
| | 5581 | cgatgcgttg ccgctgggcg gcctgcgcgg ccttcaactt ctccaccagg tcatcaccca | |
| | 5641 | gcgccgcgcc gatttgtacc gggccggatg gtttgcgacc gtcacgccga ttcctcgggc | |
| | 5701 | ttgggggttc cagtgccatt gcagggccgg cagacaaccc agccgcttac gcctggccaa | |
| | 5761 | ccgcccgttc ctccacacat ggggcattcc acggcgtcgg tgcctggttg ttcttgattt | |
| | 5821 | tccatgccgc ctcctttagc cgctaaaatt catctactca tttattcatt tgctcattta | |
| | 5881 | ctctggtagc tgcgcgatgt attcagatag cagctcggta atggtcttgc cttggcgtac | |
| | 5941 | cgcgtacatc ttcagcttgg tgtgatcctc cgccggcaac tgaaagttga cccgcttcat | |
| | 6001 | ggctggcgtg tctgccaggc tggccaacgt tgcagccttg ctgctgcgtg cgctcggacg | |
| | 6061 | gccggcactt agcgtgtttg tgcttttgct cattttctct ttacctcatt aactcaaatg | |
| | 6121 | agttttgatt taatttcagc ggccagcgcc tggacctcgc gggcagcgtc gccctcgggt | |
| | 6181 | tctgattcaa gaacggttgt gccggcggcg gcagtgcctg ggtagctcac gcgctgcgtg | |
| | 6241 | atacgggact caagaatggg cagctcgtac ccggccagcg cctcggcaac ctcaccgccg | |
| | 6301 | atgcgcgtgc ctttgatcgc ccgcgacacg acaaaggccg cttgtagcct tccatccgtg | |
| | 6361 | acctcaatgc gctgcttaac cagctccacc aggtcggcgg tggcccatat gtcgtaaggg | |
| | 6421 | cttggctgca ccggaatcag cacgaagtcg gctgccttga tcgcggacac agccaagtcc | |
| | 6481 | gccgcctggg gcgctccgtc gatcactacg aagtcgcgcc ggccgatggc cttcacgtcg | |
| | 6541 | cggtcaatcg tcgggcggtc gatgccgaca acggttagcg gttgatcttc ccgcacggcc | |
| | 6601 | gcccaatcgc gggcactgcc ctggggatcg gaatcgacta acagaacatc ggccccggcg | |
| | 6661 | agttgcaggg cgcgggctag atgggttgcg atggtcgtct tgcctgaccc gcctttctgg | |
| | 6721 | ttaagtacag cgataacctt catgcgttcc ccttgcgtat ttgtttattt actcatcgca | |
| | 6781 | tcatatacgc agcgaccgca tgacgcaagc tgttttactc aaatacacat caccttttta | |
| | 6841 | gacggcggcg ctcggtttct tcagcggcca agctggccgg ccaggccgcc agcttggcat | |
| | 6901 | cagacaaacc ggccaggatt tcatgcagcc gcacggttga gacgtgcgcg ggcggctcga | |
| | 6961 | acacgtaccc ggccgcgatc atctccgcct cgatctcttc ggtaatgaaa aacggttcgt | |
| | 7021 | cctggccgtc ctggtgcggt ttcatgcttg ttcctcttgg cgttcattct cggcggccgc | |
| | 7081 | cagggcgtcg gcctcggtca atgcgtcctc acggaaggca ccgcgccgcc tggcctcggt | |
| | 7141 | gggcgtcact tcctcgctgc gctcaagtgc gcggtacagg gtcgagcgat gcacgccaag | |
| | 7201 | cagtgcagcc gcctctttca cggtgcggcc ttcctggtcg atcagctcgc gggcgtgcgc | |
| | 7261 | gatctgtgcc ggggtgaggg tagggcgggg gccaaacttc acgcctcggg ccttggcggc | |
| | 7321 | ctcgcgcccg ctccgggtgc ggtcgatgat tagggaacgc tcgaactcgg caatgccggc | |
| | 7381 | gaacacggtc aacaccatgc ggccggccgg cgtggtggtg tcggcccacg gctctgccag | |
| | 7441 | gctacgcagg cccgcgccgg cctcctggat gcgctcggca atgtccagta ggtcgcgggt | |
| | 7501 | gctgcgggcc aggcggtcta gcctggtcac tgtcacaacg tcgccagggc gtaggtggtc | |
| | 7561 | aagcatcctg gccagctccg ggcggtcgcg cctggtgccg gtgatcttct cggaaaacag | |
| | 7621 | cttggtgcag ccggccgcgt gcagttcggc ccgttggttg gtcaagtcct ggtcgtcggt | |
| | 7681 | gctgacgcgg gcatagccca gcaggccagc ggcggcgctc ttgttcatgg cgtaatgtct | |
| | 7741 | ccggttctag tcgcaagtat tctactttat gcgactaaaa cacgcgacaa gaaaacgcca | |
| | 7801 | ggaaaagggc agggcggcag cctgtcgcgt aacttaggac ttgtgcgaca tgtcgttttc | |
| | 7861 | agaagacggc tgcactgaac gtcagaagcc gactgcacta tagcagcgga ggggttggat | |
| | 7921 | caaagtactt tgatcccgag gggaaccctg tggttggcat gcacatacaa atggacgaac | |
| | 7981 | ggataaacct tttcacgccc ttttaaatat ccgttattct aataaacgct cttttctctt | |
| | 8041 | aggtttaccc gccaatatat cctgtcaaac actgatagtt taaactgaag gcgggaaacg | |
| | 8101 | acaatctgat ccaagctcaa gctaagcttg agctctccca tatggtcgac tagagccaag | |
| | 8161 | ctgatctcct ttgccccgga gatcaccatg gacgactttc tctatctcta cgatctagga | |
| | 8221 | agaaagttcg acggagaagg tgacgatacc atgttcacca ccgataatga gaagattagc | |
| | 8281 | ctcttcaatt tcagaaagaa tgctgaccca cagatggtta gagaggccta cgcggcaggt | |
| | 8341 | ctcatcaaga cgatctaccc gagtaataat ctccaggaga tcaaatacct tcccaagaag | |
| | 8401 | gttaaagatg cagtcaaaag attcaggact aactgcatca agaacacaga gaaagatata | |
| | 8461 | tttctcaaga tcagaagtac tattccagta tggacgattc aaggcttgct tcataaacca | |
| | 8521 | aggcaagtaa tagagattgg agtctctaag aaagtagttc ctactgaatc aaaggccatg | |
| | 8581 | gagtcaaaaa ttcagatcga ggatctaaca gaactcgccg tgaagactgg cgaacagttc | |
| | 8641 | atacagagtc ttttacgact caatgacaag aagaaaatct tcgtcaacat ggtggagcac | |
| | 8701 | gacactctcg tctactccaa gaatatcaaa gatacagtct cagaagacca aagggctatt | |
| | 8761 | gagacttttc aacaaagggt aatatcggga aacctcctcg gattccattg cccagctatc | |
| | 8821 | tgtcacttca tcaaaaggac agtagaaaag gaaggtggca cctacaaatg ccatcattgc | |
| | 8881 | gataaaggaa aggctatcgt tcaagatgcc tctgccgaca gtggtcccaa agatggaccc | |
| | 8941 | ccacccacga ggagcatcgt ggaaaaagaa gacgttccaa ccacgtcttc aaagcaagtg | |
| | 9001 | gattgatgtg atatctccac tgacgtaagg gatgacgcac aatcccacta tccttcgcaa | |
| | 9061 | gacccttcct ctatataagg aagttcattt catttggaga ggactccggt atttttacaa | |
| | 9121 | caataccaca acaaaacaaa caacaaacaa cattacaatt tactattcta gtcgacctgc | |
| | 9181 | aggcggccgc actagtgata tcacaagttt gtacaaaaaa gcaggctccg cggccgcccc | |
| | 9241 | cttcaccatg gtttccggag tcgggggtag tggcggtggc cgtggcggtg gccgtggcgg | |
| | 9301 | agaagaagaa ccgtcgtcaa gtcacactcc taataaccga agaggaggag aacaagctca | |
| | 9361 | atcgtcggga acgaaatctc tcagaccaag aagcaacact gaatcaatga gcaaagcaat | |
| | 9421 | tcaacagtac accgtcgacg caagactcca cgccgttttc gaacaatccg gcgaatcagg | |
| | 9481 | gaaatcattc gactactcac aatcactcaa aacgacgacg tacggttcct ctgtacctga | |
| | 9541 | gcaacagatc acagcttatc tctctcgaat ccagcgaggt ggttacattc agcctttcgg | |
| | 9601 | atgtatgatc gccgtcgatg aatccagttt ccggatcatc ggttacagtg aaaacgccag | |
| | 9661 | agaaatgtta gggattatgc ctcaatctgt tcctactctt gagaaacctg agattctagc | |
| | 9721 | tatgggaact gatgtgagat ctttgttcac ttcttcgagc tcgattctac tcgagcgtgc | |
| | 9781 | tttcgttgct cgagagatta ccttgttaaa tccggtttgg atccattcca agaatactgg | |
| | 9841 | taaaccgttt tacgccattc ttcataggat tgatgttggt gttgttattg atttagagcc | |
| | 9901 | agctagaact gaagatcctg cgctttctat tgctggtgct gttcaatcgc agaaactcgc | |
| | 9961 | ggttcgtgcg atttctcagt tacaggctct tcctggtgga gatattaagc ttttgtgtga | |
| | 10021 | cactgtcgtg gaaagtgtga gggacttgac tggttatgat cgtgttatgg ttcataagtt | |
| | 10081 | tcatgaagat gagcatggag aagttgtagc tgagagtaaa cgagatgatt tagagcctta | |
| | 10141 | tattggactg cattatcctg ctactgatat tcctcaagcg tcaaggttct tgtttaagca | |
| | 10201 | gaaccgtgtc cgaatgatag tagattgcaa tgccacacct gttcttgtgg tccaggacga | |
| | 10261 | taggctaact cagtctatgt gcttggttgg ttctactctt agggctcctc atggttgtca | |
| | 10321 | ctctcagtat atggctaaca tgggatctat tgcgtcttta gcaatggcgg ttataatcaa | |
| | 10381 | tggaaatgaa gatgatggga gcaatgtagc tagtggaaga agctcgatga ggctttgggg | |
| | 10441 | tttggttgtt tgccatcaca cttcttctcg ctgcataccg tttccgctaa ggtatgcttg | |
| | 10501 | tgagtttttg atgcaggctt tcggtttaca gttaaacatg gaattgcagt tagctttgca | |
| | 10561 | aatgtcagag aaacgcgttt tgagaacgca gacactgtta tgtgatatgc ttctgcgtga | |
| | 10621 | ctcgcctgct ggaattgtta cacagagtcc cagtatcatg gacttagtga aatgtgacgg | |
| | 10681 | tgcagcattt ctttaccacg ggaagtatta cccgttgggt gttgctccta gtgaagttca | |
| | 10741 | gataaaagat gttgtggagt ggttgcttgc gaatcatgcg gattcaaccg gattaagcac | |
| | 10801 | tgatagttta ggcgatgcgg ggtatcccgg tgcagctgcg ttaggggatg ctgtgtgcgg | |
| | 10861 | tatggcagtt gcatatatca caaaaagaga ctttcttttt tggtttcgat ctcacactgc | |
| | 10921 | gaaagaaatc aaatggggag gcgctaagca tcatccggag gataaagatg atgggcaacg | |
| | 10981 | aatgcatcct cgttcgtcct ttcaggcttt tcttgaagtt gttaagagcc ggagtcagcc | |
| | 11041 | atgggaaact gcggaaatgg atgcgattca ctcgctccag cttattctga gagactcttt | |
| | 11101 | taaagaatct gaggcggcta tgaactctaa agttgtggat ggtgtggttc agccatgtag | |
| | 11161 | ggatatggcg ggggaacagg ggattgatga gttaggtgca gttgcaagag agatggttag | |
| | 11221 | gctcattgag actgcaactg ttcctatatt cgctgtggat gccggaggct gcatcaatgg | |
| | 11281 | atggaacgct aagattgcag agttgacagg tctctcagtt gaagaagcta tggggaagtc | |
| | 11341 | tctggtttct gatttaatat acaaagagaa tgaagcaact gtcaataagc ttctttctcg | |
| | 11401 | tgctttgaga ggggacgagg aaaagaatgt ggaggttaag ctgaaaactt tcagccccga | |
| | 11461 | actacaaggg aaagcagttt ttgtggttgt gaatgcttgt tccagcaagg actacttgaa | |
| | 11521 | caacattgtc ggcgtttgtt ttgttggaca agacgttact agtcagaaaa tcgtaatgga | |
| | 11581 | taagttcatc aacatacaag gagattacaa ggctattgta catagcccaa accctctaat | |
| | 11641 | cccgccaatt tttgctgctg acgagaacac gtgctgcctg gaatggaaca tggcgatgga | |
| | 11701 | aaagcttacg ggttggtctc gcagtgaagt gattgggaaa atgattgtcg gggaagtgtt | |
| | 11761 | tgggagctgt tgcatgctaa agggtcctga tgctttaacc aagttcatga ttgtattgca | |
| | 11821 | taatgcgatt ggtggccaag atacggataa gttccctttc ccattctttg accgcaatgg | |
| | 11881 | gaagtttgtt caggctctat tgactgcaaa caagcgggtt agcctcgagg gaaaggttat | |
| | 11941 | tggggctttc tgtttcttgc aaatcccgag ccctgagctg cagcaagctt tagcagtcca | |
| | 12001 | acggaggcag gacacagagt gtttcacgaa ggcaaaagag ttggcttata tttgtcaggt | |
| | 12061 | gataaagaat cctttgagcg gtatgcgttt cgcaaactca ttgttggagg ccacagactt | |
| | 12121 | gaacgaggac cagaagcagt tacttgaaac aagtgtttct tgcgagaaac agatctcaag | |
| | 12181 | gatcgtcggg gacatggatc ttgaaagcat tgaagacggt tcatttgtgc taaagaggga | |
| | 12241 | agagtttttc cttggaagtg tcataaacgc gattgtaagt caagcgatgt tcttattaag | |
| | 12301 | ggacagaggt cttcagctga tccgtgacat tcccgaagag atcaaatcaa tagaggtttt | |
| | 12361 | tggagaccag ataaggattc aacagctcct ggctgagttt ctgctgagta taatccggta | |
| | 12421 | tgcaccatct caagagtggg tggagatcca tttaagccaa ctttcaaagc aaatggctga | |
| | 12481 | tggattcgcc gccatccgca cagaattcag aatggcgtgt ccaggtgaag gtctgcctcc | |
| | 12541 | agagctagtc cgagacatgt tccatagcag caggtggaca agccctgaag gtttaggtct | |
| | 12601 | aagcgtatgt cgaaagattt taaagctaat gaacggtgag gttcaataca tccgagaatc | |
| | 12661 | agaacggtcc tatttcctca tcattctgga actccctgta cctcgaaagc gaccattgtc | |
| | 12721 | aactgctagt ggaagtggtg acatgatgct gatgatgcca tataagggtg ggcgcgccga | |
| | 12781 | cccagctttc ttgtacaaag tggtgcgaat ggtgaacaag ggcgaggagc tgttcaccgg | |
| | 12841 | ggtggtgccc atcctggtcg agctggacgg cgacgtaaac ggccacaagt tcagcgtgtc | |
| | 12901 | cggcgagggc gagggcgatg ccacctacgg caagctgacc ctgaagttca tctgcaccac | |
| | 12961 | cggcaagctg cccgtgccct ggcccaccct cgtgaccacc ctgacctggg gcgtgcagtg | |
| | 13021 | cttcgcccgc taccccgacc acatgaagca gcacgacttc ttcaagtccg ccatgcccga | |
| | 13081 | aggctacgtc caggagcgca ccatcttctt caaggacgac ggcaactaca agacccgcgc | |
| | 13141 | cgaggtgaag ttcgagggcg acaccctggt gaaccgcatc gagctgaagg gcatcgactt | |
| | 13201 | caaggaggac ggcaacatcc tggggcacaa gctggagtac aacgccatca gcgacaacgt | |
| | 13261 | ctatatcacc gccgacaagc agaagaacgg catcaaggcc aacttcaaga tccgccacaa | |
| | 13321 | catcgaggac ggcagcgtgc agctcgccga ccactaccag cagaacaccc ccatcggcga | |
| | 13381 | cggccccgtg ctgctgcccg acaaccacta cctgagcacc cagtccaagc tgagcaaaga | |
| | 13441 | ccccaacgag aagcgcgatc acatggtcct gctggagttc gtgaccgccg ccgggatcac | |
| | 13501 | tctcggcatg gacgagctgt acaagtctag gggtggaagc tggagccacc ctcaatttga | |
| | 13561 | aaagggagga ggatctggag gtggttctgg tggtggttct tggtctcacc cacaattcga | |
| | 13621 | aaagggttct tacccatacg atgttccaga ttacgctcat caccatcacc atcactaggg | |
| | 13681 | tggtgatatc ccgcggccat gctagagtcc gcaaaaatca ccagtctctc tctacaaatc | |
| | 13741 | tatctctctc tatttttctc cagaataatg tgtgagtagt tcccagataa gggaattagg | |
| | 13801 | gttcttatag ggtttcgctc atgtgttgag catataagaa acccttagta tgtatttgta | |
| | 13861 | tttgtaaaat acttctatca ataaaatttc taattcctaa aaccaaaatc cagtgacctg | |
| | 13921 | caggcatgcg acgtcg | |
| 20 | 1 | atgaagagag ggaaatatga ggagaagata cttgagccga tgtttcctcg acttcatgtg | Thlaspi arvensi |
| | 61 | aacgatgctg ataaaggagg gcctagagct cctccaagaa acaagatggc tctctatgag | wt ELF3 cDNA |
| | 121 | caacttagca tcccttctca gaggtttagt agtgatcatg gatcgttgcc tcgtaacacc | |
| | 181 | agcactttgg ttcctcctgt tggatcatct agtcagcctt gtagcgtgga aagaaactta | |
| | 241 | tctgcccagc atcttgattc ttcagctgca gtccatgcaa ctgagaagtt tgtctcccaa | |
| | 301 | atgtcgttca tggaaaatgt gagaattttg gcacagcatg atcagaaaaa attagtaaga | |
| | 361 | gaggaggatg atttcgcagt tccagtattt attaactcaa gaagatttca gtctcatggc | |
| | 421 | agaaccaaga gaggtgttga gaaggaaaga cacaccacat tggtcaaggg gagcgctaag | |
| | 481 | cgaggtggct ttgtaaactc tgtggatgta tcagctacag aggaaatgga tcttgaaaaa | |
| | 541 | tcatcatcga gttgtgacag agttaatgat tgtaatgatt ccttgagaga ggagtctaga | |
| | 601 | aataggttag gccgagatgg tggtgaaact cgagcgatgg acactgataa tggagttgaa | |
| | 661 | tctcacttgg gatcagaatg tcattcagaa gagggtcatg acaatcctaa tgacgttgat | |
| | 721 | aatggtcgtg aatactgcac aagtagagga tatgtctctc tgcagcagat aaatgaagag | |
| | 781 | gcgagcgatg atgtttctga tgattcgatg gtggattcta tatccagcat agatgtctct | |
| | 841 | cctgatgacg ttgtgggagt attaggtcaa aaacgtttct ggagagcaag gaaagctatt | |
| | 901 | gccaatcaac aaagagtatt tgctgttcaa ctatttgagt tgcacagact gattaaggta | |
| | 961 | caaagactta ttgctgcatc accggatgtc ttgctcgatg acatcagtta tcttggaaaa | |
| | 1021 | gtcgctgtta aaagctttcc agtgaagaag ctccttccat cagagaagcc tcctctacca | |
| | 1081 | caagttatca aacacagcgg ccgcgactcc gcggagaaga ccgaccaaca taaaatggaa | |
| | 1141 | tgctcggctg agaacgtagt cgggaagatg tcaaaccaag ttcatcatca accttccaac | |
| | 1201 | tacatgcctt ttccgagcaa ccaagccact gctaatggat gttactttcc tccccagcct | |
| | 1261 | cctacttcag gaggaaatca gcaatggtta atccctgtaa tgtctccttc cgaaggtctg | |
| | 1321 | atatacaagc cgcatccagg tccaggacac acaggacctc cggtctgtgg agggtattat | |
| | 1381 | ggtcattaca tgcctgctcc tatggtaatg ggtaatttca tgggcggtgg tggtcaacct | |
| | 1441 | ccgtttcacc cggggatggg ccttgcatct ctgggtaatg gctacttccc tccttatggt | |
| | 1501 | ataatgccac acaccatgat gaatacttat tgctccggcc aacaacaaca acaacaaccc | |
| | 1561 | aatgagcata tgaatcagtt tgggcattca gtaaatcttc agaacgcatc aggtttgaac | |
| | 1621 | aaccaacaac agagctcggt taacgaagag gcggcgcaac aacagccggc gaaatcatat | |
| | 1681 | catcgggcta gaaagagcag gcaagggagc acaggaagca gcagttcaga gggaatctcc | |
| | 1741 | ggtaagaact cctttcggcc attctcagcg gttgatgagg acagcatcaa cgaccaacct | |
| | 1801 | gagcaaatga tgacgacaac gacgaccaca acaagaacaa ctgttactca gacgacaaga | |
| | 1861 | gatggagtag gaggagtgac gagagtgatc aaagtggttc cacacaacgc gaagctggcg | |
| | 1921 | agtgagtatg ctgcaaggat tttccggtca attcaagaag aacgtaaaca ctacgactcc | |
| | 1981 | ttctctaacc actcttaa | |
| 21 | MKRGKYEEKILEPMFPRLHVNDADKGGPRAPPRNKMALYEQLSIPSQRFSSDHGSLPRNTSTLVPPVGSSS | | Thlaspi arvensi |
| | QPCSVERNLSAQHLDSSAAVHATEKFVSQMSFMENVRILAQHDQKKLVREEDDFAVPVFINSRRFQSHGRT | | wt ELF3 protein |
| | KRGVEKERHTTLVKGSAKRGGFVNSVDVSATEEMDLEKSSSSCDRVNDCNDSLREESRNRLGRDGGETRAM | | |
| | DTDNGVESHLGSECHSEEGHDNPNDVDNGREYCTSRGYVSLOQINEEASDDVSDDSMVDSISSIDVSPDDV | | |
| | VGVLGQKRFWRARKAIANQQRVFAVOLFELHRLIKVORLIAASPDVLLDDISYLGKVAVKSFPVKKLLPSE | | |
| | KPPLPQVIKHSGRDSAEKTDQHKMECSAENVVGKMSNQVHHQPSNYMPFPSNQATANGCYFPPOPPTSGGN | | |
| | QQWLIPVMSPSEGLIYKPHPGPGHTGPPVCGGYYGHYMPAPMVMGNFMGGGGQPPFHPGMGLASLGNGYFP | | |
| | PYGIMPHTMMNTYCSGQQQQQQPNEHMNQFGHSVNLONASGLNNOQQSSVNEEAAQQQPAKSYHRARKSRQ | | |
| | GSTGSSSSEGISGKNSFRPFSAVDEDSINDQPEQMMTTTTTTTRTTVTQTTRDGVGGVTRVIKVVPHNAKL | | |
| | ASEYAARIFRSIQEERKHYDSFSNHS- | | |
| 22 | AGCGAGGTGGCTTTGTAAACTCTGTGGATGTATCAGCTACAGAGGAAATG | | Large deletion |
| | GATCTTGAAAAATCATCATCGAGTTGTGACAGAGTTAATGATTGTAATGATTCCTTGA | | in Thlaspi |
| | GAGAGGAGTCTAGAAATAGGTTAGGCCGAGATGGTGGTGAAACTCGAGCGATGGACAC | | arvensi ELF3 |
| | TGATAATGGAGTTGAATCTCACTTGGGATCAGAATGTCATTCAGAAGAGGGTCATGAC | | (Taelf3-1). |
| | AATCCTAATGACGTTGATAATGGTCGTGAATACTGCACAAGTAGAGGATATGTCTCTC | | Shows sequence |
| | TGCAGCAGATAAATGAAGAGGCGAGCGATGATGTTTCTGATGATTCGATGGTGGATTC | | deleted from |
| | TATATCCAGCATAGATGTCTCTCCTGATGACGTTGTGGGAGTATTAGGTCAAAAACGT | | ELF3. See FIG. |
| | TTCTGGAGAGCAAGGAAAGCTATTGCCAATCAACAAAGAGTATTTGCTGTTCAACTAT | | 13 |
| | TTGAGTTGCACAGACTGATTAAGGTACAAAGACTTATTGCTGCATCACCGGATGTCTT | | |
| | GCTCGATGACATCAGTTATCTTGGAAAAGTCGCTGTTAAAAGCTTTCCAGTGAAGAAG | | |
| | CTCCTTCCATCAGAGAAGCCTCCTCTACCACAAGTTATCAAAC | | |
| 23 | ATTGTATGAAGAGAGGGAATGATGTTTTAGAGCTAGAAATAGCAAGTTAAAATAAGGCTAGTCCGTTATCA | | Expression |
| | ACTTGAAAAAGTGGCACCGAGTCGGTGCTTTTTTTTGCAAAATTTTCCAGATCGATTTCTTCTTCCTCTGT | | construct for |
| | TCTTCGGCGTTCAATTTCTGGGGTTTTCTCTTCGTTTTCTGTAACTGAAACCTAAAATTTGACCTAAAAAA | | expressing |
| | AATCTCAAATAATATGATTCAGTGGTTTTGTACTTTTCAGTTAGTTGAGTTTTGCAGTTCCGATGAGATAA | | gRNAs of |
| | ACCAATATTAATCCAAACTACTGCAGCCTGACAGACAAATGAGGATGCAAACAATTTTAAAGTTTATCTAA | | phse401-basil- |
| | CGCTAGCTGTTTTGTTTCTTCTCTCTGGTGCACCAACGACGGCGTTTTCTCAATCATAAAGAGGCTTGTTT | | x4 CRISPR |
| | TACTTAAGGCCAATAATGTTGATGGATCGAAAGAAGAGGGCTTTTAATAAACGAGCCCGTTTAAGCTGTAA | | construct (SEQ |
| | ACGATGTCAAAAACATCCCACATCGTTCAGTTGAAAATAGAAGCTCTGTTTATATATTGGTAGAGTCGACT | | ID NO: 37) to |
| | AAGAGATTGGCGTTGTTAGTCGGGTTTCGTTTTAGAGCTAGAAATAGCAAGTTAAAATAAGGCTAGTCCGT | | disrupt elf3 in |
| | TATCAACTTGAAAAAGTGGCACCGAGTCGGTGCTTTTTTTGGATAGAATTTCCCAGCTTTTTTGCGTGTTT | | basil |
| | CAGCTCTCATGATCCTTGGCCAATGGGTGTAGTAAATTTTCTGCACATTCATTGGATGGAAAATAATGGTT | | |
| | TTAGCTTTAGGGAATAAGAAAAGTGTATAGGAAGGGGATTTTTGTACAATCACATTTGAATTAGGTCTTTG | | |
| | AAATGACAGGGAATGAGGACATATGATGAGACGGTCATTGTTTTAGTTCCACCACGATTATATTTGAAATT | | |
| | TACGTGAGTGTGAGTGAGACTTGCATAAGAAAATAAAATCTTTAGTTGGGAAAAAATTCAATAATATAAAT | | |
| | GGGCTTGAGAAGGAAGCGAGGGATAGGCCTTTTTCTAAAATAGGCCCATTTAAGCTATTAACAATCTTCAA | | |
| | AAGTACCACAGCGCTTAGGTAAAGAAAGCAGCTGAGTTTATATATGGTTAGAGACGAAGTAGTGATTGAAT | | |
| | CGGTGAGTCTGTGGGAGTTTTAGAGCTAGAAATAGCAAGTTAAAATAAGGCTAGTCCGTTATCAACTTGAA | | |
| | AAAGTGGCACCGAGTCGGTGCTTTTTTTTGGCAAAAATTTTCAGATTTTTTCTTCATCTGTAGATTTCTGG | | |
| | GTTTTTTTTTCCGTTTCGTGAATCATAAGTGAAGTTTTGGATGCAAATCTGCGCGAAAAAAGTTGGACCTG | | |
| | CAATGAGCTTATTTAGATAGCTAAGACAAAGTGATTGGTCCGTTCGACTTGCCTTCCGCACAATACATCAT | | |
| | TTCTTCTTAGCTTTTTTTCTTCTTCTTCGTTCATACAGTTTTTTTTTGTTTATCAGCTTACATTTTCTTGA | | |
| | ACCGTAGCTTTCGTTTTCTTCTTTTTAACTTTCCATTCGGAGTTTTTGTATCTTGTTTCATAGTTTGTCCC | | |
| | AGGATTAGAATGATTAGGCATCGAACCTTCAAGAATTTGATTGAATAAAACATCTTCATTCTTAAGATATG | | |
| | AAGATAATCTTCAAAAGGCCCCTGGGAATCTGAAAGAAGAGAAGCAGGCCCATTTATATGGGAAAGAACAA | | |
| | TAGTATTTCTTATATAGGCCCATTTAAGTTGAAAACAATCTTCAAAAGTCCCACATCGCTTAGATAAGAAA | | |
| | ACGAAGCTGAGTTTATATACAGCTAGAGTCGAAGTAGTGATTGACGGACGATTGAGACTAGAGTTTTAGAG | | |
| | CTAGAAATAGCAAGTTAAAATAAGGCTAGTCCGTTATCAACTTGAAAAAGTGGCACCGAGTCGGTGC | | |
| 24 | GACTAGAGCCAAGCTGATCTCCTTTGCCCCGGAGATCACCATGGACGACTTTCTCTATCTCTACGATCTAG | | A.t. YHB |
| | GAAGAAAGTTCGACGGAGAAGGTGACGATACCATGTTCACCACCGATAATGAGAAGATTAGCCTCTTCAAT | | expression |
| | TTCAGAAAGAATGCTGACCCACAGATGGTTAGAGAGGCCTACGCGGCAGGTCTCATCAAGACGATCTACCC | | construct from |
| | GAGTAATAATCTCCAGGAGATCAAATACCTTCCCAAGAAGGTTAAAGATGCAGTCAAAAGATTCAGGACTA | | SEQ ID NO 19 |
| | ACTGCATCAAGAACACAGAGAAAGATATATTTCTCAAGATCAGAAGTACTATTCCAGTATGGACGATTCAA | | |
| | GGCTTGCTTCATAAACCAAGGCAAGTAATAGAGATTGGAGTCTCTAAGAAAGTAGTTCCTACTGAATCAAA | | |
| | GGCCATGGAGTCAAAAATTCAGATCGAGGATCTAACAGAACTCGCCGTGAAGACTGGCGAACAGTTCATAC | | |
| | AGAGTCTTTTACGACTCAATGACAAGAAGAAAATCTTCGTCAACATGGTGGAGCACGACACTCTCGTCTAC | | |
| | TCCAAGAATATCAAAGATACAGTCTCAGAAGACCAAAGGGCTATTGAGACTTTTCAACAAAGGGTAATATC | | |
| | GGGAAACCTCCTCGGATTCCATTGCCCAGCTATCTGTCACTTCATCAAAAGGACAGTAGAAAAGGAAGGTG | | |
| | GCACCTACAAATGCCATCATTGCGATAAAGGAAAGGCTATCGTTCAAGATGCCTCTGCCGACAGTGGTCCC | | |
| | AAAGATGGACCCCCACCCACGAGGAGCATCGTGGAAAAAGAAGACGTTCCAACCACGTCTTCAAAGCAAGT | | |
| | GGATTGATGTGATATCTCCACTGACGTAAGGGATGACGCACAATCCCACTATCCTTCGCAAGACCCTTCCT | | |
| | CTATATAAGGAAGTTCATTTCATTTGGAGAGGACTCCGGTATTTTTACAACAATACCACAACAAAACAAAC | | |
| | AACAAACAACATTACAATTTACTATTCTAGTCGACCTGCAGGCGGCCGCACTAGTGATATCACAAGTTTGT | | |
| | ACAAAAAAGCAGGCTCCGCGGCCGCCCCCTTCACCATGGTTTCCGGAGTCGGGGGTAGTGGCGGTGGCCGT | | |
| | GGCGGTGGCCGTGGCGGAGAAGAAGAACCGTCGTCAAGTCACACTCCTAATAACCGAAGAGGAGGAGAACA | | |
| | AGCTCAATCGTCGGGAACGAAATCTCTCAGACCAAGAAGCAACACTGAATCAATGAGCAAAGCAATTCAAC | | |
| | AGTACACCGTCGACGCAAGACTCCACGCCGTTTTCGAACAATCCGGCGAATCAGGGAAATCATTCGACTAC | | |
| | TCACAATCACTCAAAACGACGACGTACGGTTCCTCTGTACCTGAGCAACAGATCACAGCTTATCTCTCTCG | | |
| | AATCCAGCGAGGTGGTTACATTCAGCCTTTCGGATGTATGATCGCCGTCGATGAATCCAGTTTCCGGATCA | | |
| | TCGGTTACAGTGAAAACGCCAGAGAAATGTTAGGGATTATGCCTCAATCTGTTCCTACTCTTGAGAAACCT | | |
| | GAGATTCTAGCTATGGGAACTGATGTGAGATCTTTGTTCACTTCTTCGAGCTCGATTCTACTCGAGCGTGC | | |
| | TTTCGTTGCTCGAGAGATTACCTTGTTAAATCCGGTTTGGATCCATTCCAAGAATACTGGTAAACCGTTTT | | |
| | ACGCCATTCTTCATAGGATTGATGTTGGTGTTGTTATTGATTTAGAGCCAGCTAGAACTGAAGATCCTGCG | | |
| | CTTTCTATTGCTGGTGCTGTTCAATCGCAGAAACTCGCGGTTCGTGCGATTTCTCAGTTACAGGCTCTTCC | | |
| | TGGTGGAGATATTAAGCTTTTGTGTGACACTGTCGTGGAAAGTGTGAGGGACTTGACTGGTTATGATCGTG | | |
| | TTATGGTTCATAAGTTTCATGAAGATGAGCATGGAGAAGTTGTAGCTGAGAGTAAACGAGATGATTTAGAG | | |
| | CCTTATATTGGACTGCATTATCCTGCTACTGATATTCCTCAAGCGTCAAGGTTCTTGTTTAAGCAGAACCG | | |
| | TGTCCGAATGATAGTAGATTGCAATGCCACACCTGTTCTTGTGGTCCAGGACGATAGGCTAACTCAGTCTA | | |
| | TGTGCTTGGTTGGTTCTACTCTTAGGGCTCCTCATGGTTGTCACTCTCAGTATATGGCTAACATGGGATCT | | |
| | ATTGCGTCTTTAGCAATGGCGGTTATAATCAATGGAAATGAAGATGATGGGAGCAATGTAGCTAGTGGAAG | | |
| | AAGCTCGATGAGGCTTTGGGGTTTGGTTGTTTGCCATCACACTTCTTCTCGCTGCATACCGTTTCCGCTAA | | |
| | GGTATGCTTGTGAGTTTTTGATGCAGGCTTTCGGTTTACAGTTAAACATGGAATTGCAGTTAGCTTTGCAA | | |
| | ATGTCAGAGAAACGCGTTTTGAGAACGCAGACACTGTTATGTGATATGCTTCTGCGTGACTCGCCTGCTGG | | |
| | AATTGTTACACAGAGTCCCAGTATCATGGACTTAGTGAAATGTGACGGTGCAGCATTTCTTTACCACGGGA | | |
| | AGTATTACCCGTTGGGTGTTGCTCCTAGTGAAGTTCAGATAAAAGATGTTGTGGAGTGGTTGCTTGCGAAT | | |
| | CATGCGGATTCAACCGGATTAAGCACTGATAGTTTAGGCGATGCGGGGTATCCCGGTGCAGCTGCGTTAGG | | |
| | GGATGCTGTGTGCGGTATGGCAGTTGCATATATCACAAAAAGAGACTTTCTTTTTTGGTTTCGATCTCACA | | |
| | CTGCGAAAGAAATCAAATGGGGAGGCGCTAAGCATCATCCGGAGGATAAAGATGATGGGCAACGAATGCAT | | |
| | CCTCGTTCGTCCTTTCAGGCTTTTCTTGAAGTTGTTAAGAGCCGGAGTCAGCCATGGGAAACTGCGGAAAT | | |
| | GGATGCGATTCACTCGCTCCAGCTTATTCTGAGAGACTCTTTTAAAGAATCTGAGGCGGCTATGAACTCTA | | |
| | AAGTTGTGGATGGTGTGGTTCAGCCATGTAGGGATATGGCGGGGGAACAGGGGATTGATGAGTTAGGTGCA | | |
| | GTTGCAAGAGAGATGGTTAGGCTCATTGAGACTGCAACTGTTCCTATATTCGCTGTGGATGCCGGAGGCTG | | |
| | CATCAATGGATGGAACGCTAAGATTGCAGAGTTGACAGGTCTCTCAGTTGAAGAAGCTATGGGGAAGTCTC | | |
| | TGGTTTCTGATTTAATATACAAAGAGAATGAAGCAACTGTCAATAAGCTTCTTTCTCGTGCTTTGAGAGGG | | |
| | GACGAGGAAAAGAATGTGGAGGTTAAGCTGAAAACTTTCAGCCCCGAACTACAAGGGAAAGCAGTTTTTGT | | |
| | GGTTGTGAATGCTTGTTCCAGCAAGGACTACTTGAACAACATTGTCGGCGTTTGTTTTGTTGGACAAGACG | | |
| | TTACTAGTCAGAAAATCGTAATGGATAAGTTCATCAACATACAAGGAGATTACAAGGCTATTGTACATAGC | | |
| | CCAAACCCTCTAATCCCGCCAATTTTTGCTGCTGACGAGAACACGTGCTGCCTGGAATGGAACATGGCGAT | | |
| | GGAAAAGCTTACGGGTTGGTCTCGCAGTGAAGTGATTGGGAAAATGATTGTCGGGGAAGTGTTTGGGAGCT | | |
| | GTTGCATGCTAAAGGGTCCTGATGCTTTAACCAAGTTCATGATTGTATTGCATAATGCGATTGGTGGCCAA | | |
| | GATACGGATAAGTTCCCTTTCCCATTCTTTGACCGCAATGGGAAGTTTGTTCAGGCTCTATTGACTGCAAA | | |
| | CAAGCGGGTTAGCCTCGAGGGAAAGGTTATTGGGGCTTTCTGTTTCTTGCAAATCCCGAGCCCTGAGCTGC | | |
| | AGCAAGCTTTAGCAGTCCAACGGAGGCAGGACACAGAGTGTTTCACGAAGGCAAAAGAGTTGGCTTATATT | | |
| | TGTCAGGTGATAAAGAATCCTTTGAGCGGTATGCGTTTCGCAAACTCATTGTTGGAGGCCACAGACTTGAA | | |
| | CGAGGACCAGAAGCAGTTACTTGAAACAAGTGTTTCTTGCGAGAAACAGATCTCAAGGATCGTCGGGGACA | | |
| | TGGATCTTGAAAGCATTGAAGACGGTTCATTTGTGCTAAAGAGGGAAGAGTTTTTCCTTGGAAGTGTCATA | | |
| | AACGCGATTGTAAGTCAAGCGATGTTCTTATTAAGGGACAGAGGTCTTCAGCTGATCCGTGACATTCCCGA | | |
| | AGAGATCAAATCAATAGAGGTTTTTGGAGACCAGATAAGGATTCAACAGCTCCTGGCTGAGTTTCTGCTGA | | |
| | GTATAATCCGGTATGCACCATCTCAAGAGTGGGTGGAGATCCATTTAAGCCAACTTTCAAAGCAAATGGCT | | |
| | GATGGATTCGCCGCCATCCGCACAGAATTCAGAATGGCGTGTCCAGGTGAAGGTCTGCCTCCAGAGCTAGT | | |
| | CCGAGACATGTTCCATAGCAGCAGGTGGACAAGCCCTGAAGGTTTAGGTCTAAGCGTATGTCGAAAGATTT | | |
| | TAAAGCTAATGAACGGTGAGGTTCAATACATCCGAGAATCAGAACGGTCCTATTTCCTCATCATTCTGGAA | | |
| | CTCCCTGTACCTCGAAAGCGACCATTGTCAACTGCTAGTGGAAGTGGTGACATGATGCTGATGATGCCATA | | |
| | TAAGGGTGGGCGCGCCGACCCAGCTTTCTTGTACAAAGTGGTGCGAATGGTGAACAAGGGCGAGGAGCTGT | | |
| | TCACCGGGGTGGTGCCCATCCTGGTCGAGCTGGACGGCGACGTAAACGGCCACAAGTTCAGCGTGTCCGGC | | |
| | GAGGGCGAGGGCGATGCCACCTACGGCAAGCTGACCCTGAAGTTCATCTGCACCACCGGCAAGCTGCCCGT | | |
| | GCCCTGGCCCACCCTCGTGACCACCCTGACCTGGGGCGTGCAGTGCTTCGCCCGCTACCCCGACCACATGA | | |
| | AGCAGCACGACTTCTTCAAGTCCGCCATGCCCGAAGGCTACGTCCAGGAGCGCACCATCTTCTTCAAGGAC | | |
| | GACGGCAACTACAAGACCCGCGCCGAGGTGAAGTTCGAGGGCGACACCCTGGTGAACCGCATCGAGCTGAA | | |
| | GGGCATCGACTTCAAGGAGGACGGCAACATCCTGGGGCACAAGCTGGAGTACAACGCCATCAGCGACAACG | | |
| | TCTATATCACCGCCGACAAGCAGAAGAACGGCATCAAGGCCAACTTCAAGATCCGCCACAACATCGAGGAC | | |
| | GGCAGCGTGCAGCTCGCCGACCACTACCAGCAGAACACCCCCATCGGCGACGGCCCCGTGCTGCTGCCCGA | | |
| | CAACCACTACCTGAGCACCCAGTCCAAGCTGAGCAAAGACCCCAACGAGAAGCGCGATCACATGGTCCTGC | | |
| | TGGAGTTCGTGACCGCCGCCGGGATCACTCTCGGCATGGACGAGCTGTACAAGTCTAGGGGTGGAAGCTGG | | |
| | AGCCACCCTCAATTTGAAAAGGGAGGAGGATCTGGAGGTGGTTCTGGTGGTGGTTCTTGGTCTCACCCACA | | |
| | ATTCGAAAAGGGTTCTTACCCATACGATGTTCCAGATTACGCTCATCACCATCACCATCACTAG | | |
| 25 | 1 | atggtttccg gaggtaacgg cggtagtggc cgcggacgtg gtgaagaagc agcgtcgtca | Thlaspi arvensi |
| | 61 | agtcaccgtg ccgaacgcag tctccacacc acaacttatc accggagaga acaagctcaa | version of YHB |
| | 121 | tcttctggga cgaaatcgct taggccgcaa aatcagccgc agagccacac tgaatcagtg | allele of phyB. |
| | 181 | agcaaagcca ttcaacagta caccgtcgac gcgaggctcc acgccgtctt cgaacagtct | cDNA |
| | 241 | ggcgagtccg gcaagtcgtt tgattactca cagtccctca aaacgacgac gtacgggtcc | TaPHYB_YHB_cDNA |
| | 301 | tctgtaccgg agcagcagat cacggcgtat ctctctagga tccagcgagg aggctacatc | |
| | 361 | cagcccttcg gatgtatgat cgccgtcgat gaatccacct tcagaatcat cggctacagc | |
| | 421 | gaaaacgcgc gggaaatgtt aggccttact cctcagtccg ttcctagcct cgagaaaccg | |
| | 481 | gagattctag cgatgggaac agatgtgcgc tctctcttca cggcgtcgag ctcaattctc | |
| | 541 | ctcgagcgtg ctttcgtggc tcgagaaatc acactgctga atccggtttg gatccactcc | |
| | 601 | aagaacaccg gtaagccctt ttacgcaatt ctccatagga tcgacgttgg agtcgttatc | |
| | 661 | gatttagagc cagctagaac cgaagatccg gcgctttcga tcgccggagc agtccaatcg | |
| | 721 | cagaagctcg cggttcgcgc gatttcacag ttacagtcgc ttcccggtgg ggacatcaag | |
| | 781 | cttctctgcg acactgttgt ggaaagcgtg agagacctca ctggacacga ccgtgtaatg | |
| | 841 | gtctacaagt ttcacgaaga cgagcacgga gaagttgtcg ccgagagccg gcgagaagat | |
| | 901 | ttagaacctt acatcggtct acattatccg gcaactgata ttcctcaagc gtcacggttc | |
| | 961 | ttattcaagc agaaccgtgt ccgtatgatc gtagattgcc acgccacacc ggttcttgtg | |
| | 1021 | gtccaggacg ataggctcac tcagtctatg tgcttggttg gttctactct aagggctcct | |
| | 1081 | cacggatgtc actctcagta tatggctaac atgggatcta ttgcatcttt agctatggca | |
| | 1141 | gttataataa acggcaacga agaagacggg ggcaatgtcg gtggtgggag aaactcaatg | |
| | 1201 | aggctttggg gtttagtcgt ttgccatcac acctcttctc gctgcatacc gtttcctcta | |
| | 1261 | aggtatgctt gcgagtttct aatgcaggca tttggtttac agttaaacat ggaactgcag | |
| | 1321 | ttagcattgc aaatgtcaga gaaacgcgtt ttgagaacgc agacactgtt atgcgatatg | |
| | 1381 | cttctgcgtg attctcctgc tgggattgtt acacagagtc ctagtatcat ggacttagtg | |
| | 1441 | aaatgtgacg gtgcagcgtt tctttaccat gggaagtact acccgttggg tgttgctccg | |
| | 1501 | acggaagttc aaataaaaga cgttgtcgag tggttgcttg cgagtcatgc ggattcgaca | |
| | 1561 | gggttaagca cggatagttt aggcgatgca ggatatcccg gtgcagctgc gttaggggat | |
| | 1621 | gctgtgtgcg gtatggcggt tgcgtatatc acaaaaagag actttctttt ctggttccgt | |
| | 1681 | tctcatactg ccaaagaaat caaatgggga ggcgctaagc atcatccaga ggacaaagat | |
| | 1741 | gatgggcaac ggatgcatcc tcgttcgtcc ttcaaggctt ttcttgaagt tgttaagagc | |
| | 1801 | aggagccagc catgggaaac tgcagaaatg gatgcgattc actcgctcca gcttattcta | |
| | 1861 | agagactctt ttaaagaatc agaggcagct atgaactcta aaaatgcaga gggtgcggtt | |
| | 1921 | cagccttgta gggatattgc gggaatgcag gggattgatg agttaggggc agttgcaaga | |
| | 1981 | gagatggtta ggctcattga aaccgctact gttcctatat ttgctgtgga cgctggaggc | |
| | 2041 | tgcattaatg gatggaacgc taagatggcg gaactgacag gtctctcggt tgaagaagct | |
| | 2101 | atggggaagt ctctggtttc tgatctaata tacaaagaga acgaagaaac tgttgataag | |
| | 2161 | ctcatttctc gtgctctgag aggggacgag gacaagaatg tggaggtgaa gctgaaaact | |
| | 2221 | ttcatccccg aacttgaagg gaatgcagtt tttgtggttg tgaatgcgtg ttcgagcaag | |
| | 2281 | gactacttga acaacattgt cggcgtctgt tttgtcggac aggatgtcac tggtcagaaa | |
| | 2341 | atcgtaatgg ataagttcat caacatacaa ggagactaca aggccattgt ccatagccca | |
| | 2401 | aacgcgctga ttccgccaat ctttgcggcg gacgagaaca cgtgctgcct tgagtggaac | |
| | 2461 | atcgcgatgg aaaagctcac gggttggtcc cgcagtgaag ttattgggaa aatgcttgtt | |
| | 2521 | ggggaagtgt ttggtagctg ttgcaggcta aagggtcatg atgctttaac caagttcatg | |
| | 2581 | attgcattgc ataacgcgat gggtggccaa gaaacggata agttcccttt cccgttcttt | |
| | 2641 | gaccgcaacg ggaagtttgt tcaggctcta ttgactgcaa acaagcgggt tagcctcgac | |
| | 2701 | ggaaaggtta ttggggcttt ctgcttcttg cagatcccga gtccggaact gcagcaagct | |
| | 2761 | ttagcagtcc aacggaggca ggacacagag tgtttcacga aggcgaaaga gctagcttac | |
| | 2821 | atttgtcagg tgataaagaa ccctttgagt ggtttgcgtt tcacaaactc actgctggaa | |
| | 2881 | gctacagact tgaacgaaga tcagaaagag ttccttgaaa cgagtgtttc ttgcgagaaa | |
| | 2941 | cagatctcga ggatcgtcag tgacatggat cttgaaagaa ttgaagacgg ttcatttgag | |
| | 3001 | ctagagagag cagagttttt cctcggaagt gtcataaacg cgatagtaag ccaggcgatg | |
| | 3061 | ttcttattaa gggagagggc tcttcagctg atccgtgaca ttcccgaaga gataaaatct | |
| | 3121 | atagctgttt atggagacca gacaaggatt caacagatcc tggctgagtt tcttctgagt | |
| | 3181 | ataatccggt atgcaccgtc tcaagagtgg gtggagatcc atctaagcca agttccgaag | |
| | 3241 | caaatggctg acggattctc cgctatccgc acagagttca gaatggcgtg cccaggtgaa | |
| | 3301 | ggtctgcctc cagagctggt ccgagacatg ttccatagca gcaggtggac gagtcctgaa | |
| | 3361 | gggttaggtc taagcgtgtg tcgaaagatt ctgaagctaa tgaacggtga ggttcaatac | |
| | 3421 | atccgagagt cggaacggtc ctatttcctc atcattctcg aactccctgt tcctctgaaa | |
| | 3481 | cgcccttttt caaccgctag cggcagcggc gacatgatgt tgatgatgcc atag | |
| 26 | MVSGGNGGSGRGRGEEAASSSHRAERSLHTTTYHRREQAQSSGTKSLRPQNQPQSHTESVSKAIQQYTVDA | | Thlaspi arvensi |
| | RLHAVFEQSGESGKSFDYSQSLKTTTYGSSVPEQQITAYLSRIORGGYIQPFGCMIAVDESTFRIIGYSEN | | version of YHB |
| | AREMLGLTPQSVPSLEKPEILAMGTDVRSLFTASSSILLERAFVAREITLLNPVWIHSKNTGKPFYAILHR | | allele of phyB. |
| | IDVGVVIDLEPARTEDPALSIAGAVQSQKLAVRAISQLQSLPGGDIKLLCDTVVESVRDLTGHDRVMVYKF | | Protein |
| | HEDEHGEVVAESRREDLEPYIGLHYPATDIPOASRFLFKONRVRMIVDCHATPVLVVODDRLTQSMCLVGS | | translated from |
| | TLRAPHGCHSQYMANMGSIASLAMAVIINGNEEDGGNVGGGRNSMRLWGLVVCHHTSSRCIPFPLRYACEF | | TaPHYB_YHB_cDNA |
| | LMQAFGLQLNMELQLALQMSEKRVLRTQTLLCDMLLRDSPAGIVTQSPSIMDLVKCDGAAFLYHGKYYPLG | | |
| | VAPTEVQIKDVVEWLLASHADSTGLSTDSLGDAGYPGAAALGDAVCGMAVAYITKRDFLFWFRSHTAKEIK | | |
| | WGGAKHHPEDKDDGORMHPRSSFKAFLEVVKSRSOPWETAEMDAIHSLOLILRDSFKESEAAMNSKNAEGA | | |
| | VQPCRDIAGMOGIDELGAVAREMVRLIETATVPIFAVDAGGCINGWNAKMAELTGLSVEEAMGKSLVSDLI | | |
| | YKENEETVDKLISRALRGDEDKNVEVKLKTFIPELEGNAVFVVVNACSSKDYLNNIVGVCFVGQDVTGQKI | | |
| | VMDKFINIQGDYKAIVHSPNALIPPIFAADENTCCLEWNIAMEKLTGWSRSEVIGKMLVGEVFGSCCRLKG | | |
| | HDALTKFMIALHNAMGGQETDKFPFPFFDRNGKFVCALLTANKRVSLDGKVIGAFCFLQIPSPELQQALAV | | |
| | QRRQDTECFTKAKELAYICQVIKNPLSGLRFTNSLLEATDLNEDQKEFLETSVSCEKQISRIVSDMDLERI | | |
| | EDGSFELERAEFFLGSVINAIVSQAMFLLRERALOLIRDIPEEIKSIAVYGDOTRIQQILAEFLLSIIRYA | | |
| | PSQEWVEIHLSQVPKOMADGFSAIRTEFRMACPGEGLPPELVRDMFHSSRWTSPEGLGLSVCRKILKLMNG | | |
| | EVQYIRESERSYFLIILELPVPLKRPFSTASGSGDMMLMMP- | | |
| 27 | 1 | atggtttccg gaggtaacgg cggtagtggc cgcggacgtg gtgaagaagc agcgtcgtca | WT TaPHYB_cDNA. |
| | 61 | agtcaccgtg ccgaacgcag tctccacacc acaacttatc accggagaga acaagctcaa | Thlaspi arvensi |
| | 121 | tcttctggga cgaaatcgct taggccgcaa aatcagccgc agagccacac tgaatcagtg | phyB wt cDNA |
| | 181 | agcaaagcca ttcaacagta caccgtcgac gcgaggctcc acgccgtctt cgaacagtct | |
| | 241 | ggcgagtccg gcaagtcgtt tgattactca cagtccctca aaacgacgac gtacgggtcc | |
| | 301 | tctgtaccgg agcagcagat cacggcgtat ctctctagga tccagcgagg aggctacatc | |
| | 361 | cagcccttcg gatgtatgat cgccgtcgat gaatccacct tcagaatcat cggctacagc | |
| | 421 | gaaaacgcgc gggaaatgtt aggccttact cctcagtccg ttcctagcct cgagaaaccg | |
| | 481 | gagattctag cgatgggaac agatgtgcgc tctctcttca cggcgtcgag ctcaattctc | |
| | 541 | ctcgagcgtg ctttcgtggc tcgagaaatc acactgctga atccggtttg gatccactcc | |
| | 601 | aagaacaccg gtaagccctt ttacgcaatt ctccatagga tcgacgttgg agtcgttatc | |
| | 661 | gatttagagc cagctagaac cgaagatccg gcgctttcga tcgccggagc agtccaatcg | |
| | 721 | cagaagctcg cggttcgcgc gatttcacag ttacagtcgc ttcccggtgg ggacatcaag | |
| | 781 | cttctctgcg acactgttgt ggaaagcgtg agagacctca ctggatacga ccgtgtaatg | |
| | 841 | gtctacaagt ttcacgaaga cgagcacgga gaagttgtcg ccgagagccg gcgagaagat | |
| | 901 | ttagaacctt acatcggtct acattatccg gcaactgata ttcctcaagc gtcacggttc | |
| | 961 | ttattcaagc agaaccgtgt ccgtatgatc gtagattgcc acgccacacc ggttcttgtg | |
| | 1021 | gtccaggacg ataggctcac tcagtctatg tgcttggttg gttctactct aagggctcct | |
| | 1081 | cacggatgtc actctcagta tatggctaac atgggatcta ttgcatcttt agctatggca | |
| | 1141 | gttataataa acggcaacga agaagacggg ggcaatgtcg gtggtgggag aaactcaatg | |
| | 1201 | aggctttggg gtttagtcgt ttgccatcac acctcttctc gctgcatacc gtttcctcta | |
| | 1261 | aggtatgctt gcgagtttct aatgcaggca tttggtttac agttaaacat ggaactgcag | |
| | 1321 | ttagcattgc aaatgtcaga gaaacgcgtt ttgagaacgc agacactgtt atgcgatatg | |
| | 1381 | cttctgcgtg attctcctgc tgggattgtt acacagagtc ctagtatcat ggacttagtg | |
| | 1441 | aaatgtgacg gtgcagcgtt tctttaccat gggaagtact acccgttggg tgttgctccg | |
| | 1501 | acggaagttc aaataaaaga cgttgtcgag tggttgcttg cgagtcatgc ggattcgaca | |
| | 1561 | gggttaagca cggatagttt aggcgatgca ggatatcccg gtgcagctgc gttaggggat | |
| | 1621 | gctgtgtgcg gtatggcggt tgcgtatatc acaaaaagag actttctttt ctggttccgt | |
| | 1681 | tctcatactg ccaaagaaat caaatgggga ggcgctaagc atcatccaga ggacaaagat | |
| | 1741 | gatgggcaac ggatgcatcc tcgttcgtcc ttcaaggctt ttcttgaagt tgttaagagc | |
| | 1801 | aggagccagc catgggaaac tgcagaaatg gatgcgattc actcgctcca gcttattcta | |
| | 1861 | agagactctt ttaaagaatc agaggcagct atgaactcta aaaatgcaga gggtgcggtt | |
| | 1921 | cagccttgta gggatattgc gggaatgcag gggattgatg agttaggggc agttgcaaga | |
| | 1981 | gagatggtta ggctcattga aaccgctact gttcctatat ttgctgtgga cgctggaggc | |
| | 2041 | tgcattaatg gatggaacgc taagatggcg gaactgacag gtctctcggt tgaagaagct | |
| | 2101 | atggggaagt ctctggtttc tgatctaata tacaaagaga acgaagaaac tgttgataag | |
| | 2161 | ctcatttctc gtgctctgag aggggacgag gacaagaatg tggaggtgaa gctgaaaact | |
| | 2221 | ttcatccccg aacttgaagg gaatgcagtt tttgtggttg tgaatgcgtg ttcgagcaag | |
| | 2281 | gactacttga acaacattgt cggcgtctgt tttgtcggac aggatgtcac tggtcagaaa | |
| | 2341 | atcgtaatgg ataagttcat caacatacaa ggagactaca aggccattgt ccatagccca | |
| | 2401 | aacgcgctga ttccgccaat ctttgcggcg gacgagaaca cgtgctgcct tgagtggaac | |
| | 2461 | atcgcgatgg aaaagctcac gggttggtcc cgcagtgaag ttattgggaa aatgcttgtt | |
| | 2521 | ggggaagtgt ttggtagctg ttgcaggcta aagggtcatg atgctttaac caagttcatg | |
| | 2581 | attgcattgc ataacgcgat gggtggccaa gaaacggata agttcccttt cccgttcttt | |
| | 2641 | gaccgcaacg ggaagtttgt tcaggctcta ttgactgcaa acaagcgggt tagcctcgac | |
| | 2701 | ggaaaggtta ttggggcttt ctgcttcttg cagatcccga gtccggaact gcagcaagct | |
| | 2761 | ttagcagtcc aacggaggca ggacacagag tgtttcacga aggcgaaaga gctagcttac | |
| | 2821 | atttgtcagg tgataaagaa ccctttgagt ggtttgcgtt tcacaaactc actgctggaa | |
| | 2881 | gctacagact tgaacgaaga tcagaaagag ttccttgaaa cgagtgtttc ttgcgagaaa | |
| | 2941 | cagatctcga ggatcgtcag tgacatggat cttgaaagaa ttgaagacgg ttcatttgag | |
| | 3001 | ctagagagag cagagttttt cctcggaagt gtcataaacg cgatagtaag ccaggcgatg | |
| | 3061 | ttcttattaa gggagagggc tcttcagctg atccgtgaca ttcccgaaga gataaaatct | |
| | 3121 | atagctgttt atggagacca gacaaggatt caacagatcc tggctgagtt tcttctgagt | |
| | 3181 | ataatccggt atgcaccgtc tcaagagtgg gtggagatcc atctaagcca agttccgaag | |
| | 3241 | caaatggctg acggattctc cgctatccgc acagagttca gaatggcgtg cccaggtgaa | |
| | 3301 | ggtctgcctc cagagctggt ccgagacatg ttccatagca gcaggtggac gagtcctgaa | |
| | 3361 | gggttaggtc taagcgtgtg tcgaaagatt ctgaagctaa tgaacggtga ggttcaatac | |
| | 3421 | atccgagagt cggaacggtc ctatttcctc atcattctcg aactccctgt tcctctgaaa | |
| | 3481 | cgcccttttt caaccgctag cggcagcggc gacatgatgt tgatgatgcc atag | |
| 28 | MVSGGNGGSGRGRGEEAASSSHRAERSLHTTTYHRREQAQSSGTKSLRPQNQPQSHTESVSKAIQQYTVDA | | TaPHYB_protein_ |
| | RLHAVFEQSGESGKSFDYSQSLKTTTYGSSVPEQQITAYLSRIORGGYIQPFGCMIAVDESTFRIIGYSEN | | sequence. |
| | AREMLGLTPQSVPSLEKPEILAMGTDVRSLFTASSSILLERAFVAREITLLNPVWIHSKNTGKPFYAILHR | | Encoded by SEQ |
| | IDVGVVIDLEPARTEDPALSIAGAVQSQKLAVRAISQLQSLPGGDIKLLCDTVVESVRDLTGYDRVMVYKF | | ID NO: 27 |
| | HEDEHGEVVAESRREDLEPYIGLHYPATDIPOASRFLFKONRVRMIVDCHATPVLVVODDRLTQSMCLVGS | | |
| | TLRAPHGCHSQYMANMGSIASLAMAVIINGNEEDGGNVGGGRNSMRLWGLVVCHHTSSRCIPFPLRYACEF | | |
| | LMQAFGLQLNMELQLALQMSEKRVLRTQTLLCDMLLRDSPAGIVTQSPSIMDLVKCDGAAFLYHGKYYPLG | | |
| | VAPTEVQIKDVVEWLLASHADSTGLSTDSLGDAGYPGAAALGDAVCGMAVAYITKRDFLFWFRSHTAKEIK | | |
| | WGGAKHHPEDKDDGORMHPRSSFKAFLEVVKSRSQPWETAEMDAIHSLQLILRDSFKESEAAMNSKNAEGA | | |
| | VQPCRDIAGMOGIDELGAVAREMVRLIETATVPIFAVDAGGCINGWNAKMAELTGLSVEEAMGKSLVSDLI | | |
| | YKENEETVDKLISRALRGDEDKNVEVKLKTFIPELEGNAVFVVVNACSSKDYLNNIVGVCFVGQDVTGQKI | | |
| | VMDKFINIQGDYKAIVHSPNALIPPIFAADENTCCLEWNIAMEKLTGWSRSEVIGKMLVGEVFGSCCRLKG | | |
| | HDALTKFMIALHNAMGGQETDKFPFPFFDRNGKFVCALLTANKRVSLDGKVIGAFCFLQIPSPELQQALAV | | |
| | QRRQDTECFTKAKELAYICQVIKNPLSGLRFTNSLLEATDLNEDQKEFLETSVSCEKQISRIVSDMDLERI | | |
| | EDGSFELERAEFFLGSVINAIVSQAMFLLRERALQLIRDIPEEIKSIAVYGDQTRIQQILAEFLLSIIRYA | | |
| | PSQEWVEIHLSQVPKOMADGFSAIRTEFRMACPGEGLPPELVRDMFHSSRWTSPEGLGLSVCRKILKLMNG | | |
| | EVOYIRESERSYFLIILELPVPLKRPFSTASGSGDMMLMMP | | |
| 29 | ATGGATTACAAGGACCACGACGGGGATTACAAGGACCACGACATTGATTACAAGGATGATGATGACAAGAT | | Cas9 DNA |
| | GGCTCCGAAGAAGAAGAGGAAGGTTGGCATCCACGGGGTGCCAGCTGCTGACAAGAAGTACTCGATCGGCC | | sequence |
| | TCGATATTGGGACTAACTCTGTTGGCTGGGCCGTGATCACCGACGAGTACAAGGTGCCCTCAAAGAAGTTC | | |
| | AAGGTCCTGGGCAACACCGATCGGCATTCCATCAAGAAGAATCTCATTGGCGCTCTCCTGTTCGACAGCGG | | |
| | CGAGACGGCTGAGGCTACGCGGCTCAAGCGCACCGCCCGCAGGCGGTACACGCGCAGGAAGAATCGCATCT | | |
| | GCTACCTGCAGGAGATTTTCTCCAACGAGATGGCGAAGGTTGACGATTCTTTCTTCCACAGGCTGGAGGAG | | |
| | TCATTCCTCGTGGAGGAGGATAAGAAGCACGAGCGGCATCCAATCTTCGGCAACATTGTCGACGAGGTTGC | | |
| | CTACCACGAGAAGTACCCTACGATCTACCATCTGCGGAAGAAGCTCGTGGACTCCACAGATAAGGCGGACC | | |
| | TCCGCCTGATCTACCTCGCTCTGGCCCACATGATTAAGTTCAGGGGCCATTTCCTGATCGAGGGGGATCTC | | |
| | AACCCGGACAATAGCGATGTTGACAAGCTGTTCATCCAGCTCGTGCAGACGTACAACCAGCTCTTCGAGGA | | |
| | GAACCCCATTAATGCGTCAGGCGTCGACGCGAAGGCTATCCTGTCCGCTAGGCTCTCGAAGTCTCGGCGCC | | |
| | TCGAGAACCTGATCGCCCAGCTGCCGGGCGAGAAGAAGAACGGCCTGTTCGGGAATCTCATTGCGCTCAGC | | |
| | CTGGGGCTCACGCCCAACTTCAAGTCGAATTTCGATCTCGCTGAGGACGCCAAGCTGCAGCTCTCCAAGGA | | |
| | CACATACGACGATGACCTGGATAACCTCCTGGCCCAGATCGGCGATCAGTACGCGGACCTGTTCCTCGCTG | | |
| | CCAAGAATCTGTCGGACGCCATCCTCCTGTCTGATATTCTCAGGGTGAACACCGAGATTACGAAGGCTCCG | | |
| | CTCTCAGCCTCCATGATCAAGCGCTACGACGAGCACCATCAGGATCTGACCCTCCTGAAGGCGCTGGTCAG | | |
| | GCAGCAGCTCCCCGAGAAGTACAAGGAGATCTTCTTCGATCAGTCGAAGAACGGCTACGCTGGGTACATTG | | |
| | ACGGCGGGGCCTCTCAGGAGGAGTTCTACAAGTTCATCAAGCCGATTCTGGAGAAGATGGACGGCACGGAG | | |
| | GAGCTGCTGGTGAAGCTCAATCGCGAGGACCTCCTGAGGAAGCAGCGGACATTCGATAACGGCAGCATCCC | | |
| | ACACCAGATTCATCTCGGGGAGCTGCACGCTATCCTGAGGAGGCAGGAGGACTTCTACCCTTTCCTCAAGG | | |
| | ATAACCGCGAGAAGATCGAGAAGATTCTGACTTTCAGGATCCCGTACTACGTCGGCCCACTCGCTAGGGGC | | |
| | AACTCCCGCTTCGCTTGGATGACCCGCAAGTCAGAGGAGACGATCACGCCGTGGAACTTCGAGGAGGTGGT | | |
| | CGACAAGGGCGCTAGCGCTCAGTCGTTCATCGAGAGGATGACGAATTTCGACAAGAACCTGCCAAATGAGA | | |
| | AGGTGCTCCCTAAGCACTCGCTCCTGTACGAGTACTTCACAGTCTACAACGAGCTGACTAAGGTGAAGTAT | | |
| | GTGACCGAGGGCATGAGGAAGCCGGCTTTCCTGTCTGGGGAGCAGAAGAAGGCCATCGTGGACCTCCTGTT | | |
| | CAAGACCAACCGGAAGGTCACGGTTAAGCAGCTCAAGGAGGACTACTTCAAGAAGATTGAGTGCTTCGATT | | |
| | CGGTCGAGATCTCTGGCGTTGAGGACCGCTTCAACGCCTCCCTGGGGACCTACCACGATCTCCTGAAGATC | | |
| | ATTAAGGATAAGGACTTCCTGGACAACGAGGAGAATGAGGATATCCTCGAGGACATTGTGCTGACACTCAC | | |
| | TCTGTTCGAGGACCGGGAGATGATCGAGGAGCGCCTGAAGACTTACGCCCATCTCTTCGATGACAAGGTCA | | |
| | TGAAGCAGCTCAAGAGGAGGAGGTACACCGGCTGGGGGAGGCTGAGCAGGAAGCTCATCAACGGCATTCGG | | |
| | GACAAGCAGTCCGGGAAGACGATCCTCGACTTCCTGAAGAGCGATGGCTTCGCGAACCGCAATTTCATGCA | | |
| | GCTGATTCACGATGACAGCCTCACATTCAAGGAGGATATCCAGAAGGCTCAGGTGAGCGGCCAGGGGGACT | | |
| | CGCTGCACGAGCATATCGCGAACCTCGCTGGCTCGCCAGCTATCAAGAAGGGGATTCTGCAGACCGTGAAG | | |
| | GTTGTGGACGAGCTGGTGAAGGTCATGGGCAGGCACAAGCCTGAGAACATCGTCATTGAGATGGCCCGGGA | | |
| | GAATCAGACCACGCAGAAGGGCCAGAAGAACTCACGCGAGAGGATGAAGAGGATCGAGGAGGGCATTAAGG | | |
| | AGCTGGGGTCCCAGATCCTCAAGGAGCACCCGGTGGAGAACACGCAGCTGCAGAATGAGAAGCTCTACCTG | | |
| | TACTACCTCCAGAATGGCCGCGATATGTATGTGGACCAGGAGCTGGATATTAACAGGCTCAGCGATTACGA | | |
| | CGTCGATCATATCGTTCCACAGTCATTCCTGAAGGATGACTCCATTGACAACAAGGTCCTCACCAGGTCGG | | |
| | ACAAGAACCGGGGCAAGTCTGATAATGTTCCTTCAGAGGAGGTCGTTAAGAAGATGAAGAACTACTGGCGC | | |
| | CAGCTCCTGAATGCCAAGCTGATCACGCAGCGGAAGTTCGATAACCTCACAAAGGCTGAGAGGGGGGGGCT | | |
| | CTCTGAGCTGGACAAGGCGGGCTTCATCAAGAGGCAGCTGGTCGAGACACGGCAGATCACTAAGCACGTTG | | |
| | CGCAGATTCTCGACTCACGGATGAACACTAAGTACGATGAGAATGACAAGCTGATCCGCGAGGTGAAGGTC | | |
| | ATCACCCTGAAGTCAAAGCTCGTCTCCGACTTCAGGAAGGATTTCCAGTTCTACAAGGTTCGGGAGATCAA | | |
| | CAATTACCACCATGCCCATGACGCGTACCTGAACGCGGTGGTCGGCACAGCTCTGATCAAGAAGTACCCAA | | |
| | AGCTCGAGAGCGAGTTCGTGTACGGGGACTACAAGGTTTACGATGTGAGGAAGATGATCGCCAAGTCGGAG | | |
| | CAGGAGATTGGCAAGGCTACCGCCAAGTACTTCTTCTACTCTAACATTATGAATTTCTTCAAGACAGAGAT | | |
| | CACTCTGGCCAATGGCGAGATCCGGAAGCGCCCCCTCATCGAGACGAACGGCGAGACGGGGGAGATCGTGT | | |
| | GGGACAAGGGCAGGGATTTCGCGACCGTCAGGAAGGTTCTCTCCATGCCACAAGTGAATATCGTCAAGAAG | | |
| | ACAGAGGTCCAGACTGGCGGGTTCTCTAAGGAGTCAATTCTGCCTAAGCGGAACAGCGACAAGCTCATCGC | | |
| | CCGCAAGAAGGACTGGGATCCGAAGAAGTACGGCGGGTTCGACAGCCCCACTGTGGCCTACTCGGTCCTGG | | |
| | TTGTGGCGAAGGTTGAGAAGGGCAAGTCCAAGAAGCTCAAGAGCGTGAAGGAGCTGCTGGGGATCACGATT | | |
| | ATGGAGCGCTCCAGCTTCGAGAAGAACCCGATCGATTTCCTGGAGGCGAAGGGCTACAAGGAGGTGAAGAA | | |
| | GGACCTGATCATTAAGCTCCCCAAGTACTCACTCTTCGAGCTGGAGAACGGCAGGAAGCGGATGCTGGCTT | | |
| | CCGCTGGCGAGCTGCAGAAGGGGAACGAGCTGGCTCTGCCGTCCAAGTATGTGAACTTCCTCTACCTGGCC | | |
| | TCCCACTACGAGAAGCTCAAGGGCAGCCCCGAGGACAACGAGCAGAAGCAGCTGTTCGTCGAGCAGCACAA | | |
| | GCATTACCTCGACGAGATCATTGAGCAGATTTCCGAGTTCTCCAAGCGCGTGATCCTGGCCGACGCGAATC | | |
| | TGGATAAGGTCCTCTCCGCGTACAACAAGCACCGCGACAAGCCAATCAGGGAGCAGGCTGAGAATATCATT | | |
| | CATCTCTTCACCCTGACGAACCTCGGCGCCCCTGCTGCTTTCAAGTACTTCGACACAACTATCGATCGCAA | | |
| | GAGGTACACAAGCACTAAGGAGGTCCTGGACGCGACCCTCATCCACCAGTCGATTACCGGCCTCTACGAGA | | |
| | CGCGCATCGACCTGTCTCAGCTCGGGGGCGACAAGCGGCCAGCGGCGACGAAGAAGGCGGGGCAGGCGAAG | | |
| | AAGAAGAAGTGA | | |
| 30 | GTCAAGGGGAGCGCTAAGCG | | gRNA1 from |
| | | | TaELF3 genomic |
| | | | sequence |
| 31 | CCACAAGTTATCAAACACAG | | gRNA2 from |
| | | | TaELF3 genomic |
| | | | sequence |
| 32 | 1 | gtttacccgc caatatatcc tgtcaaacac tgatagttta aactgaaggc gggaaacgac | pHEE401_TaELF3g |
| | 61 | aatctgatcc aagctcaagc tgctctagca ttcgccattc aggctgcgca actgttggga | RNA CRISPR |
| | 121 | agggcgatcg gtgcgggcct cttcgctatt acgccagctg gcgaaagggg gatgtgctgc | construct. |
| | 181 | aaggcgatta agttgggtaa cgccagggtt ttcccagtca cgacgttgta aaacgacggc | Base 254 to |
| | 241 | cagtgccaag cttcgacttg ccttccgcac aatacatcat ttcttcttag ctttttttct | base 1287 |
| | 301 | tcttcttcgt tcatacagtt tttttttgtt tatcagctta cattttcttg aaccgtagct | contains the U6 |
| | 361 | ttcgttttct tctttttaac tttccattcg gagtttttgt atcttgtttc atagtttgtc | promoter gRNA1 |
| | 421 | ccaggattag aatgattagg catcgaacct tcaagaattt gattgaataa aacatcttca | gRNA scaffold- |
| | 481 | ttcttaagat atgaagataa tcttcaaaag gcccctggga atctgaaaga agagaagcag | U6 terminator, |
| | 541 | gcccatttat atgggaaaga acaatagtat ttcttatata ggcccattta agttgaaaac | U6 promoter- |
| | 601 | aatcttcaaa agtcccacat cgcttagata agaaaacgaa gctgagttta tatacagcta | gRNA2 gRNA |
| | 661 | gagtcgaagt agtgattggt caaggggagc gctaagcggt tttagagcta gaaatagcaa | scaffold U6 |
| | 721 | gttaaaataa ggctagtccg ttatcaactt gaaaaagtgg caccgagtcg gtgctttttt | terminator. |
| | 781 | ttgcaaaatt ttccagatcg atttcttctt cctctgttct tcggcgttca atttctgggg | From 1552-7881 |
| | 841 | ttttctcttc gttttctgta actgaaacct aaaatttgac ctaaaaaaaa tctcaaataa | is the EC1 |
| | 901 | tatgattcag tggttttgta cttttcagtt agttgagttt tgcagttccg atgagataaa | promoter zCAS9 |
| | 961 | ccaatattaa tccaaactac tgcagcctga cagacaaatg aggatgcaaa caattttaaa | rbcS E9 |
| | 1021 | gtttatctaa cgctagctgt tttgtttctt ctctctggtg caccaacgac ggcgttttct | terminator |
| | 1081 | caatcataaa gaggcttgtt ttacttaagg ccaataatgt tgatggatcg aaagaagagg | |
| | 1141 | gcttttaata aacgagcccg tttaagctgt aaacgatgtc aaaaacatcc cacatcgttc | |
| | 1201 | agttgaaaat agaagctctg tttatatatt ggtagagtcg actaagagat tgccacaagt | |
| | 1261 | tatcaaacac aggttttaga gctagaaata gcaagttaaa ataaggctag tccgttatca | |
| | 1321 | acttgaaaaa gtggcaccga gtcggtgctt ttttttgcaa aattttccag atcgatttct | |
| | 1381 | tcttcctctg ttcttcggcg ttcaatttct ggggttttct cttcgttttc tgtaactgaa | |
| | 1441 | acctaaaatt tgacctaaaa aaaatctcaa ataatatgat tcagtggttt tgtacttttc | |
| | 1501 | agttagttga gttttgcagt tccgatgaga taaaccaata ccatggttat actagtgaat | |
| | 1561 | aaaagcattt gcgtttggtt tatcattgcg tttatacaag gacagagatc cactgagctg | |
| | 1621 | gaatagctta aaaccattat cagaacaaaa taaaccattt tttgttaaga atcagagcat | |
| | 1681 | agtaaacaac agaaacaacc taagagaggt aacttgtcca agaagatagc taattatatc | |
| | 1741 | tattttataa aagttatcat agtttgtaag tcacaaaaga tgcaaataac agagaaacta | |
| | 1801 | ggagacttga gaatatacat tcttgtatat ttgtattcga gattgtgaaa atttgaccat | |
| | 1861 | aagtttaaat tcttaaaaag atatatctga tctaggtgat ggttatagac tgtaatttta | |
| | 1921 | ccacatgttt aatgatggat agtgacacac atgacacatc gacaacacta tagcatctta | |
| | 1981 | tttagattac aacatgaaat ttttctgtaa tacatgtctt tgtacataat ttaaaagtaa | |
| | 2041 | ttcctaagaa atatatttat acaaggagtt taaagaaaac atagcataaa gttcaatgag | |
| | 2101 | tagtaaaaac catatacagt atatagcata aagttcaatg agtttattac aaaagcattg | |
| | 2161 | gttcactttc tgtaacacga cgttaaacct tcgtctccaa taggagcgct actgattcaa | |
| | 2221 | catgccaata tatactaaat acgtttctac agtcaaatgc tttaacgttt catgattaag | |
| | 2281 | tgactattta ccgtcaatcc tttcccattc ctcccactaa tccaactttt taattactct | |
| | 2341 | taaatcacca ctaagctagt aacgcctatc atgaattagc tctactaaat ctagcaacct | |
| | 2401 | ttcaaatttg cagtattgca ggtgtctctg tgtctttaaa atagttgcct tatgatttct | |
| | 2461 | tcggtttcaa gatgatcaaa tagttataga tttcatgctc acacatgctc attagatgtg | |
| | 2521 | tacatacttt acttacccaa atctattttc tcgcaaagat tttgatggta aagctgattt | |
| | 2581 | ggttctattg aactaaatca aacgagtttc agactgagtg attctaatcc ggcccattag | |
| | 2641 | cccctaaaca gacccactaa ttacgcagct tttaatagag taattacacc tagtttaccc | |
| | 2701 | actaaaccac taagcactaa ttatctcaca atctaatgag cttccctcgt aattacttgg | |
| | 2761 | gctttcactc taccatttat ttgtaacagt caagtctcta ctgtctctat ataaactctc | |
| | 2821 | taaagttaac acacaattct catcacaaac aaatcaacca aagcaacttc tactctttct | |
| | 2881 | tctttcgacc ttatcaatct gttgagaaat ctagatggat tacaaggacc acgacgggga | |
| | 2941 | ttacaaggac cacgacattg attacaagga tgatgatgac aagatggctc cgaagaagaa | |
| | 3001 | gaggaaggtt ggcatccacg gggtgccagc tgctgacaag aagtactcga tcggcctcga | |
| | 3061 | tattgggact aactctgttg gctgggccgt gatcaccgac gagtacaagg tgccctcaaa | |
| | 3121 | gaagttcaag gtcctgggca acaccgatcg gcattccatc aagaagaatc tcattggcgc | |
| | 3181 | tctcctgttc gacagcggcg agacggctga ggctacgcgg ctcaagcgca ccgcccgcag | |
| | 3241 | gcggtacacg cgcaggaaga atcgcatctg ctacctgcag gagattttct ccaacgagat | |
| | 3301 | ggcgaaggtt gacgattctt tcttccacag gctggaggag tcattcctcg tggaggagga | |
| | 3361 | taagaagcac gagcggcatc caatcttcgg caacattgtc gacgaggttg cctaccacga | |
| | 3421 | gaagtaccct acgatctacc atctgcggaa gaagctcgtg gactccacag ataaggcgga | |
| | 3481 | cctccgcctg atctacctcg ctctggccca catgattaag ttcaggggcc atttcctgat | |
| | 3541 | cgagggggat ctcaacccgg acaatagcga tgttgacaag ctgttcatcc agctcgtgca | |
| | 3601 | gacgtacaac cagctcttcg aggagaaccc cattaatgcg tcaggcgtcg acgcgaaggc | |
| | 3661 | tatcctgtcc gctaggctct cgaagtctcg gcgcctcgag aacctgatcg cccagctgcc | |
| | 3721 | gggcgagaag aagaacggcc tgttcgggaa tctcattgcg ctcagcctgg ggctcacgcc | |
| | 3781 | caacttcaag tcgaatttcg atctcgctga ggacgccaag ctgcagctct ccaaggacac | |
| | 3841 | atacgacgat gacctggata acctcctggc ccagatcggc gatcagtacg cggacctgtt | |
| | 3901 | cctcgctgcc aagaatctgt cggacgccat cctcctgtct gatattctca gggtgaacac | |
| | 3961 | cgagattacg aaggctccgc tctcagcctc catgatcaag cgctacgacg agcaccatca | |
| | 4021 | ggatctgacc ctcctgaagg cgctggtcag gcagcagctc cccgagaagt acaaggagat | |
| | 4081 | cttcttcgat cagtcgaaga acggctacgc tgggtacatt gacggcgggg cctctcagga | |
| | 4141 | ggagttctac aagttcatca agccgattct ggagaagatg gacggcacgg aggagctgct | |
| | 4201 | ggtgaagctc aatcgcgagg acctcctgag gaagcagcgg acattcgata acggcagcat | |
| | 4261 | cccacaccag attcatctcg gggagctgca cgctatcctg aggaggcagg aggacttcta | |
| | 4321 | ccctttcctc aaggataacc gcgagaagat cgagaagatt ctgactttca ggatcccgta | |
| | 4381 | ctacgtcggc ccactcgcta ggggcaactc ccgcttcgct tggatgaccc gcaagtcaga | |
| | 4441 | ggagacgatc acgccgtgga acttcgagga ggtggtcgac aagggcgcta gcgctcagtc | |
| | 4501 | gttcatcgag aggatgacga atttcgacaa gaacctgcca aatgagaagg tgctccctaa | |
| | 4561 | gcactcgctc ctgtacgagt acttcacagt ctacaacgag ctgactaagg tgaagtatgt | |
| | 4621 | gaccgagggc atgaggaagc cggctttcct gtctggggag cagaagaagg ccatcgtgga | |
| | 4681 | cctcctgttc aagaccaacc ggaaggtcac ggttaagcag ctcaaggagg actacttcaa | |
| | 4741 | gaagattgag tgcttcgatt cggtcgagat ctctggcgtt gaggaccgct tcaacgcctc | |
| | 4801 | cctggggacc taccacgatc tcctgaagat cattaaggat aaggacttcc tggacaacga | |
| | 4861 | ggagaatgag gatatcctcg aggacattgt gctgacactc actctgttcg aggaccggga | |
| | 4921 | gatgatcgag gagcgcctga agacttacgc ccatctcttc gatgacaagg tcatgaagca | |
| | 4981 | gctcaagagg aggaggtaca ccggctgggg gaggctgagc aggaagctca tcaacggcat | |
| | 5041 | tcgggacaag cagtccggga agacgatcct cgacttcctg aagagcgatg gcttcgcgaa | |
| | 5101 | ccgcaatttc atgcagctga ttcacgatga cagcctcaca ttcaaggagg atatccagaa | |
| | 5161 | ggctcaggtg agcggccagg gggactcgct gcacgagcat atcgcgaacc tcgctggctc | |
| | 5221 | gccagctatc aagaagggga ttctgcagac cgtgaaggtt gtggacgagc tggtgaaggt | |
| | 5281 | catgggcagg cacaagcctg agaacatcgt cattgagatg gcccgggaga atcagaccac | |
| | 5341 | gcagaagggc cagaagaact cacgcgagag gatgaagagg atcgaggagg gcattaagga | |
| | 5401 | gctggggtcc cagatcctca aggagcaccc ggtggagaac acgcagctgc agaatgagaa | |
| | 5461 | gctctacctg tactacctcc agaatggccg cgatatgtat gtggaccagg agctggatat | |
| | 5521 | taacaggctc agcgattacg acgtcgatca tatcgttcca cagtcattcc tgaaggatga | |
| | 5581 | ctccattgac aacaaggtcc tcaccaggtc ggacaagaac cggggcaagt ctgataatgt | |
| | 5641 | tccttcagag gaggtcgtta agaagatgaa gaactactgg cgccagctcc tgaatgccaa | |
| | 5701 | gctgatcacg cagcggaagt tcgataacct cacaaaggct gagaggggcg ggctctctga | |
| | 5761 | gctggacaag gcgggcttca tcaagaggca gctggtcgag acacggcaga tcactaagca | |
| | 5821 | cgttgcgcag attctcgact cacggatgaa cactaagtac gatgagaatg acaagctgat | |
| | 5881 | ccgcgaggtg aaggtcatca ccctgaagtc aaagctcgtc tccgacttca ggaaggattt | |
| | 5941 | ccagttctac aaggttcggg agatcaacaa ttaccaccat gcccatgacg cgtacctgaa | |
| | 6001 | cgcggtggtc ggcacagctc tgatcaagaa gtacccaaag ctcgagagcg agttcgtgta | |
| | 6061 | cggggactac aaggtttacg atgtgaggaa gatgatcgcc aagtcggagc aggagattgg | |
| | 6121 | caaggctacc gccaagtact tcttctactc taacattatg aatttcttca agacagagat | |
| | 6181 | cactctggcc aatggcgaga tccggaagcg ccccctcatc gagacgaacg gcgagacggg | |
| | 6241 | ggagatcgtg tgggacaagg gcagggattt cgcgaccgtc aggaaggttc tctccatgcc | |
| | 6301 | acaagtgaat atcgtcaaga agacagaggt ccagactggc gggttctcta aggagtcaat | |
| | 6361 | tctgcctaag cggaacagcg acaagctcat cgcccgcaag aaggactggg atccgaagaa | |
| | 6421 | gtacggcggg ttcgacagcc ccactgtggc ctactcggtc ctggttgtgg cgaaggttga | |
| | 6481 | gaagggcaag tccaagaagc tcaagagcgt gaaggagctg ctggggatca cgattatgga | |
| | 6541 | gcgctccagc ttcgagaaga acccgatcga tttcctggag gcgaagggct acaaggaggt | |
| | 6601 | gaagaaggac ctgatcatta agctccccaa gtactcactc ttcgagctgg agaacggcag | |
| | 6661 | gaagcggatg ctggcttccg ctggcgagct gcagaagggg aacgagctgg ctctgccgtc | |
| | 6721 | caagtatgtg aacttcctct acctggcctc ccactacgag aagctcaagg gcagccccga | |
| | 6781 | ggacaacgag cagaagcagc tgttcgtcga gcagcacaag cattacctcg acgagatcat | |
| | 6841 | tgagcagatt tccgagttct ccaagcgcgt gatcctggcc gacgcgaatc tggataaggt | |
| | 6901 | cctctccgcg tacaacaagc accgcgacaa gccaatcagg gagcaggctg agaatatcat | |
| | 6961 | tcatctcttc accctgacga acctcggcgc ccctgctgct ttcaagtact tcgacacaac | |
| | 7021 | tatcgatcgc aagaggtaca caagcactaa ggaggtcctg gacgcgaccc tcatccacca | |
| | 7081 | gtcgattacc ggcctctacg agacgcgcat cgacctgtct cagctcgggg gcgacaagcg | |
| | 7141 | gccagcggcg acgaagaagg cggggcaggc gaagaagaag aagtgagctc agagctttcg | |
| | 7201 | ttcgtatcat cggtttcgac aacgttcgtc aagttcaatg catcagtttc attgcgcaca | |
| | 7261 | caccagaatc ctactgagtt tgagtattat ggcattggga aaactgtttt tcttgtacca | |
| | 7321 | tttgttgtgc ttgtaattta ctgtgttttt tattcggttt tcgctatcga actgtgaaat | |
| | 7381 | ggaaatggat ggagaagagt taatgaatga tatggtcctt ttgttcattc tcaaattaat | |
| | 7441 | attatttgtt ttttctctta tttgttgtgt gttgaatttg aaattataag agatatgcaa | |
| | 7501 | acattttgtt ttgagtaaaa atgtgtcaaa tcgtggcctc taatgaccga agttaatatg | |
| | 7561 | aggagtaaaa cacttgtagt tgtaccatta tgcttattca ctaggcaaca aatatatttt | |
| | 7621 | cagacctaga aaagctgcaa atgttactga atacaagtat gtcctcttgt gttttagaca | |
| | 7681 | tttatgaact ttcctttatg taattttcca gaatccttgt cagattctaa tcattgcttt | |
| | 7741 | ataattatag ttatactcat ggatttgtag ttgagtatga aaatattttt taatgcattt | |
| | 7801 | tatgacttgc caattgattg acaacgaatt cgtaatcatg tcatagctgt ttcctgtgtg | |
| | 7861 | aaattgttat ccgctcacaa ttccacacaa catacgagcc ggaagcataa agtgtaaagc | |
| | 7921 | ctggggtgcc taatgagtga gctaactcac attaattgcg ttgcgctcac tgcccgcttt | |
| | 7981 | ccagtcggga aacctgtcgt gccagctgca ttaatgaatc ggccaacgcg cggggagagg | |
| | 8041 | cggtttgcgt attggctaga gcagcttgcc aacatggtgg agcacgacac tctcgtctac | |
| | 8101 | tccaagaata tcaaagatac agtctcagaa gaccaaaggg ctattgagac ttttcaacaa | |
| | 8161 | agggtaatat cgggaaacct cctcggattc cattgcccag ctatctgtca cttcatcaaa | |
| | 8221 | aggacagtag aaaaggaagg tggcacctac aaatgccatc attgcgataa aggaaaggct | |
| | 8281 | atcgttcaag atgcctctgc cgacagtggt cccaaagatg gacccccacc cacgaggagc | |
| | 8341 | atcgtggaaa aagaagacgt tccaaccacg tcttcaaagc aagtggattg atgtgataac | |
| | 8401 | atggtggagc acgacactct cgtctactcc aagaatatca aagatacagt ctcagaagac | |
| | 8461 | caaagggcta ttgagacttt tcaacaaagg gtaatatcgg gaaacctcct cggattccat | |
| | 8521 | tgcccagcta tctgtcactt catcaaaagg acagtagaaa aggaaggtgg cacctacaaa | |
| | 8581 | tgccatcatt gcgataaagg aaaggctatc gttcaagatg cctctgccga cagtggtccc | |
| | 8641 | aaagatggac ccccacccac gaggagcatc gtggaaaaag aagacgttcc aaccacgtct | |
| | 8701 | tcaaagcaag tggattgatg tgatatctcc actgacgtaa gggatgacgc acaatcccac | |
| | 8761 | tatccttcgc aagaccttcc tctatataag gaagttcatt tcatttggag aggacacgct | |
| | 8821 | gaaatcacca gtctctctct acaaatctat ctctctcgag ctttcgcaga tcccgggggg | |
| | 8881 | caatgagata tgaaaaagcc tgaactcacc gcgacgtctg tcgagaagtt tctgatcgaa | |
| | 8941 | aagttcgaca gcgtctccga cctgatgcag ctctcggagg gcgaagaatc tcgtgctttc | |
| | 9001 | agcttcgatg taggagggcg tggatatgtc ctgcgggtaa atagctgcgc cgatggtttc | |
| | 9061 | tacaaagatc gttatgttta tcggcacttt gcatcggccg cgctcccgat tccggaagtg | |
| | 9121 | cttgacattg gggagtttag cgagagcctg acctattgca tctcccgccg tgcacagggt | |
| | 9181 | gtcacgttgc aagacctgcc tgaaaccgaa ctgcccgctg ttctacaacc ggtcgcggag | |
| | 9241 | gctatggatg cgatcgctgc ggccgatctt agccagacga gcgggttcgg cccattcgga | |
| | 9301 | ccgcaaggaa tcggtcaata cactacatgg cgtgatttca tatgcgcgat tgctgatccc | |
| | 9361 | catgtgtatc actggcaaac tgtgatggac gacaccgtca gtgcgtccgt cgcgcaggct | |
| | 9421 | ctcgatgagc tgatgctttg ggccgaggac tgccccgaag tccggcacct cgtgcacgcg | |
| | 9481 | gatttcggct ccaacaatgt cctgacggac aatggccgca taacagcggt cattgactgg | |
| | 9541 | agcgaggcga tgttcgggga ttcccaatac gaggtcgcca acatcttctt ctggaggccg | |
| | 9601 | tggttggctt gtatggagca gcagacgcgc tacttcgagc ggaggcatcc ggagcttgca | |
| | 9661 | ggatcgccac gactccgggc gtatatgctc cgcattggtc ttgaccaact ctatcagagc | |
| | 9721 | ttggttgacg gcaatttcga tgatgcagct tgggcgcagg gtcgatgcga cgcaatcgtc | |
| | 9781 | cgatccggag ccgggactgt cgggcgtaca caaatcgccc gcagaagcgc ggccgtctgg | |
| | 9841 | accgatggct gtgtagaagt actcgccgat agtggaaacc gacgccccag cactcgtccg | |
| | 9901 | agggcaaaga aatagagtag atgccgaccg gatctgtcga tcgacaagct cgagtttctc | |
| | 9961 | cataataatg tgtgagtagt tcccagataa gggaattagg gttcctatag ggtttcgctc | |
| | 10021 | atgtgttgag catataagaa acccttagta tgtatttgta tttgtaaaat acttctatca | |
| | 10081 | ataaaatttc taattcctaa aaccaaaatc cagtactaaa atccagatcc cccgaattaa | |
| | 10141 | ttcggcgtta attcagtaca ttaaaaacgt ccgcaatgtg ttattaagtt gtctaagcgt | |
| | 10201 | caatttgttt acaccacaat atatcctgcc accagccagc caacagctcc ccgaccggca | |
| | 10261 | gctcggcaca aaatcaccac tcgatacagg cagcccatca gtccgggacg gcgtcagcgg | |
| | 10321 | gagagccgtt gtaaggcggc agactttgct catgttaccg atgctattcg gaagaacggc | |
| | 10381 | aactaagctg ccgggtttga aacacggatg atctcgcgga gggtagcatg ttgattgtaa | |
| | 10441 | cgatgacaga gcgttgctgc ctgtgatcac cgcggtttca aaatcggctc cgtcgatact | |
| | 10501 | atgttatacg ccaactttga aaacaacttt gaaaaagctg ttttctggta tttaaggttt | |
| | 10561 | tagaatgcaa ggaacagtga attggagttc gtcttgttat aattagcttc ttggggtatc | |
| | 10621 | tttaaatact gtagaaaaga ggaaggaaat aataaatggc taaaatgaga atatcaccgg | |
| | 10681 | aattgaaaaa actgatcgaa aaataccgct gcgtaaaaga tacggaagga atgtctcctg | |
| | 10741 | ctaaggtata taagctggtg ggagaaaatg aaaacctata tttaaaaatg acggacagcc | |
| | 10801 | ggtataaagg gaccacctat gatgtggaac gggaaaagga catgatgcta tggctggaag | |
| | 10861 | gaaagctgcc tgttccaaag gtcctgcact ttgaacggca tgatggctgg agcaatctgc | |
| | 10921 | tcatgagtga ggccgatggc gtcctttgct cggaagagta tgaagatgaa caaagccctg | |
| | 10981 | aaaagattat cgagctgtat gcggagtgca tcaggctctt tcactccatc gacatatcgg | |
| | 11041 | attgtcccta tacgaatagc ttagacagcc gcttagccga attggattac ttactgaata | |
| | 11101 | acgatctggc cgatgtggat tgcgaaaact gggaagaaga cactccattt aaagatccgc | |
| | 11161 | gcgagctgta tgatttttta aagacggaaa agcccgaaga ggaacttgtc ttttcccacg | |
| | 11221 | gcgacctggg agacagcaac atctttgtga aagatggcaa agtaagtggc tttattgatc | |
| | 11281 | ttgggagaag cggcagggcg gacaagtggt atgacattgc cttctgcgtc cggtcgatca | |
| | 11341 | gggaggatat cggggaagaa cagtatgtcg agctattttt tgacttactg gggatcaagc | |
| | 11401 | ctgattggga gaaaataaaa tattatattt tactggatga attgttttag tacctagaat | |
| | 11461 | gcatgaccaa aatcccttaa cgtgagtttt cgttccactg agcgtcagac cccgtagaaa | |
| | 11521 | agatcaaagg atcttcttga gatccttttt ttctgcgcgt aatctgctgc ttgcaaacaa | |
| | 11581 | aaaaaccacc gctaccagcg gtggtttgtt tgccggatca agagctacca actctttttc | |
| | 11641 | cgaaggtaac tggcttcagc agagcgcaga taccaaatac tgtccttcta gtgtagccgt | |
| | 11701 | agttaggcca ccacttcaag aactctgtag caccgcctac atacctcgct ctgctaatcc | |
| | 11761 | tgttaccagt ggctgctgcc agtggcgata agtcgtgtct taccgggttg gactcaagac | |
| | 11821 | gatagttacc ggataaggcg cagcggtcgg gctgaacggg gggttcgtgc acacagccca | |
| | 11881 | gcttggagcg aacgacctac accgaactga gatacctaca gcgtgagcta tgagaaagcg | |
| | 11941 | ccacgcttcc cgaagggaga aaggcggaca ggtatccggt aagcggcagg gtcggaacag | |
| | 12001 | gagagcgcac gagggagctt ccagggggaa acgcctggta tctttatagt cctgtcgggt | |
| | 12061 | ttcgccacct ctgacttgag cgtcgatttt gttgatgctc gtcagggggg cggagcctat | |
| | 12121 | ggaaaaacgc cagcaacgcg gcctttttac ggttcctggc cttttgctgg ccttttgctc | |
| | 12181 | acatgttctt tcctgcgtta tcccctgatt ctgtggataa ccgtattacc gcctttgagt | |
| | 12241 | gagctgatac cgctcgccgc agccgaacga ccgagcgcag cgagtcagtg agcgaggaag | |
| | 12301 | cggaagagcg cctgatgcgg tattttctcc ttacgcatct gtgcggtatt tcacaccgca | |
| | 12361 | tatggtgcac tctcagtaca atctgctctg atgccgcata gttaagccag tatacactcc | |
| | 12421 | gctatcgcta cgtgactggg tcatggctgc gccccgacac ccgccaacac ccgctgacgc | |
| | 12481 | gccctgacgg gcttgtctgc tcccggcatc cgcttacaga caagctgtga ccgtctccgg | |
| | 12541 | gagctgcatg tgtcagaggt tttcaccgtc atcaccgaaa cgcgcgaggc agggtgcctt | |
| | 12601 | gatgtgggcg ccggcggtcg agtggcgacg gcgcggcttg tccgcgccct ggtagattgc | |
| | 12661 | ctggccgtag gccagccatt tttgagcggc cagcggccgc gataggccga cgcgaagcgg | |
| | 12721 | cggggcgtag ggagcgcagc gaccgaaggg taggcgcttt ttgcagctct tcggctgtgc | |
| | 12781 | gctggccaga cagttatgca caggccaggc gggttttaag agttttaata agttttaaag | |
| | 12841 | agttttaggc ggaaaaatcg ccttttttct cttttatatc agtcacttac atgtgtgacc | |
| | 12901 | ggttcccaat gtacggcttt gggttcccaa tgtacgggtt ccggttccca atgtacggct | |
| | 12961 | ttgggttccc aatgtacgtg ctatccacag gaaacagacc ttttcgacct ttttcccctg | |
| | 13021 | ctagggcaat ttgccctagc atctgctccg tacattagga accggcggat gcttcgccct | |
| | 13081 | cgatcaggtt gcggtagcgc atgactagga tcgggccagc ctgccccgcc tcctccttca | |
| | 13141 | aatcgtactc cggcaggtca tttgacccga tcagcttgcg cacggtgaaa cagaacttct | |
| | 13201 | tgaactctcc ggcgctgcca ctgcgttcgt agatcgtctt gaacaaccat ctggcttctg | |
| | 13261 | ccttgcctgc ggcgcggcgt gccaggcggt agagaaaacg gccgatgccg ggatcgatca | |
| | 13321 | aaaagtaatc ggggtgaacc gtcagcacgt ccgggttctt gccttctgtg atctcgcggt | |
| | 13381 | acatccaatc agctagctcg atctcgatgt actccggccg cccggtttcg ctctttacga | |
| | 13441 | tcttgtagcg gctaatcaag gcttcaccct cggataccgt caccaggcgg ccgttcttgg | |
| | 13501 | ccttcttcgt acgctgcatg gcaacgtgcg tggtgtttaa ccgaatgcag gtttctacca | |
| | 13561 | ggtcgtcttt ctgctttccg ccatcggctc gccggcagaa cttgagtacg tccgcaacgt | |
| | 13621 | gtggacggaa cacgcggccg ggcttgtctc ccttcccttc ccggtatcgg ttcatggatt | |
| | 13681 | cggttagatg ggaaaccgcc atcagtacca ggtcgtaatc ccacacactg gccatgccgg | |
| | 13741 | ccggccctgc ggaaacctct acgtgcccgt ctggaagctc gtagcggatc acctcgccag | |
| | 13801 | ctcgtcggtc acgcttcgac agacggaaaa cggccacgtc catgatgctg cgactatcgc | |
| | 13861 | gggtgcccac gtcatagagc atcggaacga aaaaatctgg ttgctcgtcg cccttgggcg | |
| | 13921 | gcttcctaat cgacggcgca ccggctgccg gcggttgccg ggattctttg cggattcgat | |
| | 13981 | cagcggccgc ttgccacgat tcaccggggc gtgcttctgc ctcgatgcgt tgccgctggg | |
| | 14041 | cggcctgcgc ggccttcaac ttctccacca ggtcatcacc cagcgccgcg ccgatttgta | |
| | 14101 | ccgggccgga tggtttgcga ccgctcacgc cgattcctcg ggcttggggg ttccagtgcc | |
| | 14161 | attgcagggc cggcagacaa cccagccgct tacgcctggc caaccgcccg ttcctccaca | |
| | 14221 | catggggcat tccacggcgt cggtgcctgg ttgttcttga ttttccatgc cgcctccttt | |
| | 14281 | agccgctaaa attcatctac tcatttattc atttgctcat ttactctggt agctgcgcga | |
| | 14341 | tgtattcaga tagcagctcg gtaatggtct tgccttggcg taccgcgtac atcttcagct | |
| | 14401 | tggtgtgatc ctccgccggc aactgaaagt tgacccgctt catggctggc gtgtctgcca | |
| | 14461 | ggctggccaa cgttgcagcc ttgctgctgc gtgcgctcgg acggccggca cttagcgtgt | |
| | 14521 | ttgtgctttt gctcattttc tctttacctc attaactcaa atgagttttg atttaatttc | |
| | 14581 | agcggccagc gcctggacct cgcgggcagc gtcgccctcg ggttctgatt caagaacggt | |
| | 14641 | tgtgccggcg gcggcagtgc ctgggtagct cacgcgctgc gtgatacggg actcaagaat | |
| | 14701 | gggcagctcg tacccggcca gcgcctcggc aacctcaccg ccgatgcgcg tgcctttgat | |
| | 14761 | cgcccgcgac acgacaaagg ccgcttgtag ccttccatcc gtgacctcaa tgcgctgctt | |
| | 14821 | aaccagctcc accaggtcgg cggtggccca tatgtcgtaa gggcttggct gcaccggaat | |
| | 14881 | cagcacgaag tcggctgcct tgatcgcgga cacagccaag tccgccgcct ggggcgctcc | |
| | 14941 | gtcgatcact acgaagtcgc gccggccgat ggccttcacg tcgcggtcaa tcgtcgggcg | |
| | 15001 | gtcgatgccg acaacggtta gcggttgatc ttcccgcacg gccgcccaat cgcgggcact | |
| | 15061 | gccctgggga tcggaatcga ctaacagaac atcggccccg gcgagttgca gggcgcgggc | |
| | 15121 | tagatgggtt gcgatggtcg tcttgcctga cccgcctttc tggttaagta cagcgataac | |
| | 15181 | cttcatgcgt tccccttgcg tatttgttta tttactcatc gcatcatata cgcagcgacc | |
| | 15241 | gcatgacgca agctgtttta ctcaaataca catcaccttt ttagacggcg gcgctcggtt | |
| | 15301 | tcttcagcgg ccaagctggc cggccaggcc gccagcttgg catcagacaa accggccagg | |
| | 15361 | atttcatgca gccgcacggt tgagacgtgc gcgggcggct cgaacacgta cccggccgcg | |
| | 15421 | atcatctccg cctcgatctc ttcggtaatg aaaaacggtt cgtcctggcc gtcctggtgc | |
| | 15481 | ggtttcatgc ttgttcctct tggcgttcat tctcggcggc cgccagggcg tcggcctcgg | |
| | 15541 | tcaatgcgtc ctcacggaag gcaccgcgcc gcctggcctc ggtgggcgtc acttcctcgc | |
| | 15601 | tgcgctcaag tgcgcggtac agggtcgagc gatgcacgcc aagcagtgca gccgcctctt | |
| | 15661 | tcacggtgcg gccttcctgg tcgatcagct cgcgggcgtg cgcgatctgt gccggggtga | |
| | 15721 | gggtagggcg ggggccaaac ttcacgcctc gggccttggc ggcctcgcgc ccgctccggg | |
| | 15781 | tgcggtcgat gattagggaa cgctcgaact cggcaatgcc ggcgaacacg gtcaacacca | |
| | 15841 | tgcggccggc cggcgtggtg gtgtcggccc acggctctgc caggctacgc aggcccgcgc | |
| | 15901 | cggcctcctg gatgcgctcg gcaatgtcca gtaggtcgcg ggtgctgcgg gccaggcggt | |
| | 15961 | ctagcctggt cactgtcaca acgtcgccag ggcgtaggtg gtcaagcatc ctggccagct | |
| | 16021 | ccgggcggtc gcgcctggtg ccggtgatct tctcggaaaa cagcttggtg cagccggccg | |
| | 16081 | cgtgcagttc ggcccgttgg ttggtcaagt cctggtcgtc ggtgctgacg cgggcatagc | |
| | 16141 | ccagcaggcc agcggcggcg ctcttgttca tggcgtaatg tctccggttc tagtcgcaag | |
| | 16201 | tattctactt tatgcgacta aaacacgcga caagaaaacg ccaggaaaag ggcagggcgg | |
| | 16261 | cagcctgtcg cgtaacttag gacttgtgcg acatgtcgtt ttcagaagac ggctgcactg | |
| | 16321 | aacgtcagaa gccgactgca ctatagcagc ggaggggttg gatcaaagta ctttgatccc | |
| | 16381 | gaggggaacc ctgtggttgg catgcacata caaatggacg aacggataaa ccttttcacg | |
| | 16441 | cccttttaaa tatccgttat tctaataaac gctcttttct cttag | |
| 33 | GTATGAAGAGAGGGAATGAT | | A gRNA cf |
| | | | phse401-basil- |
| | | | x4 CRISPR |
| | | | construct (SEQ |
| | | | ID NO: 37) to |
| | | | disrupt elf3 in |
| | | | basil. |
| 34 | GGCGTTGTTAGTCGGGTTTC | | A gRNA of |
| | | | phse401-basil- |
| | | | x4 CRISPR |
| | | | construct (SEQ |
| | | | ID NO: 37) to |
| | | | disrupt elf3 in |
| | | | basil. |
| 35 | GAATCGGTGAGTCTGTGGGA | | A gRNA of |
| | | | phse401-basil- |
| | | | x4 CRISPR |
| | | | construct (SEQ |
| | | | ID NO: 37) to |
| | | | disrupt elf3 in |
| | | | basil. |
| 36 | GACGGACGATTGAGACTAGA | | A gRNA of |
| | | | phse401-basil- |
| | | | x4 CRISPR |
| | | | construct (SEQ |
| | | | ID NO: 37) to |
| | | | disrupt elf3 in |
| | | | basil. |
| 37 | 1 | TAAACGCTCT TTTCTCTTAG GTTTACCCGC CAATATATCC TGTCAAACAC TGATAGTTTA | pHSE401_basil_ |
| | 61 | AACTGAAGGC GGGAAACGAC AATCTGATCC AAGCTCAAGC TGCTCTAGCA TTCGCCATTC | x4 |
| | 121 | AGGCTGCGCA ACTGTTGGGA AGGGCGATCG GTGCGGGCCT CTTCGCTATT ACGCCAGCTG | Plasmid |
| | 181 | GCGAAAGGGG GATGTGCTGC AAGGCGATTA AGTTGGGTAA CGCCAGGGTT TTCCCAGTCA | comprising |
| | 241 | CGACGTTGTA AAACGACGGC CAGTGCCAAG CTTCGACTTG CCTTCCGCAC AATACATCAT | CRISPR system |
| | 301 | TTCTTCTTAG CTTTTTTTCT TCTTCTTCGT TCATACAGTT TTTTTTTGTT TATCAGCTTA | to disrupt elf3 |
| | 361 | CATTTTCTTG AACCGTAGCT TTCGTTTTCT TCTTTTTAAC TTTCCATTCG GAGTTTTTGT | in basil. |
| | 421 | ATCTTGTTTC ATAGTTTGTC CCAGGATTAG AATGATTAGG CATCGAACCT TCAAGAATTT | |
| | 481 | GATTGAATAA AACATCTTCA TTCTTAAGAT ATGAAGATAA TCTTCAAAAG GCCCCTGGGA | |
| | 541 | ATCTGAAAGA AGAGAAGCAG GCCCATTTAT ATGGGAAAGA ACAATAGTAT TTCTTATATA | |
| | 601 | GGCCCATTTA AGTTGAAAAC AATCTTCAAA AGTCCCACAT CGCTTAGATA AGAAAACGAA | |
| | 661 | GCTGAGTTTA TATACAGCTA GAGTCGAAGT AGTGATTGTA TGAAGAGAGG GAATGATGTT | |
| | 721 | TTAGAGCTAG AAATAGCAAG TTAAAATAAG GCTAGTCCGT TATCAACTTG AAAAAGTGGC | |
| | 781 | ACCGAGTCGG TGCTTTTTTT TGCAAAATTT TCCAGATCGA TTTCTTCTTC CTCTGTTCTT | |
| | 841 | CGGCGTTCAA TTTCTGGGGT TTTCTCTTCG TTTTCTGTAA CTGAAACCTA AAATTTGACC | |
| | 901 | TAAAAAAAAT CTCAAATAAT ATGATTCAGT GGTTTTGTAC TTTTCAGTTA GTTGAGTTTT | |
| | 961 | GCAGTTCCGA TGAGATAAAC CAATATTAAT CCAAACTACT GCAGCCTGAC AGACAAATGA | |
| | 1021 | GGATGCAAAC AATTTTAAAG TTTATCTAAC GCTAGCTGTT TTGTTTCTTC TCTCTGGTGC | |
| | 1081 | ACCAACGACG GCGTTTTCTC AATCATAAAG AGGCTTGTTT TACTTAAGGC CAATAATGTT | |
| | 1141 | GATGGATCGA AAGAAGAGGG CTTTTAATAA ACGAGCCCGT TTAAGCTGTA AACGATGTCA | |
| | 1201 | AAAACATCCC ACATCGTTCA GTTGAAAATA GAAGCTCTGT TTATATATTG GTAGAGTCGA | |
| | 1261 | CTAAGAGATT GGCGTTGTTA GTCGGGTTTC GTTTTAGAGC TAGAAATAGC AAGTTAAAAT | |
| | 1321 | AAGGCTAGTC CGTTATCAAC TTGAAAAAGT GGCACCGAGT CGGTGCTTTT TTTGGATAGA | |
| | 1381 | ATTTCCCAGC TTTTTTGCGT GTTTCAGCTC TCATGATCCT TGGCCAATGG GTGTAGTAAA | |
| | 1441 | TTTTCTGCAC ATTCATTGGA TGGAAAATAA TGGTTTTAGC TTTAGGGAAT AAGAAAAGTG | |
| | 1501 | TATAGGAAGG GGATTTTTGT ACAATCACAT TTGAATTAGG TCTTTGAAAT GACAGGGAAT | |
| | 1561 | GAGGACATAT GATGAGACGG TCATTGTTTT AGTTCCACCA CGATTATATT TGAAATTTAC | |
| | 1621 | GTGAGTGTGA GTGAGACTTG CATAAGAAAA TAAAATCTTT AGTTGGGAAA AAATTCAATA | |
| | 1681 | ATATAAATGG GCTTGAGAAG GAAGCGAGGG ATAGGCCTTT TTCTAAAATA GGCCCATTTA | |
| | 1741 | AGCTATTAAC AATCTTCAAA AGTACCACAG CGCTTAGGTA AAGAAAGCAG CTGAGTTTAT | |
| | 1801 | ATATGGTTAG AGACGAAGTA GTGATTGAAT CGGTGAGTCT GTGGGAGTTT TAGAGCTAGA | |
| | 1861 | AATAGCAAGT TAAAATAAGG CTAGTCCGTT ATCAACTTGA AAAAGTGGCA CCGAGTCGGT | |
| | 1921 | GCTTTTTTTT GGCAAAAATT TTCAGATTTT TTCTTCATCT GTAGATTTCT GGGTTTTTTT | |
| | 1981 | TTCCGTTTCG TGAATCATAA GTGAAGTTTT GGATGCAAAT CTGCGCGAAA AAAGTTGGAC | |
| | 2041 | CTGCAATGAG CTTATTTAGA TAGCTAAGAC AAAGTGATTG GTCCGTTCGA CTTGCCTTCC | |
| | 2101 | GCACAATACA TCATTTCTTC TTAGCTTTTT TTCTTCTTCT TCGTTCATAC AGTTTTTTTT | |
| | 2161 | TGTTTATCAG CTTACATTTT CTTGAACCGT AGCTTTCGTT TTCTTCTTTT TAACTTTCCA | |
| | 2221 | TTCGGAGTTT TTGTATCTTG TTTCATAGTT TGTCCCAGGA TTAGAATGAT TAGGCATCGA | |
| | 2281 | ACCTTCAAGA ATTTGATTGA ATAAAACATC TTCATTCTTA AGATATGAAG ATAATCTTCA | |
| | 2341 | AAAGGCCCCT GGGAATCTGA AAGAAGAGAA GCAGGCCCAT TTATATGGGA AAGAACAATA | |
| | 2401 | GTATTTCTTA TATAGGCCCA TTTAAGTTGA AAACAATCTT CAAAAGTCCC ACATCGCTTA | |
| | 2461 | GATAAGAAAA CGAAGCTGAG TTTATATACA GCTAGAGTCG AAGTAGTGAT TGACGGACGA | |
| | 2521 | TTGAGACTAG AGTTTTAGAG CTAGAAATAG CAAGTTAAAA TAAGGCTAGT CCGTTATCAA | |
| | 2581 | CTTGAAAAAG TGGCACCGAG TCGGTGCTTT TTTTTGCAAA ATTTTCCAGA TCGATTTCTT | |
| | 2641 | CTTCCTCTGT TCTTCGGCGT TCAATTTCTG GGGTTTTCTC TTCGTTTTCT GTAACTGAAA | |
| | 2701 | CCTAAAATTT GACCTAAAAA AAATCTCAAA TAATATGATT CAGTGGTTTT GTACTTTTCA | |
| | 2761 | GTTAGTTGAG TTTTGCAGTT CCGATGAGAT AAACCAATAA GCTTGCATGC CTGCAGGTCA | |
| | 2821 | ACATGGTGGA GCACGACACA CTTGTCTACT CCAAAAATAT CAAAGATACA GTCTCAGAAG | |
| | 2881 | ACCAAAGGGC AATTGAGACT TTTCAACAAA GGGTAATATC CGGAAACCTC CTCGGATTCC | |
| | 2941 | ATTGCCCAGC TATCTGTCAC TTTATTGTGA AGATAGTGGA AAAGGAAGGT GGCTCCTACA | |
| | 3001 | AATGCCATCA TTGCGATAAA GGAAAGGCCA TCGTTGAAGA TGCCTCTGCC GACAGTGGTC | |
| | 3061 | CCAAAGATGG ACCCCCACCC ACGAGGAGCA TCGTGGAAAA AGAAGACGTT CCAACCACGT | |
| | 3121 | CTTCAAAGCA AGTGGATTGA TGTGATAACA TGGTGGAGCA CGACACACTT GTCTACTCCA | |
| | 3181 | AAAATATCAA AGATACAGTC TCAGAAGACC AAAGGGCAAT TGAGACTTTT CAACAAAGGG | |
| | 3241 | TAATATCCGG AAACCTCCTC GGATTCCATT GCCCAGCTAT CTGTCACTTT ATTGTGAAGA | |
| | 3301 | TAGTGGAAAA GGAAGGTGGC TCCTACAAAT GCCATCATTG CGATAAAGGA AAGGCCATCG | |
| | 3361 | TTGAAGATGC CTCTGCCGAC AGTGGTCCCA AAGATGGACC CCCACCCACG AGGAGCATCG | |
| | 3421 | TGGAAAAAGA AGACGTTCCA ACCACGTCTT CAAAGCAAGT GGATTGATGT GATATCTCCA | |
| | 3481 | CTGACGTAAG GGATGACGCA CAATCCCACT ATCCTTCGCA AGACCCTTCC TCTATATAAG | |
| | 3541 | GAAGTTCATT TCATTTGGAG AGGACCTCGA CCTCAACACA ACATATACAA AACAAACGAA | |
| | 3601 | TCTCAAGCAA TCAAGCATTC TACTTCTATT GCAGCAATTT AAATCATTTC TTTTAAAGCA | |
| | 3661 | AAAGCAATTT TCTGAAAATT TTCACCATTT ACGAACGATA CTCGAGTAAT CTAGATGGAT | |
| | 3721 | TACAAGGACC ACGACGGGGA TTACAAGGAC CACGACATTG ATTACAAGGA TGATGATGAC | |
| | 3781 | AAGATGGCTC CGAAGAAGAA GAGGAAGGTT GGCATCCACG GGGTGCCAGC TGCTGACAAG | |
| | 3841 | AAGTACTCGA TCGGCCTCGA TATTGGGACT AACTCTGTTG GCTGGGCCGT GATCACCGAC | |
| | 3901 | GAGTACAAGG TGCCCTCAAA GAAGTTCAAG GTCCTGGGCA ACACCGATCG GCATTCCATC | |
| | 3961 | AAGAAGAATC TCATTGGCGC TCTCCTGTTC GACAGCGGCG AGACGGCTGA GGCTACGCGG | |
| | 4021 | CTCAAGCGCA CCGCCCGCAG GCGGTACACG CGCAGGAAGA ATCGCATCTG CTACCTGCAG | |
| | 4081 | GAGATTTTCT CCAACGAGAT GGCGAAGGTT GACGATTCTT TCTTCCACAG GCTGGAGGAG | |
| | 4141 | TCATTCCTCG TGGAGGAGGA TAAGAAGCAC GAGCGGCATC CAATCTTCGG CAACATTGTC | |
| | 4201 | GACGAGGTTG CCTACCACGA GAAGTACCCT ACGATCTACC ATCTGCGGAA GAAGCTCGTG | |
| | 4261 | GACTCCACAG ATAAGGCGGA CCTCCGCCTG ATCTACCTCG CTCTGGCCCA CATGATTAAG | |
| | 4321 | TTCAGGGGCC ATTTCCTGAT CGAGGGGGAT CTCAACCCGG ACAATAGCGA TGTTGACAAG | |
| | 4381 | CTGTTCATCC AGCTCGTGCA GACGTACAAC CAGCTCTTCG AGGAGAACCC CATTAATGCG | |
| | 4441 | TCAGGCGTCG ACGCGAAGGC TATCCTGTCC GCTAGGCTCT CGAAGTCTCG GCGCCTCGAG | |
| | 4501 | AACCTGATCG CCCAGCTGCC GGGCGAGAAG AAGAACGGCC TGTTCGGGAA TCTCATTGCG | |
| | 4561 | CTCAGCCTGG GGCTCACGCC CAACTTCAAG TCGAATTTCG ATCTCGCTGA GGACGCCAAG | |
| | 4621 | CTGCAGCTCT CCAAGGACAC ATACGACGAT GACCTGGATA ACCTCCTGGC CCAGATCGGC | |
| | 4681 | GATCAGTACG CGGACCTGTT CCTCGCTGCC AAGAATCTGT CGGACGCCAT CCTCCTGTCT | |
| | 4741 | GATATTCTCA GGGTGAACAC CGAGATTACG AAGGCTCCGC TCTCAGCCTC CATGATCAAG | |
| | 4801 | CGCTACGACG AGCACCATCA GGATCTGACC CTCCTGAAGG CGCTGGTCAG GCAGCAGCTC | |
| | 4861 | CCCGAGAAGT ACAAGGAGAT CTTCTTCGAT CAGTCGAAGA ACGGCTACGC TGGGTACATT | |
| | 4921 | GACGGCGGGG CCTCTCAGGA GGAGTTCTAC AAGTTCATCA AGCCGATTCT GGAGAAGATG | |
| | 4981 | GACGGCACGG AGGAGCTGCT GGTGAAGCTC AATCGCGAGG ACCTCCTGAG GAAGCAGCGG | |
| | 5041 | ACATTCGATA ACGGCAGCAT CCCACACCAG ATTCATCTCG GGGAGCTGCA CGCTATCCTG | |
| | 5101 | AGGAGGCAGG AGGACTTCTA CCCTTTCCTC AAGGATAACC GCGAGAAGAT CGAGAAGATT | |
| | 5161 | CTGACTTTCA GGATCCCGTA CTACGTCGGC CCACTCGCTA GGGGCAACTC CCGCTTCGCT | |
| | 5221 | TGGATGACCC GCAAGTCAGA GGAGACGATC ACGCCGTGGA ACTTCGAGGA GGTGGTCGAC | |
| | 5281 | AAGGGCGCTA GCGCTCAGTC GTTCATCGAG AGGATGACGA ATTTCGACAA GAACCTGCCA | |
| | 5341 | AATGAGAAGG TGCTCCCTAA GCACTCGCTC CTGTACGAGT ACTTCACAGT CTACAACGAG | |
| | 5401 | CTGACTAAGG TGAAGTATGT GACCGAGGGC ATGAGGAAGC CGGCTTTCCT GTCTGGGGAG | |
| | 5461 | CAGAAGAAGG CCATCGTGGA CCTCCTGTTC AAGACCAACC GGAAGGTCAC GGTTAAGCAG | |
| | 5521 | CTCAAGGAGG ACTACTTCAA GAAGATTGAG TGCTTCGATT CGGTCGAGAT CTCTGGCGTT | |
| | 5581 | GAGGACCGCT TCAACGCCTC CCTGGGGACC TACCACGATC TCCTGAAGAT CATTAAGGAT | |
| | 5641 | AAGGACTTCC TGGACAACGA GGAGAATGAG GATATCCTCG AGGACATTGT GCTGACACTC | |
| | 5701 | ACTCTGTTCG AGGACCGGGA GATGATCGAG GAGCGCCTGA AGACTTACGC CCATCTCTTC | |
| | 5761 | GATGACAAGG TCATGAAGCA GCTCAAGAGG AGGAGGTACA CCGGCTGGGG GAGGCTGAGC | |
| | 5821 | AGGAAGCTCA TCAACGGCAT TCGGGACAAG CAGTCCGGGA AGACGATCCT CGACTTCCTG | |
| | 5881 | AAGAGCGATG GCTTCGCGAA CCGCAATTTC ATGCAGCTGA TTCACGATGA CAGCCTCACA | |
| | 5941 | TTCAAGGAGG ATATCCAGAA GGCTCAGGTG AGCGGCCAGG GGGACTCGCT GCACGAGCAT | |
| | 6001 | ATCGCGAACC TCGCTGGCTC GCCAGCTATC AAGAAGGGGA TTCTGCAGAC CGTGAAGGTT | |
| | 6061 | GTGGACGAGC TGGTGAAGGT CATGGGCAGG CACAAGCCTG AGAACATCGT CATTGAGATG | |
| | 6121 | GCCCGGGAGA ATCAGACCAC GCAGAAGGGC CAGAAGAACT CACGCGAGAG GATGAAGAGG | |
| | 6181 | ATCGAGGAGG GCATTAAGGA GCTGGGGTCC CAGATCCTCA AGGAGCACCC GGTGGAGAAC | |
| | 6241 | ACGCAGCTGC AGAATGAGAA GCTCTACCTG TACTACCTCC AGAATGGCCG CGATATGTAT | |
| | 6301 | GTGGACCAGG AGCTGGATAT TAACAGGCTC AGCGATTACG ACGTCGATCA TATCGTTCCA | |
| | 6361 | CAGTCATTCC TGAAGGATGA CTCCATTGAC AACAAGGTCC TCACCAGGTC GGACAAGAAC | |
| | 6421 | CGGGGCAAGT CTGATAATGT TCCTTCAGAG GAGGTCGTTA AGAAGATGAA GAACTACTGG | |
| | 6481 | CGCCAGCTCC TGAATGCCAA GCTGATCACG CAGCGGAAGT TCGATAACCT CACAAAGGCT | |
| | 6541 | GAGAGGGGCG GGCTCTCTGA GCTGGACAAG GCGGGCTTCA TCAAGAGGCA GCTGGTCGAG | |
| | 6601 | ACACGGCAGA TCACTAAGCA CGTTGCGCAG ATTCTCGACT CACGGATGAA CACTAAGTAC | |
| | 6661 | GATGAGAATG ACAAGCTGAT CCGCGAGGTG AAGGTCATCA CCCTGAAGTC AAAGCTCGTC | |
| | 6721 | TCCGACTTCA GGAAGGATTT CCAGTTCTAC AAGGTTCGGG AGATCAACAA TTACCACCAT | |
| | 6781 | GCCCATGACG CGTACCTGAA CGCGGTGGTC GGCACAGCTC TGATCAAGAA GTACCCAAAG | |
| | 6841 | CTCGAGAGCG AGTTCGTGTA CGGGGACTAC AAGGTTTACG ATGTGAGGAA GATGATCGCC | |
| | 6901 | AAGTCGGAGC AGGAGATTGG CAAGGCTACC GCCAAGTACT TCTTCTACTC TAACATTATG | |
| | 6961 | AATTTCTTCA AGACAGAGAT CACTCTGGCC AATGGCGAGA TCCGGAAGCG CCCCCTCATC | |
| | 7021 | GAGACGAACG GCGAGACGGG GGAGATCGTG TGGGACAAGG GCAGGGATTT CGCGACCGTC | |
| | 7081 | AGGAAGGTTC TCTCCATGCC ACAAGTGAAT ATCGTCAAGA AGACAGAGGT CCAGACTGGC | |
| | 7141 | GGGTTCTCTA AGGAGTCAAT TCTGCCTAAG CGGAACAGCG ACAAGCTCAT CGCCCGCAAG | |
| | 7201 | AAGGACTGGG ATCCGAAGAA GTACGGCGGG TTCGACAGCC CCACTGTGGC CTACTCGGTC | |
| | 7261 | CTGGTTGTGG CGAAGGTTGA GAAGGGCAAG TCCAAGAAGC TCAAGAGCGT GAAGGAGCTG | |
| | 7321 | CTGGGGATCA CGATTATGGA GCGCTCCAGC TTCGAGAAGA ACCCGATCGA TTTCCTGGAG | |
| | 7381 | GCGAAGGGCT ACAAGGAGGT GAAGAAGGAC CTGATCATTA AGCTCCCCAA GTACTCACTC | |
| | 7441 | TTCGAGCTGG AGAACGGCAG GAAGCGGATG CTGGCTTCCG CTGGCGAGCT GCAGAAGGGG | |
| | 7501 | AACGAGCTGG CTCTGCCGTC CAAGTATGTG AACTTCCTCT ACCTGGCCTC CCACTACGAG | |
| | 7561 | AAGCTCAAGG GCAGCCCCGA GGACAACGAG CAGAAGCAGC TGTTCGTCGA GCAGCACAAG | |
| | 7621 | CATTACCTCG ACGAGATCAT TGAGCAGATT TCCGAGTTCT CCAAGCGCGT GATCCTGGCC | |
| | 7681 | GACGCGAATC TGGATAAGGT CCTCTCCGCG TACAACAAGC ACCGCGACAA GCCAATCAGG | |
| | 7741 | GAGCAGGCTG AGAATATCAT TCATCTCTTC ACCCTGACGA ACCTCGGCGC CCCTGCTGCT | |
| | 7801 | TTCAAGTACT TCGACACAAC TATCGATCGC AAGAGGTACA CAAGCACTAA GGAGGTCCTG | |
| | 7861 | GACGCGACCC TCATCCACCA GTCGATTACC GGCCTCTACG AGACGCGCAT CGACCTGTCT | |
| | 7921 | CAGCTCGGGG GCGACAAGCG GCCAGCGGCG ACGAAGAAGG CGGGGCAGGC GAAGAAGAAG | |
| | 7981 | AAGTGAGCTC AGAGCTTTCG TTCGTATCAT CGGTTTCGAC AACGTTCGTC AAGTTCAATG | |
| | 8041 | CATCAGTTTC ATTGCGCACA CACCAGAATC CTACTGAGTT TGAGTATTAT GGCATTGGGA | |
| | 8101 | AAACTGTTTT TCTTGTACCA TTTGTTGTGC TTGTAATTTA CTGTGTTTTT TATTCGGTTT | |
| | 8161 | TCGCTATCGA ACTGTGAAAT GGAAATGGAT GGAGAAGAGT TAATGAATGA TATGGTCCTT | |
| | 8221 | TTGTTCATTC TCAAATTAAT ATTATTTGTT TTTTCTCTTA TTTGTTGTGT GTTGAATTTG | |
| | 8281 | AAATTATAAG AGATATGCAA ACATTTTGTT TTGAGTAAAA ATGTGTCAAA TCGTGGCCTC | |
| | 8341 | TAATGACCGA AGTTAATATG AGGAGTAAAA CACTTGTAGT TGTACCATTA TGCTTATTCA | |
| | 8401 | CTAGGCAACA AATATATTTT CAGACCTAGA AAAGCTGCAA ATGTTACTGA ATACAAGTAT | |
| | 8461 | GTCCTCTTGT GTTTTAGACA TTTATGAACT TTCCTTTATG TAATTTTCCA GAATCCTTGT | |
| | 8521 | CAGATTCTAA TCATTGCTTT ATAATTATAG TTATACTCAT GGATTTGTAG TTGAGTATGA | |
| | 8581 | AAATATTTTT TAATGCATTT TATGACTTGC CAATTGATTG ACAACGAATT CGTAATCATG | |
| | 8641 | TCATAGCTGT TTCCTGTGTG AAATTGTTAT CCGCTCACAA TTCCACACAA CATACGAGCC | |
| | 8701 | GGAAGCATAA AGTGTAAAGC CTGGGGTGCC TAATGAGTGA GCTAACTCAC ATTAATTGCG | |
| | 8761 | TTGCGCTCAC TGCCCGCTTT CCAGTCGGGA AACCTGTCGT GCCAGCTGCA TTAATGAATC | |
| | 8821 | GGCCAACGCG CGGGGAGAGG CGGTTTGCGT ATTGGCTAGA GCAGCTTGCC AACATGGTGG | |
| | 8881 | AGCACGACAC TCTCGTCTAC TCCAAGAATA TCAAAGATAC AGTCTCAGAA GACCAAAGGG | |
| | 8941 | CTATTGAGAC TTTTCAACAA AGGGTAATAT CGGGAAACCT CCTCGGATTC CATTGCCCAG | |
| | 9001 | CTATCTGTCA CTTCATCAAA AGGACAGTAG AAAAGGAAGG TGGCACCTAC AAATGCCATC | |
| | 9061 | ATTGCGATAA AGGAAAGGCT ATCGTTCAAG ATGCCTCTGC CGACAGTGGT CCCAAAGATG | |
| | 9121 | GACCCCCACC CACGAGGAGC ATCGTGGAAA AAGAAGACGT TCCAACCACG TCTTCAAAGC | |
| | 9181 | AAGTGGATTG ATGTGAACAT GGTGGAGCAC GACACTCTCG TCTACTCCAA GAATATCAAA | |
| | 9241 | GATACAGTCT CAGAAGACCA AAGGGCTATT GAGACTTTTC AACAAAGGGT AATATCGGGA | |
| | 9301 | AACCTCCTCG GATTCCATTG CCCAGCTATC TGTCACTTCA TCAAAAGGAC AGTAGAAAAG | |
| | 9361 | GAAGGTGGCA CCTACAAATG CCATCATTGC GATAAAGGAA AGGCTATCGT TCAAGATGCC | |
| | 9421 | TCTGCCGACA GTGGTCCCAA AGATGGACCC CCACCCACGA GGAGCATCGT GGAAAAAGAA | |
| | 9481 | GACGTTCCAA CCACGTCTTC AAAGCAAGTG GATTGATGTG ATATCTCCAC TGACGTAAGG | |
| | 9541 | GATGACGCAC AATCCCACTA TCCTTCGCAA GACCCTTCCT CTATATAAGG AAGTTCATTT | |
| | 9601 | CATTTGGAGA GGACACGCTG AAATCACCAG TCTCTCTCTA CAAATCTATC TCTCTCGAGC | |
| | 9661 | TTTCGCAGAT CCGGGGGGCA ATGAGATATG AAAAAGCCTG AACTCACCGC GACGTCTGTC | |
| | 9721 | GAGAAGTTTC TGATCGAAAA GTTCGACAGC GTCTCCGACC TGATGCAGCT CTCGGAGGGC | |
| | 9781 | GAAGAATCTC GTGCTTTCAG CTTCGATGTA GGAGGGCGTG GATATGTCCT GCGGGTAAAT | |
| | 9841 | AGCTGCGCCG ATGGTTTCTA CAAAGATCGT TATGTTTATC GGCACTTTGC ATCGGCCGCG | |
| | 9901 | CTCCCGATTC CGGAAGTGCT TGACATTGGG GAGTTTAGCG AGAGCCTGAC CTATTGCATC | |
| | 9961 | TCCCGCCGTT CACAGGGTGT CACGTTGCAA GACCTGCCTG AAACCGAACT GCCCGCTGTT | |
| | 10021 | CTACAACCGG TCGCGGAGGC TATGGATGCG ATCGCTGCGG CCGATCTTAG CCAGACGAGC | |
| | 10081 | GGGTTCGGCC CATTCGGACC GCAAGGAATC GGTCAATACA CTACATGGCG TGATTTCATA | |
| | 10141 | TGCGCGATTG CTGATCCCCA TGTGTATCAC TGGCAAACTG TGATGGACGA CACCGTCAGT | |
| | 10201 | GCGTCCGTCG CGCAGGCTCT CGATGAGCTG ATGCTTTGGG CCGAGGACTG CCCCGAAGTC | |
| | 10261 | CGGCACCTCG TGCACGCGGA TTTCGGCTCC AACAATGTCC TGACGGACAA TGGCCGCATA | |
| | 10321 | ACAGCGGTCA TTGACTGGAG CGAGGCGATG TTCGGGGATT CCCAATACGA GGTCGCCAAC | |
| | 10381 | ATCTTCTTCT GGAGGCCGTG GTTGGCTTGT ATGGAGCAGC AGACGCGCTA CTTCGAGCGG | |
| | 10441 | AGGCATCCGG AGCTTGCAGG ATCGCCACGA CTCCGGGCGT ATATGCTCCG CATTGGTCTT | |
| | 10501 | GACCAACTCT ATCAGAGCTT GGTTGACGGC AATTTCGATG ATGCAGCTTG GGCGCAGGGT | |
| | 10561 | CGATGCGACG CAATCGTCCG ATCCGGAGCC GGGACTGTCG GGCGTACACA AATCGCCCGC | |
| | 10621 | AGAAGCGCGG CCGTCTGGAC CGATGGCTGT GTAGAAGTAC TCGCCGATAG TGGAAACCGA | |
| | 10681 | CGCCCCAGCA CTCGTCCGAG GGCAAAGAAA TAGAGTAGAT GCCGACCGGG ATCTGTCGAT | |
| | 10741 | CGACAAGCTC GAGTTTCTCC ATAATAATGT GTGAGTAGTT CCCAGATAAG GGAATTAGGG | |
| | 10801 | TTCCTATAGG GTTTCGCTCA TGTGTTGAGC ATATAAGAAA CCCTTAGTAT GTATTTGTAT | |
| | 10861 | TTGTAAAATA CTTCTATCAA TAAAATTTCT AATTCCTAAA ACCAAAATCC AGTACTAAAA | |
| | 10921 | TCCAGATCCC CCGAATTAAT TCGGCGTTAA TTCAGTACAT TAAAAACGTC CGCAATGTGT | |
| | 10981 | TATTAAGTTG TCTAAGCGTC AATTTGTTTA CACCACAATA TATCCTGCCA CCAGCCAGCC | |
| | 11041 | AACAGCTCCC CGACCGGCAG CTCGGCACAA AATCACCACT CGATACAGGC AGCCCATCAG | |
| | 11101 | TCCGGGACGG CGTCAGCGGG AGAGCCGTTG TAAGGCGGCA GACTTTGCTC ATGTTACCGA | |
| | 11161 | TGCTATTCGG AAGAACGGCA ACTAAGCTGC CGGGTTTGAA ACACGGATGA TCTCGCGGAG | |
| | 11221 | GGTAGCATGT TGATTGTAAC GATGACAGAG CGTTGCTGCC TGTGATCACC GCGGTTTCAA | |
| | 11281 | AATCGGCTCC GTCGATACTA TGTTATACGC CAACTTTGAA AACAACTTTG AAAAAGCTGT | |
| | 11341 | TTTCTGGTAT TTAAGGTTTT AGAATGCAAG GAACAGTGAA TTGGAGTTCG TCTTGTTATA | |
| | 11401 | ATTAGCTTCT TGGGGTATCT TTAAATACTG TAGAAAAGAG GAAGGAAATA ATAAATGGCT | |
| | 11461 | AAAATGAGAA TATCACCGGA ATTGAAAAAA CTGATCGAAA AATACCGCTG CGTAAAAGAT | |
| | 11521 | ACGGAAGGAA TGTCTCCTGC TAAGGTATAT AAGCTGGTGG GAGAAAATGA AAACCTATAT | |
| | 11581 | TTAAAAATGA CGGACAGCCG GTATAAAGGG ACCACCTATG ATGTGGAACG GGAAAAGGAC | |
| | 11641 | ATGATGCTAT GGCTGGAAGG AAAGCTGCCT GTTCCAAAGG TCCTGCACTT TGAACGGCAT | |
| | 11701 | GATGGCTGGA GCAATCTGCT CATGAGTGAG GCCGATGGCG TCCTTTGCTC GGAAGAGTAT | |
| | 11761 | GAAGATGAAC AAAGCCCTGA AAAGATTATC GAGCTGTATG CGGAGTGCAT CAGGCTCTTT | |
| | 11821 | CACTCCATCG ACATATCGGA TTGTCCCTAT ACGAATAGCT TAGACAGCCG CTTAGCCGAA | |
| | 11881 | TTGGATTACT TACTGAATAA CGATCTGGCC GATGTGGATT GCGAAAACTG GGAAGAAGAC | |
| | 11941 | ACTCCATTTA AAGATCCGCG CGAGCTGTAT GATTTTTTAA AGACGGAAAA GCCCGAAGAG | |
| | 12001 | GAACTTGTCT TTTCCCACGG CGACCTGGGA GACAGCAACA TCTTTGTGAA AGATGGCAAA | |
| | 12061 | GTAAGTGGCT TTATTGATCT TGGGAGAAGC GGCAGGGCGG ACAAGTGGTA TGACATTGCC | |
| | 12121 | TTCTGCGTCC GGTCGATCAG GGAGGATATC GGGGAAGAAC AGTATGTCGA GCTATTTTTT | |
| | 12181 | GACTTACTGG GGATCAAGCC TGATTGGGAG AAAATAAAAT ATTATATTTT ACTGGATGAA | |
| | 12241 | TTGTTTTAGT ACCTAGAATG CATGACCAAA ATCCCTTAAC GTGAGTTTTC GTTCCACTGA | |
| | 12301 | GCGTCAGACC CCGTAGAAAA GATCAAAGGA TCTTCTTGAG ATCCTTTTTT TCTGCGCGTA | |
| | 12361 | ATCTGCTGCT TGCAAACAAA AAAACCACCG CTACCAGCGG TGGTTTGTTT GCCGGATCAA | |
| | 12421 | GAGCTACCAA CTCTTTTTCC GAAGGTAACT GGCTTCAGCA GAGCGCAGAT ACCAAATACT | |
| | 12481 | GTCCTTCTAG TGTAGCCGTA GTTAGGCCAC CACTTCAAGA ACTCTGTAGC ACCGCCTACA | |
| | 12541 | TACCTCGCTC TGCTAATCCT GTTACCAGTG GCTGCTGCCA GTGGCGATAA GTCGTGTCTT | |
| | 12601 | ACCGGGTTGG ACTCAAGACG ATAGTTACCG GATAAGGCGC AGCGGTCGGG CTGAACGGGG | |
| | 12661 | GGTTCGTGCA CACAGCCCAG CTTGGAGCGA ACGACCTACA CCGAACTGAG ATACCTACAG | |
| | 12721 | CGTGAGCTAT GAGAAAGCGC CACGCTTCCC GAAGGGAGAA AGGCGGACAG GTATCCGGTA | |
| | 12781 | AGCGGCAGGG TCGGAACAGG AGAGCGCACG AGGGAGCTTC CAGGGGGAAA CGCCTGGTAT | |
| | 12841 | CTTTATAGTC CTGTCGGGTT TCGCCACCTC TGACTTGAGC GTCGATTTTT GTGATGCTCG | |
| | 12901 | TCAGGGGGGC GGAGCCTATG GAAAAACGCC AGCAACGCGG CCTTTTTACG GTTCCTGGCC | |
| | 12961 | TTTTGCTGGC CTTTTGCTCA CATGTTCTTT CCTGCGTTAT CCCCTGATTC TGTGGATAAC | |
| | 13021 | CGTATTACCG CCTTTGAGTG AGCTGATACC GCTCGCCGCA GCCGAACGAC CGAGCGCAGC | |
| | 13081 | GAGTCAGTGA GCGAGGAAGC GGAAGAGCGC CTGATGCGGT ATTTTCTCCT TACGCATCTG | |
| | 13141 | TGCGGTATTT CACACCGCAT ATGGTGCACT CTCAGTACAA TCTGCTCTGA TGCCGCATAG | |
| | 13201 | TTAAGCCAGT ATACACTCCG CTATCGCTAC GTGACTGGGT CATGGCTGCG CCCCGACACC | |
| | 13261 | CGCCAACACC CGCTGACGCG CCCTGACGGG CTTGTCTGCT CCCGGCATCC GCTTACAGAC | |
| | 13321 | AAGCTGTGAC CGTCTCCGGG AGCTGCATGT GTCAGAGGTT TTCACCGTCA TCACCGAAAC | |
| | 13381 | GCGCGAGGCA GGGTGCCTTG ATGTGGGCGC CGGCGGTCGA GTGGCGACGG CGCGGCTTGT | |
| | 13441 | CCGCGCCCTG GTAGATTGCC TGGCCGTAGG CCAGCCATTT TTGAGCGGCC AGCGGCCGCG | |
| | 13501 | ATAGGCCGAC GCGAAGCGGC GGGGCGTAGG GAGCGCAGCG ACCGAAGGGT AGGCGCTTTT | |
| | 13561 | TGCAGCTCTT CGGCTGTGCG CTGGCCAGAC AGTTATGCAC AGGCCAGGCG GGTTTTAAGA | |
| | 13621 | GTTTTAATAA GTTTTAAAGA GTTTTAGGCG GAAAAATCGC CTTTTTTCTC TTTTATATCA | |
| | 13681 | GTCACTTACA TGTGTGACCG GTTCCCAATG TACGGCTTTG GGTTCCCAAT GTACGGGTTC | |
| | 13741 | CGGTTCCCAA TGTACGGCTT TGGGTTCCCA ATGTACGTGC TATCCACAGG AAACAGACCT | |
| | 13801 | TTTCGACCTT TTTCCCCTGC TAGGGCAATT TGCCCTAGCA TCTGCTCCGT ACATTAGGAA | |
| | 13861 | CCGGCGGATG CTTCGCCCTC GATCAGGTTG CGGTAGCGCA TGACTAGGAT CGGGCCAGCC | |
| | 13921 | TGCCCCGCCT CCTCCTTCAA ATCGTACTCC GGCAGGTCAT TTGACCCGAT CAGCTTGCGC | |
| | 13981 | ACGGTGAAAC AGAACTTCTT GAACTCTCCG GCGCTGCCAC TGCGTTCGTA GATCGTCTTG | |
| | 14041 | AACAACCATC TGGCTTCTGC CTTGCCTGCG GCGCGGCGTG CCAGGCGGTA GAGAAAACGG | |
| | 14101 | CCGATGCCGG GATCGATCAA AAAGTAATCG GGGTGAACCG TCAGCACGTC CGGGTTCTTG | |
| | 14161 | CCTTCTGTGA TCTCGCGGTA CATCCAATCA GCTAGCTCGA TCTCGATGTA CTCCGGCCGC | |
| | 14221 | CCGGTTTCGC TCTTTACGAT CTTGTAGCGG CTAATCAAGG CTTCACCCTC GGATACCGTC | |
| | 14281 | ACCAGGCGGC CGTTCTTGGC CTTCTTCGTA CGCTGCATGG CAACGTGCGT GGTGTTTAAC | |
| | 14341 | CGAATGCAGG TTTCTACCAG GTCGTCTTTC TGCTTTCCGC CATCGGCTCG CCGGCAGAAC | |
| | 14401 | TTGAGTACGT CCGCAACGTG TGGACGGAAC ACGCGGCCGG GCTTGTCTCC CTTCCCTTCC | |
| | 14461 | CGGTATCGGT TCATGGATTC GGTTAGATGG GAAACCGCCA TCAGTACCAG GTCGTAATCC | |
| | 14521 | CACACACTGG CCATGCCGGC CGGCCCTGCG GAAACCTCTA CGTGCCCGTC TGGAAGCTCG | |
| | 14581 | TAGCGGATCA CCTCGCCAGC TCGTCGGTCA CGCTTCGACA GACGGAAAAC GGCCACGTCC | |
| | 14641 | ATGATGCTGC GACTATCGCG GGTGCCCACG TCATAGAGCA TCGGAACGAA AAAATCTGGT | |
| | 14701 | TGCTCGTCGC CCTTGGGCGG CTTCCTAATC GACGGCGCAC CGGCTGCCGG CGGTTGCCGG | |
| | 14761 | GATTCTTTGC GGATTCGATC AGCGGCCGCT TGCCACGATT CACCGGGGCG TGCTTCTGCC | |
| | 14821 | TCGATGCGTT GCCGCTGGGC GGCCTGCGCG GCCTTCAACT TCTCCACCAG GTCATCACCC | |
| | 14881 | AGCGCCGCGC CGATTTGTAC CGGGCCGGAT GGTTTGCGAC CGCTCACGCC GATTCCTCGG | |
| | 14941 | GCTTGGGGGT TCCAGTGCCA TTGCAGGGCC GGCAGGCAAC CCAGCCGCTT ACGCCTGGCC | |
| | 15001 | AACCGCCCGT TCCTCCACAC ATGGGGCATT CCACGGCGTC GGTGCCTGGT TGTTCTTGAT | |
| | 15061 | TTTCCATGCC GCCTCCTTTA GCCGCTAAAA TTCATCTACT CATTTATTCA TTTGCTCATT | |
| | 15121 | TACTCTGGTA GCTGCGCGAT GTATTCAGAT AGCAGCTCGG TAATGGTCTT GCCTTGGCGT | |
| | 15181 | ACCGCGTACA TCTTCAGCTT GGTGTGATCC TCCGCCGGCA ACTGAAAGTT GACCCGCTTC | |
| | 15241 | ATGGCTGGCG TGTCTGCCAG GCTGGCCAAC GTTGCAGCCT TGCTGCTGCG TGCGCTCGGA | |
| | 15301 | CGGCCGGCAC TTAGCGTGTT TGTGCTTTTG CTCATTTTCT CTTTACCTCA TTAACTCAAA | |
| | 15361 | TGAGTTTTGA TTTAATTTCA GCGGCCAGCG CCTGGACCTC GCGGGCAGCG TCGCCCTCGG | |
| | 15421 | GTTCTGATTC AAGAACGGTT GTGCCGGCGG CGGCAGTGCC TGGGTAGCTC ACGCGCTGCG | |
| | 15481 | TGATACGGGA CTCAAGAATG GGCAGCTCGT ACCCGGCCAG CGCCTCGGCA ACCTCACCGC | |
| | 15541 | CGATGCGCGT GCCTTTGATC GCCCGCGACA CGACAAAGGC CGCTTGTAGC CTTCCATCCG | |
| | 15601 | TGACCTCAAT GCGCTGCTTA ACCAGCTCCA CCAGGTCGGC GGTGGCCCAT ATGTCGTAAG | |
| | 15661 | GGCTTGGCTG CACCGGAATC AGCACGAAGT CGGCTGCCTT GATCGCGGAC ACAGCCAAGT | |
| | 15721 | CCGCCGCCTG GGGCGCTCCG TCGATCACTA CGAAGTCGCG CCGGCCGATG GCCTTCACGT | |
| | 15781 | CGCGGTCAAT CGTCGGGCGG TCGATGCCGA CAACGGTTAG CGGTTGATCT TCCCGCACGG | |
| | 15841 | CCGCCCAATC GCGGGCACTG CCCTGGGGAT CGGAATCGAC TAACAGAACA TCGGCCCCGG | |
| | 15901 | CGAGTTGCAG GGCGCGGGCT AGATGGGTTG CGATGGTCGT CTTGCCTGAC CCGCCTTTCT | |
| | 15961 | GGTTAAGTAC AGCGATAACC TTCATGCGTT CCCCTTGCGT ATTTGTTTAT TTACTCATCG | |
| | 16021 | CATCATATAC GCAGCGACCG CATGACGCAA GCTGTTTTAC TCAAATACAC ATCACCTTTT | |
| | 16081 | TAGACGGCGG CGCTCGGTTT CTTCAGCGGC CAAGCTGGCC GGCCAGGCCG CCAGCTTGGC | |
| | 16141 | ATCAGACAAA CCGGCCAGGA TTTCATGCAG CCGCACGGTT GAGACGTGCG CGGGCGGCTC | |
| | 16201 | GAACACGTAC CCGGCCGCGA TCATCTCCGC CTCGATCTCT TCGGTAATGA AAAACGGTTC | |
| | 16261 | GTCCTGGCCG TCCTGGTGCG GTTTCATGCT TGTTCCTCTT GGCGTTCATT CTCGGCGGCC | |
| | 16321 | GCCAGGGCGT CGGCCTCGGT CAATGCGTCC TCACGGAAGG CACCGCGCCG CCTGGCCTCG | |
| | 16381 | GTGGGCGTCA CTTCCTCGCT GCGCTCAAGT GCGCGGTACA GGGTCGAGCG ATGCACGCCA | |
| | 16441 | AGCAGTGCAG CCGCCTCTTT CACGGTGCGG CCTTCCTGGT CGATCAGCTC GCGGGCGTGC | |
| | 16501 | GCGATCTGTG CCGGGGTGAG GGTAGGGCGG GGGCCAAACT TCACGCCTCG GGCCTTGGCG | |
| | 16561 | GCCTCGCGCC CGCTCCGGGT GCGGTCGATG ATTAGGGAAC GCTCGAACTC GGCAATGCCG | |
| | 16621 | GCGAACACGG TCAACACCAT GCGGCCGGCC GGCGTGGTGG TGTCGGCCCA CGGCTCTGCC | |
| | 16681 | AGGCTACGCA GGCCCGCGCC GGCCTCCTGG ATGCGCTCGG CAATGTCCAG TAGGTCGCGG | |
| | 16741 | GTGCTGCGGG CCAGGCGGTC TAGCCTGGTC ACTGTCACAA CGTCGCCAGG GCGTAGGTGG | |
| | 16801 | TCAAGCATCC TGGCCAGCTC CGGGCGGTCG CGCCTGGTGC CGGTGATCTT CTCGGAAAAC | |
| | 16861 | AGCTTGGTGC AGCCGGCCGC GTGCAGTTCG GCCCGTTGGT TGGTCAAGTC CTGGTCGTCG | |
| | 16921 | GTGCTGACGC GGGCATAGCC CAGCAGGCCA GCGGCGGCGC TCTTGTTCAT GGCGTAATGT | |
| | 16981 | CTCCGGTTCT AGTCGCAAGT ATTCTACTTT ATGCGACTAA AACACGCGAC AAGAAAACGC | |
| | 17041 | CAGGAAAAGG GCAGGGCGGC AGCCTGTCGC GTAACTTAGG ACTTGTGCGA CATGTCGTTT | |
| | 17101 | TCAGAAGACG GCTGCACTGA ACGTCAGAAG CCGACTGCAC TATAGCAGCG GAGGGGTTGG | |
| | 17161 | ATCAAAGTAC TTTGATCCCG AGGGGAACCC TGTGGTTGGC ATGCACATAC AAATGGACGA | |
| | 17221 | ACGGATAAAC CTTTTCACGC CCTTTTAAAT ATCCGATTAT TCTAA | |

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Combination of YHB and Elf3 Alleles Restricts Photomorphogenesis and Flowering Variation in Response to Environmental Cues

Plants are very sensitive to their environment and respond to changes in light and temperature to optimize their growth unlike animals, they demonstrate developmental plasticity. Developmental plasticity is advantageous in natural conditions (where resources and environmental stresses vary across seasons and location) but is disadvantageous in modern crop monoculture where fertilizers, pesticides, irrigation, etc., can be provided. Climate change offers additional challenges, with more varied weather and increasing temperatures disrupting plant development. Importantly, flowering in many crops is dependent on day length and highly influenced by ambient temperatures. This is disruptive to farmers who can only harvest at defined times of the year.

One goal of modern breeding programs is to increase the uniformity of crops so that harvesting time is more predictable and quality is consistent. This is true for intensive, precision outdoor farming and Total Controlled Environment Agriculture (TCEA, or vertical farming). Candidate targets include genes encoding for photoreceptors and thermoreceptors (proteins responsive to light and/or temperature) and genes that disrupt the circadian clock (an endogenous molecular timekeeper that governs responses to environmental signals at different times of day). As the circadian system allows measurement of daylength, disruption of the clock should produce day-neutral plants that flower regardless of season.

Using the model plant Arabidopsis thaliana, two genetic traits were combined to limit plants' response to environmental signals, disruption of EARLY FLOWERING3 (ELF3) and the addition of the constitutively-active Y276H allele of the photoreceptor phytochromeB (which is referred to herein as YHB). Loss of ELF3 accelerates flowering, disrupts circadian rhythms and potentially impairs temperature signaling, but induces an etiolated phenotype (that produces poor yield). YHB also induces earlier flowering and provides a consistent light signal regardless of irradiance, reducing etiolation. However, the combination of these two alleles has yet to be exploited.

Crucially, the inventors show that the combination of these two alleles [YHB(elf3)] has advantages over each allele individually. YHB(elf3) plants are not etiolated and flower earlier (so are good candidates for vegetative crops in TCEA). YHB(elf3) plants have reduced cellular elongation response to increasing temperature, which will reduce listing in the field. YHB(elf3) plants also flower at the same time regardless of day length-suggesting that crops can be developed that will flower consistently regardless of differences in day length at varied latitudes or in the face of climate change increasing temperatures and disrupting weather patterns. Similar traits can be exploited in TCEA to limit energy costs incurred when growing plants under artificial lighting, for example, shifted lighting regimes can be used to avoid peak energy supply rates or grow multiple species under a single lighting condition. Many secondary metabolite pathways are regulated by the clock, therefore valuable product production could be increased by “locking” the oscillator into an “on” position.

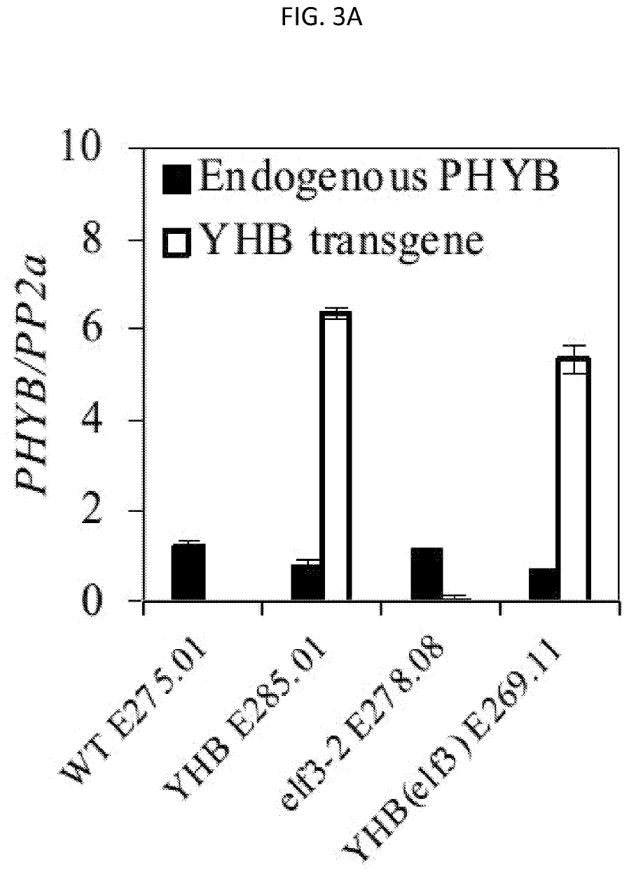

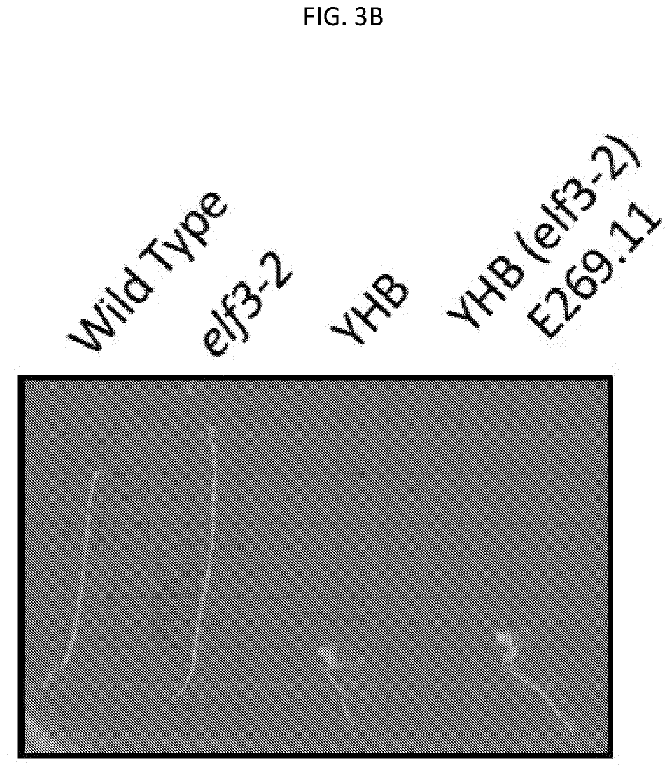

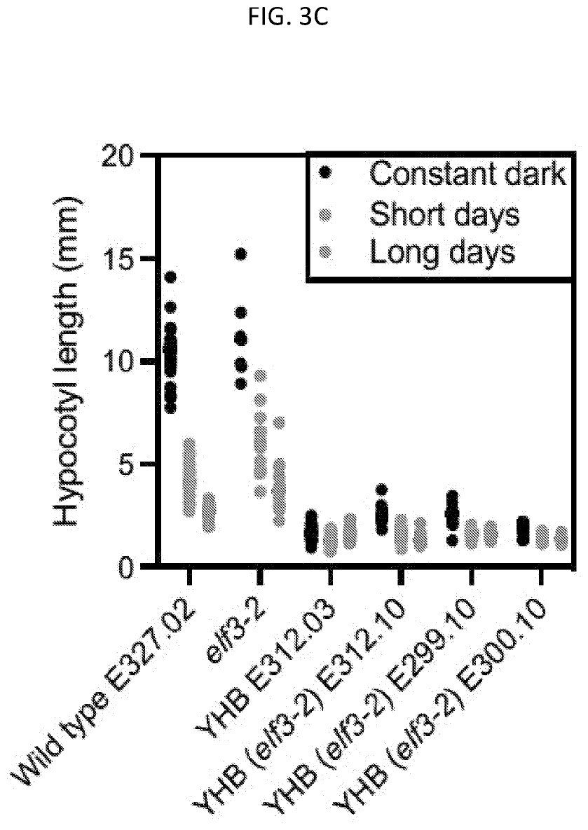

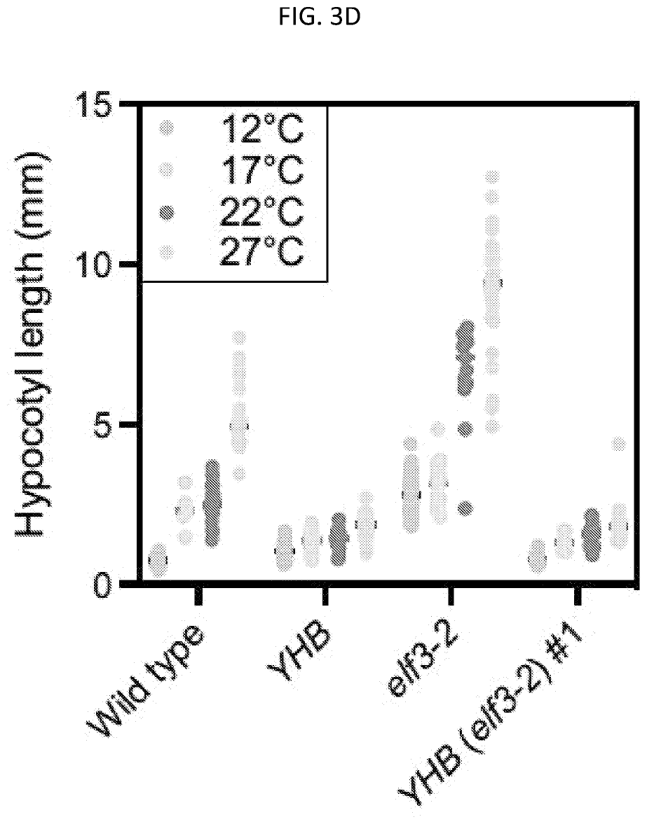

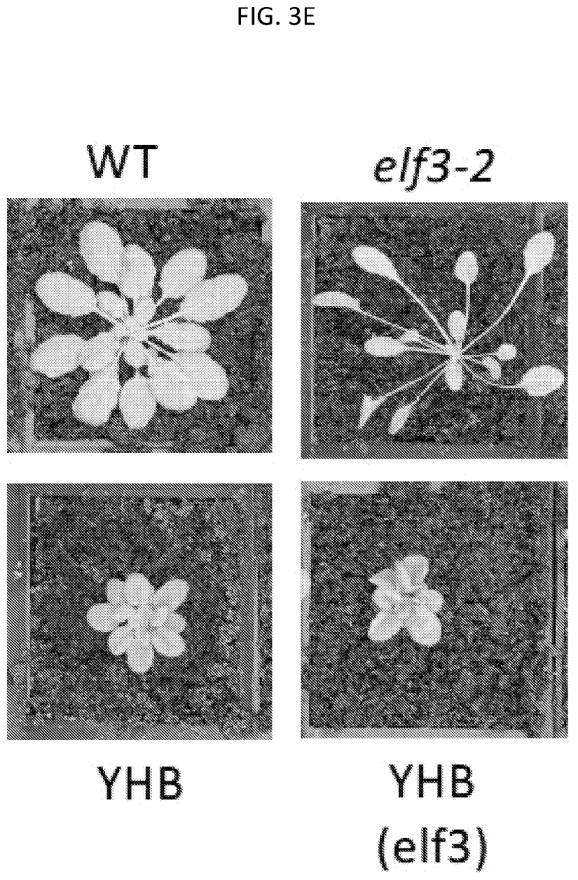

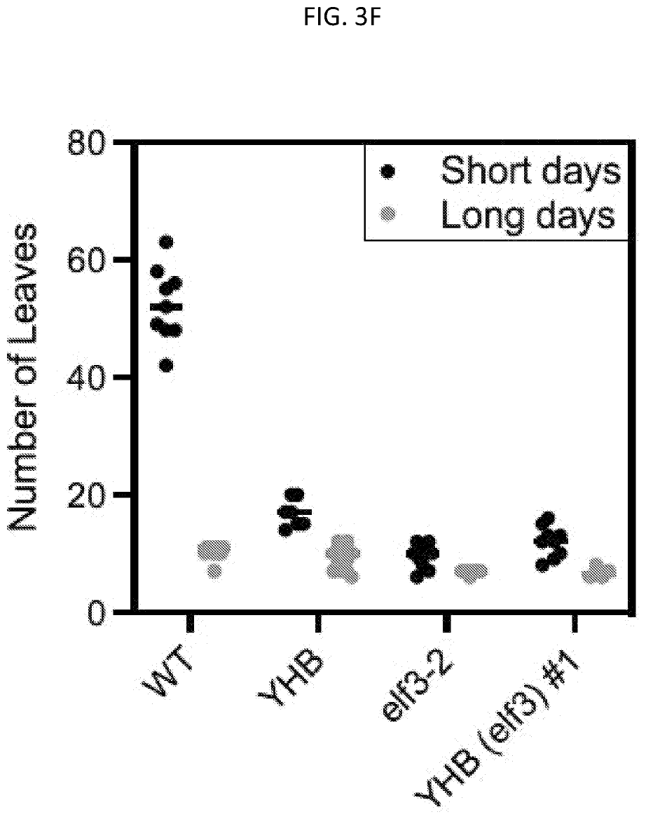

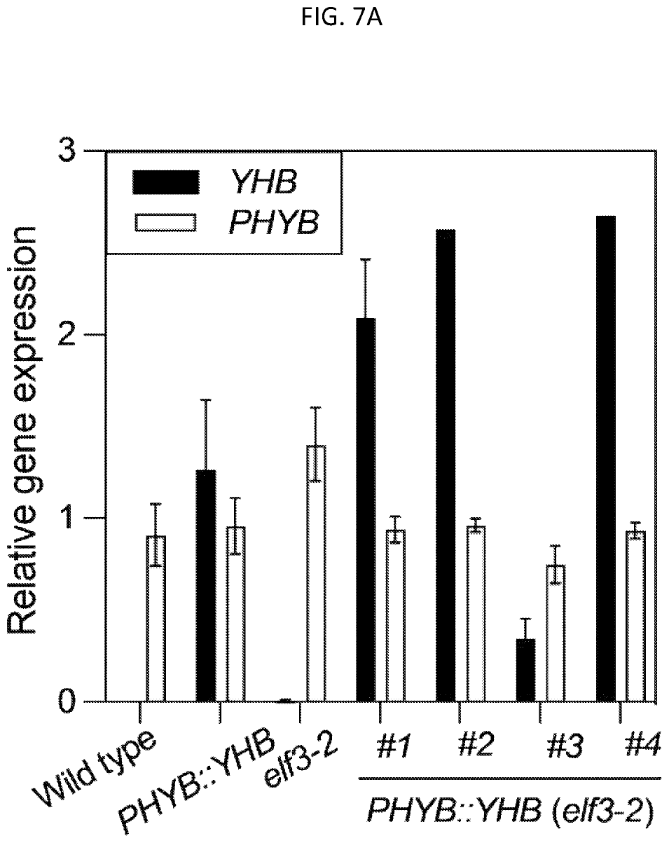

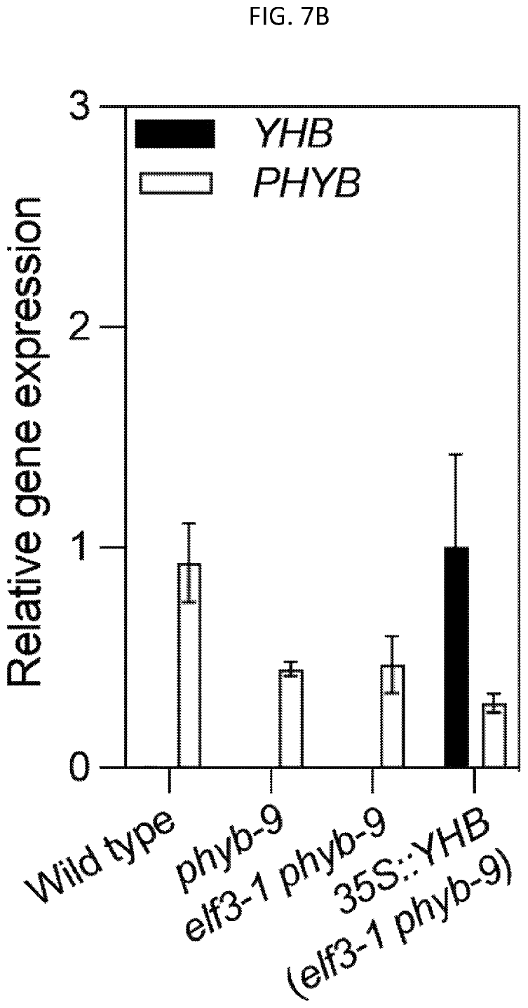

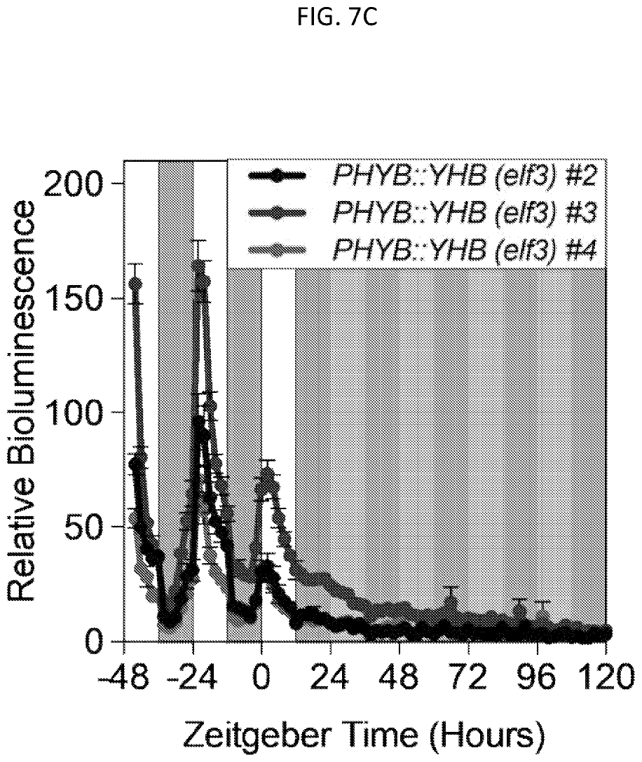

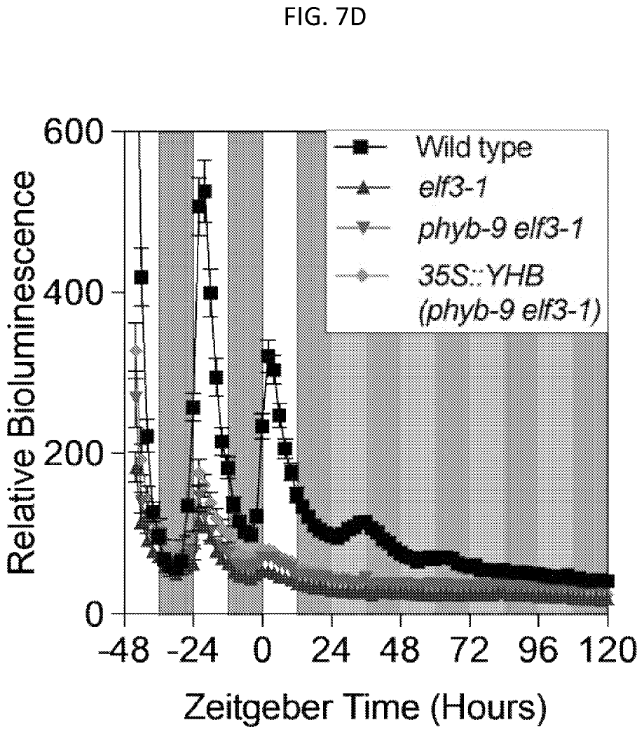

Results. Loss-of-function and over-expression analysis suggest that phytochrome- and ELF3-signaling pathways are genetically separable, although multiple lines of evidence demonstrate a functional interaction between these signaling pathways. To further characterize this relationship, the YHB allele was transformed into an elf3-2 background (FIG. 3A). Importantly, YHB was sufficient to induce photomorphogenesis in PHYB::YHB(elf3) seedlings grown in constant darkness, with the degree of photomorphogenesis observed correlating with the expression level of YHB (FIGS. 3B and 7A). Similar data were obtained using independently transformed 35S::YHB (phyb-9 elf3-1) seedlings (phyb-9; loss of function allele of PHYB; SEQ ID NO: 9. Produces truncated protein comprising SEQ ID NO: 10). These data agree with previous work suggesting that ELF3 and phyB make additive contributions to photomorphogenesis.

Seedling hypocotyl growth is exquisitely sensitive to light and temperature. As YHB (elf3-2) seedlings retained a constitutively photomorphogenetic (cop) phenotype when grown in constant darkness (FIGS. 3B-C) it was tested whether these seedlings were able to regulate their development in response to environmental factors, including daylength and temperature. Hypocotyl growth is negatively correlated with photoperiod in wild-type seedlings (FIG. 3C). Consistent with data gathered from etiolated seedlings (FIGS. 3B-3C), YHB (elf3) hypocotyls were not significantly extended in either long-day or short-day conditions (FIG. 3C). These data suggest that YHB (elf3) seedlings are not able to adjust their development in response to varied light conditions.

Phytochromes and ELF3 have both been reported to serve as integrators of temperature signals, and therefore, how YHB (elf3) seedling growth was affected by growth in different ambient temperatures was examined (FIG. 3D). elf3-2 responded in a similar fashion to wild type in the conditions used herein, while YHB seedlings failed to demonstrate hypocotyl extension when grown at 27° C., in line with previous reports (FIG. 3D). However, it was noted that the hypocotyls of YHB (elf3) seedlings were unresponsive to temperature (FIG. 3D). These data demonstrate that constitutively active phyB is sufficient to limit heat-induced hypocotyl elongation even in the absence of ELF3.

Next, the adult phenotypes of the YHB (elf3) plants (FIGS. 3E and 3F) were assessed. Following growth in short day conditions YHB (elf3) lines had a compact rosette comparable to YHB (FIG. 3E), demonstrating that ELF3 is not necessary for phyB-mediated changes in adult morphology (as in seedlings; FIGS. 3B-3D). In addition, the developmental time to flowering in short days was greatly accelerated in YHB, elf3-2, and YHB (elf3) plants despite the differences in rosette morphology observed (FIGS. 3E-3F). In long days (16:8), flowering time was accelerated in elf3-2 seedlings compared to wild type, although no differences in flowering time were observed between elf3-2 and YHB(elf3) plants (FIG. 3F).

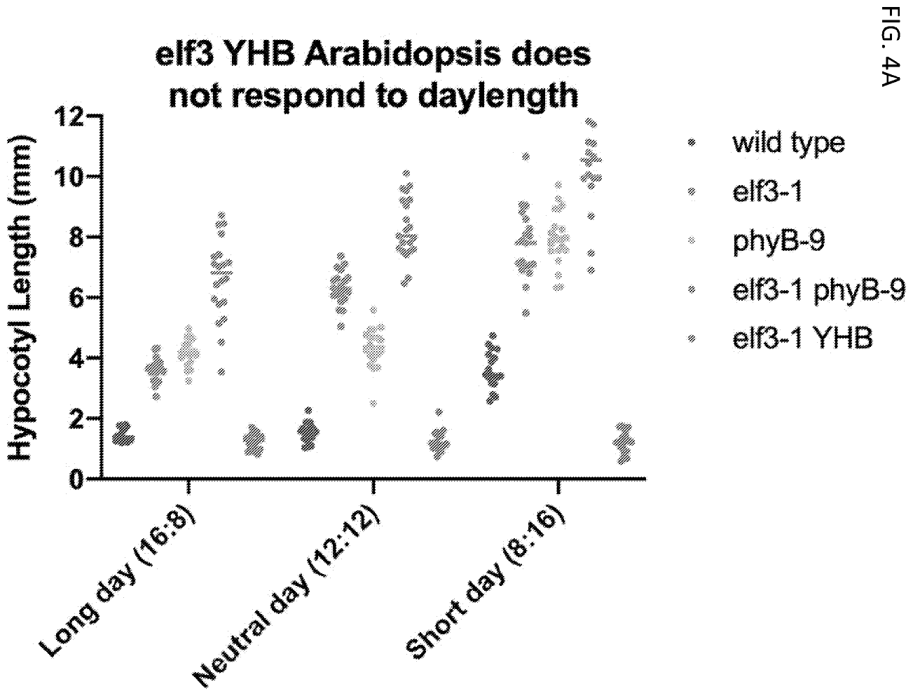

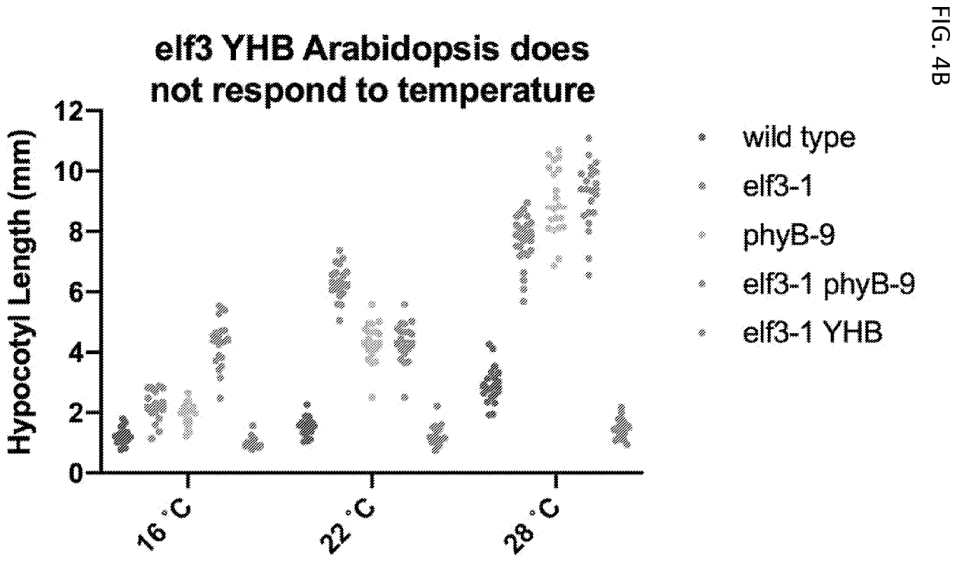

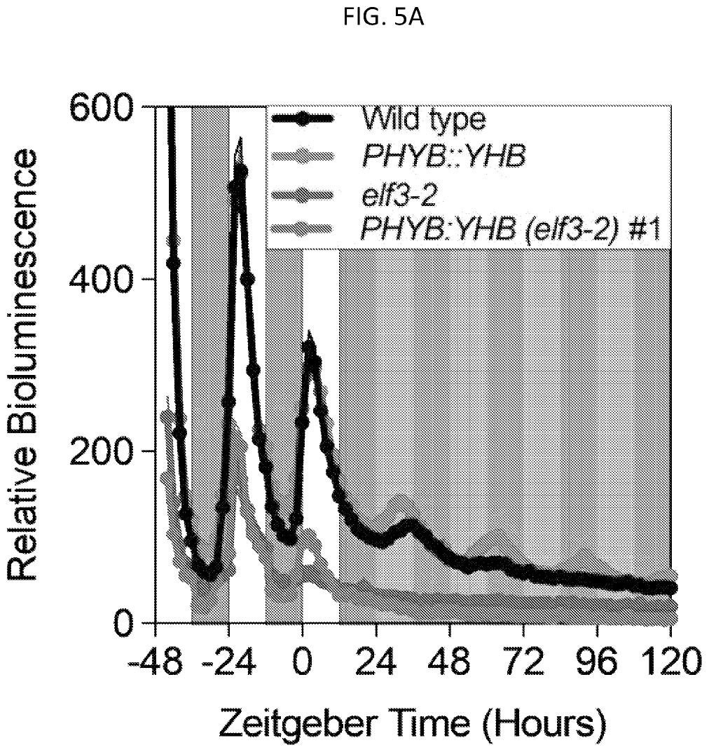

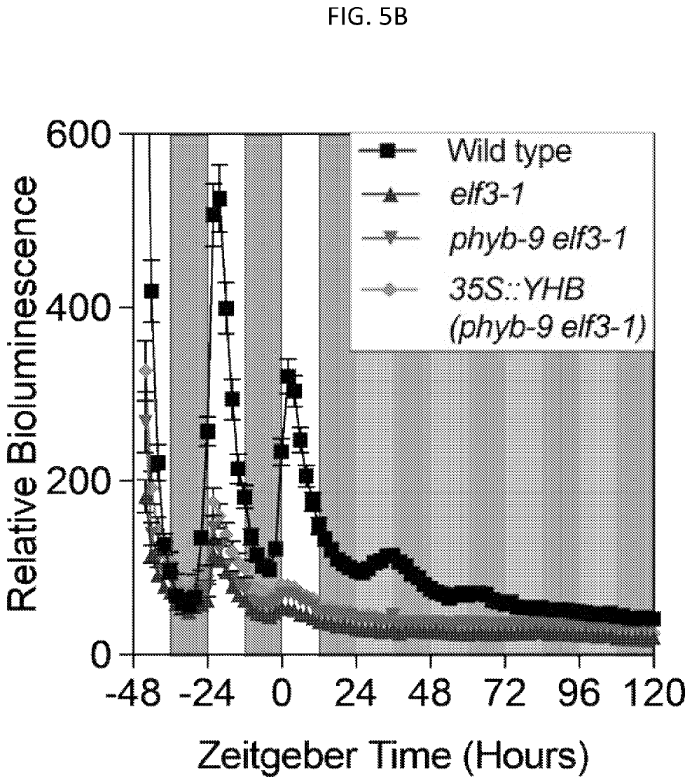

Importantly, constitutively expressed YHB (elf3) plants also showed strongly reduced plant elongation responses to daylength (FIG. 4A) and temperature (FIG. 4B). Further, YHB (elf3) plants lacked circadian rhythms, flattening internal metabolic cycles under constant conditions either when expressed from its own promoter (FIG. 5A) or when constitutively expressed (FIG. 5B).

Discussion Plants are remarkably sensitive to environmental signals, adapting their growth and development to optimize survival and fecundity in the prevailing conditions. The flexibility of plant development greatly promotes survival in natural conditions but is sub-optimal for commercial farming where growth and harvesting schedules must be adjusted to match crop progression. It was sought to limit plants' inherent developmental plasticity by manipulating the PHYTOCHROMEB (PHYB) and EARLY FLOWERING3 (ELF3) loci within the Arabidopsis genome. It was demonstrated that these plants are unresponsive to environmental stimuli yet retain beneficial agronomic phenotypes that are lost in other manipulations.

### Example 2. Combination of YHB and Elf3 Alleles Restricts Photomorphogenesis and Flowering Variation in Response to Environmental Cues

A pennycress YHB (elf3) double mutant was also generated. The response of the double mutant plants to light and temperature and presence of absence of circadian rhythms is investigated. Pennycress YHB (elf3) double mutants show strongly reduce plant elongation responses to daylength and temperature. Further, YHB (elf3) pennycress lack circadian rhythms, flattening internal metabolic cycles.

### Example 3. Characterization of Plasticity of YHB Elf3 Plants

Plants demonstrate developmental plasticity which enables adaptation to prevailing environmental cues. Although valuable to maximize survival in nature, developmental plasticity restricts modern agricultural practices since economically important traits including vegetative growth and flowering time vary dependent upon local environmental conditions, preventing accurate prediction of harvesting times. In this study, it was sought to restrict developmental plasticity through manipulation of two signaling systems that govern these responses. Constitutive activation of the plant photoreceptor phyB, along with disruption of the circadian system created plants that are less responsive to environmental signals, enabling the creation of crops with uniform flowering time and productivity. This has significant implications for future agriculture in both open fields and controlled environments.

It was first hypothesized that developmental plasticity in plants could be restricted by disrupting signaling pathways that modulate plant development in response to environmental signals. Plants respond to light and temperature signals differentially dependent upon time of day, utilizing photo- and thermo-sensors in combination with the circadian system which provides an internal timing reference to relative to dawn and dusk. A suite of photoreceptors including phytochromes, cryptochromes, zeitlupe (ZTL), and UVR8 each integrate light signals into the circadian clock. Photoreceptors integrate with the circadian system at multiple levels, with phytochromes physically interacting with EARLY FLOWERING3 (ELF3), a gene which is essential to maintain circadian rhythms in constant light. Both phyB and ELF3 have been shown to be responsive to temperature in addition to their roles in photoperception and the circadian system. Accordingly, an aim of this study was to determine whether manipulation of these two proteins could and would be sufficient to restrict developmental plasticity.

### Phytochrome Drives Circadian Rhythms Via EARLY FLOWERING3

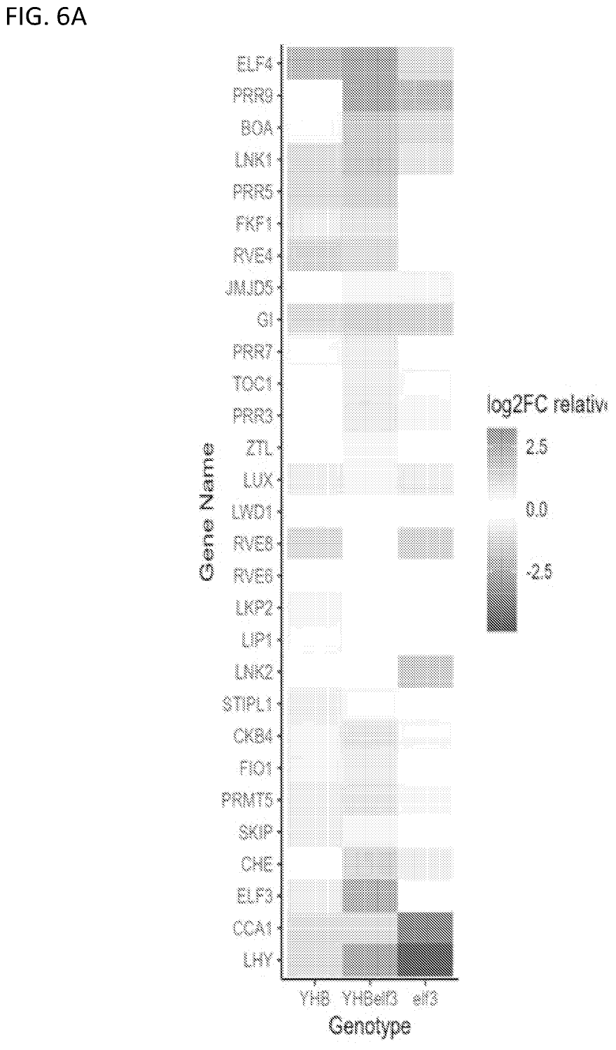

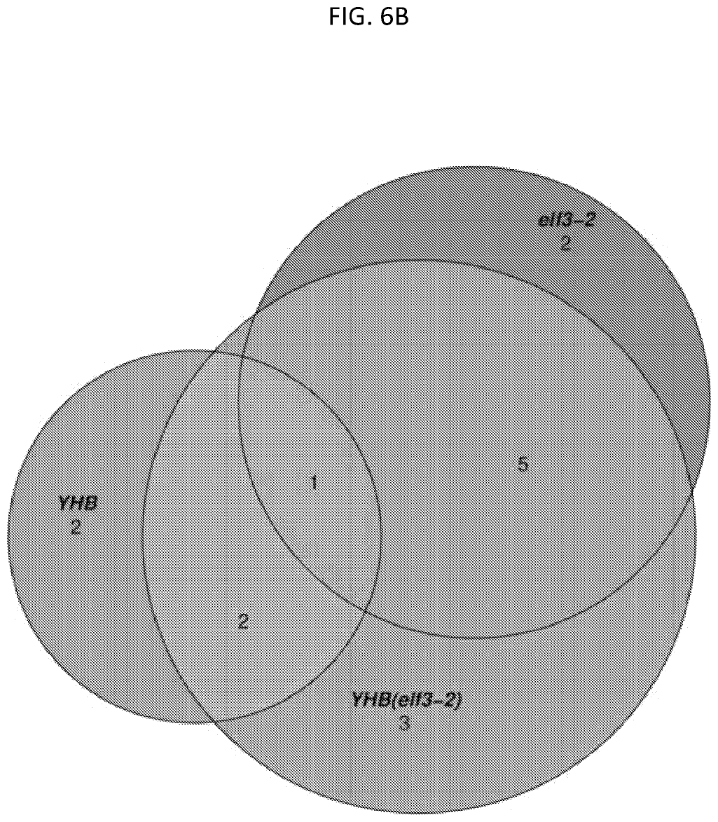

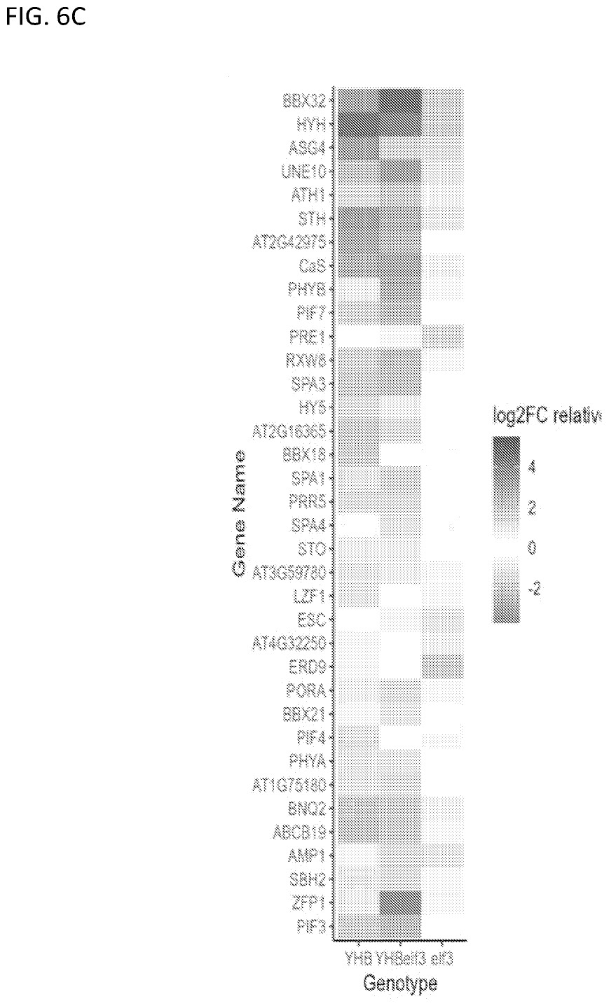

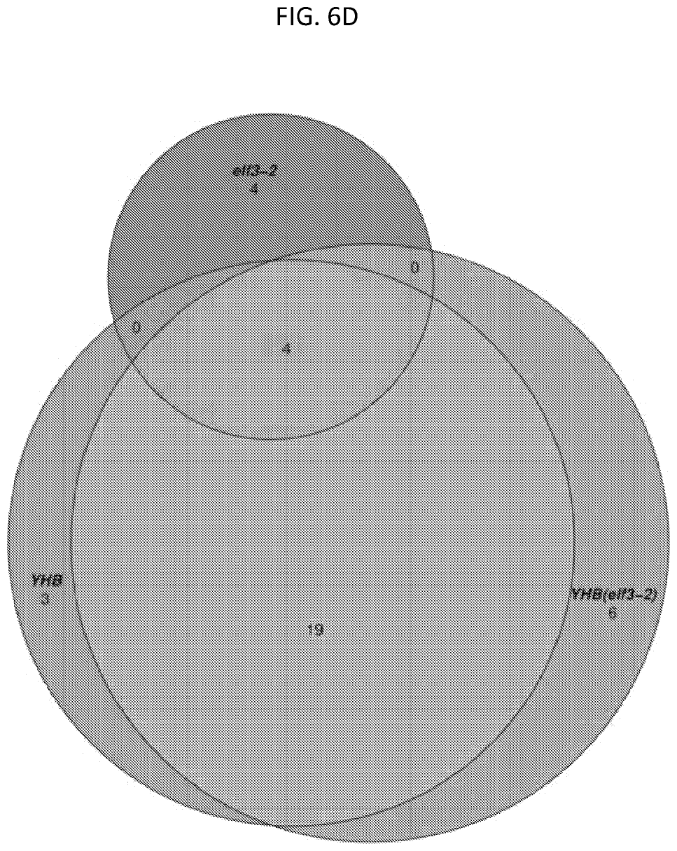

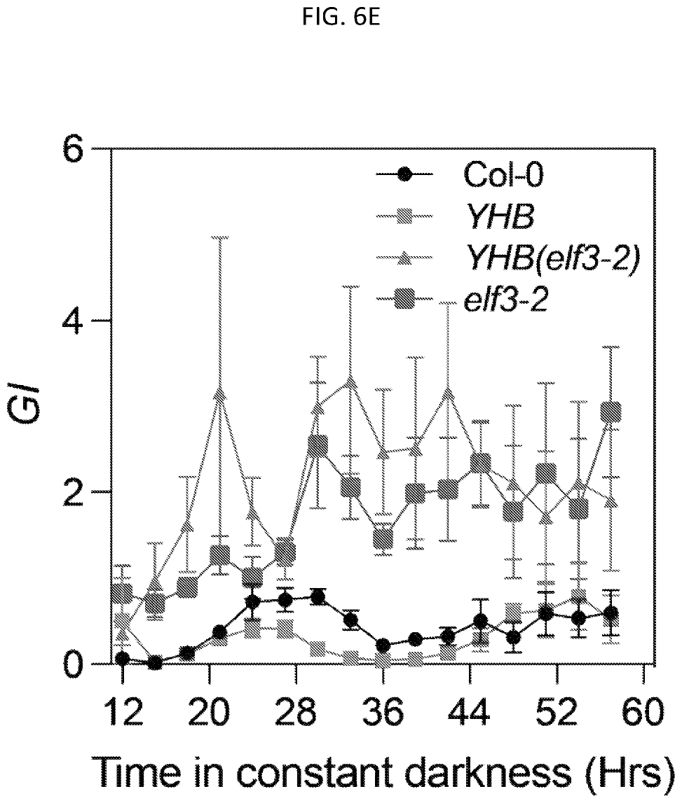

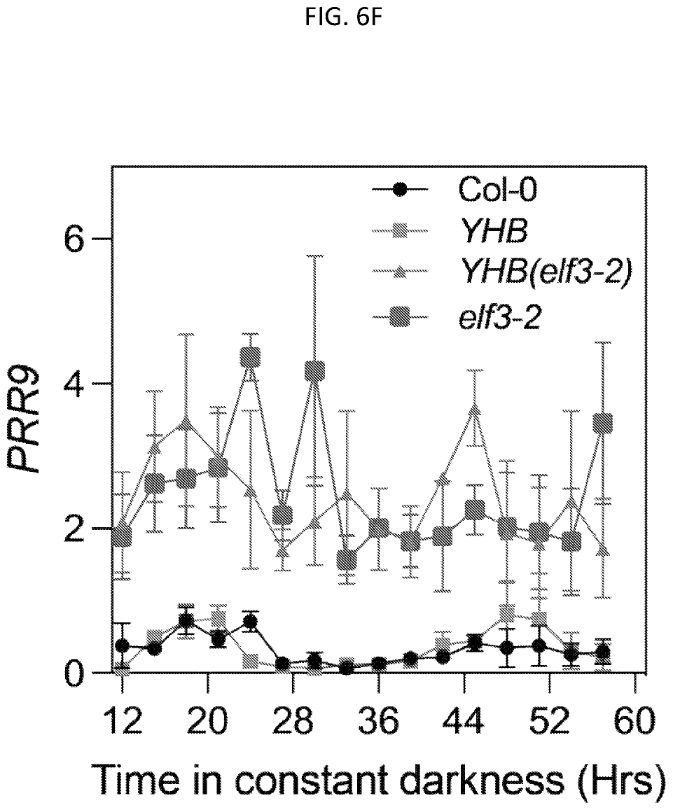

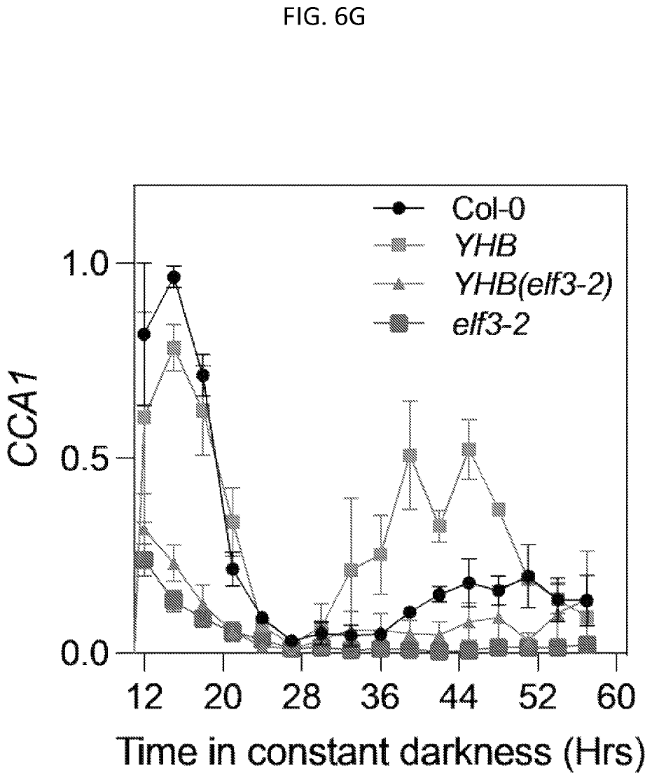

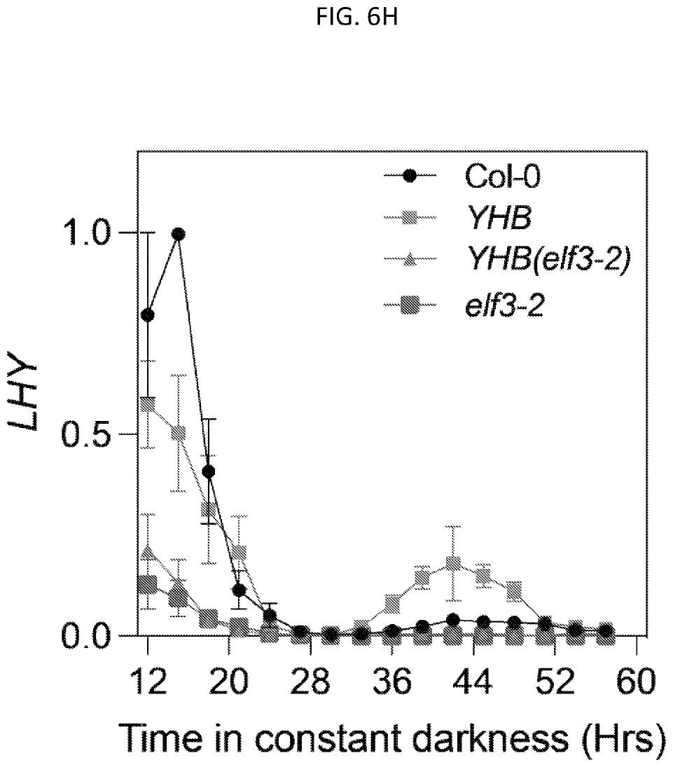

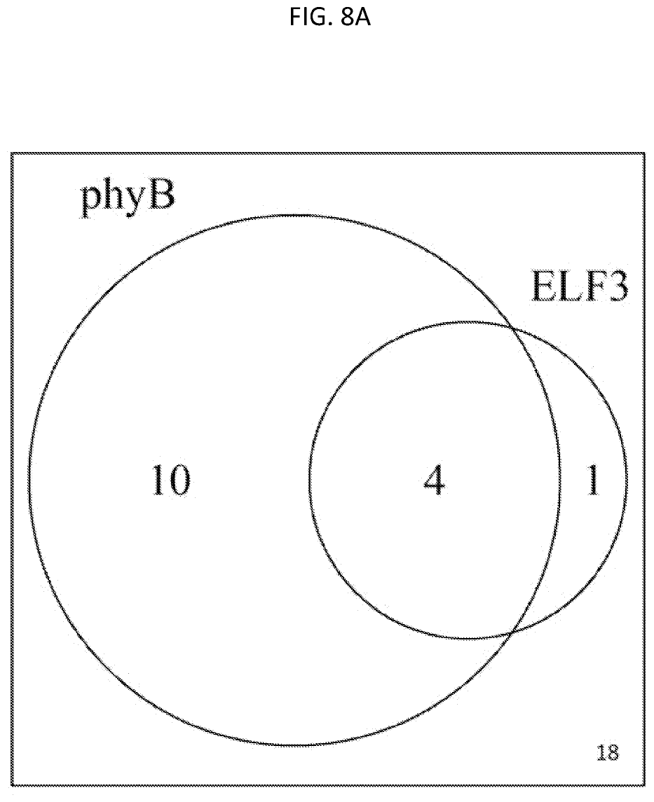

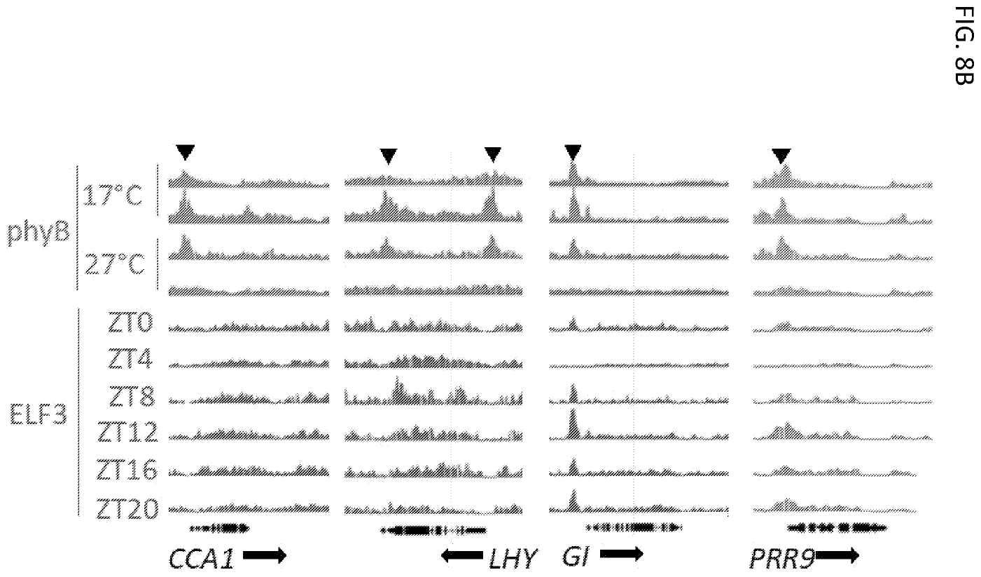

Loss-of-function and over-expression analysis suggest that phytochrome- and ELF3-signaling pathways are genetically separable, although multiple lines of evidence demonstrate a functional interaction between these signaling pathways. To further characterize this relationship a PHYB promoter driven YHB allele of PhyB was transformed into an elf3-2 background or a constitutively expressed YHB (35S::YHB) was transformed into an elf3-1 phyb-9 mutant background (FIG. 3-5) Since phyB and ELF3 physically interact, it was tested whether ELF3 is necessary for phyB signaling into the circadian system (FIG. 5A-5B). Neither PHYB::YHB (elf3-2) nor 35S::YHB (elf3-1 phyb-9) lines were able to maintain circadian rhythms of CCA1-driven bioluminescence in constant darkness, with only 15% of YHB elf3-2 lines being assessed as rhythmic (FIG. 5A-5B). Subsequent qRT-PCR analysis was consistent with the bioluminescence data, with both CCA1 and its orthologue LATE ELONGATED HYPOCOTYL (LHY) being damped in YHBelf3 seedlings compared to YHB plants (FIGS. 6G-6H). By contrast, it was noted that clock genes expressed later in the day, including GIGANTEA and PRR9, were elevated in elf3 and YHBelf3 lines compared to wild-type and YHB controls (FIGS. 6E-6F). Despite the interaction between phyB and ELF3, not all genomic regions occupied by phyB are also bound by ELF3 (FIG. 8A-B and Table 1). In contrast to CCA1 and LHY (which are bound by phyB only), GIGANTEA and PRR9 are bound by both phyB and ELF3 (FIG. 8B). This data suggest that the role of ELF3 as a transcriptional repressor is epistatic to phyB function in these cases (FIGS. 6E-F and FIGS. 8A-B and Table 1).

TABLE 1
Genes bound by;

| PHYB | ELF3 | PHYB and ELF3 |
|---|---|---|
| CCA1 | ELF3 | PRR9 |
| LHY | | PRR7 |
| RVE6 | | GI |
| PRR5 | | LNK1 |
| TOC1 | | |
| TCP21 | | |
| LUX | | |
| ELF4 | | |
| LNK2 | | |
| TIC | | |

The luciferase and bioluminescence data suggest that the clock becomes arrhythmic in constant darkness in YHBelf3 lines (FIGS. 5A-B, 6E-H and FIGS. 7C-D). This observed difference in expression after loss of rhythmicity was further assessed in all 29 core circadian clock genes using RNA sequencing (RNAseq; FIGS. 6A-B). To this end, plants were sampled after 48 hours of dark exposure at respective dusk (ZT60), a point at which wild type seedlings were assumed to have become arrhythmic and would therefore have relatively stable levels of circadian-controlled transcript abundance. Transcript abundance in wild type seedlings was used as a baseline to assess differential expression of core circadian clock genes in YHB, YHB(elf3-2) and elf3-2 seedlings. Fold change abundance of each gene was presented as a heat map (FIG. 6A) showing that more circadian genes are differentially expressed in YHB(elf3-2) seedlings than in YHB or elf3-2 seedlings, relative to wild type. The most highly differentially expressed genes (log 2FC>1.0 or <−1.0) are highlighted in FIG. 6A-B, showing that 11 of the 29 core clock genes are highly differentially expressed in YHB(elf3-2) relative to wild type, while only 7 genes are highly differentially expressed in elf3-2 and 5 in YHB. Of these highly differentially expressed genes, only ELF4 is highly differentially expressed in all three mutants. Additionally, the overexpression of ELF4 is stronger in YHB(elf3-2) than in YHB or elf3-2 (FIG. 6A). RVE4 and PRR5 are upregulated to a similar degree in both YHB and YHB(elf3-2) but not highly differentially expressed in elf3-2. GI, PRR9 and BOA are all highly upregulated and CCA1 and LHY are highly downregulated in both YHB(elf3-2) and elf3-2 but are not highly differentially expressed in YHB seedlings at ZT60 in the dark. Notably, RVE8 is highly upregulated in YHB and highly downregulated in elf3-2 but not highly differentially expressed in YHB(elf3-2) seedlings, suggesting that regulation of RVE8 by phyB and ELF3 acts via distinct, opposing mechanisms.

### ELF3 is not Required for YHB Plants to Initiate Photomorphogenesis in the Dark.

After observing that YHB seedlings require ELF3 to maintain circadian function under constant darkness, it was examined whether YHBelf3 seedlings retained the constitutively photomorphogenic phenotype of YHB seedlings. In 5 day old seedlings grown under constant darkness (FIG. 3B-C, 10A), YHB and YHB(elf3-2) plants exhibited significantly shorter hypocotyls (p<0.0005) and more uniform growth compared to wild type, whereas elf3-2 seedlings are significantly longer (p<0.0001). The average hypocotyl length of YHB(elf3-2) plants was comparable to YHB plants (p=0.2864), while the average hypocotyl length of elf3-2 mutants was significantly longer than all other genotypes (p<0.0001).

To further address how the effects of YHB and ELF3 influence photomorphogenesis, differential expression of photomorphogenesis associated genes was analyzed (list of 115 photomorphogenesis or photomorphogenesis associated genes was taken from TAIR a arabidopsis.org) in the RNAseq dataset obtained as described herein above of seedlings sampled at dusk after 48 hours of constant darkness. The fold change expression of the 36 photomorphogenesis associated genes which were highly differentially expressed (log 2FC>1.0 or <−1.0) in at least one mutant genotype relative to wild type is shown as a heatmap in FIG. 6C. The frequency of these genes being highly differentially expressed in YHB, YHB(elf3-2) and/or elf3-2 respectively is presented as a venn diagram in FIG. 6D, which showed that 4 genes in elf3-2 mutants experienced at least +/−1.0 log 2 fold change in transcript abundance relative to wild type, while YHB and YHB(elf3-2) mutants had a respective total of 22 or 25 highly differentially expressed genes. Of these genes, ASG4 and ATH1 are both highly upregulated in all three mutants, while HYH and STH are both highly upregulated in YHB and YHB(elf3-2) but highly downregulated in elf3-2. No other photomorphogenesis associated genes are highly differentially expressed in either YHB and elf3-2 or YHBelf3-2 and elf3-2, but 19 genes are co-differentially expressed in YHB and YHB(elf3-2). These data suggest that disruption of ELF3 has comparatively little effect upon YHB-regulated photomorphogenesis genes in contrast to the essential role for ELF3 in clock function.

### YHBelf3 Seedlings are Less Responsive to Varied Light and Temperature Conditions

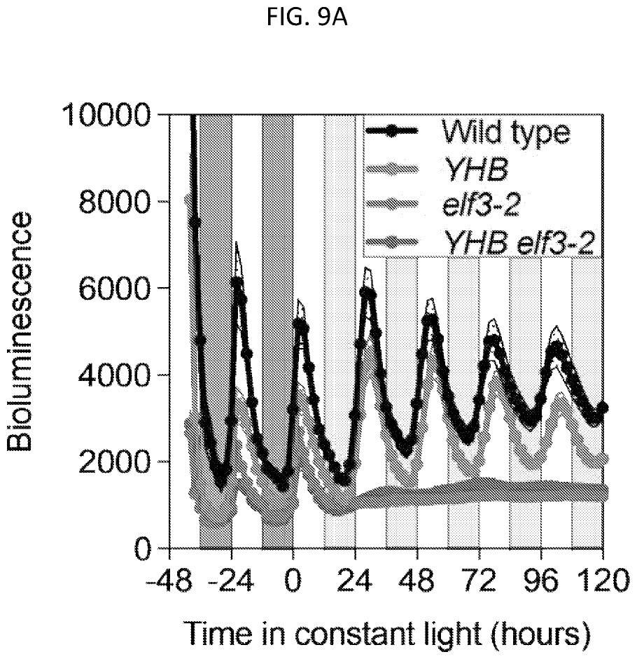

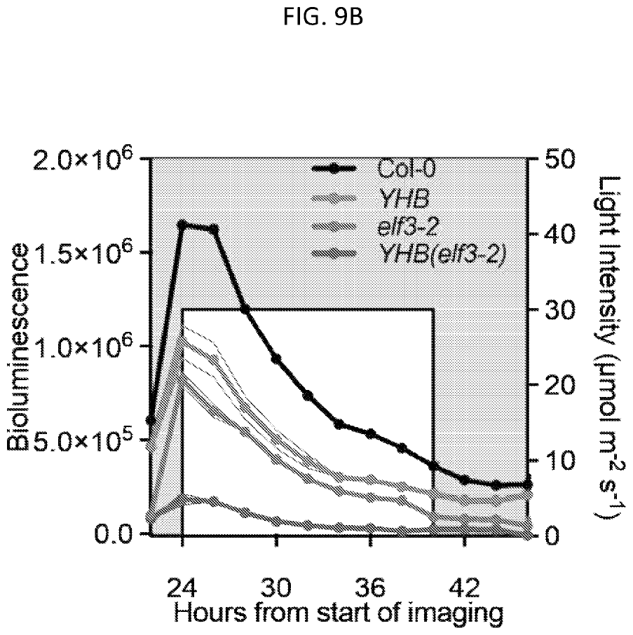

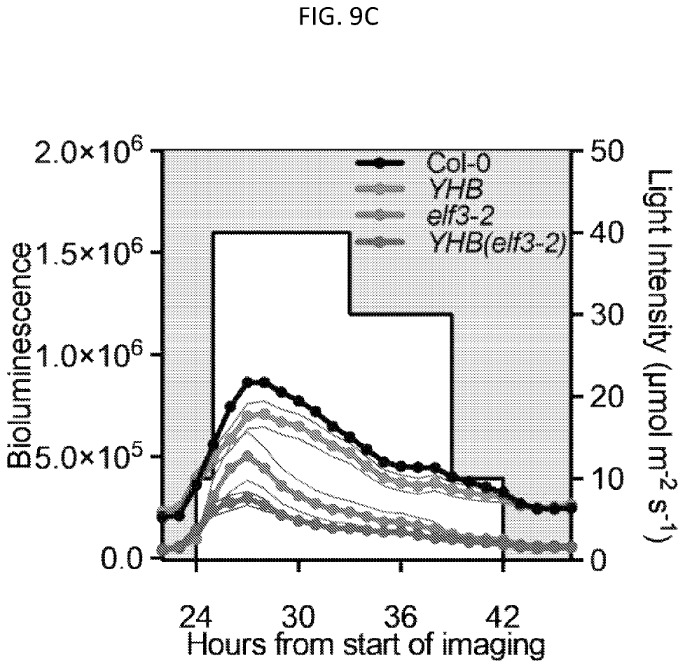

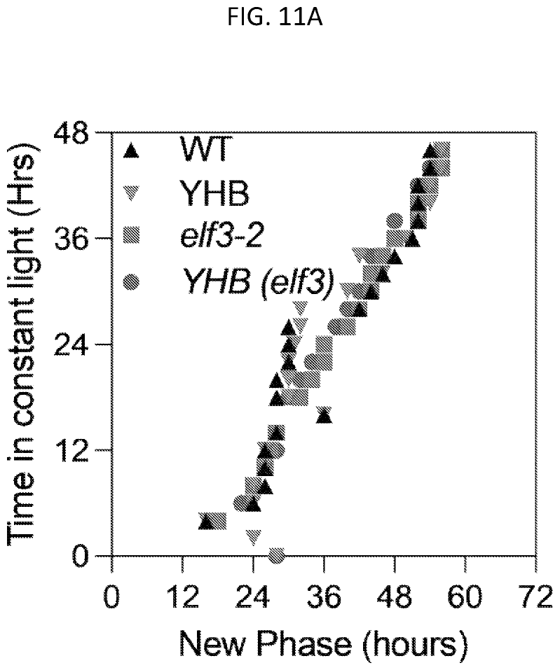

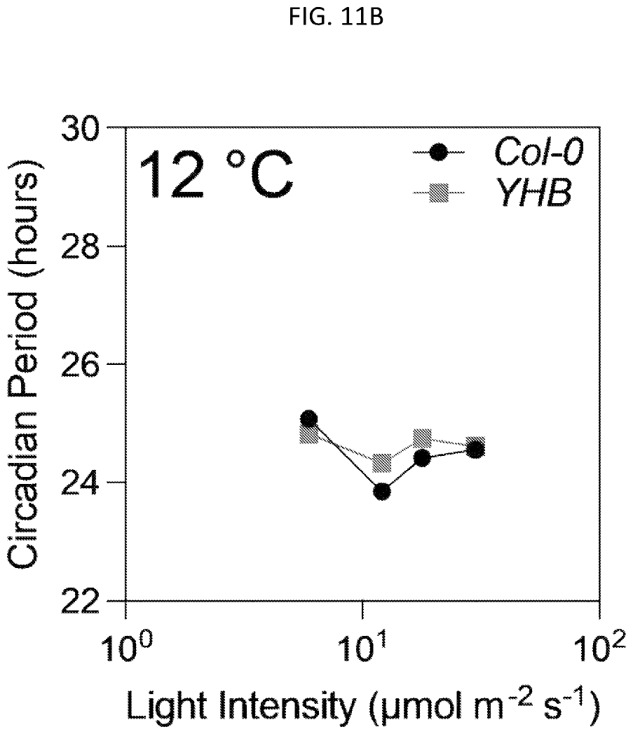

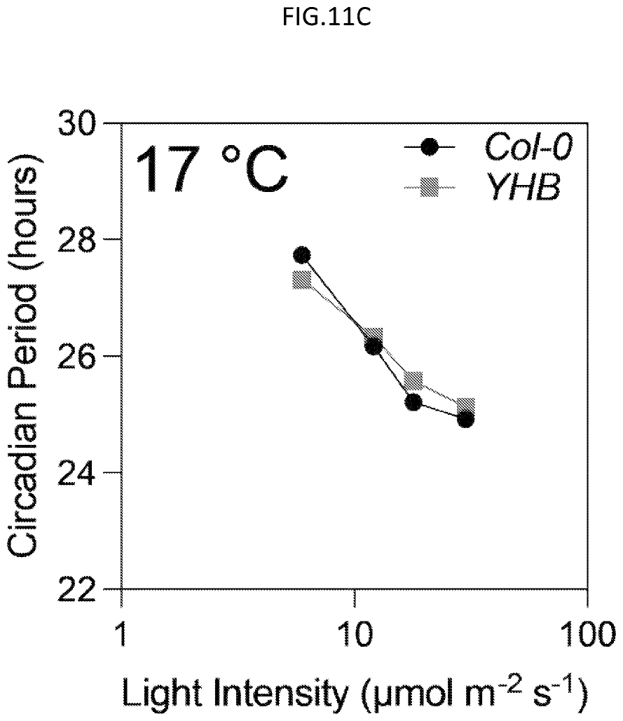

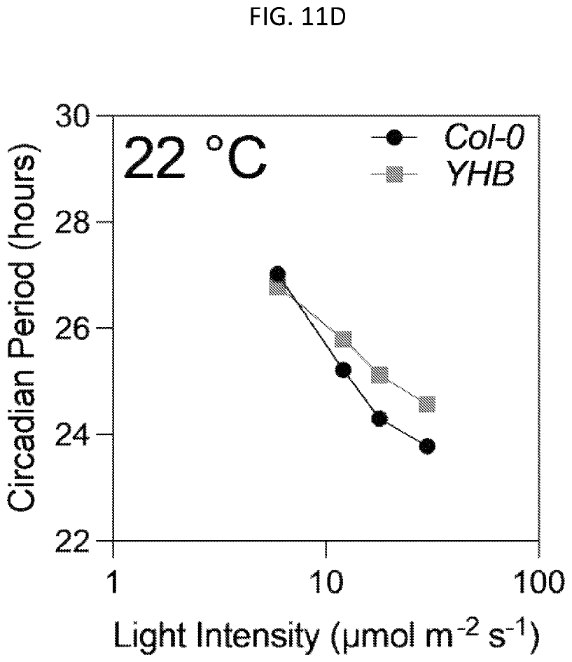

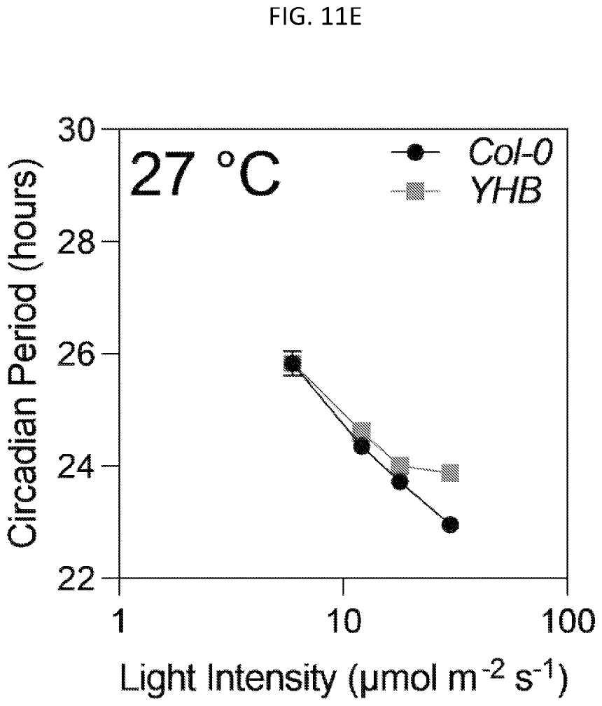

Since the YHBelf3 seedlings lack circadian rhythms yet demonstrate constitutive photomorphogenesis, how these plants responded to daily patterns of environmental change was of interest (FIGS. 9, 10, 11). Each of the genotypes retained daily patterns of CCA1::LUC2 bioluminescence in driven light:dark cycles, with this rhythmicity lost in elf3-2 and YHBelf3 seedlings within 24 hours of transfer to constant white light (FIG. 9A). It was interesting to note that in contrast to previous reports under monochromatic red light, YHB seedlings had an extended circadian period under these conditions (FIG. 9A). A phase response curve was next completed to establish whether YHBelf3 lines were truly arhythmic in constant light (like elf3) or whether the lack of bioluminescence rhythms was masking an underlying oscillation (FIG. 11A). Seedlings were held in constant light for different periods of time and then released into constant darkness to assess the new phase imposed upon circadian the circadian system. In contrast to wild type and YHB seedlings, which demonstrated circadian control of CCA1::LUC phasing following the transfer into constant darkness, both elf3 and YHBelf3 seedlings displayed a linear response to the release into constant darkness (FIG. 11A). These data reiterate that ELF3 is essential for maintaining circadian rhythms in constant light, although daily rhythms are maintained in elf3 and YHBelf3 seedings in driven conditions.

Next, how YHBelf3 seedlings responded to varied light intensity during the photoperiod was examined (FIGS. 9B and 9C). In ‘square wave’ conditions, with lights either on or off, each of the genotypes was able to accurately anticipate dawn, with CCA1::LUC activity peaking at dawn in each case (FIG. 9B). In variable light conditions (FIG. 9C), all lines displayed a much weaker anticipation of dawn with peak CCA1-driven bioluminescence not being reached until hours after the onset of light. Under more varied lighting conditions, with low light at dawn being followed by an increase in fluence rate later in the day, it was found that elf3-2 and YHBelf3-2 seedlings demonstrated a peak in bioluminescence shortly after dawn, whereas wild-type and YHB seedlings peaked later in the morning (FIG. 9C). elf3-2 and YHB(elf3-2) mutants displayed peak CCA1-driven luciferase expression on the examined day at around 3 hours after dawn (27 hours after the start of imaging), before wild type and YHB lines which peaked at around 4 hours after dawn (28 hours after the start of imaging). These differences between ‘square wave’ and ‘varied’ light conditions likely represent the contribution of metabolic dawn to circadian timing.

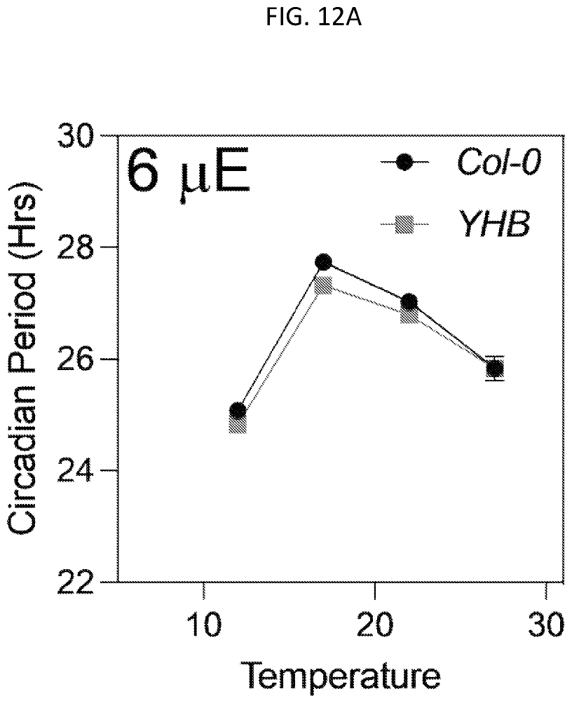

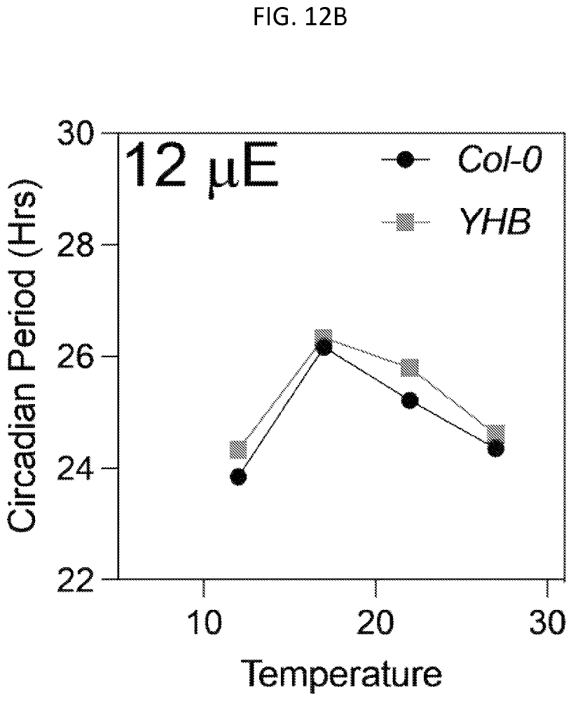

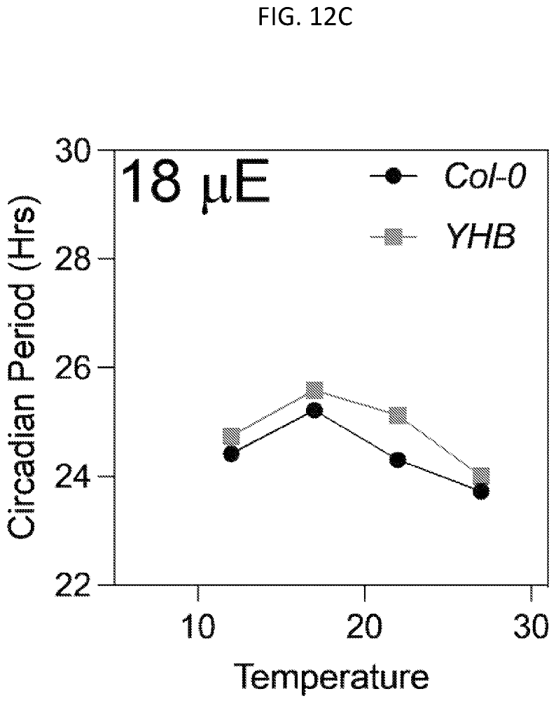

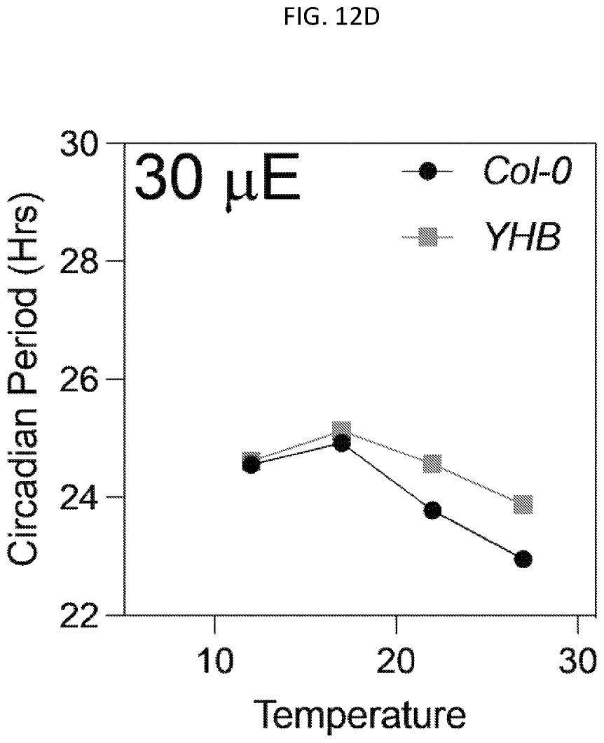

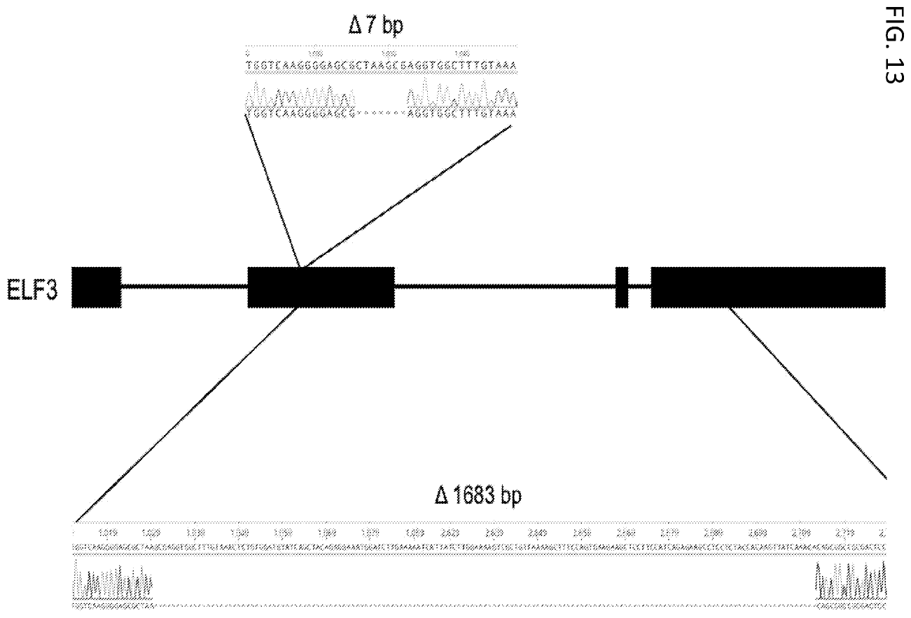

Since phyB signaling is responsive to temperature it was next assessed whether the plant circadian system was differentially sensitive to light at different ambient temperatures (FIGS. 11B-11E, 12A-D). It was found that the circadian system was unresponsive to light at 12° C., although responses to fluence rate were retained at 17° C., 22° C., and 27° C. It was also interesting to note that the YHB circadian phenotype under constant white light was dependent upon both light and temperature; the extension of circadian period in YHB lines was only observed at higher fluence rates and temperatures (FIGS. 11B-11E, 12A-D). These data are consistent with YHB seedlings being less responsive to light and temperature.

### YHBelf3 Flower Early Regardless of Daylength

The combination of YHB and elf3 alleles decouples the circadian system from photomorphogenesis, although YHBelf3 plants retain reduced daily patterns of gene expression when grown in light:dark cycles (FIGS. 3B-F, 4A-B, 5A-b, 6A-H, 7C-D). It was therefore of interest how the genetic manipulations described herein affected developmental traits in varied environmental conditions (FIGS. 9A-C, 10A-H, 11B-E, 12A-D). As anticipated, YHBelf3 seedlings retained a short hypocotyl phenotype regardless of the light condition utilized for growth (FIG. 3B-D, 4A-B, 10A). Similarly, YHBelf3 hypocotyl growth was unaffected by temperature, with no significant difference in hypocotyl growth between 12 and 27° C. (FIG. 3D).

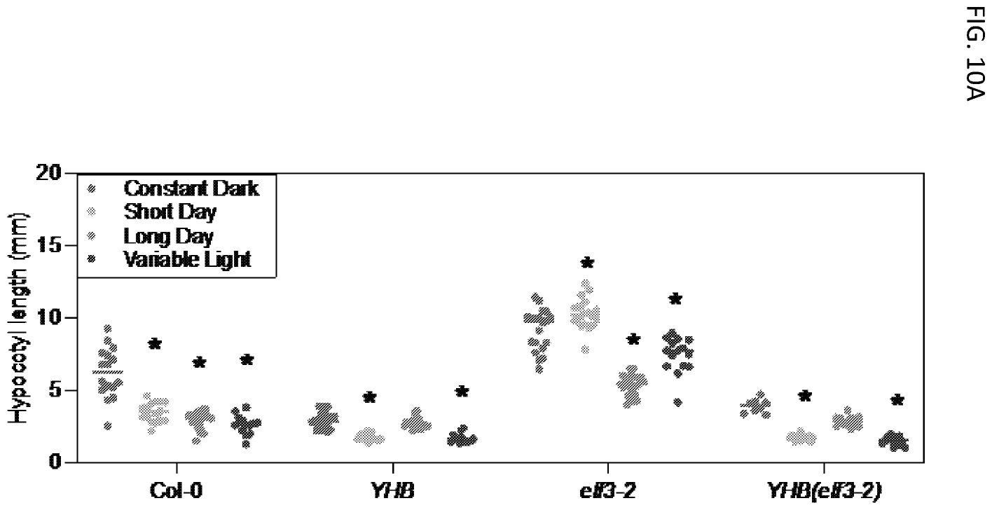

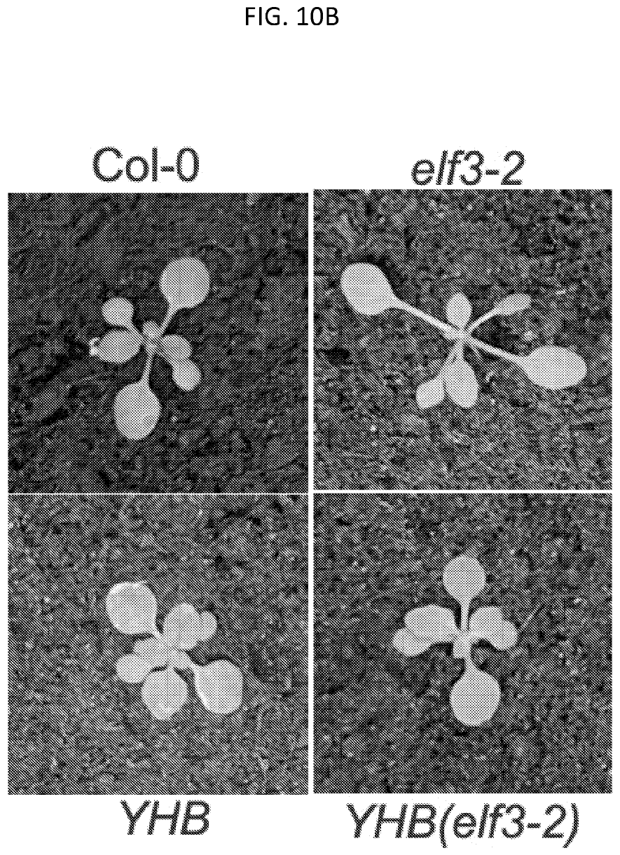

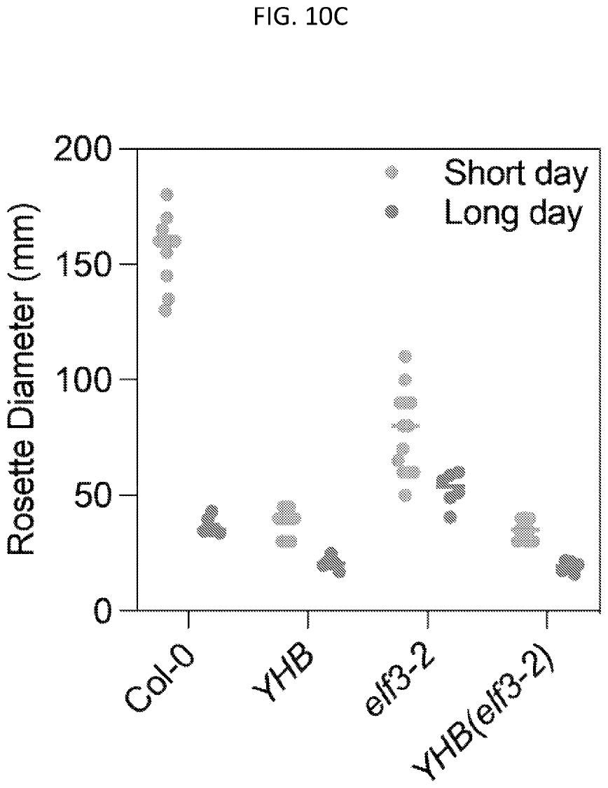

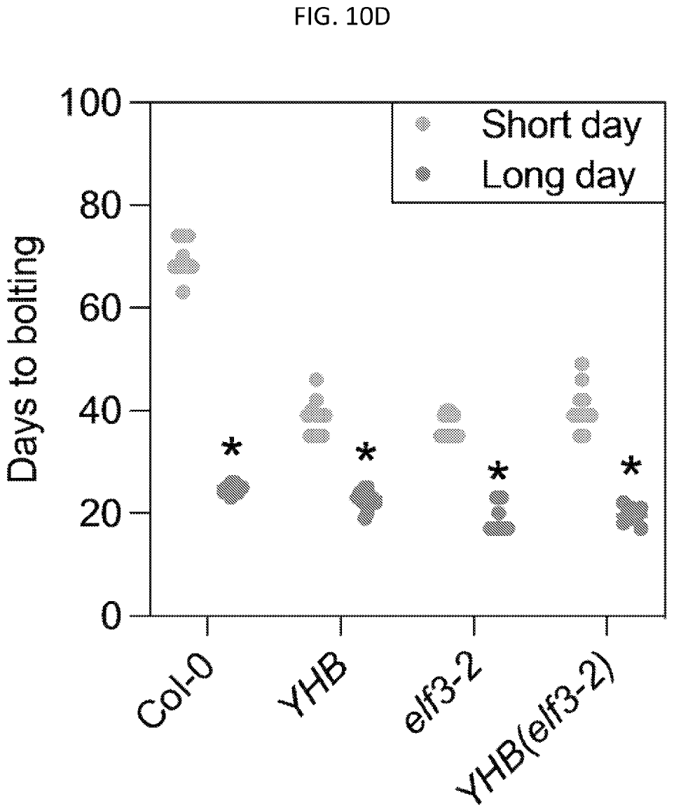

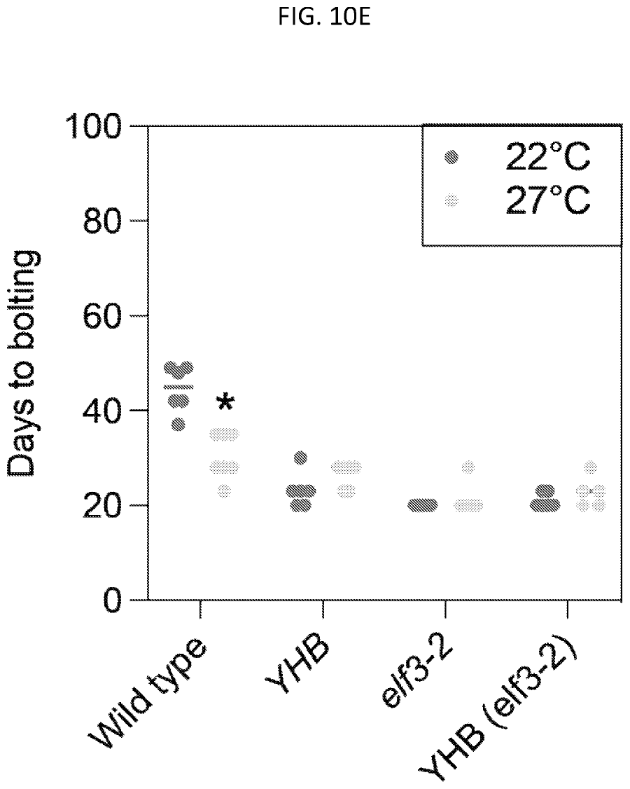

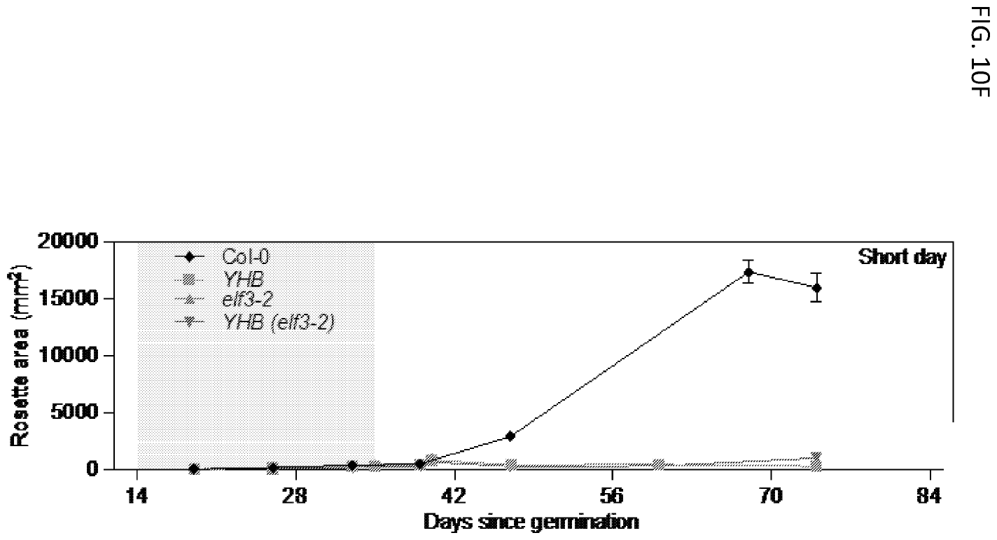

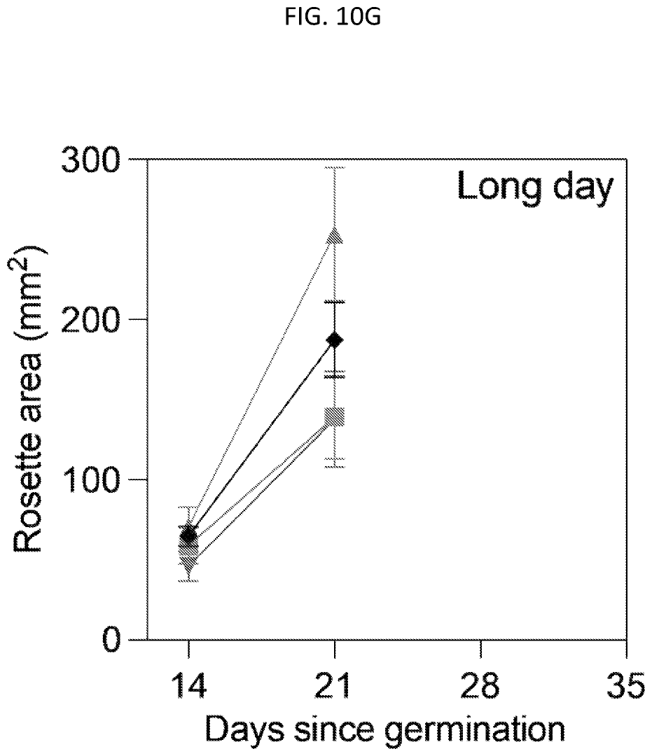

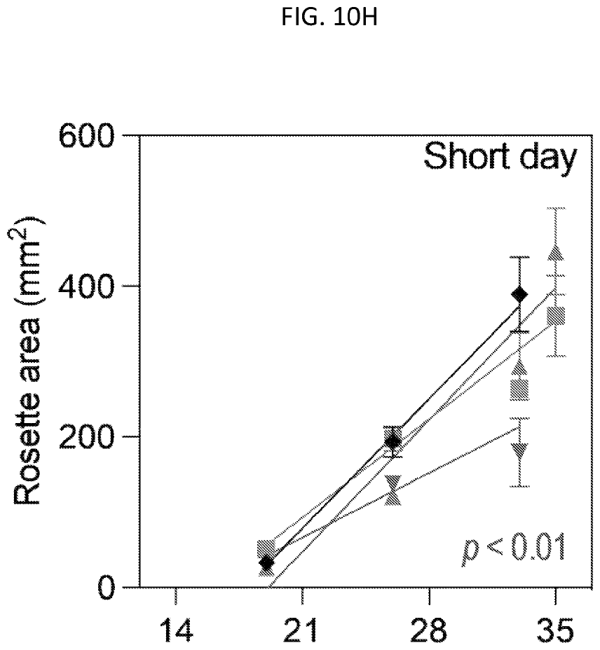

It was next examined how YHB and elf3 manipulations affected flowering time. YHB and elf3 plants flowered earlier than wild type in all conditions tested (under either long days or short days, as well as under elevated temperatures, FIGS. 3F, 10D, 10E). Differences in flowering time were especially apparent under short day conditions, where flowering was substantially delayed in wild-type plants. YHB plants flowered slightly later at 27° C. compared to 22° C. (FIG. 10E). YHBelf3 seedlings retained the early flowering phenotype of YHB and elf3 lines regardless of condition (FIGS. 3F, 10D, 10E). Despite these differences in flowering time, it was interesting to note that there was no difference in growth rate between genotypes in long-day conditions (FIG. 10G). In short-day conditions, YHBelf3 rosettes expand at a slower rate than the other genotypes (p<0.01, FIG. 10H), although wild type plants are not significantly bigger than the other genotypes until later in their lifecycle as they continue to grow due to the absence of flowering.

### Conclusions

The combination of YHB and elf3 alleles decouples the circadian system from plant photoperception and produces plants less responsive to environmental signals. YHBelf3 plants retain some molecular and developmental responses to light and temperature, although YHBelf3 plants retain consistently earlier flowering times without inhibiting vegetative growth.

### Example 4. Comparison of Plant Growth Responses

Wild-type pennycress is compared with elf3 gene mutant, YHB-transgene containing, and elf3 YHB transgenic pennycress lines for their growth in response to a variety of photoperiod conditions [e.g., short days (8 hours light/16 hours darkness), day-neutral (12 hours light/12 hours darkness), or long days (16 hours light/8 hours darkness) during juvenile and adult stages. The YHB and elf3 YHB transgenic pennycress lines show a reduced response to changing photoperiod conditions compared to the wild type, as it was demonstrated in Arabidopsis thaliana in the Examples herein above.

The above lines' growth responses to increased ambient temperature conditions (between 20-30 degrees Celsius) are also measured. The YHB and elf3 YHB transgenic pennycress lines also show a reduced elongation response to increases in temperature compared to wild type, as it was shown in Arabidopsis thaliana in the examples herein above. Combining the elf3 mutation with YHB shows reduced plasticity in growth to changing environment (light or temperature) conditions, producing a more uniform growth pattern regardless of day length or temperature.

### Example 5. Comparison of Plant Flowering Responses

Wild-type pennycress with elf3 gene mutant, YHB-transgene containing, and elf3 YHB transgenic pennycress lines are compared for their flowering in response to a variety of photoperiod conditions [e.g., short days (8 hours light/16 hours darkness), day-neutral (12 hours light/12 hours darkness), or long days (16 hours light/8 hours darkness). The elf3 mutant and elf3 YHB transgenic pennycress lines show a shorter time to flowering when compared to wild type or YHB transgenic lines, as it was shown in Arabidopsis thaliana in the examples herein above.

Time to flowering under increased ambient temperature (20-30 degrees Celsius) is also measured. As with above, the elf3 mutant and elf3 YHB transgenic pennycress lines show a reduced response to increasing temperature and flower at similar times regardless of temperature when compared to wild type, as it was shown in Arabidopsis thaliana in the examples herein above. Specifically combining the elf3 mutation with YHB shows reduced plasticity in flowering to changing environment (light or temperature) conditions, flowering at similar days after planting regardless of day length or temperature.

### Example 6. Comparison of Diurnal Metabolome

The clock arrhythmia produced by the elf3 mutation, combined with constant light signaling from the constitutive YHB transgene, reduces the daily oscillations in gene expression and the resulting proteins, decreasing the variation in the daily production of metabolites. The metabolomes of wild-type and elf3 YHB transgenic pennycress lines are measured over a 48-hour time course using mass spectrometry-based metabolome profiling. Daily rhythms of hundreds of metabolites in the elf3 YHB transgenic lines are flattened, with metabolites now clamped at constitutively high, intermediate, or low levels. The reduced dynamics of protein and metabolite production in the elf3 YHB transgenic lines makes it easier to predict and manipulate the production of high-value proteins or metabolites in plants.

What is claimed is:

1. A modified plant or part thereof, plant cell, or seed exhibiting reduced developmental plasticity, comprising:
a. a modification of an endogenous polynucleotide sequence encoding a component of the circadian clock; and
b. an exogenous polynucleotide sequence encoding an environmental signal sensor;
wherein the modification of the endogenous polynucleotide sequence encoding the component of the circadian clock modifies expression of the component of the circadian clock; and
wherein the modification of the endogenous polynucleotide encoding the environmental signal sensor modifies expression of the environmental signal sensor; and
wherein the modified expression of the component of the circadian clock and of the environmental signal sensor causes the plant to exhibit (i) reduced developmental plasticity in response to changes in environmental conditions during growth and (ii) modified circadian function.

2. The modified plant or part thereof, plant cell, or seed of claim 1, wherein the component of the circadian clock is a component of the evening complex (EC).

3. The modified plant or part thereof, plant cell, or seed of claim 1 or claim 2, wherein the component of the circadian clock is selected from EARLY FLOWERING 3 (ELF3), EARLY FLOWERING 4 (ELF4), LUX ARRHYTHMO (LUX), or any combination thereof.

4. The modified plant or part thereof, plant cell, or seed of any of the preceding claims, wherein the component of the circadian clock is an ELF3 protein.

5. The modified plant or part thereof, plant cell, or seed of claim 4, wherein the modification of a polynucleotide encoding the ELF3 protein is a loss-of-function mutation.

6. The modified plant or part thereof, plant cell, or seed of claim 5, wherein the plant is Arabidopsis thaliana (A. thaliana).

7. The modified plant or part thereof, plant cell, or seed of claim 6, wherein the ELF3 protein comprises an amino acid sequence comprising at least about 75% or more, at least about 85% or more, at least about 95% or more, or 100% sequence identity with an amino acid sequence of SEQ ID NO: 2.

8. The modified plant or part thereof, plant cell, or seed of claim 6, wherein the polynucleotide encoding ELF3 comprises a nucleic acid sequence comprising at least about 75% or more, at least about 85% or more, at least about 95% or more, or 100% sequence identity with a nucleic acid sequence of SEQ ID NO: 1 and an ELF3 loss-of-function mutation comprises about 75% or more, at least about 85% or more, at least about 95% or more, or 100% sequence identity with a nucleic acid sequence of SEQ ID NO: 3 (elf3-1), SEQ ID NO: 4 (elf3-2), or a combination thereof.

9. The modified plant or part thereof, plant cell, or seed of claim 5, wherein the modified plant is Thlaspi arvense (T. arvense).

10. The modified plant or part thereof, plant cell, or seed of claim 9, wherein the ELF3 protein comprises an amino acid sequence comprising at least about 75% or more, at least about 85% or more, at least about 95% or more, or 100% sequence identity with an amino acid sequence of SEQ ID NO: 21.

11. The modified plant or part thereof, plant cell, or seed of claim 10, wherein the polynucleotide encoding the ELF3 protein comprises a nucleic acid sequence comprising at least about 75% or more, at least about 85% or more, at least about 95% or more, or 100% sequence identity with a nucleic acid sequence of SEQ ID NO: 20.

12. The modified plant or part thereof, plant cell, or seed of claim 10, wherein the modification of the polynucleotide encoding the ELF3 protein comprises a deletion of a nucleic acid segment in the polynucleotide encoding the ELF3 protein, wherein the nucleic acid segment comprises about 75% or more, at least about 85% or more, at least about 95% or more, or 100% sequence identity with a nucleic acid sequence of SEQ ID NO: 22, a sequence comprising the AGCGCTA nucleic acid sequence, or a combination thereof.

13. The modified plant or part thereof, plant cell, or seed of claim 10, wherein the modification of the polynucleotide encoding the ELF3 protein comprises a modification introduced by a programmable nucleic acid modification system comprising a CRISPR/Cas nuclease system, wherein the CRISPR/Cas nuclease system comprises a CAS9 protein (dCAS9) and a guide RNA (gRNA).

14. The modified plant or part thereof, plant cell, or seed of claim 13, wherein the Cas9 protein is encoded by a nucleic acid sequence comprising at least about 75% or more, at least about 85% or more, at least about 95% or more, or 100% sequence identity with a nucleic acid sequence of SEQ ID NO: 29 and the gRNA comprises a nucleic acid sequence of SEQ ID NO: 30, SEQ ID NO: 31, or a combination thereof.

15. The modified plant or part thereof, plant cell, or seed of claim 13, wherein an expression construct expressing the Cas9 protein comprises a nucleic acid sequence comprising at least about 75% or more, at least about 85% or more, at least about 95% or more, or 100% sequence identity with a nucleic acid sequence starting at base number 1552 to base number 7881 of SEQ ID NO: 32.

16. The modified plant or part thereof, plant cell, or seed of claim 5, wherein the plant is Basil.

17. The modified plant or part thereof, plant cell, or seed of claim 16, wherein the polynucleotide encoding ELF3 comprises a nucleic acid sequence comprising at least about 75% or more, at least about 85% or more, at least about 95% or more, or 100% sequence identity with a nucleic acid sequence of SEQ ID NO: 13.

18. The modified plant or part thereof, plant cell, or seed of claim 17, wherein the modification of the polynucleotide encoding the ELF3 protein comprises a modification introduced by a programmable nucleic acid modification system comprising a CRISPR/Cas nuclease system, wherein the CRISPR/Cas nuclease system comprises a CAS9 protein (dCAS9) and a guide RNA (gRNA).

19. The modified plant or part thereof, plant cell, or seed of claim 18, wherein the Cas9 protein is encoded by a nucleic acid sequence comprising at least about 75% or more, at least about 85% or more, at least about 95% or more, or 100% sequence identity with a nucleic acid sequence of SEQ ID NO: 29 and the gRNA comprises a nucleic acid sequence of SEQ ID NOs: 33-36, or any combination thereof.

20. The modified plant or part thereof, plant cell, or seed of claim 19, wherein an expression construct expressing the Cas9 protein comprises a nucleic acid sequence comprising at least about 75% or more, at least about 85% or more, at least about 95% or more, or 100% sequence identity with a nucleic acid sequence starting at base number 1552 to base number 7881 of SEQ ID NO: 32.

21. The modified plant or part thereof, plant cell, or seed of any of the preceding claims, wherein the environmental signal comprises photoperiod, light intensity and quality, temperature, chemicals, gravity, moisture, biotic or abiotic stress, oxygen and carbon dioxide concentrations, carbohydrate availability, or any combination thereof.

22. The modified plant or part thereof, plant cell, or seed of any of the preceding claims, wherein the environmental signal sensor is a photoreceptor, temperature sensor, CO₂ sensor, O₂ sensor, ethylene sensor, gravitropic sensor, or any combination thereof.

23. The modified plant or part thereof, plant cell, or seed of any of the preceding claims, wherein the environmental signal sensor is a photoreceptor.

24. The modified plant or part thereof, plant cell, or seed of claim 23, wherein the photoreceptor is a phytochrome; a cryptochrome, a phototropin, an F-box containing Flavin binding proteins; UVR8, or any combination thereof.

25. The modified plant or part thereof, plant cell, or seed of claim 23 or 24, wherein the photoreceptor is a phytochrome photoreceptor.

26. The modified plant or part thereof, plant cell, or seed of any of the preceding claims, wherein the polynucleotide encoding an environmental signal sensor encodes a phytochrome B photoreceptor.

27. The modified plant or part thereof, plant cell, or seed of claim 26, wherein the phytochrome B photoreceptor comprises an amino acid sequence comprising at least about 75% or more, at least about 85% or more, at least about 95% or more, or 100% sequence identity with an amino acid sequence of SEQ ID NO: 6 or SEQ ID NO: 28.

28. The modified plant or part thereof, plant cell, or seed of claim 27, wherein the polynucleotide encoding the phytochrome B photoreceptor comprises least about 75% or more, at least about 85% or more, at least about 95% or more, or 100% sequence identity with a nucleic acid sequence of SEQ ID NO: 5 or SEQ ID NO: 28.

29. The modified plant or part thereof, plant cell, or seed of claim 26, wherein the phytochrome B photoreceptor is overexpressed, constitutively expressed, or constitutively active.

30. The modified plant or part thereof, plant cell, or seed of claim 29, wherein the modification of a polynucleotide encoding a phyB photoreceptor encodes a constitutively active phyB photoreceptor.

31. The modified plant or part thereof, plant cell, or seed of claim 30, wherein the phytochrome B photoreceptor comprises an amino acid sequence comprising at least about 75% or more, at least about 85% or more, at least about 95% or more, or 100% sequence identity with an amino acid sequence of SEQ ID NO: 8 SEQ ID NO: 12, SEQ ID NO: 26, or any combination thereof.

32. The modified plant or part thereof, plant cell, or seed of claim 30, wherein the polynucleotide encoding the phytochrome B photoreceptor comprises least about 75% or more, at least about 85% or more, at least about 95% or more, or 100% sequence identity with a nucleic acid sequence of SEQ ID NO: 7, SEQ ID NO: 11, SEQ ID NO: 25, or any combination thereof.

33. The modified plant or part thereof, plant cell, or seed of claim 30, wherein the modified plant is Arabidopsis thaliana (A. thaliana).

34. The modified plant or part thereof, plant cell, or seed of claim 33, wherein the modified A. thaliana plant comprises a genetic modification of a polynucleotide encoding PhyB, wherein the polynucleotide comprising the genetic modification encodes a constitutively active PhyB photoreceptor.

35. The modified plant or part thereof, plant cell, or seed of claim 34, wherein constitutively active PhyB photoreceptor comprises an amino acid sequence comprising at least about 75% or more, at least about 85% or more, at least about 95% or more, or 100% sequence identity with an amino acid sequence of SEQ ID NO: 8 SEQ ID NO: 12, SEQ ID NO: 26, or any combination thereof.

36. The modified plant or part thereof, plant cell, or seed of claim 34, wherein constitutively active PhyB photoreceptor is encoded by a nucleic acid sequence comprising at least about 75% or more, at least about 85% or more, at least about 95% or more, or 100% sequence identity with an amino acid sequence of SEQ ID NO: 7, SEQ ID NO: 11, SEQ ID NO: 25, or any combination thereof.

37. The modified plant or part thereof, plant cell, or seed of claim 34, wherein constitutively active PhyB photoreceptor comprises an amino acid sequence comprising at least about 75% or more, at least about 85% or more, at least about 95% or more, or 100% sequence identity with an amino acid sequence of SEQ ID NO: 8, SEQ ID NO: 12, or both.

38. The modified plant or part thereof, plant cell, or seed of claim 34, wherein the constitutively active PhyB photoreceptor is encoded by a nucleic acid sequence comprising at least about 75% or more, at least about 85% or more, at least about 95% or more, or 100% sequence identity with an amino acid sequence of SEQ ID NO: 7, SEQ ID NO: 11, or both.

39. The modified plant or part thereof, plant cell, or seed of claim 30, wherein the modified plant is Thlaspi arvensis (T. arvensis).

40. The modified plant or part thereof, plant cell, or seed of claim 39, wherein the modified T. arvensis plant comprises a genetic modification of a polynucleotide encoding PhyB, wherein the polynucleotide comprising the genetic modification encodes a constitutively active PhyB photoreceptor.

41. The modified plant or part thereof, plant cell, or seed of claim 40, wherein the constitutively active PhyB photoreceptor comprises an amino acid sequence comprising at least about 75% or more, at least about 85% or more, at least about 95% or more, or 100% sequence identity with an amino acid sequence of SEQ ID NO: 8 SEQ ID NO: 12, SEQ ID NO: 26, or any combination thereof.

42. The modified plant or part thereof, plant cell, or seed of claim 40, wherein the constitutively active PhyB photoreceptor is encoded by a nucleic acid sequence comprising at least about 75% or more, at least about 85% or more, at least about 95% or more, or 100% sequence identity with an amino acid sequence of SEQ ID NO: 7, SEQ ID NO: 11, SEQ ID NO: 25, or SEQ ID NO: 27.

43. The modified plant or part thereof, plant cell, or seed of claim 40, wherein the constitutively active PhyB photoreceptor comprises an amino acid sequence comprising at least about 75% or more, at least about 85% or more, at least about 95% or more, or 100% sequence identity with an amino acid sequence of SEQ ID NO: 26.

44. The modified plant or part thereof, plant cell, or seed of claim 40, wherein the constitutively active PhyB photoreceptor is encoded by a nucleic acid sequence comprising at least about 75% or more, at least about 85% or more, at least about 95% or more, or 100% sequence identity with an amino acid sequence of SEQ ID NO: 25.

45. The modified plant or part thereof, plant cell, or seed of any of the preceding claims, comprising:
a. a modification of a polynucleotide encoding an ELF3 protein wherein the polynucleotide encoding the ELF3 protein comprises an ELF3 loss-of-function mutation, and
b. a modification of a polynucleotide encoding a phyB photoreceptor wherein the polynucleotide encodes a constitutively active PhyB photoreceptor.

46. The modified plant or part thereof, plant cell, or seed of any of the preceding claims, wherein the modified plant comprises constitutively inactivated temperature input to the circadian clock and a constitutively activated light input to the circadian clock.

47. The modified plant or part thereof, plant cell, or seed of any of the preceding claims, wherein the modified plant is not etiolated, flowers earlier and consistently regardless of changes in day length and has a reduced cellular elongation response to increasing temperature thereby reducing listing in the field, and any combination thereof.

48. The modified plant or part thereof, plant cell, or seed of claim 1, wherein the modified plant is plant is A. thaliana comprising:
a. a polynucleotide encoding an ELF3 protein wherein the polynucleotide encoding the ELF3 protein comprises an ELF3 loss-of-function mutation, wherein the polynucleotide comprises a nucleic acid sequence comprising at least about 75% or more, at least about 85% or more, at least about 95% or more, or 100% sequence identity with an amino acid sequence of SEQ ID NO: 3 (elf3-1), SEQ ID NO: 4 (elf3-2), or a combination thereof; and
b. a polynucleotide encoding a constitutively active PhyB photoreceptor, wherein the polynucleotide comprises a nucleic acid sequence comprising at least about 75% or more, at least about 85% or more, at least about 95% or more, or 100% sequence identity with an amino acid sequence of SEQ ID NO: 7, SEQ ID NO: 11, or a combination thereof.

49. The modified plant or part thereof, plant cell, or seed of claim 1, wherein the modified plant is plant is T. arvensis comprising:
a. a polynucleotide encoding an ELF3 protein wherein the polynucleotide encoding the ELF3 protein comprises an ELF3 loss-of-function mutation, wherein the polynucleotide comprises a nucleic acid sequence comprising at least about 75% or more, at least about 85% or more, at least about 95% or more, or 100% sequence identity with an amino acid sequence of SEQ ID NO: 22, a sequence comprising the AGCGCTA nucleic acid sequence, or a combination thereof; and
b. a polynucleotide encoding a constitutively active PhyB photoreceptor, wherein the polynucleotide comprises a nucleic acid sequence comprising at least about 75% or more, at least about 85% or more, at least about 95% or more, or 100% sequence identity with an amino acid sequence of SEQ ID NO: 25.

50. A system for modifying a component of the circadian clock, an environmental signal sensor, or both in a plant or part thereof, plant cell, or seed, the system comprising a nucleic acid construct comprising at least one or both of:
a. a programmable nucleic acid modification system comprising a targeting nucleic acid sequence targeting a nucleic acid sequence in a polynucleotide sequence encoding a component of the circadian clock; and
b. an expression construct comprising a promoter operably linked to a polynucleotide encoding a polynucleotide encoding an environmental signal sensor;
wherein (a) introduces a loss of function mutation into the polynucleotide sequence encoding the component of the circadian clock, and
wherein (b) increases expression of the environmental signal sensor, thereby causing the plant to exhibit reduced developmental plasticity in response to changes in environmental conditions during growth and modified circadian function.

51. The system of claim 50, wherein the programmable nucleic acid modification system introduces a loss of function mutation into a polynucleotide encoding an ELF3 protein.

52. The system of claim 51, wherein the programmable nucleic acid modification system comprises a CRISPR/Cas nuclease system comprising a CAS9 protein (dCAS9) and a guide RNA (gRNA).

53. The system of claim 52, wherein the plant is Thlaspi arvense.

54. The system of claim 53, wherein the Cas9 protein is encoded by a nucleic acid sequence comprising at least about 75% or more, at least about 85% or more, at least about 95% or more, or 100% sequence identity with a nucleic acid sequence of SEQ ID NO: 29 and the gRNA comprises a nucleic acid sequence of SEQ ID NO: 30, SEQ ID NO: 31, or both.

55. The system of claim 52, wherein the plant is Basil.

56. The system of claim 55, wherein the Cas9 protein is encoded by a nucleic acid sequence comprising at least about 75% or more, at least about 85% or more, at least about 95% or more, or 100% sequence identity with a nucleic acid sequence of SEQ ID NO: 29 and the gRNA comprises a nucleic acid sequence of SEQ ID NOs: 33-36, or any combination thereof.

57. The system of claim 50, wherein the expression construct comprises a promoter operably linked to a polynucleotide encoding a polynucleotide encoding a PhyB photoreceptor, wherein the expression construct increases expression of PhyB.

58. The system of claim 57, wherein the expression construct comprising a promoter operably linked to a polynucleotide encoding an environmental signal sensor comprises a 35S promoter operably linked to a polynucleotide encoding the PhyB photoreceptor and wherein the construct constitutively expresses the PhyB photoreceptor.

59. The system of claim 57, wherein the PhyB photoreceptor is constitutively active.

60. The system of claim 59, wherein the expression construct comprises a nucleic acid sequence comprising about 75% or more, at least about 85% or more, at least about 95% or more, or 100% sequence identity with SEQ ID NO: 24.

61. The system of claim 60, wherein the expression construct comprises a nucleic acid sequence comprising a nucleic acid sequence comprising about 75% or more, at least about 85% or more, at least about 95% or more, or 100% of a nucleic acid sequence starting at base 8148 to base 12020 of SEQ ID NO: 19.

62. One or more expression constructs for modifying a plant or part thereof, plant cell, or seed to exhibit reduced developmental plasticity, the one or more expression constructs comprising at least one or both of:
a. an expression construct for modifying the expression of a component of the circadian clock; and
b. an expression construct for modifying the expression of an environmental signal sensor.

63. The one or more expression constructs of claim 62, wherein (a) comprises a promoter operably linked to a polynucleotide encoding a programmable nucleic acid modification system targeted to a nucleic acid sequence in a polynucleotide encoding a component of the circadian clock.

64. The one or more expression constructs of claim 63, wherein the programmable nucleic acid modification system comprises a CRISPR/Cas nuclease system and wherein the CRISPR/Cas nuclease system comprises a CAS9 protein (dCAS9) and a guide RNA (gRNA).

65. The one or more expression constructs of claim 64, wherein an expression construct expressing the Cas9 protein comprises a nucleic acid sequence comprising at least about 75% or more, at least about 85% or more, at least about 95% or more, or 100% sequence identity with a nucleic acid sequence starting at base number 1552 to base number 7881 of SEQ ID NO: 32.

66. The one or more expression constructs of claim 64, wherein the plant is Thlaspi arvense.

67. The one or more expression constructs of claim 66, wherein the polynucleotide encoding a component of the circadian clock encodes ELF3 and wherein the polynucleotide encoding ELF3 comprises a nucleic acid sequence comprising at least about 75% or more, at least about 85% or more, at least about 95% or more, or 100% sequence identity with a nucleic acid sequence of SEQ ID NO: 20 and wherein the gRNA comprises a nucleic acid sequence of SEQ ID NO: 30, SEQ ID NO: 31, or both.

68. The one or more expression constructs of claim 66, wherein the expression construct comprises a nucleic acid sequence comprising about 75% or more, at least about 85% or more, at least about 95% or more, or 100% sequence identity with a nucleic acid sequence starting at base 254 to base 1287 of SEQ ID NO: 32.

69. The one or more expression constructs of claim 64, wherein the plant is Basil.

70. The one or more expression constructs of claim 69, wherein the polynucleotide encoding a component of the circadian clock encodes ELF3 and wherein the polynucleotide encoding ELF3 comprises a nucleic acid sequence comprising at least about 75% or more, at least about 85% or more, at least about 95% or more, or 100% sequence identity with a nucleic acid sequence of SEQ ID NO: 13 and wherein the gRNA comprises a nucleic acid sequence of SEQ ID NOs: 33-36, or any combination thereof.

71. The one or more expression constructs of claim 70, wherein the expression construct expressing the gRNA of SEQ ID NOs: 33-36 comprises a nucleic acid sequence comprising about 75% or more, at least about 85% or more, at least about 95% or more, or 100% sequence identity with the gRNA expression construct of SEQ ID NO: 37.

72. The one or more expression constructs of claim 70, wherein an expression construct expressing the gRNA of SEQ ID NOs: 33-36 comprises about 75% or more, at least about 85% or more, at least about 95% or more, or 100% sequence identity with SEQ ID NO: 23.

73. The one or more expression constructs of claim 70, wherein an expression construct expressing the gRNA of SEQ ID NOs: 33-36 comprises about 75% or more, at least about 85% or more, at least about 95% or more, or 100% of a nucleic acid sequence starting at base 695 to base 2799 of SEQ ID NO: 37.

74. The one or more expression constructs of claim 62, wherein the expression construct comprising a promoter operably linked to a polynucleotide encoding an environmental signal sensor comprises a constitutive expression promoter operably linked to a polynucleotide encoding a PhyB photoreceptor.

75. The one or more expression constructs of claim 74, wherein the expression construct comprising a promoter operably linked to a polynucleotide encoding an environmental signal sensor comprises a 35S promoter operably linked to a polynucleotide encoding a constitutively active PhyB photoreceptor.

76. The one or more expression constructs of claim 75, wherein the constitutively active PhyB photoreceptor is encoded by a nucleic acid sequence comprising about 75% or more, at least about 85% or more, at least about 95% or more, or 100% sequence identity with a nucleic acid sequence of SEQ ID NO: 7, SEQ ID NO: 11, or both.

77. The one or more expression constructs of claim 75, wherein the expression construct comprises a nucleic acid sequence comprising about 75% or more, at least about 85% or more, at least about 95% or more, or 100% sequence identity with SEQ ID NO: 24.

78. The one or more expression constructs of claim 75, wherein the expression construct comprising a nucleic acid sequence comprising about 75% or more, at least about 85% or more, at least about 95% or more, or 100% of a nucleic acid sequence starting at base 8148 to base 12020 of SEQ ID NO: 19.

79. A modified plant or part thereof, plant cell, or seed thereof comprising one or more expression constructs of claims 62-78.

80. A method of improving performance of a plant or part thereof, plant cell, or seed grown under intensive, precision outdoor farming conditions, Total Controlled Environment Agriculture (TCEA), or vertical farming, the method comprising:
a. obtaining or having obtained a modified plant of any one of claims 1-49; and
b. cultivating the plant under intensive, precision outdoor farming conditions, TCEA, or vertical farming.

81. A method of reducing developmental plasticity of a plant or part thereof, plant cell, or seed, the method comprising generating a modified plant of any one of claims 1-49.

82. The method of claim 81, wherein the plant is resistant to changes in growth conditions of the plant.

83. The method of claim 82, wherein the growth conditions comprise daylength and temperature.

84. A method of reducing a plastic response to competition among a co-cultivated group of modified plants or parts thereof, plant cells, or seeds, the method comprising, co-cultivating a group of plants of any of claims 1-49.

85. A method of stabilizing production of a metabolite, nucleic acid, or protein in a plant or part thereof, plant cell, or seed, the method comprising:
a. obtaining or having obtained a modified plant or part thereof, plant cell, or seed of any of claims 1-49;
b. cultivating the plant for a time sufficient to generate the metabolite, nucleic acid, or protein; and
c. harvesting the metabolite, nucleic acid, or protein from the plant or part thereof, plant cell, or seed.

86. The method of claim 85, wherein the plant or part thereof, plant cell, or seed is grown under intensive, precision outdoor farming conditions, TCEA, or vertical farming.

87. The method of claim 85, wherein stabilizing production of a metabolite, nucleic acid, or protein comprises equal production of the metabolite, nucleic acid, or protein irrespective of light period, temperature variation or both.

88. A kit for modifying a plant or part thereof, plant cell, or seed exhibiting reduced developmental plasticity, the kit comprising:
a. one or more modified plants or parts thereof, plant cells, or seeds of any one of claims 1-49;
b. one or more expression constructs of any one of claims 62-78:
c. one or more modified plants, plant cells, or parts thereof of claim 79; or
d. any combination of (a) to (c).
